(19) United States
(12) Patent Application Publication
(10) Pub. No.: US 2015/0119373 A1
(43) Pub. Date: Apr. 30, 2015

(54) NOVEL BETULINIC ACID DERIVATIVES AS HIV INHIBITORS

(71) Applicant: HETERO RESEARCH FOUNDATION, Balanagar, Hyderabad, Andhra Pradesh (IN)

(72) Inventors: Bandi Parthasaradhi Reddy, Hyderabad (IN); Vedula Manohar Sharma, Hyderabad (IN); Kura Rathnakar Reddy, Hyderabad (IN); Mamnoor Prem Kumar, Hyderabad (IN); Kasireddy Bhaskar Reddy, Hyderabad (IN); Mogili Narsingam, Hyderabad (IN); Mukkera Venkati, Hyderabad (IN); Lanka VL Subrahmanyam, Hyderabad (IN); Ravi Mallikarjun Reddy, Hyderabad (IN)

(21) Appl. No.: 14/395,668

(22) PCT Filed: Apr. 19, 2013
(86) PCT No.: PCT/IB13/53120

§ 371 Date: Oct. 20, 2014

(30) Foreign Application Priority Data
Apr. 24, 2012 (IN) 1613/CHE/2012
Dec. 31, 2012 (IN) 5528/CHE/2012

(51) Int. Cl. C07J 53/00

(52) U.S. Cl. CPC C07J 53/002
USPC 514/176; 552/510; 514/169; 546/195; 544/380; 548/143; 544/154; 544/121; 544/360; 544/130

(57) ABSTRACT

The invention relates to novel betulinic acid derivatives and related compounds, and pharmaceutical compositions useful for therapeutic treatment of viral diseases and particularly HIV mediated diseases.

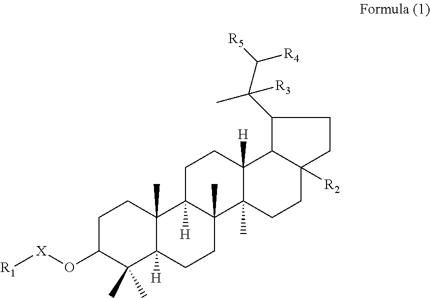

### CROSS REFERENCE TO RELATED APPLICATIONS

This application is a U.S. national stage of international application No. PCT/IB2013053120 filed on 19 Apr. 2013 which claims the benefit of Indian Provisional Patent Application Nos. 1613CHE2012 filed on 24 Apr. 2012 and 5528CHE2012 filed on 31 Dec. 2012; all of which are incorporated herein by reference in their entirety.

### FIELD OF THE INVENTION

The present invention relates to novel betulinic acid derivatives and related compounds, compositions useful for therapeutic treatment of viral diseases and particularly HIV mediated diseases.

### BACKGROUND OF THE INVENTION

The Human Immunodeficiency Virus (HIV) has now been established as the causative agent of the Acquired Immunodeficiency Syndrome (AIDS) for over 20 years (Science 1983, 220,868-871; N. Eng. J. Med. 1984, 311,1292-1297). AIDS is characterized by the destruction of the immune system, particularly of CD4+T-cells. HIV is a retrovirus, and the HIV life cycle encompasses several crucial steps, starting from the attachment of the virus to the host cell membrane and finishing with the release of progeny virons from the cell.

The natural compound betulinic acid, isolated from Syzygium clavifolium and several other plant species was found to possess anti-HIV activity. Chemical modifications were undertaken by several research groups in an attempt to identify potent anti-HIV agents by making semi-synthetic analogs of betulinic acid, leading to the discovery of bevirimat as a compound with a novel mechanism of action (J. Nat. Prod. 199457(2):243-7; J. Med. Chem. 1996, 39(5),1016). Further studies shown that bevirimat acts by disrupting Gag processing (Proc. Natl. Acad. Sci. USA 2003, 100(23):13555-60; Antimicrob. Agents. Chemother. 2001, 45(4),1225-30; J. Virol. 2004, 78(2): 922-9; J. Biol. Chem. 2005, 280(51):42149-55; J. Virol. 2006, 80(12): 5716-22) and to be a first-in-class maturation inhibitor with a potent activity against HIV-1. Bevirimaet went upto phase 2 clinical trials, in clinic despite optimal plasma concentrations, not all patients given bevirimat have a robust viral load reduction. It was reported that non-respondent patients had more frequent base line Gag polymorphisms near the capsid SP-1 cleavage site than responders. (HIV gag polymorphism determines treatment response to bevirimat. XVII inter national HIV drug resistance work shop Jun. 10-14, 2008, Stiges, Spain).

Encouraged by these developments, medicinal chemists started exploring betulinic acid derivatives and related compounds intensively for their therapeutic activities. For example, WO 2011153319, WO 2011153315, WO 2011007230, WO 2009082819, and WO 2009100532 disclosed novel 17 β lupine derivatives as anti-HIV agents in an attempt to overcome gag poly morphism issues mentioned above. The patent publication WO 2008057420 describes extended triterpene derivatives as antiretroviral agents; WO 2007141391 describes Betulin derived compounds useful as antiprotozoal agents; WO 2007141390 describes preparation of betulin derived compounds as antiviral agents; WO 2008127364 describes preparation of betulinic acid derivatives for use in antiviral and anticancer pharmaceutical compositions; US 20080207573 describes preparation of triterpene derivatives for therapeutic use in the treatment of viral infections; WO 2007141389 describes preparation of betulin derived compounds as antibacterial agents; US 20040204389 describes anti-HIV agents with dual sites of action; WO 2007002411 describes antiviral compounds; CN 1861627 describes antitumor agents; WO 2006053255 describes novel betulin derivatives, preparation and use thereof; WO 2009082818 describes novel C-21 keto lupine derivatives preparation and use thereof; and WO 2006105356 describes methods of manufacturing bioactive 3-esters of betulinic aldehyde and betulinic acid.

Some more references disclose betulinic acid related compounds. For example, WO 2007141383 describes betulin derivatives as antifeedants for plant pests; U.S. Pat. No. 6,670,345 describes use of betulinic acid and its derivatives for inhibiting cancer growth and process for the manufacture of betulinic acid; WO 2002091858 describes anxiolytic marcgraviaceae compositions containing betulinic acid, betulinic acid derivatives, and methods of preparation and use; WO 2000046235 describes preparation of novel betulinic acid derivatives for use as cancer growth inhibitors; WO 2007141392 describes cosmetic and pharmaceutical compositions comprising betulonic acid and betulin derivatives; and Pharmaceutical chemistry journal, 2002, 36(9), 29-32 describes synthesis and anti-inflammatory activity of new acylated betulin derivatives.

Given the fact of the world wide epidemic level of AIDS, there is a strong continued need for new effective drugs for treatment of HIV infected patentis, disease conditions and/or disorders mediated by HIV by discovering new compounds with novel structures and/or mechanism of action(s).

### SUMMARY OF THE INVENTION

The present invention relates to the compounds of the formula (1):

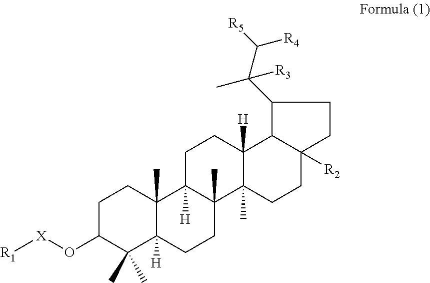

wherein,

X can be a bond, or —C(O)—;

R₁ can be H, substituted or unsubstituted alkyl,

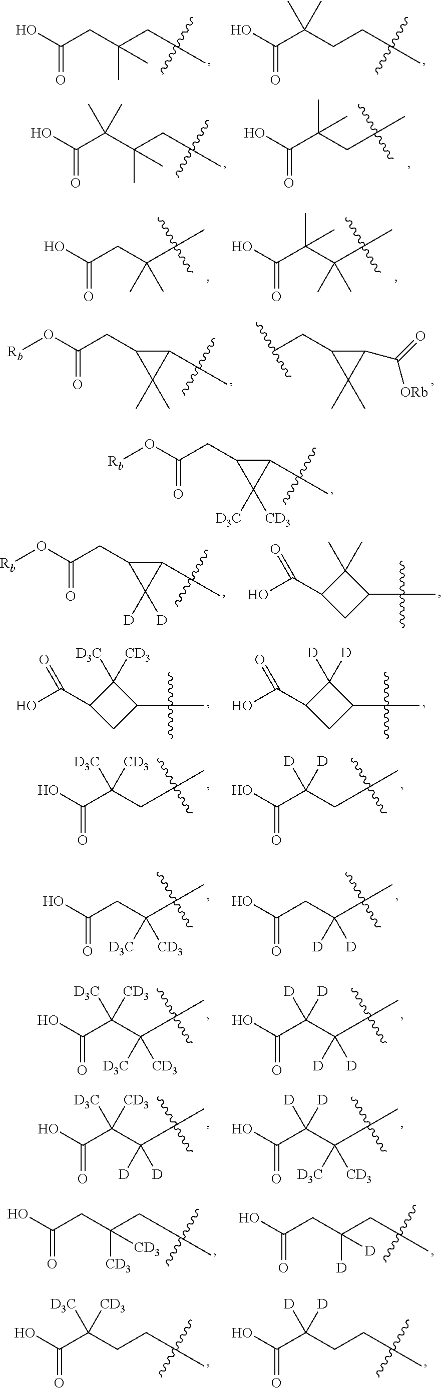

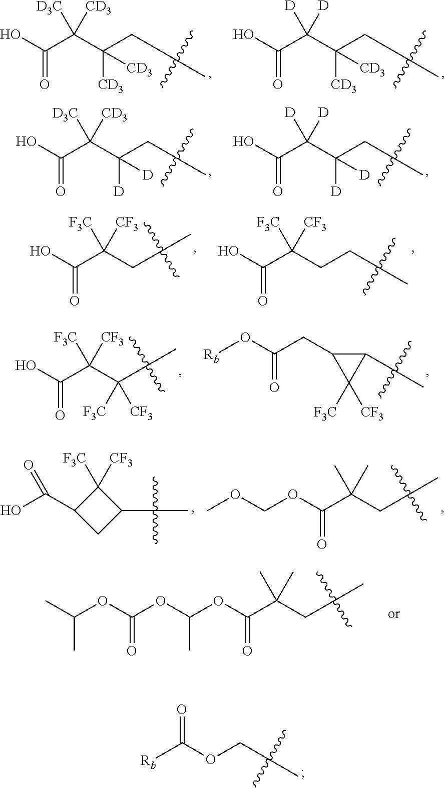

R₂ can be Het¹,

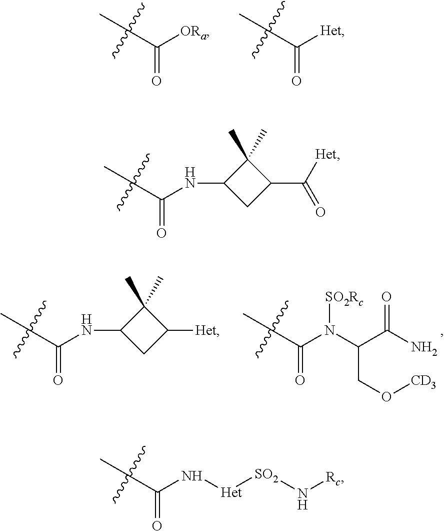

and preferably Het¹ and Het can be independently selected from oxazole, oxadiazole, triazole, isoxazole, pyrazole, pyridine, piperidine, Morpholine, pyrazine, or piperazine and preferably Het¹ and Het can be substituted by R′.

R₃ and R₄ can be independently selected from H, OH, substituted or unsubstituted alkyl, substituted or unsubstituted alkoxy, substituted or unsubstituted alkoxylalkoxy, or substituted or unsubstituted aminoacids and preferably amino acids are substituted by substituted or unsubstituted alkyl, phosphoric acid, or phosphorus prodrugs or R₃ and R₄ can be together with their adjacent carbons form a bond or R₃ and R₄ can be together with their adjacent carbons form cyclopropl or epoxide;

R₅ can be H, D, CD₃, CH₂CD₃, CH(CD₃)₂, CO₂R_{d}, or substituted or unsubstituted alkyl;

Rₐ, R_{b}, R_{c} and R_{d} can be independently selected from H, or substituted or unsubstituted alkyl;

each R′ can be independently selected from H, CN, D, CD₃, CH₂CD₃, CH(CD₃)₂, CO₂H, CO₂-alkyl, C(O)-alkyl, OC(O)-alkyl, C(O)NH-alkyl, substituted or unsubstituted alkyl, substituted or unsubstituted alkoxy, substituted or unsubstituted phosphinate, substituted or unsubstituted oxime, substituted or unsubstituted amino, substituted or unsubstituted aryl, substituted or unsubstituted cycloalkyl, or substituted or unsubstituted heterocyclyl.

Pharmaceutically acceptable salts of the compounds of the formula (1) are also contemplated. Likewise, pharmaceutically acceptable solvates, including hydrates, of the compounds of the formula (1) are contemplated.

It should be understood that the formula (1) structurally encompasses all stereoisomers, including enatiomers, diastereomers, racemates, and combinations thereof which may be contemplated from the chemical structure of the genus described herein.

Also contemplated are prodrugs of the compounds of the formula (1), including ester prodrugs.

According to one embodiment, there is provided a compound of formula (1), wherein X is —C(O)—.

According to one embodiment, there is provided a compound of formula (1), wherein R₁ is

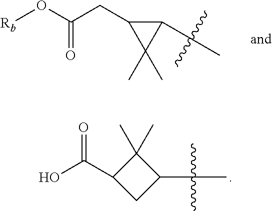

According to one embodiment, there is provided a compound of formula (1), wherein R₁ is

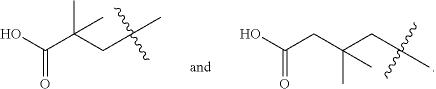

According to one embodiment, there is provided a compound of formula (1), wherein R₁ is

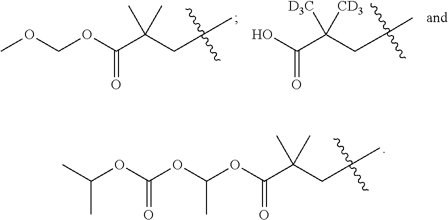 [SMILES: *CC(C)(C)C(=O)OCOC ; *CC(C(=O)O)(C([2H])([2H])[2H])C([2H])([2H])[2H] ; *CC(C)(C)C(=O)OC(C)OC(=O)OC(C)C]

According to one embodiment, there is provided a compound of formula (1), wherein R_{b} is H.

According to one embodiment, there is provided a compound of formula (1), wherein R₂ is

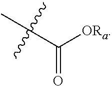

According to one embodiment, there is provided a compound of formula (1), wherein Rₐ is H.

According to one embodiment, there is provided a compound of formula (1), wherein R₂ is Het¹.

According to one embodiment, there is provided a compound of formula (1), wherein Het¹ is 5-phenyl oxadiazol and 5-pyridinyl oxadiazolyl.

According to one embodiment, there is provided a compound of formula (1), wherein R₂ is

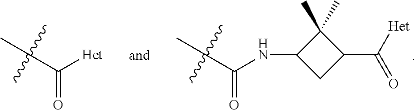

According to one embodiment, there is provided a compound of formula (1), wherein Het is piperidine, 4-(1-methylethyl-2,2,2,1′,1′,1′-D6)piperazine, 5-methyl oxadiazol, piperzine, 5-isobutyl oxadiazol, and Morpholine.

According to one embodiment, there is provided a compound of formula (1), wherein R₂ is

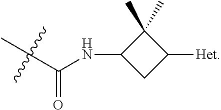

According to one embodiment, there is provided a compound of formula (1), wherein Het is 5-methyl oxadiazol.

According to one embodiment, there is provided a compound of formula (1), wherein R₃ and R₄ can be together with their adjacent carbons form a bond.

According to one embodiment, there is provided a compound of formula (1), wherein R₃ and R₄ can be together with their adjacent carbons form cyclopropl.

According to one embodiment, there is provided a compound of formula (1), wherein R₃ and R₄ are together with their adjacent carbons form epoxide.

According to one embodiment, there is provided a compound of formula (1), wherein R₅ is H.

According to one embodiment, there is provided a compound of formula (1), wherein R′ is phenyl and pyridine.

According to one embodiment, there is provided a compound of formula (1), wherein R′ is H, CH(CD₃)₂, CN, methyl, ethyl, methoxy, isobutyl, phenyl, 2-morpholinoethyl, 3-morpholinopropyl, 3-hydroxy-2-(hydroxymethyl)-2-methylpropanoyl, 2,2-dimethyl-4-oxobutanoic acid, 2-(2-methoxyethoxyl)ethyl, 2-methoxyethoxyl, 2-(2-methoxyethoxyl)ethoxyl, 4-ethylpiperazin-1-yl, (2-ethoxyl)Morpholine, (2-morpholinoethyl)carbamoyl, 3-hydroxy-2-(hydroxymethyl)-2-methylpropanoate, dimethylphosphinate and ethoxyimino.

Accordingly, one other aspect of the present invention provides compounds of formula (1A):

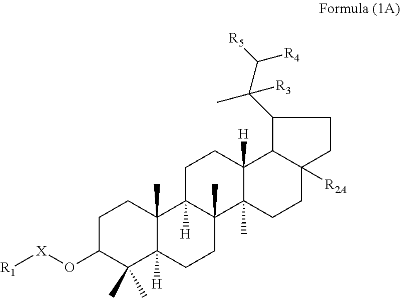

wherein,

X can be a bond, or —C(O)—;

R₁ can be H, substituted or unsubstituted alkyl,

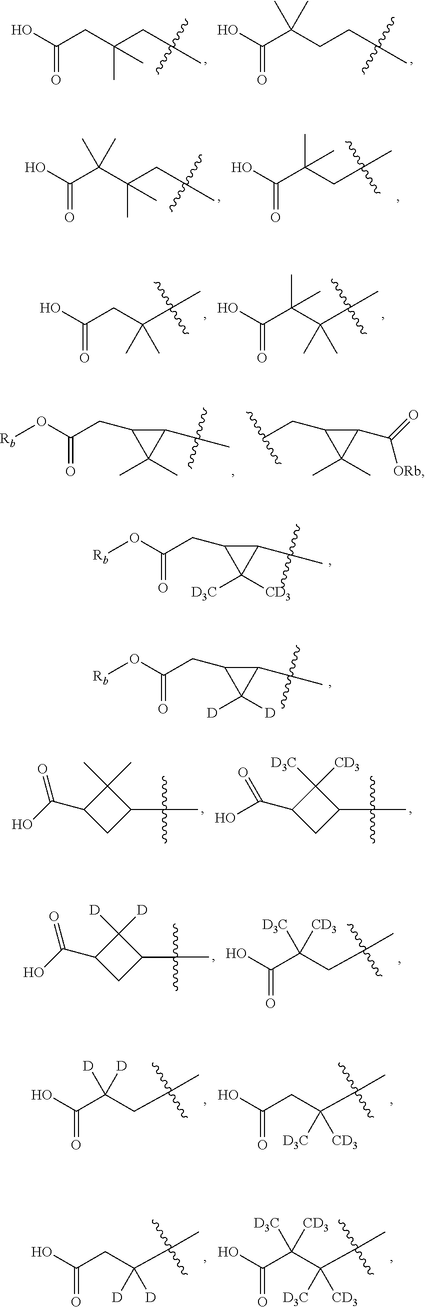

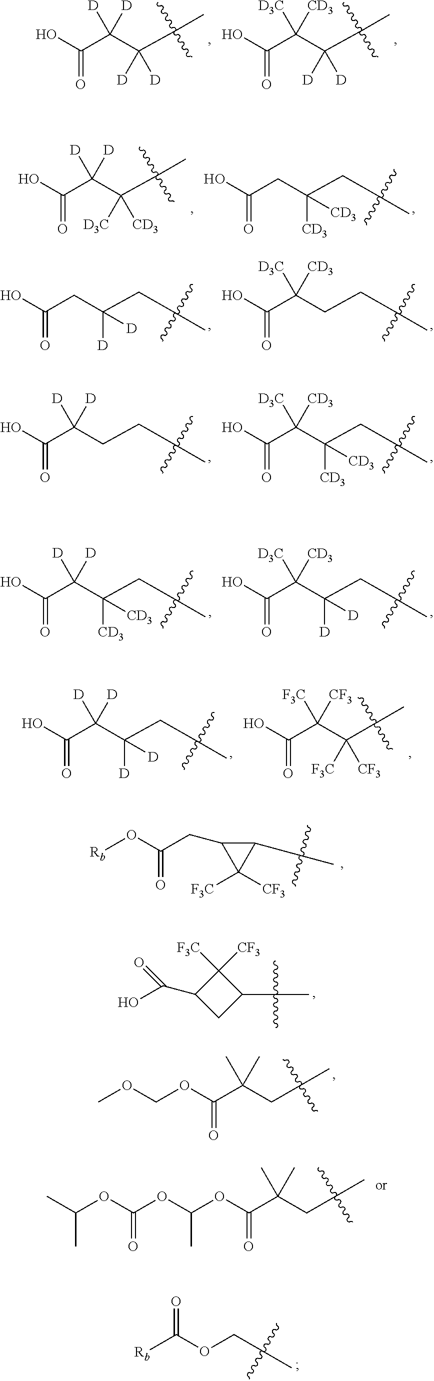

R_{2A} can be

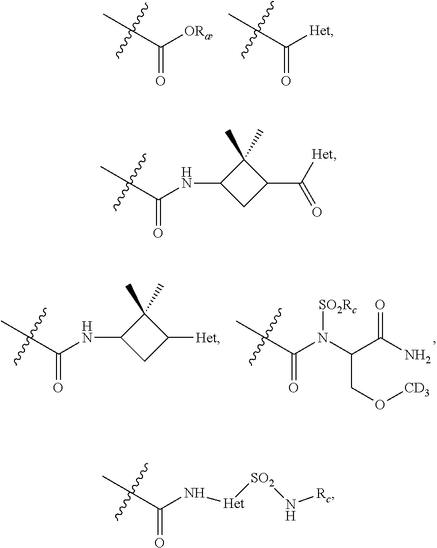

and preferably Het can be selected from oxazole, oxadiazole, triazole, isoxazole, pyrazole, pyridine, piperidine, Morpholine, pyrazine, or piperazine and preferably Het can be substituted by R′.

R₃ and R₄ can be independently selected from H, OH, substituted or unsubstituted alkyl, substituted or unsubstituted alkoxy, substituted or unsubstituted alkoxylalkoxy, or substituted or unsubstituted aminoacids and preferably amino acids are substituted by substituted or unsubstituted alkyl, phosphoric acid, or phosphorus prodrugs or R₃ and R₄ can be together with their adjacent carbons form a bond or R₃ and R₄ can be together with their adjacent carbons form cyclopropl or epoxide;

R₅ can be H, D, CD₃, CH₂CD₃, CH(CD₃)₂, CO₂R_{d}, or substituted or unsubstituted alkyl;

Rₐ, R_{b}, R_{c} and R_{d} can be independently selected from H, or substituted or unsubstituted alkyl;

each R′ can be independently selected from H, CN, D, CD₃, CH₂CD₃, CH(CD₃)₂, CO₂H, CO₂-alkyl, C(O)-alkyl, OC(O)-alkyl, C(O)NH-alkyl, substituted or unsubstituted alkyl, substituted or unsubstituted alkoxy, substituted or unsubstituted phosphinate, substituted or unsubstituted oxime, substituted or unsubstituted amino, substituted or unsubstituted aryl, substituted or unsubstituted cycloalkyl, or substituted or unsubstituted heterocyclyl.

Pharmaceutically acceptable salts of the compounds of the formula (1A) are also contemplated. Likewise, pharmaceutically acceptable solvates, including hydrates, of the compounds of the formula (1A) are contemplated.

It should be understood that the formula (1A) structurally encompasses all stereoisomers, including enatiomers, diastereomers, racemates, and combinations thereof which may be contemplated from the chemical structure of the genus described herein.

Also contemplated are prodrugs of the compounds of the formula (1A), including ester prodrugs.

According to one embodiment, there is provided a compound of formula (1A), wherein X is —C(O)—.

According to one embodiment, there is provided a compound of formula (1A), wherein R₁ is

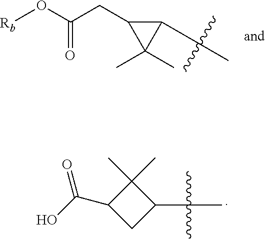

According to one embodiment, there is provided a compound of formula (1A), wherein R₁ is

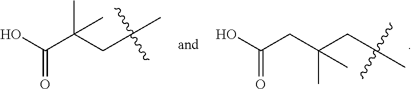

According to one embodiment, there is provided a compound of formula (1A), wherein R₁ is

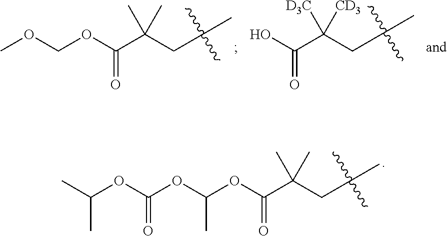 [SMILES: *CC(C)(C)C(=O)OCOC ; *CC(C(=O)O)(C([2H])([2H])[2H])C([2H])([2H])[2H] ; *CC(C)(C)C(=O)OC(C)OC(=O)OC(C)C]

According to one embodiment, there is provided a compound of formula (1A), wherein R_{b} is H.

According to one embodiment, there is provided a compound of formula (1A), wherein R₂ is

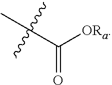

According to one embodiment, there is provided a compound of formula (1A), wherein Rₐ is H.

According to one embodiment, there is provided a compound of formula (1A), wherein R_{2A} is

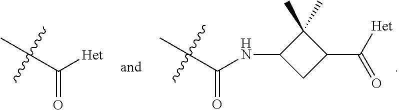

According to one embodiment, there is provided a compound of formula (1A), wherein Het is piperidine, 4-(1-methylethyl-2,2,2,1′,1′,1′-D6)piperazine, 5-methyl oxadiazol, piperzine, 5-isobutyl oxadiazol, and Morpholine.

According to one embodiment, there is provided a compound of formula (1A), wherein R_{2A} is

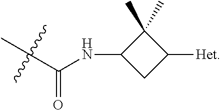

According to one embodiment, there is provided a compound of formula (1A), wherein Het is 5-methyl oxadiazol.

According to one embodiment, there is provided a compound of formula (1A), wherein R₃ and R₄ can be together with their adjacent carbons form a bond.

According to one embodiment, there is provided a compound of formula (1A), wherein R₃ and R₄ can be together with their adjacent carbons form cyclopropl.

According to one embodiment, there is provided a compound of formula (1A), wherein R₃ and R₄ are together with their adjacent carbons form epoxide.

According to one embodiment, there is provided a compound of formula (1A), wherein R₅ is H.

According to one embodiment, there is provided a compound of formula (1A), wherein R′ is phenyl and pyridine.

According to one embodiment, there is provided a compound of formula (1A), wherein R′ is H, CH(CD₃)₂, CN, methyl, ethyl, methoxy, isobutyl, phenyl, 2-morpholinoethyl, 3-morpholinopropyl, 3-hydroxy-2-(hydroxymethyl)-2-methylpropanoyl, 2,2-dimethyl-4-oxobutanoic acid, 2-(2-methoxyethoxyl)ethyl, 2-methoxyethoxyl, 2-(2-methoxyethoxyl)ethoxyl, 4-ethylpiperazin-1-yl, (2-ethoxyl)Morpholine, (2-morpholinoethyl)carbamoyl, 3-hydroxy-2-(hydroxymethyl)-2-methylpropanoate, dimethylphosphinate and ethoxyimino.

Accordingly, one other aspect of the present invention provides compounds of formula (1B):

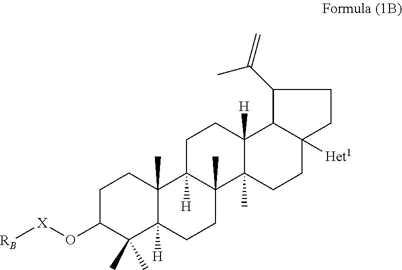

wherein,

X can be a bond, or —C(O)—;

R_{B} can be

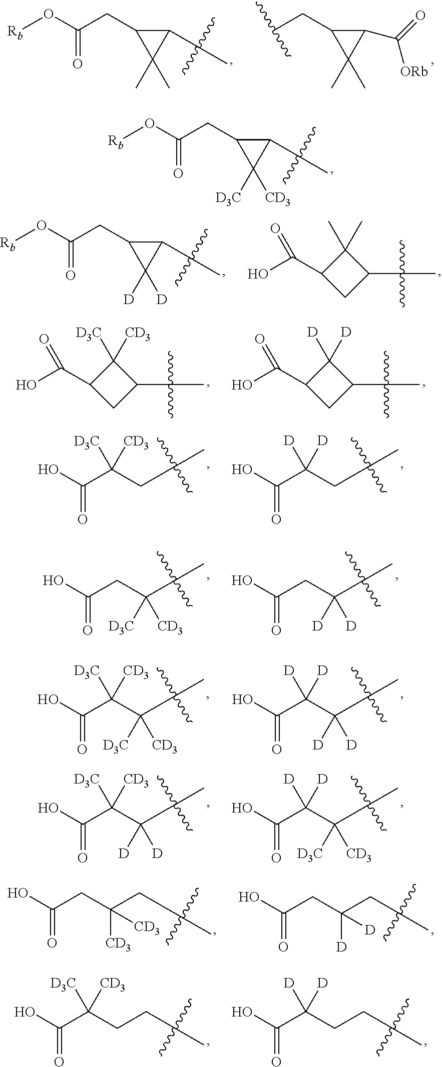

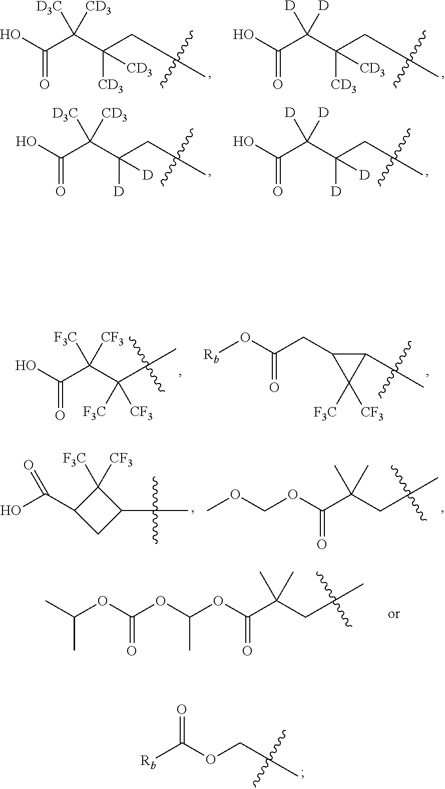

Het¹ can be oxazole, oxadiazole, triazole, isoxazole, pyrazole, pyridine, piperidine, pyrazine, Morpholine, or piperazine and preferably Het¹ can be substituted by R;

R_{b} can be H, or substituted or unsubstituted alkyl;

each R′ can be independently selected from H, CN, D, CD₃, CH₂CD₃, CH(CD₃)₂, CO₂H, CO₂-alkyl, C(O)-alkyl, OC(O)-alkyl, C(O)NH-alkyl, substituted or unsubstituted alkyl, substituted or unsubstituted alkoxy, substituted or unsubstituted phosphinate, substituted or unsubstituted oxime, substituted or unsubstituted amino, substituted or unsubstituted aryl, substituted or unsubstituted cycloalkyl, or substituted or unsubstituted heterocyclyl.

Pharmaceutically acceptable salts of the compounds of the formula (1B) are also contemplated. Likewise, pharmaceutically acceptable solvates, including hydrates, of the compounds of the formula (1B) are contemplated.

It should be understood that the formula (1B) structurally encompasses all stereoisomers, including enatiomers, diastereomers, racemates, and combinations thereof which may be contemplated from the chemical structure of the genus described herein.

Also contemplated are prodrugs of the compounds of the formula (1B), including ester prodrugs.

According to one embodiment, there is provided a compound of formula (1B), wherein X is —C(O)—.

According to one embodiment, there is provided a compound of formula (1B), wherein R_{B} is

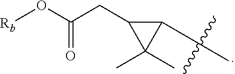

According to one embodiment, there is provided a compound of formula (1B), wherein R_{b} is H.

According to one embodiment, there is provided a compound of formula (1B), wherein Het¹ is 5-phenyl oxadiazol and 5-pyridinyl oxadiazolyl.

According to one embodiment, there is provided a compound of formula (1B), wherein R′ is phenyl and pyridinyl.

Below are the representative compounds, which are illustrative in nature only and are not intended to limit to the scope of the invention (Nomenclature has been generated from Chem. Draw Ultra 11.0 version):
(1R,3aS,5aR,5bR,7aR,9S,11aR,11bR,13aR,13bR)-9-((1S,3R)-3-(carboxymethyl)-2,2-dimethylcyclopropanecarbonyloxy)-5a,5b,8,8,11a-pentamethyl-1-(prop-1-en-2-yl)icosahydro-1H-cyclopenta[a]chrysene-3a-carboxylic acid (Compound 1),
2((1R,3S)-2,2-dimethyl-3-(((1R,3aS,5aR,5bR,7aR,9S,11aR,11bR,13aR,13bR)-5a,5b,8,8,11a-pentamethyl-3a-(piperidine-1-carbonyl)-1-(prop-1-en-2-yl)icosahydro-1H-cyclopenta[a]chrysen-9-yloxy)carbonyl)cyclopropyl)acetic acid (Compound 2),
2,2-dimethyl-4-oxo-4-((1R,3aS,5aR,5bR,7aR,9S,11aR,11bR,13aR,13bR)-5a,5b,8,8,11a-pentamethyl-1-(1-methylcyclopropyl)-3a-(piperidine-1-carbonyl)icosahydro-1H-cyclopenta[a]chrysen-9-yloxy)butanoic acid (Compound 3),
4-((1R,3aS,5aR,5bR,7aR,9S,11aR,11bR,13aR,13bR)-3a-(2,2-dimethyl-3-(piperidine-1-carbonyl)cyclobutylcarbamoyl)-5a,5b,8,8,11a-pentamethyl-1-(1-methylcyclopropyl)icosahydro-1H-cyclopenta[a]chrysen-9-yloxy)-2,2-dimethyl-4-oxobutanoicacid (Compound 4),
2((1R,3S)-2,2-dimethyl-3-(((1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosahydro-1H-cyclopenta[a]chrysen-9-yloxy)carbonyl)cyclopropyl)acetic acid (Compound 5),
2((1R,3S)-2,2-dimethyl-3-(((1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl) icosahydro-1H-cyclopenta[a]chrysen-9-yloxy)carbonyl)cyclopropyl)acetic acid (Compound 6),
4-((1R,3aS,5aR,5bR,7aR,9S,11aR,11bR,13aR,13bR)-3a-(4-(1-methylethyl-2,2,2,1′,1′,1′-D6)piperazine-1-carbonyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid (Compound 7),
4-((1R,3aS,5aR,5bR,7aR,9S,11aR,11bR,13aR,13bR)-3a-((1R,3S)-3-(4-((1-methylethyl-2,2,2,1′,1′,1′-D6)piperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid (Compound 8),
4-(1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yl 1-methoxymethyl 2,2-dimethylsuccinate (Compound 9),
4-(1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yl 1-(1-(isopropoxycarbonyloxy)ethyl)2,2-dimethylsuccinate (Compound 10),
4-((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoicacid (Compound 11),
4-((1R,3aS,5aR,5bR,7aR,9S,11aR,11bR,13aR,13bR)-3a-(4-ethylpiperazine-1-carbon yl)-5a,5b,8,8,11a-pentamethyl-1-(1-methylcyclopropyl)icosahydro-1H-cyclopenta[a]chrysen-9-yloxy)-2,2-dimethyl-4-oxobutanoicacid (Compound 12),
(1R,3S)-3-(((1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylic acid (Compound 13),
4-((1R,3aS,5aR,5bR,7aR,9S,11aR,11bR,13aR,13bS)-3a-((1R,3S)-2,2-dimethyl-3-(piperidine-1-carbonyl)cyclobutylcarbamoyl)-5a,5b,8,8,11a-pentamethyl-1-(2-methyloxiran-2-yl)icosahydro-1H-cyclopenta[a]chrysen-9-yloxy)-2,2-dimethyl-4-oxobutanoicacid (Compound 14),
4-((1R,3aS,5aR,5bR,7aR,9S,11aR,11bR,13aR,13bS)-1-(1,2-dihydroxypropan-2-yl)-3a-((1R,3S)-2,2-dimethyl-3-(piperidine-1-carbonyl)cyclobutylcarbamoyl)-5a,5b,8,8,11a-pentamethylicosahydro-1H-cyclopenta[a]chrysen-9-yloxy)-2,2-dimethyl-4-oxobutanoicacid (Compound 15),
4-((1R,3aS,5aR,5bR,7aR,9S,11aR,11bR,13aR,13bR)-3a-((1R,3S)-3-(5-isobutyl-1,3,4-oxadiazol-2-yl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid (Compound 16),
(1R,3S)-34(1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylicacid (Compound 17),
2-((1R,3S)-3-(((1R,3aS,5aR,5bR,7aR,9S,11aR,11bR,13aR,13bR)-3a-((1R,3S)-2,2-di methyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclopropyl)acetic acid (Compound 18),
2-((1R,3S)-3-(((1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yloxy)carbonyl)-2,2-dimethylcyclopropyl)aceticacid (Compound 19),
(1S,3R)-3-(((1R,3aS,5aR,5bR,7aR,9S,11aR,11bR,13aR,13bR)-3a-((1S,3R)-3-(4-ethylpiperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclobutane carboxylic acid (Compound 20),
(1R,3S)-34(1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclobutane carboxylic acid (Compound 21),
4-(((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-2,2-bis(methyl-d3)-4-oxobutanoic acid (Compound 22),
(1R,3S)-3-((((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 23),
4-(((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(prop-1-en-2-yl)icosahydro-1H-cyclopenta[a]chrysen-9-yl)oxy)-2,2-bis(methyl-d3)-4-oxobutanoic acid (Compound 24),
4-(((1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yl)oxy)-2,2-bis(methyl-d3)-4-oxobutanoic acid (Compound 25),
5-(((1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid (Compound 26),
2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yl)oxy)butanoic acid (Compound 27),
(1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yl)oxy)carbonyl)cyclobutane-1-carb oxylic acid (Compound 28),
(1R,3S)-3-((((1R,3aS,5aR,5bR,7aR,9S,11aR,11bR,13aR,13bR)-3a-(4-methoxypiperidine-1-carbonyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylic acid (Compound 29),
3-(1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl)oxy)carbonyl)-3-(methyl-d3)butanoic-4,4,4-d3 acid (Compound 30),
2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)oxy)butanoic acid (Compound 31),
(1R,3S)-2,2-dimethyl-3-(1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)oxy)carbonyl)cyclobutane-1-carboxylic acid (Compound 32),
2,2-dimethyl-4-oxo-4-(1R,3aS,5aR,5bR,7aR,9S,11aR,11bR,13aR,13bR)-5a,5b,8,8,11a-pentamethyl-1-(1-methylcyclopropyl)-3a-(4-(3-morpholinopropyl) piperazine-1-carbonyl)icosahydro-1H-cyclopenta[a]chrysen-9-yl)oxy)butanoic acid (Compound 33),
(1R,3S)-2,2-dimethyl-3-(1R,3aS,5aR,5bR,7aR,9S,11aR,11bR,13aR,13bR)-5a,5b,8,8,11a-pentamethyl-1-(1-methylcyclopropyl)-3a-(4-(3-morpholinopropyl)piperazine-1-carbonyl)icosahydro-1H-cyclopenta[a]chrysen-9-yl)oxy)carbonyl)cyclobutane-1-carboxylic acid (Compound 34),
4-(1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 35),
(1R,3S)-3-(1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)oxy) carbonyl)-2,2-dimethylcyclobutane-1-carboxylic acid (Compound 36),
4-(1R,3aS,5aR,5bR,7aR,9S,11aR,11bR,13aR,13bR)-3a-(4-(tert-butoxy carbonyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 37),
(1R,3S)-3-(1R,3aS,5aR,5bR,7aR,9S,11aR,11bR,13aR,13bR)-3a-(4-(tert-butoxycarbonyl)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 38),
4-(((1R,3aS,5aR,5bR,7aR,9S,11aR,11bR,13aR,13bR)-3a-(4-(2-(2-methoxyethoxy)ethyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 39),
(1R,3S)-3-((((1R,3aS,5aR,5bR,7aR,9S,11aR,11bR,13aR,13bR)-3a-(4-(2-(2-methoxyethoxy)ethyl)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 40),
4-(((1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 41),
(1R,3S)-3-((((1R,3aS,5aR,5bR,7aR,9S,11aR,11bR,13aR,13bR)-3a-(4-(2-methoxyethoxy)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 42),
4-(((1R,3aS,5aR,5bR,7aR,9S,11aR,11bR,13aR,13bR)-3a-(4-(2-(2-methoxyethoxy)ethoxy)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 43),
(1R,3S)-3-((((1R,3aS,5aR,5bR,7aR,9S,11aR,11bR,13aR,13bR)-3a-(4-(2-(2-methoxyethoxy)ethoxy)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 44),
4-(((1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 45),
(1R,3S)-3-((((1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 46),
2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxy)piperidine-1-carbonyl)icosahydro-1H-cyclopenta[a]chrysen-9-yl)oxy)butanoic acid (Compound 47),
(1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxy)piperidine-1-carbonyl)icosahydro-1H-cyclopenta[a]chrysen-9-yl)oxy)carbonyl)cyclobutane-1-carboxylic acid (Compound 48),
2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)oxy)butanoic acid (Compound 49),
(1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)oxy)carbonyl)cyclobutane-1-carboxylic acid (Compound 50),
4-(((1R,3aS,5aR,5bR,7aR,9S,11aR,11bR,13aR)-3a-(((1S,3R)-2,2-dimethyl-3-(morpholine-4-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 51),
5-(((1R,3aS,5aR,5bR,7aR,9S,11aR,11bR,13aR)-3a-(((1S,3R)-2,2-dimethyl-3-(morpholine-4-carbonyl)cyclobutyl)carbamoyl)-5a,5b,8,8,11a-pentamethyl-1-(1-methylcyclopro-pyl)icosahydro-1H-cyclopenta[a]chrysen-9-yl)oxy)-3,3-dimethyl-5-oxopentanoic acid (Compound 52),
(1R,3S)-3-((((1R,3aS,5aR,5bR,7aR,9 S,11aR,11bR,13aR)-3a-(((1S,3R)-2,2-dimethyl-3-(morpho-line-4-carbonyl)cyclobutyl)carbamoyl)-5a,5b,8,8,11a-pentamethyl-1-(prop-1-en-2-yl)icosa-hydro-1H-cyclopenta[a]chrysen-9-yl)oxy)carbonyl)-2,2-dimethylcyclobutane-1-carboxylic acid (Compound 53),
1-benzyl 3-((1R,3aS,5aR,5bR,7aR,9S,11aR,11bR,13aR)-3a-(((1S,3R)-3-(4-ethylpiperazine-1-car-bonyl)-2,2-dimethylcyclobutyl)carbamoyl)-5a,5b,8,8,11a-pentamethyl-1-(prop-1-en-2-yl)-ico-sahydro-1H-cyclopenta[a]chrysen-9-yl)(1R,3S)-2,2-dimethylcyclobutane-1,3-dicarboxylate (Compound 54),
4-(((1R,3aS,5aR,5bR,7aR,9S,11aR,11bR,13aR)-3a-(((1S,3R)-2,2-dimethyl-3-(piperidine-1-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 55),
4-((1R,3aS,5aR,5bR,7aR,9S,11aR,11bR,13aR,13bR)-3a-(4-cyano-4-phenylpiperidine-1-carbonyl)-5a,5b,8,8,11a-pentamethyl-1-(1-methylcyclopropyl)icosahydro-1H-cyclopenta[a]chrysen-9-yloxy)-2,2-dimethyl-4-oxobutanoic acid (Compound 56),
4-(((1R,3aS,5aR,5bR,7aR,9S,11aR,11bR,13aR,13bR)-3a-(4-((3-hydroxy-2-(hydroxymethyl)-2-methylpropanoyl)oxy)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 57),
(1R,3S)-3-((((1R,3aS,5aR,5bR,7aR,9S,11aR,11bR,13aR,13bR)-3a-(4-((3-hydroxy-2-(hydroxymethyl)-2-methylpropanoyl)oxy)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 58),
4-(((1R,3aS,5aR,5bR,7aR,9S,11aR,11bR,13aR,13bR)-3a-(4-((dimethylphosphoryl)oxy)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 59),
(1R,3S)-3-((((1R,3aS,5aR,5bR,7aR,9S,11aR,11bR,13aR,13bR)-3a-(4-((dimethylphosphoryl)oxy)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 60),
4-(((1R,3aS,5aR,5bR,7aR,9S,11aR,11bR,13aR,13bR)-3a-(((1R,3S)-3-(4-(ethoxyimino)piperidine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 61),
(1R,3S)-3-((((1R,3aS,5aR,5bR,7aR,9S,11aR,11bR,13aR,13bR)-3a-(((1R,3S)-3-(4-(ethoxyimino)piperidine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 62),
4-(((1R,3aS,5aR,5bR,7aR,9S,11aR,11bR,13aR,13bR)-3a-(((1R,3S)-3-(4-((dimethylphosphoryl)oxy)piperidine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 63),
(1R,3S)-3-((((1R,3aS,5aR,5bR,7aR,9S,11aR,11bR,13aR,13bR)-3a-(((1R,3S)-3-(4-((dimethylphosphoryl)oxy)piperidine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 64),
4-(((1R,3aS,5aR,5bR,7aR,9S,11aR,11bR,13aR,13bR)-3a-(((1S,3R)-3-(4-ethylpiperazine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid (Compound 65),
(1R,3S)-3-((((1R,3aS,5aR,5bR,7aR,9S,11aR,11bR,13aR,13bR)-3a-(((1S,3R)-3-(4-ethylpiperazine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid (Compound 66), or
   pharmaceutically acceptable salts, solvates, including hydrates and prodrugs of compounds are also contemplated.

The present invention also provides a pharmaceutical composition that includes at least one compound of described herein and at least one pharmaceutically acceptable excipient (such as a pharmaceutically acceptable carrier or diluent). Preferably, the pharmaceutical composition comprises a therapeutically effective amount of at least one compound described herein. The compound(s) present in the composition may be associated with a pharmaceutically acceptable excipient (such as a carrier or a diluent) or may be diluted by a carrier, or enclosed within a carrier which may be in the form of a capsule, sachet, or other container.

The compounds and pharmaceutical compositions described herein are useful in the treatment of diseases, conditions and/or disorders mediated by viral infections.

The present invention further provides a method of treating a disease, condition and/or disorder mediated by viral infections in a subject in need thereof by administering to the subject one or more compounds described herein in the amount effective to cause that infection.

Also provided herein are processes for preparing compounds described herein.

The invention provides a method for preventing; ameliorating or treating a HIV mediated disease, disorder or syndrome in a subject in need thereof comprising administering to the subject a therapeutically effective amount of a compound of the invention. The invention further provides a method, wherein the HIV mediated disease, disorder or syndrome is like AIDS, AIDS related complex, or a syndrome characterized by symptoms such as pesistant generalized limphadenopathy, fever and weight loss, or an etroviral infection genetically related to AIDS.

Anti HIV inhibitory potential of the compounds of present invention may be demonstrated by any one or more methodologies known in the art, such as by using the assays described in Mosmann T, December 1983, Journal of immunological methods, 65 (1-2), 55-63 and SPC Cole, cancer chemotherapy and Pharmacology, 1986, 17, 259-263.

### DETAILED DESCRIPTION OF THE INVENTION

The present invention provides betulinic acid derivatives and related compounds, which may be used as antiviral particularly as anti-HIV compounds and processes for the synthesis of these compounds. Pharmaceutically acceptable salts, pharmaceutically acceptable solvates, enantiomers, diastereomers, together with pharmaceutically acceptable carriers, excipients or diluents, which can be used for the treatment of diseases, condition and/or disorders mediated by viral infections, are also provided.

The following definitions apply to the terms as used herein:

The terms “halogen” or “halo” includes fluorine, chlorine, bromine, or iodine.

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl).

The term “alkyloxy” refers to a straight or branched hydrocarbon chain with oxygen radical consisting carbon and hydrogen atoms, containing saturation or unsaturation, having from one to eight carbon atoms, and which is attached through oxygen atom to the rest of the molecule by a single bond, e.g., methyloxy, ethyloxy, n-propyloxy, 1-methylethyloxy (isopropyloxy), n-butyloxy, n-pentyloxy, and 1,1-dimethylethyloxy (t-butyloxy).

The term “alkyloxylalkoxy” refers to a straight or branched hydrocarbon chain with oxygen radical consisting carbon atom, hydrogen atom and alkoxy groups, containing saturation or unsaturation, having from one to eight carbon atoms, and which is attached through oxygen atom to the rest of the molecule by a single bond, e.g., 2-(methyloxy)ethyloxy, 2-(ethyloxy)ethyloxy, 2-(n-propyloxy)ethyloxy, and 3-(isopropyloxy)butyloxy.

The term “aminoacid” refers to a straight or branched hydrocarbon chain with containing an amine group, a carboxylic acid group, and a side-chain that is specific to each amino acid and which is attached through Nitrogen atom to the rest of the molecule by a single bond, e.g., alanine, valine, isoleucine, leucine, phenylalanine, or tyrosine.

The term “acyl group” is used to denote a linear or branched aliphatic acyl group (preferably a C₂₋₆ alkanoyl group) or an aromatic acyl group, which contains 2 to 10 carbon atoms. Examples include an acetyl group, a propionyl group, a pivaloyl group, a butyryl group, an isobutyryl group, a valeryl group and a benzoyl group, with an acetyl group being preferred.

The term “cycloalkyl” denotes a non-aromatic mono or multicyclic ring system of from 3 to about 12 carbon atoms, such as cyclopropyl, cyclobutyl, cyclopentyl, and cyclohexyl. Examples of multicyclic cycloalkyl groups include, but are not limited to, perhydronapththyl, adamantyl and norbornyl groups, bridged cyclic groups and spirobicyclic groups, e.g., spiro(4,4)non-2-yl.

The term “cycloalkenyl” refers to a cyclic ring-containing radical having from 3 to about 8 carbon atoms with at least one carbon-carbon double bond, such as cyclopropenyl, cyclobutenyl, and cyclopentenyl.

The term “cycloalkylalkyl” refers to a cyclic ring-containing radical having from 3 to about 8 carbon atoms directly attached to an alkyl group. The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl.

The term “aryl” refers to an aromatic radical having from 6 to 14 carbon atoms such as phenyl, naphthyl, tetrahydronapthyl, indanyl, and biphenyl.

The term “arylalkyl” refers to an aryl group as defined above directly bonded to an alkyl group as defined above, e.g., —CH₂C₆H₅ and —C₂H₅C₆H₅.

“Substituted” refers to 1-3 substituents on the same position or on different positions with the same groups or different groups.

The terms “heterocyclyl” and “heterocyclic ring” refer to a stable 3- to 15-membered ring radical which consists of carbon atoms and from one to five heteroatoms selected from nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heterocyclic or heteroaryl). Examples of such heterocyclic ring radicals include, but are not limited to, tetrazoyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxazolidinyl, triazolyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzothiazolyl, benzooxazolyl, furyl, tetrahydrofurtyl, tetrahydropyranyl, thienyl, benzothienyl, thiamorpholinyl, thiamorpholinyl sulfoxide, thiamorpholinyl sulfone, dioxaphospholanyl, oxadiazolyl. The heterocyclic ring radical may be attached to the main structure at any heteroatom or carbon atom that results in the creation of a stable structure.

The term “heterocyclylalkyl” refers to a heterocyclic ring radical directly bonded to an alkyl group. The heterocyclylalkyl radical may be attached to the main structure at any carbon atom in the alkyl group that results in the creation of a stable structure.

The term “heteroaryl” refers to an aromatic heterocyclic ring radical. The heteroaryl ring radical may be attached to the main structure at any heteroatom or carbon atom that results in the creation of a stable structure.

The term “heteroarylalkyl” refers to a heteroaryl ring radical directly bonded to an alkyl group. The heteroarylalkyl radical may be attached to the main structure at any carbon atom in the alkyl group that results in the creation of a stable structure.

Unless otherwise specified, the term “substituted” as used herein refers to substitution with any one or any combination of the following substituents: hydroxy, halogen, carboxyl, cyano, nitro, oxo (═O), thio (═S), substituted or unsubstituted alkyl, haloalkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenylalkyl, substituted or unsubstituted cycloalkenyl, substituted or unsubstituted amino, substituted or unsubstituted aryl, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COOR^{x}, —C(O)R^{x}, — C(S)R^{x}, —C(O)NR^{x}R^{y}, —C(O)ONR^{x}R^{y}, —NR^{x}CONR^{y}R^{z}, —N(R^{x})SOR^{y}, —N(R^{x})SO₂R^{y}, —(═N—N(R)R^{y}), —NR^{x}C(O)OR^{y}, —NR^{x}R^{y}, —NR^{x}C(O)R^{y}, —NR^{x}C(S)R^{y}, —NR^{x}C(S)NR^{y}R^{z}, —SONR^{x}R^{y}, —SO₂NR^{x}R^{y}, —OR^{x}, —OR^{x}C(O)NR^{y}R^{z}, —OR^{x}C(O)OR^{y}, —OC(O)R^{x}, —OC(O)NR^{x}R^{y}, R^{x}NR^{y}C(O)R^{z}, —R^{x}OR^{y}, —R^{x}C(O)OR^{y}, —R^{x}C(O)NR^{y}R^{z}, —R^{x}C(O)R^{y}, —R^{x}OC(O)R^{y}, —SR^{x}, SOR^{x}, —SO₂R^{x}, and —ONO₂, wherein R^{x}, R^{y} and R^{z} are independently selected from hydrogen, substituted or unsubstituted alkyl, haloalkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocyclylalkyl ring, substituted or unsubstituted heteroarylalkyl, or substituted or unsubstituted heterocyclic ring. The substituents in the aforementioned “substituted” groups cannot be further substituted. For example, when the substituent on “substituted alkyl” is “substituted aryl”, the substituent on “substituted aryl” cannot be “substituted alkenyl”.

The term “prodrug” means a compound that is transformed in vivo to yield a compound of Formula (I), (IA), (1B) or a pharmaceutically acceptable salt, hydrate or solvate, or metabolite of the compound. The transformation may occur by various mechanisms, such as through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The term “treating” or “treatment” of a state, disease, disorder or condition includes:

(1) preventing or delaying the appearance of clinical symptoms of the state, disease, disorder or condition developing in a subject that may be afflicted with or predisposed to the state, disease, disorder or condition but does not yet experience or display clinical or subclinical symptoms of the state, disease, disorder or condition;

(2) inhibiting the state, disease, disorder or condition, i.e., arresting or reducing the development of the state, disease, disorder or condition or at least one clinical or subclinical symptom thereof; or

(3) relieving the state, disease, disorder or condition, i.e., causing regression of the state, disease, disorder or condition or at least one of its clinical or subclinical symptoms.

The benefit to a subject receiving treatment is either statistically significant or at least perceptible to the subject or to the physician.

The term “subject” includes mammals (especially humans) and other animals, such as domestic animals (e.g., household pets including cats and dogs) and non-domestic animals (such as wildlife).

A “therapeutically effective amount” means the amount of a compound that, when administered to a subject for treating a state, disease, disorder or condition, is sufficient to effect such treatment. The “therapeutically effective amount” will vary depending on the compound, the state, disease, disorder or condition and its severity and the age, weight, physical condition and responsiveness of the subject receiving treatment.

The compounds of the present invention may form salts. Non-limiting examples of pharmaceutically acceptable salts forming part of this invention include salts derived from inorganic bases salts of organic bases salts of chiral bases, salts of natural amino acids and salts of non-natural amino acids. Certain compounds of the present invention are capable of existing in stereoisomeric forms (e.g., diastereomers, enantiomers, racemates, and combinations thereof). With respect to the overall compounds described by the Formula (1), (1A) or (1B), the present invention extends to these stereoisomeric forms and to mixtures thereof. To the extent prior art teaches synthesis or separation of particular stereoisomers, the different stereoisomeric forms of the present invention may be separated from one another by the methods known in the art, or a given isomer may be obtained by stereospecific or asymmetric synthesis. Tautomeric forms and mixtures of compounds described herein are also contemplated.

Pharmaceutically acceptable solvates includes hydrates and other solvents of crystallization (such as alcohols). The compounds of the present invention may form solvates with low molecular weight solvents by methods known in the art.

### Pharmaceutical Compositions

The pharmaceutical compositions provided in the present invention include at least one compound described herein and at least one pharmaceutically acceptable excipient (such as a pharmaceutically acceptable carrier or diluent). Preferably, the contemplated pharmaceutical compositions include a compound(s) described herein in an amount sufficient to treat viral infection in a subject.

The subjects contemplated include, for example, a living cell and a mammal, including human. The compound of the present invention may be associated with a pharmaceutically acceptable excipient (such as a carrier or a diluent) or be diluted by a carrier, or enclosed within a carrier which can be in the form of a capsule, sachet, or other container.

Examples of suitable carriers include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone.

The carrier or diluent may include a sustained release material, such as, for example, glyceryl monostearate or glyceryl distearate, alone or mixed with a wax.

The pharmaceutical composition may also include one or more pharmaceutically acceptable auxiliary agents, wetting agents, emulsifying agents, suspending agents, preserving agents, salts for influencing osmotic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing procedures known in the art.

The pharmaceutical compositions described herein may be prepared, e.g., as described in Remington: The Science and Practice of Pharmacy, 20^{th} Ed., 2003 (Lippincott Williams & Wilkins). For example, the active compound can be mixed with a carrier, or diluted by a carrier, or enclosed within a carrier, which may be in the form of an ampule, capsule, or sachet. When the carrier serves as a diluent, it may be a solid, semi-solid, or liquid material that acts as a vehicle, excipient, or medium for the active compound.

The pharmaceutical compositions may be, for example, capsules, tablets, aerosols, solutions, suspensions, liquids, gels, or products for topical application.

The route of administration may be any route which effectively transports the active compound to the appropriate or desired site of action. Suitable routes of administration include, but are not limited to, oral, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment). The oral route is preferred.

Solid oral formulations include, but are not limited to, tablets, capsules (soft or hard gelatin), dragees (containing the active ingredient in powder or pellet form), troches and lozenges. Tablets, dragees, or capsules having talc and/or a carbohydrate carrier or binder or the like are particularly suitable for oral application. Preferable carriers for tablets, dragees, or capsules include lactose, cornstarch, and/or potato starch. A syrup or elixir can be used in cases where a sweetened vehicle can be employed.

A typical tablet that may be prepared by conventional tabletting techniques.

Liquid formulations include, but are not limited to, syrups, emulsions, soft gelatin and sterile injectable liquids, such as aqueous or non-aqueous liquid suspensions or solutions.

For parenteral application, particularly suitable are injectable solutions or suspensions, preferably aqueous solutions with the active compound dissolved in polyhydroxylated castor oil.

### Methods of Screening

Antiviral HIV activity and cytotoxicity of compounds present invention can be measured in parallel by following the methods published in the literature.

The cytotoxic effect of compounds can be analyzed by measuring the proliferation of cells using the 3-(4,5-Dimethylthiazol-2-yl)-2,5-diphenyl tetrazlium bromide (MTT) staining. Cells (5×10³ cells/well) will be incubated in 96 well plates in the presence or absence of compounds. At the end of treatment, 20 μl of MTT (5 mg/ml in PBS) will be added to each well and incubated for an additional 4 hours at 37° C. The purple-blue MTT formazan precipitate will be dissolved in a triplex reagent containing 10% SDS, 5% isobutanol and 10 mmol/lit HCl. The activity of mitochondria, reflecting cellular growth and viability, will be evaluated by measuring the optical density at 570 nm on micro titer plate.

Action of compounds on replication of HIV in Sup-T cells can be determined by the method published by Roda Rani et al., 2006 (Archives of Biochemistry and Biophysics, Volume 456, Issue 1, 1 Dec. 2006, Pages 79-92).

Briefly, 1×10⁶ Sup-T1 cells with 100% cell viability will be seeded in RPMI 1640, 0.1% FBS four 12 well plates. Increasing concentrations of Epap-1 peptides will be added to the cells and will be infected with HIV1_{93 IN 101} each at final concentration of virus equivalent to 2 ng of p24 per ml. The infected cells will be incubated at 37 C and 5% CO2 incubator for 2 hours. After 2 hrs the cells will be pelleted at 350 g for 10 min, supernatant will be discarded and cell will be held with RPMI 1640 containing 10% FBS. The cells will be resuspended in the same medium with increasing concentrations of Epap-1 peptides and will be incubated for 96 hours. The cells will be supplemented with peptides at every 24 hours. The supernatants will be collected after 96 hours and analyzed using P24 antigen capture assay kit (SAIC Fredrick). The infection in the absence of Epap-1 will be considered to be 0% inhibition Azidothymidine (AZT) will be taken as positive control.

Action of compound on virus entry and quantification of virus entered can be done in terms of GFP expression by the following the methods published J. Virol. 72, 6988 (1998) by in Cecilia et al., and Analytical Biochemistry Volume 360, Issue 2, 15 Jan. 2007, Pages 315-317 (Dyavar S. Ravi and Debashis Mitra).

Briefly, cells will be seeded in to wells of 24 well plates 1 day prior to the experiment. The cells will be transfected with Tat-reporter. The virus inoculum will be adjusted to 1,000-4,000 TCID 50/ml in assay medium (DMEM, 10% FCS, glutamine and antibiotics), 50 μl aliquots will be incubated with serial dilutions of compounds (50 μl) for 1 hr at 37° C. The reporter expression will be quantified at appropriate time calculated inhibitory doses referrers to the concentration of these agents in this preincubation mixture.

Other relevant references useful for screening antiviral HIV activity are: Averett, D. R. 1989. Anti-HIV compound assessment by two novel high capacity assays. J. Virol. Methods 23: 263-276; Schwartz, O., et al. 1998; A rapid and simple colorimeric test for the study of anti HIV agents. AIDS Res. and Human Retroviruses, 4(6):441-447; Daluge, S. M., et al. 1994. 5-Chloro-2′,3′-deoxy-3′fluorouridine (935U83), a selective anti human immunodeficiency virus agent with an improved metabolic and toxicological profile; Antimicro. Agents and Chemotherapy, 38(7):1590-1603; H. Mitsuya and S. Border, Inhibition of the in vitro infectivity and cytopathic effect of human T-lymphotropic virus type lymphadenopathy-associated virus (HLTV-III/LAV) by 2,3′-dideoxynucleosides, Proc. Natl. Acad. Sci. USA, 83,1911-15(1986); Pennington et al., Peptides 1990; Meek T. D et al., Inhibition of HIV-1 protease in infected T-limphocytes by synthetic peptide analogues, Nature, 343, p90 (1990); Weislow et al., J. Natl. Cancer Inst. 81, 577-586, 1989; T. Mimoto et al., J. Med. Chem., 42, 1789-1802, 1999; Uckun et al 1998, Antimicobial Agents and Chemotherapy 42:383; for P24 antigen assay Erice et al., 1993, Antimicrob. Ag. Chemotherapy 37: 385-383; Koyanagi et al., Int. J. Cancer, 36, 445-451, 1985; Balzarini et al. AIDS (1991), 5, 21-28; Connor et al., Journal of virology, 1996, 70, 5306-5311; Popik et al., Journal of virology, 2002, 76, 4709-4722; Harrigton et al., Journal of Virology Methods, 2000, 88, 111-115; Roos et al., Virology 2000, 273, 307-315; Fedyuk N. V. et al; Problems of Virology 1992, (3)P135; Mosmann T, December 1983, Journal of immunological methods, 65 (1-2), 55-63; SPC Cole, cancer chemotherapy and Pharmacology, 1986, 17, 259-263.

### Methods of Treatment

The present invention provides compounds and pharmaceutical formulations thereof that are useful in the treatment of diseases, conditions and/or disorders mediated by viral infections. The connection between therapeutic effect and antiviral is illustrated. For example, PCT publication Nos. WO 01/07646, WO 01/65957, or WO 03/037908; U.S. Pat. No. 4,598,095 or US 2002/0068757; EP publication Nos. EP 0989862 or EP 0724650; Bioorganic & Medicinal Chemistry Letters, 16, (6), 1712-1715, 2006; and references cited therein, all of which are incorporated herein by reference in their entirety and for the purpose stated.

The present invention further provides a method of treating a disease, condition and/or disorder mediated by viral infections in a subject in need thereof by administering to the subject a therapeutically effective amount of a compound or a pharmaceutical composition of the present invention.

Diseases, conditions, and/or disorders that are mediated by viral infections are believed to include, but are not limited to, HIV infection, HBV, HCV, a retroviral infection genetically related to HIV, AIDS, inflammatory disease, respiratory disorders (including adult respiratory distress syndrome (ARDS), bronchitis, chronic bronchitis, chronic obstructive pulmonary disease, cystic fibrosis, asthma, emphysema, rhinitis and chronic sinusitis), inflammatory bowel disease (including Crohn's disease and ulcerative colitis), multiple sclerosis, rheumatoid arthritis, graft rejection (in particular but not limited to kidney and lung allografts), endometriosis, type I diabetes, renal diseases, chronic pancreatitis, inflammatory lung conditions, chronic heart failure and bacterial infections (in particular but not limited to tuberculosis).

The compounds of the present invention can obtain more advantageous effects than additive effects in the prevention or treatment of the above diseases when using suitably in combination with the above drugs. Also, the administration dose can be decreased in comparison with administration of either drug alone, or adverse effects of co administrated drugs other than antiviral can be avoided or declined.

### Methods of Preparation

The compounds described herein may be prepared by techniques known in the art. In addition, the compounds described herein may be prepared by following the reaction sequence as depicted in Scheme-1 to 3. Further, in the following schemes, where specific bases, acids, reagents, solvents, coupling agents, etc., are mentioned, it is understood that other bases, acids, reagents, solvents, coupling agents etc., known in the art may also be used and are therefore included within the present invention. Variations in reaction conditions, for example, temperature and/or duration of the reaction, which may be used as known in the art, are also within the scope of the present invention. All the stereo isomers of the compounds in these schemes, unless otherwise specified, are also encompassed within the scope of this invention.

Compounds of the present invention can be synthesized from naturally occurring Betulinic acid or betulinal. Key intermediates required for synthesizing analogues are either commercially available, or can be prepared by the methods published in the literature. For example, the key intermediates in the present invention were prepared by modifying the procedures published in Journal of organic chemistry 2010, 75, 1285-1288; Journal of organic chemistry 2000, 65, 3934-3940; Tetrahedron: asymmetry 2008, 19, 302-308; or Tetrahedron: asymmetry 2003, 14, 217-223.

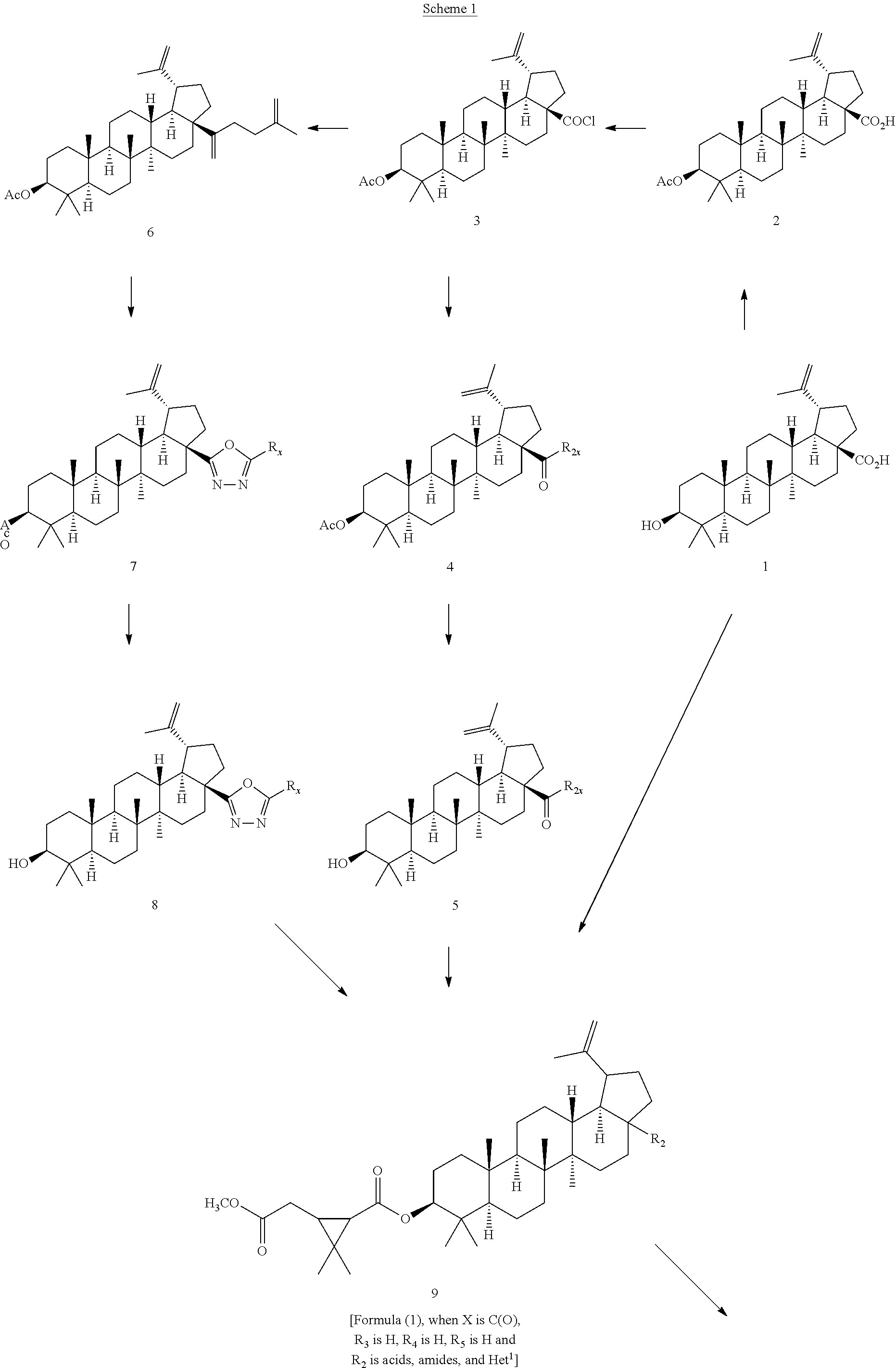

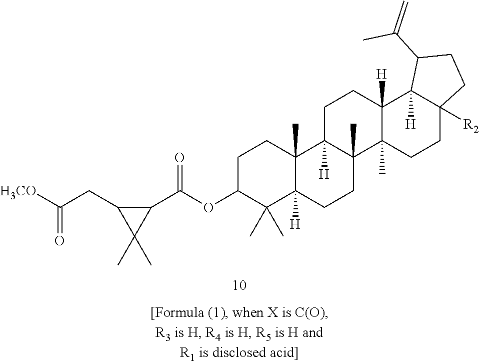

Compounds of formula 10 (wherein, Rₓ is H, or substituted or unsubstituted alkyl, substituted or unsubstituted aryl or substituted or unsubstituted alkyl heterocyclyl; R₂ₓ is substituted or unsubstituted amine, substituted or unsubstituted heterocyclyl and R₂ is same as defined above) can be prepared as described in Scheme 1. Reacting a C-3 alcohol with a suitable ester forming reagents like anhydrides, acid halides or mixed anhydrides in the presence of a base like triethyl amine, diisopropyl ethyl mine, or pyridine in an inert solvent like DCM, toluene, THF or a basic solvent like pyridine with or without addition of a catalyst like DMAP. For example a C-3 alcohol of compounds of the formula 1 can be protected by an acetyl group in the presence of acetic anhydride (as described in T. W. Greene and P. G. M. Wuts, protective groups in organic synthesis, 3^{rd} edition, John wiley & sons, New york, 1999) in the solvents such as pyridine or the like, to give the C-3 acetyl compounds of formula 2. Converting the compound of formula 2 to C-28 carboxylic halide compounds of formula 3 in the presence of acid halides such as thionyl chloride, oxalyl chloride, phosphorous bromide, phosphorous oxy bromide, phosphorous pentachloride, phosphorous tribromide, phosphorous pentabromide or the like in the solvents such as benzene, toluene, DCM or the like. The C-28 carboxylic halide compounds of formula 3 can be converted to amides with corresponding amines in the presence of bases such as triethylamine, DIPEA, pyridine or the like in the solvents such as N,N-Dimethylformamide, dichloromethane, THF or the like, to give C28-amide compounds of formula 4. The compounds of formula 4 can be hydrolised in the presence of bases such as potassiumcarbonate, sodiumhydroxide, ammonia or the like in the solvents such as methanol:THF, methanol:water, methanol or the like to give the compounds of formula 5.

Compounds of formula 9 where in R₂ is Het can be synthesized, by reacting a C-28 carboxylic halide compounds of formula 3 with a hydrazides of substituted or unsubstituted carboxylicacids in the presence of coupling agents such as O-Benzotriazole-N,N,N′,N′-tetramethyl-uronium-hexafluoro-phosphate (HBTU), 1-ethyl-3-(3-dimethylaminopropyl) carbodiimide) hydrochloride (EDCI), 1-Hydroxybenzotriazole (HOBt) monohydrate or the like with suitable bases such as triethylamine, N,N-diisopropylethylamine, pyridine or the like with nicotinylchloride or benzoylchloride in the solvents such as N,N-Dimethylformamide, dichloromethane, THF or the like to give hydrazide compounds of formula 6. The hydrazide compounds of formula 6 can be cyclised in the presence acidic agents such as para-toluenesulphonylchloride, phosphorous oxychloride, phosphorous pentoxide, phosphorous pentachloride or the like in the solvents such as dichloromethane acetonitrile, trichloromethane with added bases such as triethylamine, N,N-diisopropylethylamine, pyridine or the like to give C-28 heterocyclic compounds of formula 7. C3 ester in compounds of formula 7 can be hydrolised in the presence of bases such as potassiumcarbonate, sodiumhydroxide, ammonia or the like in the solvents such as methanol:THF, methanol:water, methanol or the like to give C3 hydroxy compounds of formula 8.

Finally, compounds of formula 8, compounds of formula 5 and compounds of the formula 1 can be reacted independently with 3-(2-methoxy-2-oxoethyl)-2,2-dimethylcyclopropanecarboxylic acid [as described in J. Org. Chem, 2010, 75, 1285-1288] in the presence of condensing agents such as 2,4,6-trichlorobenzoyl chloride or the like in the solvents such as THF, benzene, toluene or the like with added bases such as triethylamine, N,N-diisopropylethylamine, pyridine, DMAP or the like to give C3-ester compounds of formula 9 [Formula (1), when X is C(O), R₃ is H, R₄ is H, R₅ is H and R₂ is acids, amides, and Het¹]. The C3-ester compounds of formula 9 can be hydrolised in the presence of hydrolisuing agents such as lithiumhydroxide monohydrate, sodiumhydroxide, potassiumhydroxide or the like in the solvents like THF-water, methanol-water or ethanol-water to give acid compounds of formula 10 [Formula (1), when X is C(O), R₃ is H, R₄ is H, R₅ is H and R₁ is disclosed acid].

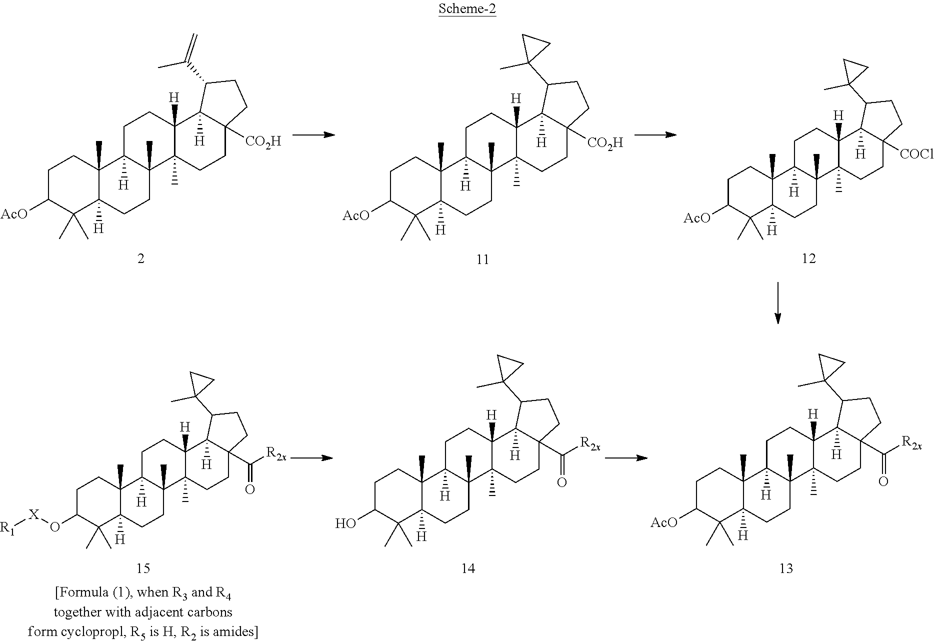

Compounds of formula 15 (wherein, R₂ₓ is substituted or unsubstituted amine, substituted or unsubstituted heterocyclyl; X and R₁ are same as defined above) can be prepared as described in Scheme 2. The C-3 acetyl compound of formula 2 can be converted to C-20 cyclopropyl compounds of formula 11 in the presence of cyclopropenating agents such as diethylzinc, dimethylzinc, Zn—Cu or the like with diidomethane in the solvents such as dichloromethane, 1,2-dichloroethane, toluene, benzene, ether or the like. The compounds of formula 11 can be converted to the halide compounds of formula 12 in the presence of halogenating agents such as thionyl chloride, oxalyl chloride, phosphorous bromide, phosphorous oxy bromide, phosphorous pentachloride, phosphorous tribromide, phosphorous pentabromide or the like in the solvents such as benzene, toluene, DCM or the like. The compounds of formula 12 can be converted to amides with corresponding amines in the presence of bases such as triethylamine, DIPEA, pyridine or the like in the solvents such as N,N-Dimethylformamide, dichloromethane, THF or the like to give the compounds of formula 13. Alternately, C28-amide compounds of formula 13 can also be prepared by using suitable coupling agents such as 1-ethyl-3-(3-dimethylaminopropyl) carbodiimide) hydrochloride (EDCI), 2-(1H-7-Azabenzotriazol-1-yl)-1,1,3,3-tetramethyl uronium hexafluorophosphate (HATU), 1-Hydroxybenzotriazole (HOBt) monohydrate or the like with suitable bases such as triethylamine, N,N-diisopropylethylamine, pyridine or the like with nicotinylchloride or benzoylchloride in the solvents such as N,N-Dimethylformamide, dichloromethane, THF or the like. The compounds of formula 13 can be deprotected in presence of bases such as potassiumcarbonate, sodiumhydroxide, ammonia or the in the solvents such as methanol:THF, methanol:water, methanol or the like to give the C3 hydroxy compounds of formula 14. The compounds of formula 14 obtained in previous step can be reacted with corresponding acid anhydrides, half protected diacids or their mixed anhydrides or acid chlorides to give the corresponding compounds of present invention represented by formula 15 [Formula (1), when R₃ and R₄ together with adjacent carbons form cyclopropl, R₅ is H, R₂ is amides] in the presence a base like triethyl amine, 4-Dimethylaminopyridine, diisopropyl ethyl mine or pyridine or the like in the solvents such as for example, DCM, toluene, EtOAc, THF or the like.

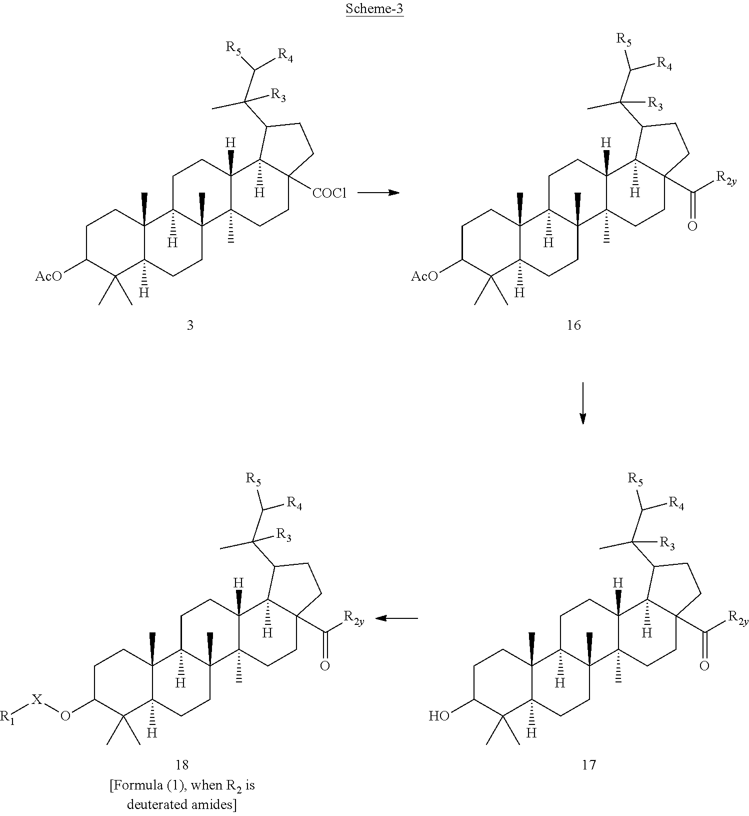

Compounds of formula 18 (wherein, R_{2y} is substituted or unsubstituted deuterated amine, deuterium substituted or unsubstituted heterocyclyl; X, R₁, R₃, R₄ and R₅ are same as defined above) can be prepared as described in Scheme 3. The compounds of formula 3 (as described in scheme 1) can be converted to amide by reacting with corresponding deuterated amines in the presence of bases such as triethylamine, DIPEA, pyridine or the like in the solvents such as N,N-Dimethylformamide, dichloromethane, THF or the like to give the compounds of formula 16. In the next step, the compounds of formula 16 can be deacetylated in presence of bases such as potassiumcarbonate, sodiumhydroxide, ammonia or the like in the solvents such as methanol:THF, methanol:water, methanol or the like to give C-28 amides of C3 hydroxy compounds of formula 17. The C3 hydroxy compounds of formula 17 can be reacted with acid anhydride compounds, half protected diacids or their mixed anhydrides or acid chlorides to give the corresponding acid compounds of formula 18 [Formula (1), when R₂ is deuterated amides] in the presence a base like triethyl amine, 4-Dimethylaminopyridine, diisopropyl ethyl mine or pyridine or the like in the solvents such as for example, DCM, toluene, EtOAc, THF or the like.

### EXPERIMENTAL

The present invention is further illustrated by the following examples, which are not to be construed in any way as imposing limitations upon the scope of this disclosure, but rather are intended to be illustrative only. On the contrary, it is to be clearly understood that resort may be had to various other embodiments, modifications, and equivalents thereof which, after reading the description herein, may suggest themselves to one of ordinary skill in the art without departing from the spirit of the present invention. Thus, the skilled artisan will appreciate how the experiments and Examples may be further implemented as disclosed by variously altering the following examples, substituents, reagents, or conditions.

### EXAMPLES

### Example 1

### Preparation of (1R,3aS,5aR,5bR,7aR,9S,11aR,11bR,13aR,13bR)-9-((1S,3R)-3-(carboxymethyl)-2,2-dimethylcyclopropanecarbonyloxy)-5a,5b,8,8,11a-pentamethyl-1-(prop-1-en-2-yl)icosahydro-1H-cyclopenta[a]chrysene-3a-carboxylic acid

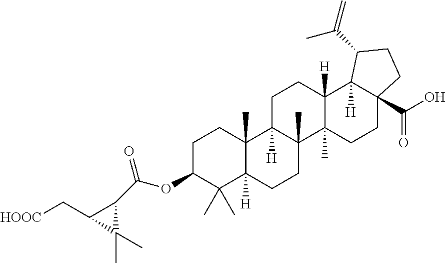

### Step 1: (1S,3R)-3-(2-methoxy-2-oxoethyl)-2,2-dimethylcyclopropanecarboxylic acid

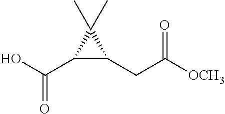

To a solution of Jones reagent (20 ml) in acetone (15 ml) cooled to 0° C., methyl 2-((1R,3S)-3-(hydroxymethyl)-2,2-dimethylcyclopropyl)acetate (3.00 g, 17.4 mmol, 1.0 eq) in acetone (15 ml) was added then the reaction mixture was allowed to stir at room temperature for overnight. TLC indicated starting material was consumed and the desired product was observed. The reaction mixture was quenched with isopropanol and solvent was evaporated under reduced pressure. The mixture was diluted with CH₂Cl₂, washed with brine solution, dried over Na₂SO₄, filtered and evaporated under reduced pressure. The residue was purified by silicagel column chromatography using 6% ethyl acetate/hexane as eluent to give the title compound (0.950 g, 30% yield) as pale yellow color oil. ¹HNMR (300 MHz, CDCl₃): δ ppm 10.9 (brs, 1H), 3.68 (s, 3H), 2.76 (d, 2H, J=6.9 Hz), 1.61-1.43 (m, 2H), 1.23 (s, 3H), 1.21 (s, 3H).

### Step 2: (1R,3aS,5aR,5bR,7aR,9S,11aR,11bR,13aR,13bR)-9-((1S,3R)-3-(2-methoxy-2-oxo ethyl)-2,2-dimethylcyclopropanecarbonyloxy)-5a,5b,8,8,11a-pentamethyl-1-(prop-1-en-2-yl)icosahydro-1H-cyclopenta[a]chrysene-3a-carboxylic acid

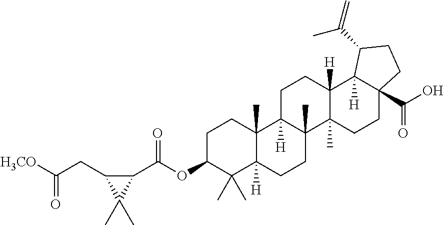

To a stirred solution of (1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysene-3a-carboxylic acid (0.500 g, 1.09 mmol, 1.0 eq) (1S,3R)-3-(2-methoxy-2-oxoethyl)-2,2-dimethylcyclopropanecarboxylic acid (step 1, 0.448 g, 2.41 mmol, 2.2 eq) in dry THF (7 ml) and cooled to 0° C. then diisopropylethyl amine (0.849 g, 6.57 mmol, 6.0 eq), 4-dimethylaminopyridine (0.334 g, 2.73 mmol, 2.5 eq) and 2,4,6-trichlorobenzoyl chloride (0.794 g, 3.28 mmol, 3.0 eq) were added sequentially and stirred at room temperature for about 36 hours. The reaction mixture was concentrated under reduced pressure, diluted with water (20 ml) and extracted with CH₂Cl₂ (3×25 ml). The combined organic extracts were dried over Na₂SO₄ and evaporated under reduced pressure. The residue was purified by silicagel column chromatography using 8% ethyl acetate/hexane as eluent to give the title compound (250 mg, 36% yield) as a white solid. ¹HNMR (300 MHz, CDCl₃): δ ppm 4.73 (s, 1H), 4.61 (s, 1H), 4.47-4.41 (m, 1H), 3.66 (s, 3H), 3.07-2.95 (m, 1H), 2.76 (d, 2H, J=7.5 Hz), 2.30-2.15 (m, 2H), 2.05-1.95 (m, 2H), 1.67-1.53 (m, 7H), 1.52-1.35 (m, 10H), 1.28-1.24 (m, 4H), 1.20 (s, 3H), 1.18 (s, 3H), 1.16-1.12 (m, 1H), 0.99-0.88 (m, 8H), 0.88-0.81 (m, 9H), 0.79-0.74 (m, 1H); ES MS: [M−H]⁻ 623.5 (100%).

### Step 3: (1R,3aS,5aR,5bR,7aR,9S,11aR,11bR,13aR,13bR)-9-((1S,3R)-3-(carboxymethyl)-2,2-dimethylcyclopropanecarbonyloxy)-5a,5b,8,8,11a-pentamethyl-1-(prop-1-en-2-yl)icosahydro-1H-cyclopenta[a]chrysene-3a-carboxylic acid

To a stirred solution of (1R,3aS,5aR,5bR,7aR,9S,11aR,11bR,13aR,13bR)-9-((1S,3R)-3-(2-methoxy-2-oxo ethyl)-2,2-dimethylcyclopropanecarbonyloxy)-5a,5b,8,8,11a-pentamethyl-1-(prop-1-en-2-yl)icosahydro-1H-cyclopenta[a]chrysene-3a-carboxylic acid (step 2, 0.250 g, 0.40 mmol, 1.0 eq) in THF:H₂O (4:1, 5 ml) cooled to 0° C., lithium hydroxide monohydrate (0.050 g, 1.20 mmol, 3.0 eq) was added then reaction was allowed to stir at room temperature for over night. TLC indicated starting material was consumed and the desired product was observed. The mixture was concentrated under reduced pressure, cooled to 0° C., acidified to pH=4-5 using 1N HCl and extracted with CH₂Cl₂ (3×25 ml). The combined organic extracts were washed with water, dried over Na₂SO₄, filtered and evaporated under reduced pressure. The residue was purified by silicagel column chromatography using 15% ethyl acetate/hexane as eluent to give the title compound (0.055 g, 23% yield) as a white solid. ¹H NMR (300 MHz, DMSO-d6): δ ppm 12.0 (brs, 2H), 4.68 (s, 1H), 4.56 (s, 1H), 4.39-4.32 (m, 1H), 3.0-2.91 (m, 1H), 2.57 (dd, 2H, J=6.9, 1.8 Hz), 2.28-2.17 (m, 2H), 2.13-2.08 (m, 1H), 1.85-1.75 (m, 2H), 1.62-1.57 (m, 2H), 1.55-1.20 (m, 18H), 1.17-1.09 (m, 6H), 1.08-1.05 (m, 1H), 1.05-1.0 (m, 1H), 0.97-0.92 (m, 4H), 0.89-0.82 (m, 3H), 0.82-0.78 (m, 10H); ES MS: [M−H]⁻ 609.3 (100%); HPLC: 92.3%.

### Example 2

### Preparation of 2-((1R,3S)-2,2-dimethyl-3-(((1R,3aS,5aR,5bR,7aR,9S,11aR,11bR,13aR,13bR)-5a,5b,8,8,11a-pentamethyl-3a-(piperidine-1-carbonyl)-1-(prop-1-en-2-yl)icosahydro-1H-cyclopenta[a]chrysen-9-yloxy)carbonyl)cyclopropyl)acetic acid

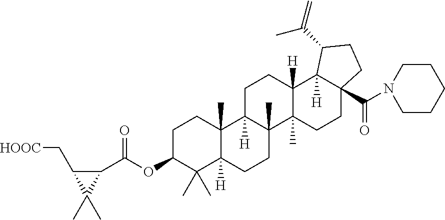

### Step 1: (1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxylic acid

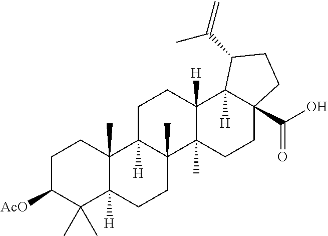

Acetic anhydride (0.86 ml, 9.2 mmol, 1.4 eq) was added to a solution of (1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysene-3a-carboxylic acid (3.0 g, 6.52 mmol, 1.0 eq) then DIPEA (2.54 g, 19.6 mmol, 3.0 eq) and DMAP (0.096 g, 0.788 mmol, 0.12 eq) in THF (50 ml) were poured and the reaction mixture was heated at 65-70° C. for 2 hours and monitored until TLC demonstrated complete consumption of the starting material. The mixture was concentrated in vacuo to dryness to yield a white solid. To hydrolyze the mixed anhydride, this solid was suspended in 0.6 M hydrochloric acid solution (10 ml) and heated at 100° C. for about 30 minutes. The suspension was cooled to room temperature and the solid was collected by filtration, washed with water (20 ml) and dried at 50° C. under reduced pressure to afford the title compound (2.5 g, 91% yield) as a white free-flowing powder. TLC R_{f} 0.75 (20% EtOAc/Hexane). ¹H NMR (300 MHz, CDCl₃): δ 4.74 (s, 1H), 4.61 (s, 1H), 4.49-4.44 (m, 1H), 3.04-2.96 (m, 1H), 2.32-2.15 (m, 2H), 2.05 (s, 3H), 2.02-1.93 (m, 2H), 1.63-1.50 (m, 11H), 1.49-1.35 (m, 6H), 1.32-1.25 (m, 2H), 1.24-1.17 (m, 1H), 1.10-1.0 (m, 2H), 0.97 (s, 3H), 0.93 (s, 3H), 0.87-0.82 (m, 9H), 0.78-0.75 (m, 1H); ES MS: [M−H]⁻ 497.0 (100%).

### Step 2: (1R,3aS,5aR,5bR,7aR,9S,11aR,11bR,13aR,13bR)-3a-(chlorocarbonyl)-5a,5b,8,8,11a-pentamethyl-1-(prop-1-en-2-yl)icosahydro-1H-cyclopenta[a]chrysen-9-yl acetate

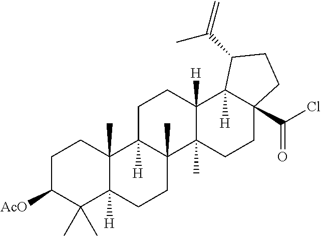

To a chilled (0° C.) solution of (1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxylic acid (step 1, 0.500 g, 1.0 mmol, 1.0 eq) in toluene (5 ml) thionyl chloride (0.596 g, 5.01 mmol, 5.0 eq) was added and the reaction mixture was heated to reflux for about 3 hours. After completion of the reaction monitored by TLC, the solvent was evaporated in vacuo providing a crude residue that was re-dissolved in toluene (10 ml) to remove the excess thionyl chloride. The solution was concentrated in vacuo to produce the desired title compound (0.51 g), which is used as such for next step.

### Step 3: (1R,3aS,5aR,5bR,7aR,9S,11aR,11bR,13aR,13bR)-5a,5b,8,8,11a-pentamethyl-3a-(piperidine-1-carbonyl)-1-(prop-1-en-2-yl)icosahydro-1H-cyclopenta[a]chrysen-9-ylacetate

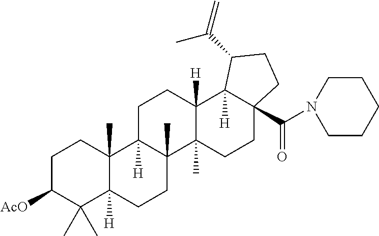

To a chilled (0° C.) suspension of piperidine (0.126 g, 1.48 mmol, 1.5 eq) in CH₂Cl₂ (5 ml) DIPEA (0.50 g, 3.9 mmol, 4.0 eq) was added then a solution of (1R,3aS,5aR,5bR,7aR,9S,11aR,11bR,13aR,13bR)-3a-(chlorocarbonyl)-5a,5b,8,8,11a-pentamethyl-1-(prop-1-en-2-yl)icosahydro-1H-cyclopenta[a]chrysen-9-yl acetate (step 2, 0.51 g, 0.98 mmol, 1.0 eq) in CH₂Cl₂ (5 ml) was added and the reaction mixture was allowed to warm to room temperature and stirred at the same temperature for overnight. The reaction mixture was diluted with CH₂Cl₂, washed with water, brine solution, dried over Na₂SO₄, filtered, evaporated and purified by silicagel column chromatography using 4% EtOAc:hexane as eluent to afford the title compound (0.5 g, 88% yield over 2 steps) as a white solid. ¹H NMR (300 MHz, CDCl₃): δ 4.72 (s, 1H), 4.57 (s, 1H), 4.49-4.44 (m, 1H), 3.60-3.40 (m, 4H), 3.07-2.84 (m, 2H), 2.15-2.08 (m, 1H), 2.04 (s, 3H), 2.0-1.92 (m, 1H), 1.91-1.80 (m, 1H), 1.64-1.60 (m, 3H), 1.60-1.55 (m, 3H), 1.53-1.47 (m, 6H), 1.43-1.40 (m, 2H), 1.40-1.32 (m, 6H), 1.30-1.25 (m, 4H), 1.23-1.20 (m, 1H), 1.18-1.10 (m, 2H), 1.05-0.95 (m, 1H), 0.94 (s, 3H), 0.94 (s, 3H), 0.87-0.80 (m, 9H), 0.80-0.78 (m, 1H); ES MS: [M+H]⁺ 566.1 (100%).

### Step 4: ((1R,3aS,5aR,5bR,7aR,9S,11aR,11bR,13aR,13bR)-9-hydroxy-5a,5b,8,8,11a-penta methyl-1-(prop-1-en-2-yl)icosahydro-1H-cyclopenta[a]chrysen-3a-yl)(piperidin-1-yl)methanone

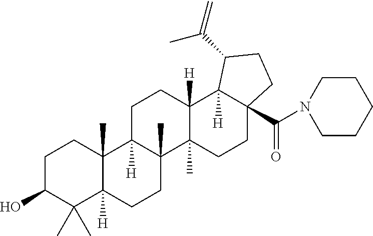

To a stirred solution of (1R,3aS,5aR,5bR,7aR,9S,11aR,11bR,13aR,13bR)-5a,5b,8,8,11a-pentamethyl-3a-(piperidine-1-carbonyl)-1-(prop-1-en-2-yl)icosahydro-1H-cyclopenta[a]chrysen-9-ylacetate (step 3, 0.51 g, 0.90 mmol, 1.0 eq) in THF:MeOH (1:1, 10 ml) potassium carbonate (0.41 g, 2.97 mmol, 3.3 eq) was added and stirred at room temperature for about 48 hours. After completion of the reaction monitored by TLC, the reaction mixture was filtered through celite and washed the salts with CH₂Cl₂, filtrate was evaporated in vacuo and purified by silicagel column chromatography using 10% EtOAc:hexane as eluent to afford the title compound (0.47 g) as a white solid. ¹H NMR (300 MHz, CDCl₃): δ 4.72 (s, 1H), 4.56 (s, 1H), 3.60-3.40 (m, 4H), 3.22-3.12 (m, 1H), 3.07-2.82 (m, 2H), 2.17-2.07 (m, 1H), 2.02-1.92 (m, 1H), 1.92-1.80 (m, 1H), 1.67-1.57 (m, 4H), 1.56-1.47 (m, 7H), 1.46-1.41 (m, 2H), 1.40-1.32 (m, 6H), 1.32-1.24 (m, 4H), 1.23-1.18 (m, 1H), 1.18-1.11 (m, 1H), 0.98-0.92 (m, 9H), 0.91-0.84 (m, 3H), 0.82 (s, 3H), 0.75 (s, 3H), 0.71-0.65 (m, 1H); ES MS: [M+H]⁺ 524.0 (100%).

### Step 5: (1S,3R)-((1R,3aS,5aS,5bR,7aR,9S,11aR,11bR,13aR,13bR)-5a,5b,8,8,11a,13a-hexa methyl-3a-(piperidine-1-carbonyl)-1-(prop-1-en-2-yl)icosahydro-1H-cyclopenta[a]chrysen-9-yl) 3-(2-methoxy-2-oxoethyl)-2,2-dimethylcyclopropanecarboxylate

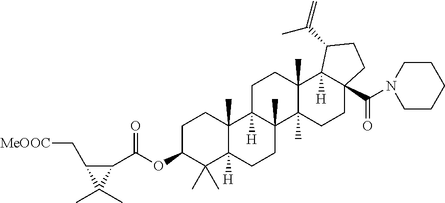

To a stirred solution of ((1R,3aS,5aR,5bR,7aR,9S,11aR,11bR,13aR,13bR)-9-hydroxy-5a,5b,8,8,11a-penta methyl-1-(prop-1-en-2-yl)icosahydro-1H-cyclopenta[a]chrysen-3a-yl)(piperidin-1-yl)methanone (step 4, 0.48 g, 0.91 mmol, 1.0 eq) (1S,3R)-3-(2-methoxy-2-oxoethyl)-2,2-dimethylcyclopropanecarboxylic acid (Example 1-step 1, 0.32 g, 1.83 mmol, 2.0 eq) in THF (10 ml), DIPEA (0.71 g, 5.5 mmol, 6.0 eq) and DMAP (0.279 g, 2.29 mmol, 2.5 eq) were added sequentially then reaction mixture was cooled to 0° C. then 2,4,6-trichlorobenzoyl chloride (0.67 g, 2.75 mmol, 3.0 eq) was added and the reaction mixture was allowed to stir at room temperature for overnight. After completion of the reaction monitored by TLC, the solvent was evaporated in vacuo. The reaction mixture was diluted with CH₂Cl₂ (50 ml), washed with water, brine solution, dried over Na₂SO₄, filtered, evaporated and the title crude compound (0.65 g) was used as such for next step.

### Step 6: 2-((1R,3S)-2,2-dimethyl-3-(((1R,3aS,5aR,5bR,7aR,9S,11aR,11bR,13aR,13bR)-5a,5b,8,8,11a-pentamethyl-3a-(piperidine-1-carbonyl)-1-(prop-1-en-2-yl)icosahydro-1H-cyclopenta[a]chrysen-9-yloxy)carbonyl)cyclopropyl)acetic acid

To a stirred solution of (1S,3R)-((1R,3aS,5aS,5bR,7aR,9S,11aR,1 bR,13aR,13bR)-5a,5b,8,8,11a,13a-hexa methyl-3a-(piperidine-1-carbonyl)-1-(prop-1-en-2-yl)icosahydro-1H-cyclopenta[a]chrysen-9-yl) 3-(2-methoxy-2-oxoethyl)-2,2-dimethylcyclopropanecarboxylate (step 5, 0.65 g, 0.93 mmol, 1.0 eq) in THF:H₂O (7:1, 8 ml) Lithium hydroxide monohydrate (0.276 g, 6.57 mmol, 7.0 eq) was added and stirred at room temperature for overnight. After completion of the reaction monitored by TLC, the solvent was evaporated under reduced pressure then the reaction mixture was cooled to 0° C. and acidified with 1N HCl to pH 5. Aqueous layer was extracted with CH₂Cl₂, the combined organic extracts were washed with water, brine solution, dried over Na₂SO₄, filtered, evaporated and purified by silicagel column chromatography using 16% EtOAc:hexane as eluent to afford the title compound (120 mg, 20% yield over 2 steps) as a white solid. ¹H NMR (300 MHz, CDCl₃): δ 9.82 (brs, 1H), 4.72 (s, 1H), 4.57 (s, 1H), 4.48-4.43 (m, 1H), 3.59-3.41 (m, 4H), 3.03-2.85 (m, 1H), 2.81 (d, 2H, J=7.5 Hz), 2.18-2.07 (m, 1H), 2.03-1.92 (m, 1H), 1.92-1.82 (m, 1H), 1.80-1.72 (m, 1H), 1.65-1.45 (m, 14H), 1.45-1.34 (m, 10H), 1.30-1.25 (m, 3H), 1.20 (s, 3H), 1.18 (s, 3H), 1.15-1.09 (m, 2H), 1.07-0.98 (m, 1H), 0.94 (s, 6H), 0.84 (s, 9H), 0.89-0.75 (m, 1H); ESI MS: [M+H]⁺ 678.1 (50%), [M+Na]⁺ 700.1 (10%); HPLC: 95.2%

### Example 3

### Preparation of 2,2-dimethyl-4-oxo-4-((1R,3aS,5aR,5bR,7aR,9S,11aR,11bR,13aR,13bR)-5a,5b,8,8,11a-pentamethyl-1-(1-methylcyclopropyl)-3a-(piperidine-1-carbonyl)icosahydro-1H-cyclopenta[a]chrysen-9-yloxy)butanoic acid

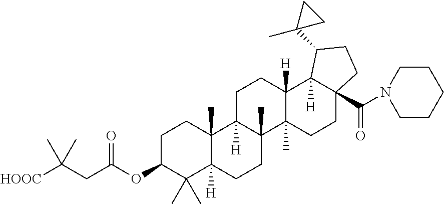

### Step 1: ((1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysen-3a-yl)(piperidin-1-yl)methanone

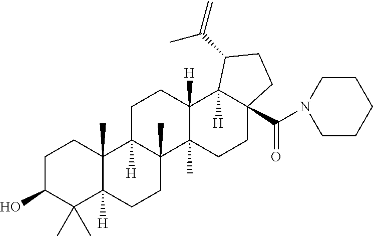

To a stirred solution of (1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysene-3a-carboxylic acid (0.7 g, 1.53 mmol, 1.0 eq) in DMF (7 ml) HATU (0.75 g, 1.99 mmol, 1.3 eq) and DIPEA (1.08 g, 8.42 mmol, 5.5 eq) were added sequentially and stirred at room temperature for about 1 hour then piperidine (0.260 g, 3.06 mmol, 2.0 eq) was added and stirred at room temperature for overnight. After completion of the reaction monitored by TLC, the reaction mixture was diluted with water, extracted with EtOAc (2×25 ml), the combined organic layers were washed with water, brine solution, dried over Na₂SO₄, filtered and evaporated. The crude residue was purified by silicagel column chromatography using 7% EtOAc:Hexane as eluent to obtain the title compound (0.750 g, 93% yield) as a white solid. ¹H NMR (300 MHz, CDCl₃): δ 4.72 (s, 1H), 4.56 (s, 1H), 3.60-3.40 (m, 4H), 3.22-3.12 (m, 1H), 3.07-2.82 (m, 2H), 2.17-2.07 (m, 1H), 2.02-1.92 (m, 1H), 1.92-1.80 (m, 1H), 1.67-1.57 (m, 4H), 1.56-1.47 (m, 7H), 1.46-1.41 (m, 2H), 1.40-1.32 (m, 6H), 1.32-1.24 (m, 4H), 1.23-1.18 (m, 1H), 1.18-1.11 (m, 1H), 0.98-0.92 (m, 9H), 0.91-0.84 (m, 3H), 0.82 (s, 3H), 0.75 (s, 3H), 0.71-0.65 (m, 1H); ES MS: [M+H]⁺ 524.0 (100%).

### Step 2: ((1R,3aS,5aR,5bR,7aR,9S,11aR,11bR,13aR,13bR)-9-hydroxy-5a,5b,8,8,11a-penta methyl-1-(1-methylcyclopropyl)icosahydro-1H-cyclopenta[a]chrysen-3a-yl)(piperidin-1-yl)methanone

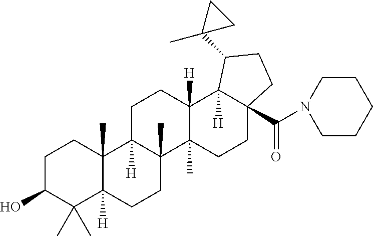

To a stirred solution of ((1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysen-3a-yl)(piperidin-1-yl)methanone (step 1, 0.500 g, 0.95 mmol, 1.0 eq) in dry CH₂Cl₂ (5 ml) cooled to −23° C. then diethylzinc (4.78 ml, 4.78 mmol, 5.0 eq, 1.0 M solution in hexane) was added under nitrogen and stirred for about 30 minutes at the same temperature then diiodomethane (2.04 g, 7.64 mmol, 8.0 eq) was added and reaction was allowed to stir at room temperature for overnight. The reaction mixture was quenched with saturated NH₄Cl solution, acidified with 1N HCl to pH 5, extracted with CH₂Cl₂, the combined organic layers were washed with water, brine solution, dried over Na₂SO₄, filtered and evaporated. The crude residue was purified by silicagel column chromatography using 10% EtOAc:Hexane as eluent to obtain the title compound (0.160 g, 31% yield) as a white solid. ¹H NMR (300 MHz, CDCl₃): δ 3.97 (brs, 1H), 3.60-3.40 (m, 4H), 3.23-3.13 (m, 1H), 2.85-2.75 (m, 1H), 2.15-1.90 (m, 3H), 1.82-1.60 (m, 6H), 1.53-1.47 (m, 6H), 1.43-1.40 (m, 2H), 1.33-1.30 (m, 3H), 1.32-1.30 (m, 2H), 1.30-1.23 (m, 5H), 1.17-1.10 (m, 2H), 0.97 (s, 3H), 0.96 (s, 3H), 0.94 (s, 3H), 0.89 (s, 3H), 0.83 (s, 3H), 0.76 (s, 3H), 0.72-0.68 (m, 1H), 0.48-0.40 (m, 1H), 0.37-0.28 (m, 1H), 0.28-0.13 (m, 2H).

### Step 3: 2,2-dimethyl-4-oxo-4-((1R,3aS,5aR,5bR,7aR,9S,11aR,11bR,13aR,13bR)-5a,5b,8,8,11a-pentamethyl-1-(1-methylcyclopropyl)-3a-(piperidine-1-carbonyl)icosahydro-1H-cyclopenta[a]chrysen-9-yloxy)butanoic acid

To a stirred solution of ((1R,3aS,5aR,5bR,7aR,9S,11aR,11bR,13aR,13bR)-9-hydroxy-5a,5b,8,8,11a-penta methyl-1-(1-methylcyclopropyl)icosahydro-1H-cyclopenta[a]chrysen-3a-yl)(piperidin-1-yl)methanone (step 2, 0.16 g, 0.29 mmol, 1.0 eq) in ethyl acetate (2 ml) 2,2-dimethylsuccinicanhydride (0.114 g, 0.89 mmol, 3.0 eq) and DMAP (0.109 g, 0.89 mmol, 3.0 eq) were added sequentially then refluxed at about 100° C. for overnight. After completion of the reaction monitored by TLC, the reaction mixture was cooled to 0° C., acidified with 1.0 N citric acid solution, organic layer was separated and extracted with ethyl acetate (2×30 ml). The combined organic layers were washed with water, brine solution, dried over Na₂SO₄, filtered, evaporated and purified by silicagel column chromatography using 10% EtOAc:hexane as eluent to afford the title compound (50 mg, 25% yield) as an off-white solid. ¹H NMR (300 MHz, DMSO-d6): δ 12.1 (s, 1H), 4.37 (dd, 1H, J=10.8, 4.8 Hz), 3.52-3.37 (m, 4H), 2.82-2.72 (m, 1H), 2.45-2.39 (m, 2H), 2.12-2.04 (m, 1H), 1.98-1.85 (m, 2H), 1.70-1.50 (m, 8H), 1.48-1.28 (m, 12H), 1.28-1.22 (m, 5H), 1.18-1.15 (m, 6H), 1.12-1.07 (m, 1H), 0.95 (s, 3H), 0.90-0.73 (m, 16H), 0.37-0.27 (m, 2H), 0.23-0.13 (m, 2H); Mass: [M+H]⁺ 666.5 (70%); HPLC: 98.6%.

### Example 4

### Preparation of 4-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(1-methyl cyclopropyl)icosahydro-1H-cyclopenta[a]chrysen-9-yloxy)-2,2-dimethyl-4-oxobutanoicacid

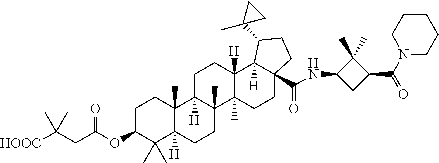

### Step 1: tert-butyl (1R,3S)-2,2-dimethyl-3-(piperidine-1-carbonyl)cyclobutylcarbamate

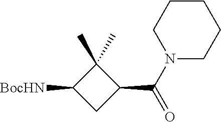

To a stirred solution of (1S,3R)-3-(tert-butoxycarbonylamino)-2,2-dimethylcyclobutanecarboxylic acid (1.0 g, 4.11 mmol, 1.0 eq) in CH₂Cl₂ (10 ml) EDC (1.57 g, 8.23 mmol, 2.0 eq), HOBt (0.83 g, 6.17 mmol, 1.5 eq) and triethylamine (1.71 ml, 12.3 mmol, 3.0 eq) were added sequentially and stirred at room temperature for about 10 minutes then piperidine (0.525 g, 6.17 mmol, 1.5 eq) was added stirred at room temperature for overnight. The reaction mixture was diluted with water, extracted with CH₂Cl₂ (2×20 ml), the combined organic layers were washed with brine solution, dried over Na₂SO₄, filtered and evaporated. The crude residue was purified by silicagel column chromatography using 2% MeOH:CH₂Cl₂ as eluent to obtain the title compound (0.750 g, 59% yield) as a white solid. ¹H NMR (300 MHz, CDCl₃): δ 4.77 (d, 1H, J=8.1 Hz), 3.83 (q, 1H, J=8.7 Hz), 3.77-3.64 (m, 1H), 3.50-3.30 (m, 3H), 2.83-2.74 (m, 1H), 2.40-2.20 (m, 2H), 1.70-1.60 (m, 3H), 1.60-1.49 (m, 3H), 1.43 (brs, 9H), 1.33 (s, 3H), 0.87 (s, 3H).

### Step 2: ((1S,3R)-3-amino-2,2-dimethylcyclobutyl)(piperidin-1-yl)methanonehydrochloride

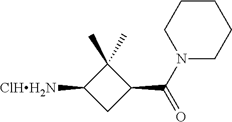

To tert-butyl (1R,3S)-2,2-dimethyl-3-(piperidine-1-carbonyl)cyclobutylcarbamate (0.75 g) 3M HCl in methanol (10 ml) was added at 0° C. and the reaction mixture was allowed to stir at room temperature for overnight. The reaction monitored by TLC shows the starting material was disappeared, solvent was evaporated under reduced pressure and used as such for next step. ¹H NMR (300 MHz, CDCl₃): δ 8.26 (brs, 3H), 3.62-3.52 (m, 1H), 3.40-3.22 (m, 4H), 3.12-3.02 (m, 1H), 2.46-2.40 (m, 1H), 2.08-1.95 (m, 1H), 1.62-1.49 (m, 3H), 1.48-1.34 (m, 3H), 1.29 (s, 3H), 0.93 (s, 3H).

### Step 3: (1R,3aS,5aR,5bR,7aR,9S,11aR,11bR,13aR,13bR)-9-acetoxy-5a,5b,8,8,11a-penta methyl-1-(1-methylcyclopropyl)icosahydro-1H-cyclopenta[a]chrysene-3a-carboxylic acid

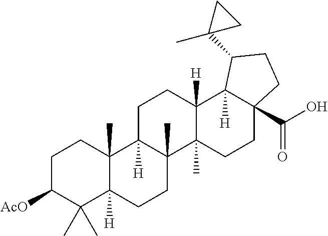

To a stirred solution of (1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxylic acid (Example 2-step 1, 0.700 g, 1.40 mmol, 1.0 eq) in dry CH₂Cl₂ (15 ml) cooled to −20° C. then diethylzinc (7 ml, 7.02 mmol, 5.0 eq, 1.0 M solution in hexane) was added under nitrogen and stirred for about 30 minutes at the same temperature then Diiodomethane (3.0 g, 11.2 mmol, 8.0 eq) was added to the reaction mixture and allowed to stir at room temperature for overnight. The reaction mixture cooled to 0° C., quenched with saturated NH₄Cl solution, extracted with CH₂Cl₂, the combined organic layers were washed with water and brine solution, dried over Na₂SO₄, filtered and evaporated. The crude residue was purified by silicagel column chromatography using 3% EtOAc:hexane as eluent to obtain the title compound (0.400 g, 55.8% yield) as a white solid. ¹H NMR (300 MHz, CDCl₃): δ 4.52-4.42 (m, 1H), 2.27-2.18 (m, 1H), 2.15-2.07 (m, 1H), 2.05 (s, 3H), 2.0-1.86 (m, 2H), 1.78-1.59 (m, 6H), 1.55-1.42 (m, 6H), 1.37-1.30 (m, 3H), 1.29-1.24 (m, 3H), 1.22-1.12 (m, 1H), 1.09-1.02 (m, 1H), 0.98 (s, 3H), 0.94-0.90 (m, 6H), 0.89-0.78 (m, 10H), 0.42-0.32 (m, 2H), 0.30-0.18 (m, 2H).

### Step 4: (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-yl acetate

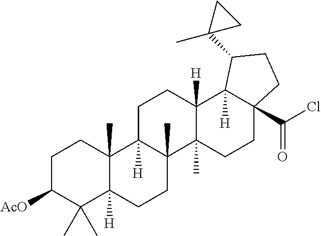

To a stirred solution of (1R,3aS,5aR,5bR,7aR,9S,11aR,11bR,13aR,13bR)-9-acetoxy-5a,5b,8,8,11a-penta methyl-1-(1-methylcyclopropyl)icosahydro-1H-cyclopenta[a]chrysene-3a-carboxylic acid (step 3, 0.300 g, 0.585 mmol, 1.0 eq) in toluene (5 ml) at 0° C. thionyl chloride (0.21 ml, 2.92 mmol, 5.0 eq) was added and the reaction was heated to reflux for about 3 hours. The solvent was evaporated in vacuo, again toluene (10 ml) was added and evaporated to obtain the title crude compound (0.3 g), which is used as such for next step.

### Step 5: (1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(1-methyl cyclopropyl)icosahydro-1H-cyclopenta[a]chrysen-9-yl acetate

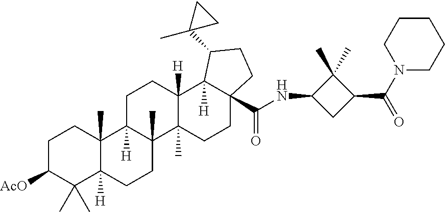

To a chilled (0° C.) suspension of ((1S,3R)-3-amino-2,2-dimethylcyclobutyl)(piperidin-1-yl)methanonehydrochloride (step 2, 0.139 g, 0.56 mmol, 1.0 eq) in CH₂Cl₂ (10 ml) DIPEA (0.48 ml, 2.82 mmol, 5.0 eq) was added then a solution of (1R,3aS,5aR,5bR,7aR,9S,11aR,11bR,13aR,13bR)-3a-(chlorocarbonyl)-5a,5b,8,8,8,11a pentamethyl-1-(1-methylcyclopropyl)icosahydro-1H-cyclopenta[a]chrysen-9-yl acetate (step 4, 0.3 g, 0.56 mmol, 1.0 eq) in CH₂Cl₂ (5 ml) was added and the reaction mixture was allowed to room temperature then stirred at the same temperature for overnight. The reaction mixture was diluted with CH₂Cl₂ (10 ml), washed with water and brine solution, dried over Na₂SO₄, filtered and evaporated. The crude residue was purified by silicagel column chromatography using 4% MeOH:CH₂Cl₂ as eluent to afford the title compound (0.29 g, 72.8% yield) as a white solid. ¹H NMR (300 MHz, CDCl₃): δ 5.82 (d, 1H, J=8.1 Hz), 4.52-4.44 (m, 1H), 4.12-4.02 (m, 1H), 3.77-3.66 (m, 1H), 3.50-3.30 (m, 3H), 2.86 (t, 1H, J=8.1 Hz), 2.43-2.19 (m, 3H), 2.04 (s, 3H), 2.0-1.8 (m, 3H), 1.80-1.60 (m, 6H), 1.53-1.42 (m, 7H), 1.40-1.31 (m, 8H), 1.30-1.24 (m, 3H), 1.20-1.09 (m, 3H), 1.05-1.0 (m, 1H), 0.97 (s, 3H), 0.95 (s, 3H), 0.92-0.89 (m, 4H), 0.89-0.81 (m, 12H), 0.80-0.78 (m, 1H), 0.47-0.38 (m, 1H), 0.38-0.30 (m, 1H), 0.29-0.13 (m, 2H).

### Step 6: (1R,3aS,5aR,5bR,7aR,9S,11aR,11bR,13aR,13bR)—N-((1R,3S)-2,2-dimethyl-3-(piperidine-1-carbonyl)cyclobutyl)-9-hydroxy-5a,5b,8,8,11a-pentamethyl-1-(1-methyl cyclopropyl)icosahydro-1H-cyclopenta[a]chrysene-3a-carboxamide

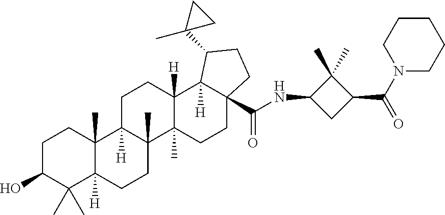

To a stirred solution of (1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(1-methyl cyclopropyl)icosahydro-1H-cyclopenta[a]chrysen-9-yl acetate (step 5, 0.28 g, 0.39 mmol, 1.0 eq) in THF:MeOH (1:1, 6 ml) potassium carbonate (0.164 g, 1.19 mmol, 3.0 eq) was added and stirred at room temperature for about 48 hours. The reaction mixture was filtered through Buchner funnel, washed with EtOAc:MeOH (20 ml), filtrate was evaporated under reduced pressure and purified by silica gel column chromatography by using 10% EtOAc:hexane as eluent to afford the title compound (0.25 g) as a white solid. ¹H NMR (300 MHz, CDCl₃): δ 5.82 (d, 1H, J=7.8 Hz), 4.12-4.03 (m, 1H), 3.78-3.67 (m, 1H), 3.48-3.30 (m, 3H), 3.25-3.14 (m, 1H), 2.86 (t, 1H, J=8.1 Hz), 2.43-2.20 (m, 3H), 2.0-1.83 (m, 3H), 1.82-1.77 (m, 2H), 1.73-1.69 (m, 6H), 1.67-1.47 (m, 6H), 1.40-1.35 (m, 7H), 1.32-1.25 (m, 4H), 1.22-1.05 (m, 3H), 0.97 (s, 3H), 0.96 (s, 3H), 0.94-0.87 (m, 7H), 0.85 (s, 3H), 0.82 (s, 3H), 0.75 (s, 3H), 0.72-0.68 (m, 1H), 0.47-0.38 (m, 1H), 0.38-0.28 (m, 1H), 0.28-0.14 (m, 2H).

### Step 7: 4-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(1-methyl cyclopropyl)icosahydro-1H-cyclopenta[a]chrysen-9-yloxy)-2,2-dimethyl-4-oxobutanoicacid

To a stirred solution of (1R,3aS,5aR,5bR,7aR,9S,11aR,11bR,13aR,13bR)—N-((1R,3S)-2,2-dimethyl-3-(piperidine-1-carbonyl)cyclobutyl)-9-hydroxy-5a,5b,8,8,11a-pentamethyl-1-(1-methyl cyclopropyl)icosahydro-1H-cyclopenta[a]chrysene-3a-carboxamide (step-6, 0.25 g, 0.37 mmol, 1.0 eq) in ethyl acetate (3 ml) 2,2-dimethylsuccinicanhydride (0.072 g, 0.56 mmol, 1.5 eq) and DMAP (0.092 g, 0.75 mmol, 2.0 eq) were added sequentially then refluxed at about 100° C. for overnight. After completion of the reaction monitored by TLC, the reaction mixture was cooled to 0° C., diluted with EtOAc (20 ml), acidified with 1 N citric acid solution to pH 5 and two layers were separated. Aqueous layer was extracted with EtOAc, the combined organic layers were washed with brine solution, dried over Na₂SO₄, filtered, evaporated and purified by silica gel column chromatography by using 22% EtOAc:hexane as eluent to afford the title compound (0.130 g, 43.6% yield) as an off-white solid. ¹H NMR (300 MHz, CDCl₃): δ 5.94 (d, 1H, J=7.8 Hz), 4.57-4.45 (m, 1H), 4.14-4.05 (m, 1H), 3.78-3.67 (m, 1H), 3.50-3.30 (m, 3H), 2.87 (t, 1H, J=8.1 Hz), 2.73-2.53 (m, 3H), 2.45-2.33 (m, 2H), 2.28-2.18 (m, 1H), 2.02-1.92 (m, 2H), 1.90-1.40 (m, 17H), 1.40-1.25 (m, 16H), 1.20-1.10 (m, 2H), 0.97 (s, 3H), 0.92 (s, 3H), 0.89 (s, 3H), 0.87-0.84 (m, 9H), 0.79 (s, 3H), 0.78-0.73 (m, 1H), 0.47-0.38 (m, 1H), 0.38-0.28 (m, 1H), 0.28-0.13 (m, 2H); ESI MS: [M+H]⁺ 791.7 (30%); HPLC: 82.4%+12.1% (regio isomers).

### Example 5

### Preparation of 2-((1R,3S)-2,2-dimethyl-3-(((1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosa hydro-1H-cyclopenta[a]chrysen-9-yloxy)carbonyl)cyclopropyl)acetic acid

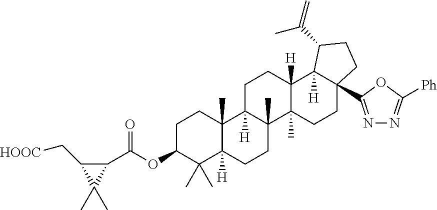

### Step 1: (1R,3aS,5aR,5bR,7aR,9S,11aR,11bR,13aR,13bR)-3a-(2-benzoylhydrazinecarbonyl)-5a,5b,8,8,11a-pentamethyl-1-(prop-1-en-2-yl)icosahydro-1H-cyclopenta[a]chrysen-9-yl acetate

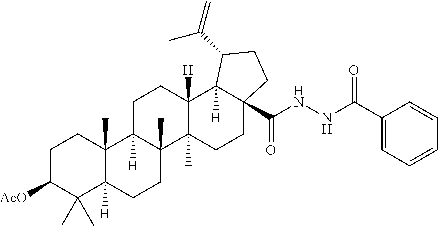

To a solution of benzoic hydrazide (0.39 g, 2.90 mmol, 1.0 eq) in DMF (7 ml) DIPEA (1.87 g, 14.5 mmol, 5.0 eq) was added at 0° C. A solution of (1R,3aS,5aR,5bR,7aR,9S,11aR,11bR,13aR,13bR)-3a-(chlorocarbonyl)-5a,5b,8,8,11a-pentamethyl--(prop-1-en-2-yl)icosahydro-1H-cyclopenta[a]chrysen-9-yl acetate (Example-step 2, 1.5 g, 2.90 mmol, 1.0 eq) in CH₂Cl₂ (7 ml) was added to the reaction mixture and allowed to stir at room temperature for overnight. TLC indicated starting material was consumed and the desired product was formed. The reaction mixture was diluted with water and extracted with CH₂Cl₂. The combined organic layers were dried over Na₂SO₄, filtered, evaporated and purified by silicagel column chromatography using 6% ethyl acetate/hexane as eluent to give the desired title compound (1.5 g, 60% yield) as a white solid. ¹H NMR (300 MHz, CDCl₃): δ ppm 8.88 (d, 1H, J=6.3 Hz), 8.51 (d, 1H, J=6.3 Hz), 7.83 (d, 2H, J=7.5 Hz), 7.60-7.52 (m, 1H), 7.50-7.44 (m, 2H), 4.74 (s, 1H), 4.61 (s, 1H), 4.50-4.43 (m, 1H), 3.15-3.05 (m, 1H), 2.45-2.35 (m, 1H), 2.15-2.07 (m, 1H), 2.05 (s, 3H), 2.0-1.9 (m, 2H), 1.70-1.68 (m, 4H), 1.67-1.60 (m, 5H), 1.54-1.41 (m, 4H), 1.40-1.38 (m, 3H), 1.34-1.30 (m, 1H), 1.10-1.0 (m, 2H), 1.0-0.95 (m, 6H), 0.92-0.87 (m, 3H), 0.85-0.82 (m, 9H), 0.82-0.75 (m, 1H); ES MS: [M−H]⁻ 615.5 (50%).

### Step-2: (1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosahydro-1H-cyclopenta[a]chrysen-9-yl acetate

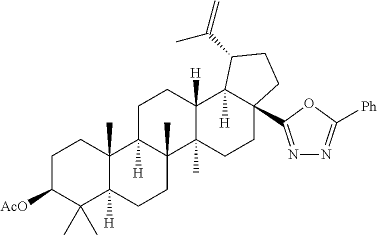

To a solution of (1R,3aS,5aR,5bR,7aR,9S,11aR,11bR,13aR,13bR)-3a-(2-benzoylhydrazinecarbonyl)-5a,5b,8,8,11a-pentamethyl-1-(prop-1-en-2-yl)icosahydro-1H-cyclopenta[a]chrysen-9-yl acetate (step-1, 2.0 g, 3.24 mmol, 1.0 eq) and triethylamine (0.98 g, 9.73 mmol, 3.0 eq) in CH₂Cl₂ (20 ml) para-toluenesulfonylchloride (0.742 g, 3.89 mmol, 1.2 eq) was added and the reaction mixture was stirred at room temperature for overnight. TLC indicated starting material was consumed and the desired product was observed. The reaction mixture was diluted with CH₂Cl₂, washed with water and brine solution. The organic layer was dried over Na₂SO₄, filtered and evaporated under reduced pressure. The residue was purified by silicagel column chromatography using 2% ethyl acetate:hexane as eluent to afford the desired title compound (1.5 g, 79% yield) as a white solid. ¹H NMR (300 MHz, CDCl₃): δ ppm 8.10-8.02 (m, 2H), 7.58-7.47 (m, 3H), 4.79 (s, 1H), 4.65 (s, 1H), 4.50-4.40 (m, 1H), 3.23-3.12 (m, 1H), 2.50-2.30 (m, 2H), 2.08-2.04 (m, 3H), 1.97-1.80 (m, 2H), 1.76-1.73 (m, 4H), 1.72-1.63 (m, 3H), 1.52-1.38 (m, 4H), 1.37-1.25 (m, 5H), 1.24-1.07 (m, 3H), 1.02 (s, 3H), 1.0-0.93 (m, 1H), 0.92-0.87 (m, 2H), 0.85-0.83 (m, 6H), 0.83-0.79 (m, 6H), 0.78-0.75 (m, 1H); ES MS: [M+H]⁺ 599.5 (100%).

### Step-3: (1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosahydro-1H-cyclopenta[a]chrysen-9-ol

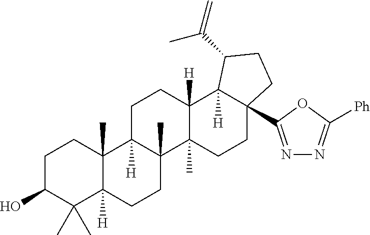

To a solution of (1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosahydro-1H-cyclopenta[a]chrysen-9-yl acetate (step-2, 1.5 g, 2.50 mmol, 1.0 eq) in THF (7 ml) and MeOH (7 ml) potassium carbonate (1.38 g, 10.0 mmol, 4.0 eq) was added and the reaction mixture was stirred at room temperature for 48 hours. TLC indicated starting material was consumed and the desired product was observed. The reaction mixture was filtered through a pad of celite, which was washed with dichloromethane. The mixture was concentrated under reduced pressure, diluted with water (1×40 ml) and extracted with DCM (2×40 ml). The combined organic layers were dried over sodiumsulphate and concentrated under reduced pressure. The residue was purified by silicagel column chromatography using 10% ethyl acetate/hexane as eluent to afford the desired title compound (0.800 g, 57% yield) as a white solid. ¹H NMR (300 MHz, CDCl₃): δ ppm 8.10-8.02 (m, 2H), 7.58-7.48 (m, 3H), 4.79 (s, 1H), 4.65 (s, 1H), 3.22-3.10 (m, 2H), 2.50-2.35 (m, 2H), 2.10-2.0 (m, 1H), 1.98-1.80 (m, 3H), 1.74 (s, 3H), 1.72-1.62 (m, 3H), 1.52-1.38 (m, 5H), 1.37-1.32 (m, 2H), 1.32-1.23 (m, 3H), 1.22-1.06 (m, 3H), 1.03 (s, 3H), 0.95 (s, 3H), 0.92-0.88 (m, 1H), 0.84 (s, 3H), 0.79 (s, 3H), 0.68 (s, 3H), 0.70-0.65 (m, 1H); ES MS: [M+H]⁺ 557.4 (100%).

### Step-4: (1S,3R)-((1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosahydro-1H-cyclopenta[a]chrysen-9-yl)3-(2-methoxy-2-oxoethyl)-2,2-dimethylcyclopropanecarboxylate

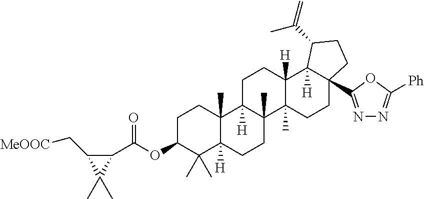

To a mixture of (1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosahydro-1H-cyclopenta[a]chrysen-9-ol (step-3, 0.300 g, 0.53 mmol, 1.0 eq), (1S,3R)-3-(2-methoxy-2-oxoethyl)-2,2-dimethylcyclopropanecarboxylic acid (Example 1-step 1, 0.200 g, 1.07 mmol, 2.0 eq), DIPEA (0.41 g, 3.21 mmol, 6.0 eq) and 4-dimethylaminopyridine (0.163 g, 1.34 mmol, 2.5 eq) in THF (5 ml) 2,4,6-trichlorobenzoyl chloride (0.39 g, 1.60 mmol, 3.0 eq) was added at 0° C. and the reaction mixture was allowed to stir at room temperature for overnight. After completion of the reaction monitored by TLC, the solvent was evaporated under reduced pressure, reaction mixture diluted with water and extracted with CH₂Cl₂ (3×15 ml). The combined organic layers were dried over Na₂SO₄, filtered and evaporated under reduced pressure. The residue was purified by silicagel column chromatography using 5% ethyl acetate/hexane as eluent to give the desired title compound (0.390 g) as a white solid. ¹H NMR (300 MHz, CDCl₃): δ ppm 8.10-8.02 (m, 2H), 7.57-7.48 (m, 3H), 4.79 (s, 1H), 4.65 (s, 1H), 4.50-4.40 (m, 1H), 3.66 (s, 3H), 3.22-3.10 (m, 1H), 2.76 (d, 2H, J=7.5 Hz), 2.50-2.35 (m, 3H), 2.09-1.81 (m, 5H), 1.74 (s, 3H), 1.70-1.62 (m, 3H), 1.54-1.53 (m, 1H), 1.52-1.45 (m, 2H), 1.44-1.38 (m, 3H), 1.37-1.29 (m, 4H), 1.28-1.24 (m, 3H), 1.23-1.17 (m, 6H), 1.16-1.05 (m, 2H), 1.04-1.01 (m, 2H), 0.97-0.85 (m, 3H), 0.84-0.79 (m, 9H), 0.79-0.73 (m, 1H); ES MS: [M+H]⁺ 725.4 (100%).

### Step-5: 2-((1R,3S)-2,2-dimethyl-3-(((1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl) icosa hydro-1H-cyclopenta[a]chrysen-9-yloxy)carbonyl)cyclopropyl)acetic acid

To a stirred solution of (1S,3R)-((1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosahydro-1H-cyclopenta[a]chrysen-9-yl)3-(2-methoxy-2-oxoethyl)-2,2-dimethylcyclopropanecarboxylate (step-4, 0.390 g, 0.53 mmol, 1.0 eq) in THF (4 ml) and H₂O (1 ml) Lithium hydroxide monohydrate (0.067 g, 1.61 mmol, 3.0 eq) was added and the reaction mixture was stirred at room temperature for overnight. After completion of the reaction monitored by TLC, the solvent was evaporated under reduced pressure. The reaction mixture was cooled to 0° C., acidified to pH=4-5 using 1N citric acid solution and extracted with CH₂Cl₂. The combined organic extracts were washed with water, dried over Na₂SO₄, filtered and evaporated under reduced pressure. The residue was purified by silicagel column chromatography using 15% ethyl acetate/hexane as eluent to give the desired title compound (0.090 g, 23% yield over 2 steps) as a white solid. ¹H NMR (300 MHz, CDCl₃): δ ppm 10.1 (brs, 1H), 8.07-8.04 (m, 2H), 7.53-7.51 (m, 3H), 4.79 (s, 1H), 4.65 (s, 1H), 4.48-4.43 (m, 1H), 3.22-3.10 (m, 1H), 2.81 (d, 2H, J=7.5 Hz), 2.48-2.36 (m, 2H), 2.10-2.0 (m, 1H), 1.98-1.80 (m, 3H), 1.78-1.72 (m, 4H), 1.70-1.62 (m, 3H), 1.60-1.54 (m, 3H), 1.52-1.30 (m, 8H), 1.30-1.25 (m, 2H), 1.20 (s, 3H), 1.18 (s, 3H), 1.16-1.05 (m, 1H), 1.02 (s, 3H), 0.98-0.92 (m, 1H), 0.87-0.81 (m, 12H), 0.79-0.75 (m, 1H); ES MS: [M+H]⁺ 711.5 (100%), [M+Na]⁺ 733.6 (20%); HPLC: 94.5%.

### Example 6

### Preparation of 2-((1R,3S)-2,2-dimethyl-3-(((1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl)icosahydro-1H-cyclopenta[a]chrysen-9-yloxy)carbonyl)cyclopropyl)aceticacid

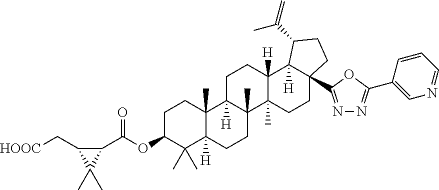

### Step-1: (1R,3aS,5R,aR,5bR,7aR,9S,1aR,1bR,3aR,13bR)-3a-(hydrazinecarbonyl)-5a,5b,8,8,11a-pentamethyl-1-(prop-1-en-2-yl)icosahydro-1H-cyclopenta[a]chrysen-9-yl acetate

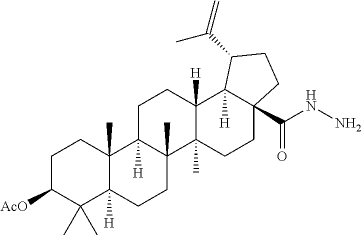

To a stirred solution of (1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxylic acid (Example 2-step 1, 2.0 g, 4.01 mmol, 1.0 eq) and DIPEA (2.84 g, 22.08 mmol, 5.5 eq) in DMF (20 ml) HBTU (1.92 g, 5.22 mmol, 1.3 eq) was added and the reaction mixture was stirred at room temperature for 1 hour then hydrazine hydrate (2.0 g, 40.16 mmol, 10.0 eq) was added and stirred at room temperature for 2 hours. TLC indicated starting material was consumed and the desired product was observed. The reaction mixture was cooled to 0° C. and quenched with ice cooled water, solid was precipitated out. The solids were collected by filtration, washed with water and dried in vacuo at 50° C. for 3 hours to give the desired title compound (2.0 g, 97% yield) as a white solid. ¹H NMR (300 MHz, CDCl₃): δ ppm 6.85 (s, 1H), 4.74 (s, 1H), 4.60 (s, 1H), 4.50-4.43 (m, 1H), 3.85 (brs, 2H), 3.15-3.03 (m, 1H), 2.44-2.32 (m, 1H), 2.04 (s, 3H), 1.98-1.88 (m, 2H), 1.80-1.72 (m, 1H), 1.72-1.68 (m, 4H), 1.65-1.54 (m, 5H), 1.55-1.45 (m, 2H), 1.46-1.30 (m, 7H), 1.30-1.25 (m, 2H), 1.20-1.10 (m, 1H), 1.07-1.0 (m, 1H), 0.98-0.92 (m, 6H), 0.87-0.82 (m, 9H), 0.82-0.75 (m, 1H).

### Nicotinoyl Chloride Preparation

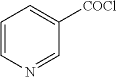

To a compound of nicotinic acid (0.420 g, 3.41 mmol, 1.0 eq) in RB flask thionyl chloride (5 ml) was added at 0° C. The reaction mixture was heated at 50° C. for 24 hours. The reaction mixture was concentrated in vacuo providing a crude residue that was re-dissolved in toluene (20 ml) to remove the excess thionyl chloride. The solution was concentrated in vacuo to produce the desired acid chloride (0.454 g), which was used as such for next step without further purification.

### Step-2: (1R,3aS,5aR,5bR,7aR,9S,11aR,11bR,13aR,13bR)-5a,5b,8,8,11a-pentamethyl-3a-(2-nicotinoylhydrazinecarbonyl)-1-(prop-1-en-2-yl)icosahydro-1H-cyclopenta[a]chrysen-9-yl acetate

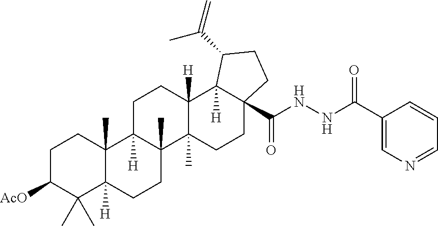

To a solution of (1R,3aS,5aR,5bR,7aR,9S,11aR,11bR,13aR,13bR)-3a-(hydrazinecarbonyl)-5a,5b,8,8,11a-pentamethyl-1-(prop-1-en-2-yl)icosahydro-1H-cyclopenta[a]chrysen-9-yl acetate (step-1, 1.5 g, 2.92 mmol, 1.0 eq) and DIPEA (2.64 g, 20.5 mmol, 7.0 eq) in CH₂Cl₂ (15 ml) at 0° C. a solution of nicotinoyl chloride (0.454 g, 3.22 mmol, 1.1 eq) in CH₂Cl₂ (7 ml) was added and the reaction mixture was allowed to stir at room temperature for overnight. The reaction mixture was diluted with water and extracted with CH₂Cl₂. The combined organic extracts were dried over Na₂SO₄, filtered and evaporated under reduced pressure. The residue was purified by silicagel column chromatography using 2% methanol/dichloromethane as eluent to give the desired title compound (1.4 g, 77% yield) as a white solid. ¹H NMR (300 MHz, CDCl₃): δ ppm 9.41 (d, 1H, J=5.1 Hz), 9.05 (s, 1H), 8.76 (d, 1H, J=3.9 Hz), 8.53 (d, 1H, J=5.1 Hz), 8.16 (d, 1H, J=7.8 Hz), 7.42-7.35 (m, 1H), 4.49 (s, 1H), 4.47 (s, 1H), 4.52-4.43 (m, 1H), 3.12-3.0 (m, 1H), 2.46-2.25 (m, 1H), 2.20-2.10 (m, 1H), 2.05 (s, 3H), 2.0-1.92 (m, 2H), 1.65-1.58 (m, 9H), 1.48-1.20 (m, 10H), 1.10-1.0 (m, 1H), 1.0-0.98 (m, 3H), 0.95-0.92 (m, 3H), 0.92-0.86 (m, 2H), 0.86-0.83 (m, 9H), 0.30-0.27 (m, 1H).

### Step-3: (1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl)icosahydro-1H-cyclopenta[a]chrysen-9-yl acetate

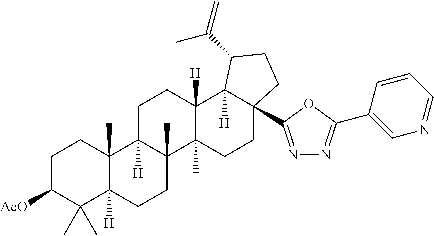

To a solution of (1R,3aS,5aR,5bR,7aR,9S,11aR,11bR,13aR,13bR)-5a,5b,8,8,11a-pentamethyl-3a-(2-nicotinoylhydrazinecarbonyl)-1-(prop-1-en-2-yl)icosahydro-1H-cyclopenta[a]chrysen-9-yl acetate (step-2, 1.4 g, 2.26 mmol, 1.0 eq) and triethylamine (0.68 g, 6.80 mmol, 3.0 eq) in CH₂Cl₂ (15 ml) para-toluenesulfonylchloride (0.519 g, 2.72 mmol, 1.2 eq) was added at 0° C. and the reaction mixture was stirred at room temperature for overnight. TLC indicated starting material was consumed and the desired product was observed. The reaction mixture was diluted with CH₂Cl₂, washed with water and brine solution. The organic extract was dried over Na₂SO₄, filtered, evaporated and purified by silicagel column chromatography using 1% MeOH:CH₂Cl₂ as eluent to afford the desired title compound (1.3 g, 95% yield) as a white solid. ¹H NMR (300 MHz, CDCl₃): δ ppm 9.26 (s, 1H), 8.77 (d, 1H, J=3.6 Hz), 8.37 (d, 1H, J=6.3 Hz), 7.47 (dd, 1H, J=5.1, 7.8 Hz), 4.80 (s, 1H), 4.66 (s, 1H), 4.50-4.42 (m, 1H), 3.18-3.09 (m, 1H), 2.50-2.35 (m, 2H), 2.04 (s, 3H), 1.97-1.85 (m, 2H), 1.84-1.77 (m, 1H), 1.75 (brs, 3H), 1.72-1.68 (m, 2H), 1.67-1.63 (m, 2H), 1.59-1.55 (m, 1H), 1.55-1.39 (m, 5H), 1.38-1.32 (m, 3H), 1.30-1.24 (m, 3H), 1.22-1.05 (m, 3H), 1.04-1.01 (m, 3H), 1.0-0.87 (m, 2H), 0.86-0.81 (m, 9H), 0.80-0.74 (m, 1H); ES MS: [M+H]⁺ 600.4 (70%), [M+Na]⁺ 622.3 (90%).

### Step-4: (1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl)icosahydro-1H-cyclopenta[a]chrysen-9-ol

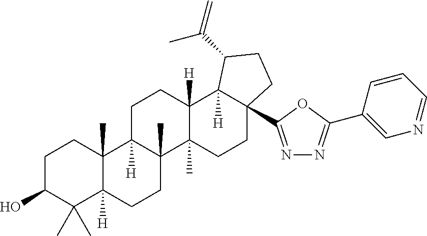

To a solution of (1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl)icosahydro-1H-cyclopenta[a]chrysen-9-yl acetate (step-3, 1.3 g, 2.17 mmol, 1.0 eq) in THF (7 ml) and MeOH (7 ml) potassium carbonate (1.49 g, 10.85 mmol, 5.0 eq) was added and the reaction mixture was stirred at room temperature for 24 hours. TLC indicated starting material was consumed and the desired product was observed. The reaction mixture was filtered through a pad of celite, which was washed with dichloromethane. The mixture was concentrated under reduced pressure, diluted with water (100 ml) and extracted with DCM (2×100 ml). The combined organic layers were dried over sodiumsulphate and concentrated under reduced pressure. The residue was purified by silicagel column chromatography using 2% methanol:dichloromethane as eluent to afford the desired title compound (0.900 g, 75% yield) as a white solid. ¹H NMR (300 MHz, CDCl₃): δ ppm 9.26 (s, 1H), 8.77 (d, 1H, J=3.9 Hz), 8.37 (d, 1H, J=8.1 Hz), 7.47 (dd, 1H, J=4.8, 8.1 Hz), 4.80 (s, 1H), 4.65 (s, 1H), 3.22-3.08 (m, 2H), 2.48-2.34 (m, 2H), 2.12-1.98 (m, 1H), 1.98-1.84 (m, 2H), 1.83-1.77 (m, 1H), 1.74 (s, 3H), 1.72-1.60 (m, 3H), 1.54-1.38 (m, 6H), 1.37-1.20 (m, 6H), 1.20-1.07 (m, 1H), 1.03 (s, 3H), 0.95 (s, 3H), 0.92-0.88 (m, 1H), 0.84 (s, 3H), 0.79 (s, 3H), 0.73 (s, 3H), 0.70-0.64 (m, 1H); ES MS: [M+H]⁺ 558.5 (100%).

### Step-5: (1S,3R)-((1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl)icosahydro-1H-cyclopenta[a]chrysen-9-yl) 3-(2-methoxy-2-oxoethyl)-2,2-dimethylcyclopropanecarboxylate

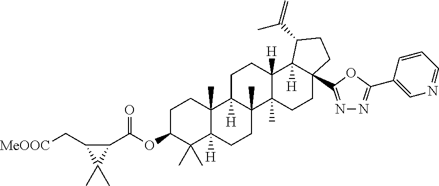

To a mixture of (1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl)icosahydro-1H-cyclopenta[a]chrysen-9-ol (step-4, 0.300 g, 0.53 mmol, 1.0 eq), (1S,3R)-3-(2-methoxy-2-oxoethyl)-2,2-dimethylcyclopropanecarboxylic acid (Example 1-step 1, 0.20 g, 1.07 mmol, 2.0 eq), DIPEA (0.41 g, 3.23 mmol, 6.0 eq) and 4-dimethylaminopyridine (0.163 g, 1.34 mmol, 2.5 eq) in THF (5 ml) 2,4,6-trichlorobenzoyl chloride (0.39 g, 1.61 mmol, 3.0 eq) was added at 0° C. The reaction mixture was allowed to stir at room temperature for overnight. After completion of the reaction monitored by TLC, solvent was evaporated under reduced pressure. The reaction mixture was diluted with water and extracted with CH₂Cl₂ (3×15 ml). The combined organic extracts were dried over Na₂SO₄, filtered and evaporated under reduced pressure. The residue was purified by silicagel column chromatography using 1% methanol:dichloromethane as eluent to give the desired title compound (0.300 g, 77% yield) as a white solid. ¹H NMR (300 MHz, CDCl₃): δ ppm 9.26 (s, 1H), 8.78 (d, 1H, J=3.9 Hz), 8.37 (d, 1H, J=5.1 Hz), 7.48 (dd, 1H, J=5.1, 7.8 Hz), 4.80 (s, 1H), 4.66 (s, 1H), 4.50-4.38 (m, 1H), 3.67 (s, 3H), 3.20-3.10 (m, 1H), 2.82-2.73 (m, 3H), 2.60-2.32 (m, 2H), 2.10-2.0 (m, 1H), 1.98-1.83 (m, 2H), 1.83-1.80 (m, 1H), 1.73-1.64 (m, 2H), 1.63-1.60 (m, 1H), 1.60-1.53 (m, 3H), 1.52-1.47 (m, 3H), 1.46-1.38 (m, 3H), 1.36-1.32 (m, 3H), 1.30-1.18 (m, 11H), 1.15-1.0 (m, 1H), 1.02 (s, 3H), 1.0-0.88 (m, 3H), 0.88-0.78 (m, 9H), 0.80-0.72 (m, 1H).

### Step-6: 2-((1R,3S)-2,2-dimethyl-3-(((1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl) icosahydro-1H-cyclopenta[a]chrysen-9-yloxy) carbonyl)cyclopropyl)aceticacid

To a stirred solution of (1S,3R)-((1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl)icosahydro-1H-cyclopenta[a]chrysen-9-yl) 3-(2-methoxy-2-oxoethyl)-2,2-dimethylcyclopropanecarboxylate (step-5, 0.300 g, 0.41 mmol, 1.0 eq) in THF (4 ml) and H₂O (1 ml) Lithium hydroxide monohydrate (0.052 g, 1.24 mmol, 3.0 eq) was added and the reaction mixture was stirred at room temperature for overnight. TLC indicated starting material was consumed and the desired product was observed. The reaction mixture was evaporated under the reduced pressure, cooled to 0° C., acidified to pH=4-5 using 1N citric acid solution and extracted with CH₂Cl₂. The combined organic extracts were washed with water, dried over Na₂SO₄, filtered and evaporated under reduced pressure.

The residue was purified by silicagel column chromatography using 1% methanol:dichloromethane as eluent to afford the desired title compound (0.160 g, 54% yield) as a white solid. ¹H NMR (300 MHz, CDCl₃): δ ppm 9.26 (brs, 1H), 8.77 (d, 1H, J=3.9 Hz), 8.37 (d, 1H, J=7.8 Hz), 7.48 (dd, 1H, J=5.1, 7.8 Hz), 4.80 (s, 1H), 4.66 (s, 1H), 4.48-4.43 (m, 1H), 3.18-3.09 (m, 1H), 2.81 (d, 2H, J=7.5 Hz), 2.49-2.34 (m, 2H), 2.10-2.02 (m, 1H), 1.97-1.83 (m, 2H), 1.83-1.78 (m, 1H), 1.75 (s, 3H), 1.72-1.63 (m, 3H), 1.60-1.54 (m, 4H), 1.54-1.45 (m, 4H), 1.44-1.37 (m, 3H), 1.35-1.32 (m, 2H), 1.30-1.25 (m, 3H), 1.20 (s, 3H), 1.18 (s, 3H), 1.15-1.06 (m, 1H), 1.02 (s, 3H), 0.98-0.88 (m, 2H), 0.85-0.80 (m, 9H), 0.80-0.72 (m, 1H); ES MS: [M−H]⁻ 710.5 (100%); HPLC: 94.9%.

### Example 7

### Preparation of 4-((1R,3aS,5aR,5bR,7aR,9S,11aR,11bR,13aR,13bR)-3a-(4-(1-methylethyl-2,2,2,1′,1′,1′-D6)piperazine-1-carbonyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid

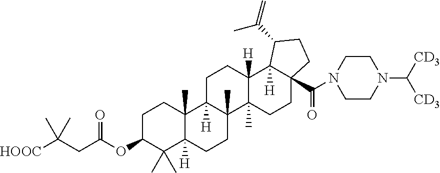

### Step 1: tert-butyl piperazine-1-carboxylate

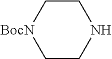 [SMILES: CC(C)(C)OC(=O)N1CCNCC1]

To a solution of piperazine (5.0 g, 58.08 mmol, 1.0 eq) in DCM (100 ml) di-tert-butyldicarbonate (6.33 g, 29.04 mmol, 0.5 eq) in DCM (50 ml) was added drop wise at 0° C. then the mixture was stirred at 0° C. for 1 hour and filtered. The filtrate was concentrated under reduced pressure and water (75 ml) was added to the resulting oil and filtered. The filtrate was saturated with potassium carbonate and extracted with diethyl ether (3×50 ml). The combined organic solvent was dried over Na₂SO₄ and filtered. The solvent was removed under reduced pressure to give title compound (4.8 g, 44% yield) as a yellow solid. ¹H NMR (300 MHz, CDCl₃): δ ppm 3.39 (t, 4H, J=5.1 Hz), 2.81 (t, 4H, J=5.1 Hz), 1.46 (s, 9H).

### Step-2: tert-butyl 4-(1-methylethyl-2,2,2,1′,1′,1′-D6)piperazine-1-carboxylate

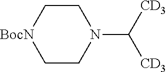 [SMILES: [2H]C([2H])([2H])C(N1CCN(C(=O)OC(C)(C)C)CC1)C([2H])([2H])[2H]]

A solution of N-Boc-piperazine (step-1, 1.0 g, 5.40 mmol, 1.0 eq) in CH₂Cl₂ (20 ml) was mixed with acetone-d6 (0.41 g, 6.48 mmol, 1.2 eq) and acetic acid (0.39 g, 6.48 mmol, 1.2 eq) at 0° C. The resulting mixture was stirred at 0° C. for 5 minutes then sodium triacetoxyborohydride (1.6 g, 7.56 mmol, 1.4 eq) was added then the reaction mixture was allowed to stir at room temperature for overnight. TLC indicated starting material was consumed and the desired product was observed. The reaction mixture was washed with 1N NaOH solution (20 ml), water and brine solution. The organic extract was dried over Na₂SO₄, filtered and evaporated under reduced pressure. The residue was purified by silicagel column chromatography using 4% methanol:dichloromethane as eluent to afford the title compound (1.1 g, 87% yield) as colorless oil. ¹H NMR (300 MHz, CDCl₃): δ ppm 3.54 (t, 4H, J=5.1 Hz), 2.87 (brs, 1H), 2.61 (t, 4H, J=5.1 Hz), 1.46 (s, 9H). ES MS: [M+H]⁺ 235.4 (100%).

### Step-3: 1-(1-methylethyl-2,2,2,1′,1′,1′-D6)piperazinehydrochloride

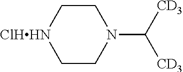

To a compound of tert-butyl 4-(1-methylethyl-2,2,2,1′,1′,1′-D6)piperazine-1-carboxylate (step-2, 1.1 g, 4.69 mmol, 1.0 eq) 4M HCl in 1,4-dioxane (15 ml) was added at 0° C. and the reaction mixture was allowed to stir at room temperature for overnight. TLC indicated starting material was consumed and the solvent was evaporated under reduced pressure to give title compound (0.85 g) as colorless solid which was used as such for next step without further purification.

### Step-4: (1R,3aS,5aR,5bR,7aR,9S,11aR,11bR,13aR,13bR)-3a-(4-(1-methylethyl-2,2,2,1′,1′,1′-D6)piperazine-1-carbonyl)-5a,5b,8,8,11a-pentamethyl-1-(prop-1-en-2-yl)icosahydro-1H-cyclopenta[a]chrysen-9-yl acetate

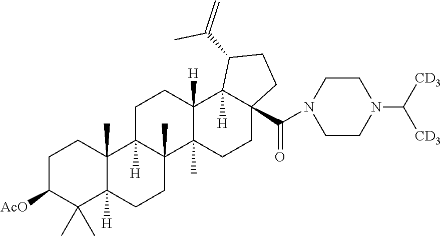

To a solution of 1-(1-methylethyl-2,2,2,1′,1′,1′-D6)piperazine hydrochloride (step-3, 0.255, 1.20 mmol, 1.2 eq) in CH₂Cl₂ (5 ml) DIPEA (0.905 g, 7.02 mmol, 7.0 eq) was added at 0° C. then a solution of (1R,3aS,5aR,5bR,7aR,9S,11aR,11bR,13aR,13bR)-3a-(chlorocarbonyl)-5a,5b,8,8,11a-pentamethyl-1-(prop-1-en-2-yl)icosahydro-1H-cyclopenta[a]chrysen-9-yl acetate (Example 2-step 2, 0.518 g, 1.0 mmol, 1.0 eq) in CH₂Cl₂ (5 ml) was added and the reaction mixture was allowed to stir at room temperature for overnight. TLC indicated starting material was consumed and the desired product was formed. The reaction mixture was diluted with water and extracted with CH₂Cl₂. The combined organic extracts were dried over Na₂SO₄, filtered, evaporated and purified by silicagel column chromatography using 2% methanol:dichloromethane as eluent to give the desired title compound (0.52 g, 66% yield) as a white solid. ¹H NMR (300 MHz, CDCl₃): δ ppm 4.72 (s, 1H), 4.57 (s, 1H), 4.52-4.43 (m, 1H), 3.61 (brs, 4H), 3.05-2.82 (m, 2H), 2.62 (brs, 1H), 2.46 (brs, 4H), 2.15-2.08 (m, 1H), 2.04 (s, 3H), 2.0-1.95 (m, 1H), 1.90-1.80 (m, 1H), 1.78-1.72 (m, 1H), 1.67-1.58 (m, 5H), 1.53-1.48 (m, 2H), 1.45-1.32 (m, 7H), 1.30-1.22 (m, 2H), 1.18-1.12 (m, 1H), 1.08-1.0 (m, 1H), 0.98-0.90 (m, 9H), 0.67-0.62 (m, 9H), 0.60-0.58 (m, 1H); ES MS: [M+H]⁺ 615.7 (100%).

### Step-5: ((1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysen-3a-yl)(4-(1-methylethyl-2,2,2,1′,1′,1′-D6)piperazin-1-yl) methanone

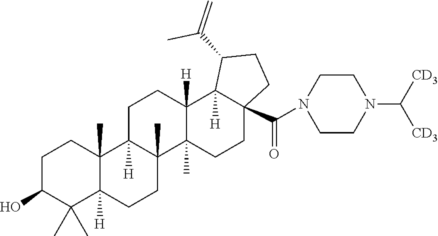

To a solution of (1R,3aS,5aR,5bR,7aR,9S,11aR,11bR,13aR,13bR)-3a-(4-(1-methylethyl-2,2,2,1′,1′,1′-D6)piperazine-1-carbonyl)-5a,5b,8,8,11a-pentamethyl-1-(prop-1-en-2-yl)icosahydro-1H-cyclopenta[a]chrysen-9-yl acetate (step-4, 0.52 g, 0.84 mmol, 1.0 eq) in THF (3 ml) and Methanol (3 ml) potassium carbonate (0.467 g, 3.38 mmol, 4.0 eq) was added and the reaction mixture was stirred at room temperature for 48 hours. TLC indicated starting material was consumed and the desired product was observed. The reaction mixture was filtered through a pad of celite, which was washed with dichloromethane. The filtrate was concentrated under reduced pressure and purified by silicagel column chromatography using 3% methanol:dichloromethane as eluent to give the desired title compound (0.400 g, 82% yield) as a white solid. ¹H NMR (300 MHz, CDCl₃): δ ppm 4.72 (s, 1H), 4.57 (s, 1H), 3.60 (brs, 4H), 3.22-3.13 (m, 1H), 3.04-2.83 (m, 2H), 2.61 (brs, 1H), 2.45 (brs, 4H), 2.16-2.07 (m, 1H), 2.02-1.94 (m, 1H), 1.92-1.80 (m, 1H), 1.75-1.70 (m, 1H), 1.68 (brs, 3H), 1.67-1.63 (m, 1H), 1.63-1.59 (m, 1H), 1.58-1.53 (m, 2H), 1.53-1.47 (m, 2H), 1.46-1.42 (m, 1H), 1.40-1.32 (m, 6H), 1.18-1.12 (m, 1H), 1.07-0.98 (m, 1H), 0.97-0.94 (m, 9H), 0.93-0.85 (m, 3H), 0.82 (brs, 3H), 0.75 (brs, 3H), 0.72-0.65 (m, 1H); ES MS: [M+H]⁺ 573.6 (100%).

### Step-6: 4-((1R,3aS,5aR,5bR,7aR,9S,11aR,11bR,13aR,13bR)-3a-(4-(1-methylethyl-2,2,2,1′,1′,1′-D6)piperazine-1-carbonyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid

To a ((1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysen-3a-yl)(4-(1-methylethyl-2,2,2,1′,1′,1′-D6)piperazin-1-yl)methanone (step-5, 0.15 g, 0.261 mmol, 1.0 eq) and 2,2-dimethylsuccinic anhydride (0.134 g, 1.04 mmol, 4.0 eq) in toluene (3 ml) 4-dimethylaminopyridine (0.064 g, 0.52 mmol, 2.0 eq) was added and the reaction mixture was heated at 90° C. for overnight. TLC indicated starting material was consumed and the desired product was observed. The reaction mixture was concentrated under reduced pressure, cooled to 0° C., acidified to pH=4-5 using 0.1N citric acid solution and extracted with CH₂Cl₂. The combined organic extracts were washed with water, dried over Na₂SO₄, filtered and evaporated under reduced pressure. The residue was purified by silicagel column chromatography using 5% methanol:dichloromethane as eluent to give the desired title compound (0.12 g, 65% yield) as a white solid. ¹H NMR (300 MHz, CDCl₃): δ ppm 4.72 (s, 1H), 4.57 (s, 1H), 4.45-4.35 (m, 1H), 3.70-3.52 (m, 4H), 3.05-2.80 (m, 3H), 2.78-2.54 (m, 3H), 2.53-2.48 (m, 5H), 2.17-1.94 (m, 2H), 1.90-1.75 (m, 2H), 1.72-1.63 (m, 3H), 1.60-1.45 (m, 4H), 1.45-1.30 (m, 7H), 1.30-1.23 (m, 4H), 1.18-1.10 (m, 6H), 1.07-1.0 (m, 1H), 0.98-0.90 (m, 7H), 0.87-0.75 (m, 9H); ES MS: [M+H]⁺ 701.7 (100%); HPLC: 82.2+9.1% (isomers).

### Example 8

### Preparation of 4-((1R,3aS,5aR,5bR,7aR,9S,11aR,11bR,13aR,13bR)-3a-((1R,3S)-3-(4-(1-methylethyl-2,2,2,1′,1′,1′-D6)piperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid

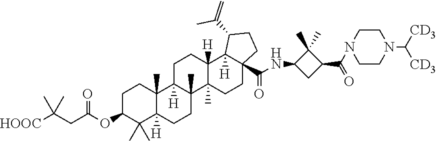

### Step-1: tert-butyl (1R,3S)-3-(4-(1-methylethyl-2,2,2,1′,1′,1′-D6)piperazine-1-carbonyl)-2,2-dimethylcyclobutylcarbamate

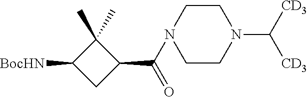

To a stirred solution of (1S,3R)-3-(tert-butoxycarbonylamino)-2,2-dimethylcyclobutanecarboxylic acid (0.571 g, 2.35 mmol, 1.0 eq) and DIPEA (2.12 g, 16.4 mmol, 7.0 eq) in DMF (7 ml) HBTU (1.158 g, 3.05 mmol, 1.3 eq) was added and the reaction mixture was stirred at room temperature for 1 hour then tert-butyl 4-(1-methylethyl-2,2,2,1′,1′,1′-D6)piperazine-1-carboxylate (Example 7-step 2, 0.400 g, 2.35 mmol, 1.0 eq) was added and stirred at room temperature for overnight. TLC indicated starting material was consumed and the desired product was observed. The reaction mixture was evaporated under reduced pressure, diluted with water (25 ml) and extracted with ethyl acetate (50 ml). The combined organic extracts were washed with water, brine solution, dried over Na₂SO₄, filtered and evaporated under reduced pressure. The residue was purified by silicagel column chromatography using 4% methanol:dichloromethane as eluent to give the desired title compound (0.53 g, 63% yield) as a brown color solid. ¹H NMR (300 MHz, CDCl₃): δ ppm 4.75 (d, 1H, J=8.7 Hz), 3.90-3.70 (m, 2H), 3.60-3.42 (m, 3H), 2.81 (t, 1H, J=8.4 Hz), 2.68 (s, 1H), 2.62-2.50 (m, 2H), 2.50-2.40 (m, 2H), 2.38-2.20 (m, 2H), 1.43 (brs, 9H), 1.33 (brs, 3H), 0.87 (s, 3H); ES MS: [M+H]⁺ 360.5 (100%).

### Step-2: ((1S,3R)-3-amino-2,2-dimethylcyclobutyl)(4-(1-methylethyl-2,2,2,1′,1′,1′-D6)piperazin-1-yl)methanone hydrochloride

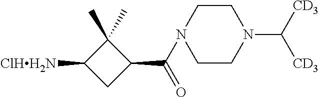

To a tert-butyl (1R,3S)-3-(4-(1-methylethyl-2,2,2,2,1′,1′,1′-D6)piperazine-1-carbonyl)-2,2-dimethylcyclobutylcarbamate (step-1, 0.53 g, 1.47 mmol, 1.0 eq) solution of 4M HCl in 1,4-dioxane (10 ml) was added at 0° C. then the reaction mixture was allowed to stir at room temperature for overnight. TLC indicated starting material was consumed and the solvent was evaporated under reduced pressure to give title compound (0.42 g) as colorless solid which was used as such for next step without further purification.

### Step-3: (1R,3aS,5aR,5bR,7aR,9S,11aR,11bR,13aR,13bR)-3a-((1R,3S)-3-(4-(1-methylethyl-2,2,2,1′,1′,1′-D6)piperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yl acetate

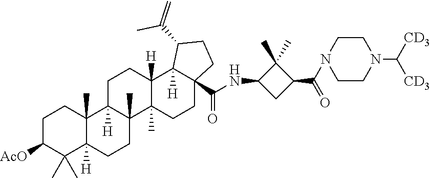

To a solution of ((1S,3R)-3-amino-2,2-dimethylcyclobutyl)(4-(1-methylethyl-2,2,2,1′,1′,1′-D6)piperazin-1-yl)methanone hydrochloride (step-2, 0.439 g, 1.48 mmol, 1.0 eq) in CH₂Cl₂ (10 ml) DIPEA (0.96 g, 7.45 mmol, 5.0 eq) was added at 0° C. then a solution of (1R,3aS,5aR,5bR,7aR,9S,11aR,11bR,13aR,13bR)-3a-(chlorocarbonyl)-5a,5b,8,8,11a-pentamethyl-1-(prop-1-en-2-yl)icosahydro-1H-cyclopenta[a]chrysen-9-yl acetate (Example 2-step 2, 0.77 g, 1.48 mmol, 1.0 eq) in CH₂Cl₂ (5 ml) was added and the reaction mixture was allowed to stir at room temperature for overnight. TLC indicated starting material was consumed and the desired product was formed. The reaction mixture was diluted with CH₂Cl₂ and washed with water. The combined organic extracts were dried over Na₂SO₄, filtered, evaporated and purified by silicagel column chromatography using 2% methanol:dichloromethane as eluent to give the desired title compound (0.75 g, 67% yield) as a white solid. ¹H NMR (300 MHz, CDCl₃): δ ppm 5.83 (d, 1H, J=7.8 Hz), 4.72 (s, 1H), 4.58 (s, 1H), 4.50-4.42 (m, 1H), 4.18-4.05 (m, 1H), 3.82-3.72 (m, 1H), 3.61-3.50 (m, 3H), 3.17-3.07 (m, 1H), 2.88 (t, 1H, J=7.8 Hz), 2.70-2.65 (m, 1H), 2.62-2.40 (m, 5H), 2.38-2.17 (m, 2H), 2.04 (s, 3H), 1.98-1.88 (m, 3H), 1.80-1.70 (m, 2H), 1.64-1.57 (m, 3H), 1.53-1.40 (m, 5H), 1.40-1.35 (m, 6H), 1.32-1.28 (m, 2H), 1.28-1.24 (m, 2H), 1.20-1.10 (m, 2H), 1.08-0.98 (m, 3H), 0.96-0.92 (m, 6H), 0.87 (s, 3H), 0.84-0.78 (m, 9H), 0.78-0.76 (m, 1H); ES MS: [M+H]⁺ 740.8 (100%).

### Step-4: (1R,3aS,5aR,5bR,7aR,9S,11aR,11bR,13aR,13bR)-9-hydroxy-N-((1R,3S)-3-(4-(1-methylethyl-2,2,2,1′,1′,1′-D6)piperazine-1-carbonyl)-2,2-dimethylcyclobutyl)-5a,5b,8,8,11a-pentamethyl-1-(prop-1-en-2-yl)icosahydro-1H-cyclopenta[a]chrysene-3a-carboxamide

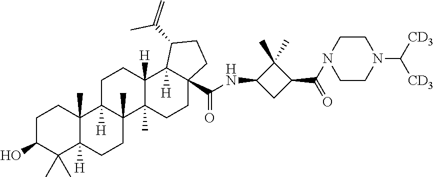

To a stirred (1R,3aS,5aR,5bR,7aR,9S,11aR,11bR,13aR,13bR)-3a-((1R,3S)-3-(4-(1-methylethyl-2,2,2,1′,1′,1′-D6)piperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yl acetate (step-3, 0.75 g, 1.02 mmol, 1.0 eq) in THF (4 ml) and MeOH (4 ml) potassium carbonate (0.559 g, 4.05 mmol, 4.0 eq) was added. The reaction mixture was stirred at room temperature for 48 hours. TLC indicated starting material was consumed and the desired product was observed. The reaction mixture was filtered through a pad of celite, which was washed with dichloromethane. The filtrate was concentrated under reduced pressure and purified by silicagel column chromatography using 3% methanol:dichloromethane as eluent to give the desired title compound (0.500 g, 70% yield) as a white solid. ¹H NMR (300 MHz, CDCl₃): δ ppm 5.84 (d, 1H, J=7.8 Hz), 4.72 (s, 1H), 4.57 (s, 1H), 4.18-4.09 (m, 1H), 3.93-3.73 (m, 1H), 3.64-3.52 (m, 1H), 3.53-3.45 (m, 2H), 3.22-3.08 (m, 2H), 2.92-2.84 (m, 1H), 2.76-2.68 (m, 1H), 2.65-2.52 (m, 5H), 2.40-2.23 (m, 3H), 2.07-2.03 (m, 1H), 2.0-1.86 (m, 3H), 1.80-1.72 (m, 1H), 1.67 (brs, 3H), 1.62-1.54 (m, 4H), 1.53-1.41 (m, 4H), 1.40-1.30 (m, 7H), 1.27-1.23 (m, 2H), 1.20-1.10 (m, 1H), 1.08-0.99 (m, 1H), 0.97-0.92 (m, 9H), 0.87 (brs, 3H), 0.83-0.79 (m, 3H), 0.74 (s, 3H), 0.71-0.64 (m, 1H); ES MS: [M+H]⁺ 698.8 (100%).

### Step-5: 4-((1R,3aS,5aR,5bR,7aR,9S,11aR,11bR,13aR,13bR)-3a-((1R,3S)-3-(4-(1-methylethyl-2,2,2,1′,1′,1′-D6)piperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid

To a (1R,3aS,5aR,5bR,7aR,9S,11aR,11bR,13aR,13bR)-9-hydroxy-N-((1R,3S)-3-(4-(1-methylethyl-2,2,2,1′,1′,1′-D6)piperazine-1-carbonyl)-2,2-dimethylcyclobutyl)-5a,5b,8,8,11a-pentamethyl-1-(prop-1-en-2-yl)icosahydro-1H-cyclopenta[a]chrysene-3a-carboxamide (step-4, 0.200 g, 0.286 mmol, 1.0 eq) and 2,2-dimethylsuccinic anhydride (0.146 g, 1.14 mmol, 4.0 eq) in toluene (3 ml) 4-dimethylaminopyridine (0.070 g, 0.57 mmol, 2.0 eq) was added and the reaction mixture was heated at 90° C. for overnight. TLC indicated starting material was consumed and the desired product was observed. The mixture was concentrated under reduced pressure, cooled to 0° C., acidified to pH=4-5 using 1N citric acid solution and extracted with CH₂Cl₂. The combined organic extracts were washed with water, dried over Na₂SO₄, filtered and evaporated under reduced pressure. The residue was purified by silicagel column chromatography using 4% methanol:dichloromethane as eluent to give the desired title compound (0.12 g, 51% yield) as an off-white solid. ¹H NMR (300 MHz, CDCl₃): δ ppm 5.89 (d, 1H, J=6.9 Hz), 4.72 (s, 1H), 4.58 (s, 1H), 4.45-4.35 (m, 1H), 4.20-4.08 (m, 1H), 3.80-3.70 (m, 1H), 3.62-3.50 (m, 4H), 3.18-3.06 (m, 1H), 2.93-2.84 (m, 1H), 2.70-2.65 (m, 1H), 2.62-2.35 (m, 8H), 2.32-2.10 (m, 5H), 2.02-1.85 (m, 3H), 1.82-1.72 (m, 2H), 1.72-1.64 (m, 4H), 1.62-1.46 (m, 7H), 1.30-1.24 (m, 3H), 1.23-1.18 (m, 4H), 1.18-1.10 (m, 4H), 1.08-1.0 (m, 2H), 0.98-0.90 (m, 5H), 0.92-0.87 (m, 4H), 0.85-0.76 (m, 9H), 0.74-0.68 (m, 1H); ES MS: [M+H]⁺ 826.8 (100%); HPLC: 81.0+12.9+4.5% (isomers).

### Example 9

### Preparation of 4-(1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yl 1-methoxymethyl 2,2-dimethylsuccinate

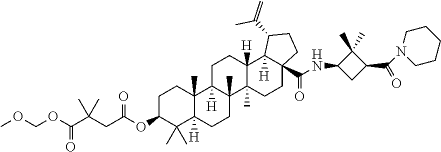

To a stirred solution of 4-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid (as described in our earlier publication WO 2011/007230, 0.2 g, 0.257 mmol, 1.0 eq) and 1,8-diazabicyclo[5,4,0]undec-7-ene (0.050 g, 0.33 mmol, 1.3 eq) in dimethyl acetamide (1.4 ml) methoxymethyl chloride (0.041 g, 0.51 mmol, 2.0 eq) at was added −10° C. slowly in a period of 10 to 15 minutes and the reaction mixture was agitated for a period of 60 minutes at the same temperature then quenched by the addition of water and extracted with ethyl acetate (2×20 ml). The combined organic extracts were washed with water, brine solution, dried over Na₂SO₄, filtered and concentrated under reduced pressure.

The residue was purified by silicagel column chromatography using 2% methanol:dichloromethane as eluent to give the desired title compound (0.15 g, 71% yield) as a white solid. ¹H NMR (300 MHz, CDCl₃): δ ppm 5.85 (d, 1H, J=7.8 Hz), 5.25 (dd, 2H, J=6.0, 8.7 Hz), 4.72 (s, 1H), 4.57 (s, 1H), 4.52-4.42 (m, 1H), 4.11 (q, 1H, J=8.4 Hz), 3.77-3.67 (m, 1H), 3.50-3.45 (m, 4H), 3.44-3.30 (m, 2H), 3.18-3.05 (m, 1H), 2.88 (t, 1H, J=8.1 Hz), 2.70-2.55 (m, 2H), 2.52-2.20 (m, 3H), 1.98-1.85 (m, 2H), 1.80-1.72 (m, 2H), 1.70-1.64 (m, 6H), 1.64-1.61 (m, 2H), 1.57-1.50 (m, 5H), 1.49-1.42 (m, 3H), 1.42-1.40 (m, 1H), 1.39-1.36 (m, 4H), 1.36-1.33 (m, 3H), 1.32-1.29 (m, 7H), 1.27-1.20 (m, 2H), 1.18-1.12 (m, 1H), 1.05-0.96 (m, 1H), 0.97-0.90 (m, 7H), 0.87 (s, 3H), 0.84-0.77 (m, 9H), 0.75-0.72 (m, 1H); ES MS: [M+H]⁺ 821.8 (100%); HPLC: 99.7%.

### Example 10

### Preparation of 4-(1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yl 1-(1-(isopropoxycarbonyloxy)ethyl)2,2-dimethylsuccinate

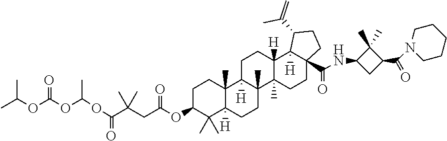

### Step-1: 1-chloroethyl carbonochloridate

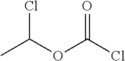

To a solution of ethyl chloroformate (11.35 g, 104.6 mmol, 1.0 eq) and sulfuryl chloride (15.38 g, 114.0 mmol, 1.09 eq) benzoyl peroxide (0.034 g, 0.14 mmol, 0.001 eq) was added and the reaction mixture was refluxed for overnight and it was distilled at atmospheric pressure (boiling range 119-140° C.) to give 1-chloroethyl chloroformate (3.5 g, 24% yield) as colorless liquid. ¹H NMR (300 MHz, CDCl₃): δ ppm 6.45 (q, 1H, J=5.7 Hz), 1.88 (d, 3H, J=5.7 Hz).

### Step-2: 1-chloroethyl isopropyl carbonate

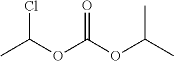

To a solution of 1-Chloroethyl chloroformate (step-1, 3.5 g, 24.4 mmol, 1.0 eq) in CH₂Cl₂ (40 ml) isopropanol (1.76 g, 29.3 mmol, 1.2 eq) was added at 0° C. then pyridine (1.25 g, 15.8 mmol, 0.65 eq) was added dropwise to the solution over a period of 10 minutes. Once the addition finished, the reaction mixture was stirred at the same temperature for 30 minutes. The reaction mixture was washed successively with water (2×30 ml), 5% potassium hydrogen sulfate (2×30 ml) and brine solution (2×30 ml). The combined organic layers were dried over anhydrous sodium sulphate, filtered and evaporated under reduced pressure to obtain the title compound (3.1 g) as colorless liquid. ¹H NMR (300 MHz, CDCl₃): δ ppm 6.43 (q, 1H, J=5.7 Hz), 5.0-4.89 (m, 1H), 1.83 (d, 3H, J=5.7 Hz), 1.34 (d, 3H, J=4.5 Hz), 1.32 (d, 3H, J=4.5 Hz).

### Step-3: 1-iodoethyl isopropyl carbonate

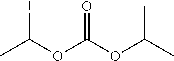

To a solution of 1-chloroethyl isopropyl carbonate (step-2, 0.5 g, 3.01 mmol, 1.0 eq) and sodium iodide (0.98 g, 6.55 mmol, 2.18 eq) in benzene (5 ml) 18-crown-6 (0.024 g, 0.09 mmol, 0.03 eq) was added and the reaction mixture was heated to reflux for overnight. The reaction mixture was diluted with water and extracted with diethyl ether (2×10 ml). The combined organic layers were washed with 5% sodium thiosulphate solution, brine solution, dried over sodium sulphate, filtered and concentrated under reduced pressure to obtain the desired title compound (0.4 g) as colorless oil. ¹H NMR (300 MHz, CDCl₃): δ ppm 6.43 (q, 1H, J=5.7 Hz), 5.0-4.90 (m, 1H), 1.83 (d, 3H, J=5.7 Hz), 1.35 (d, 3H, J=4.5 Hz), 1.33 (d, 3H, J=4.5 Hz).

### Step-4: 4-(1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yl 1-(1-(isopropoxycarbonyloxy)ethyl)2,2-dimethylsuccinate

To a solution of 4-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid (as described in our earlier publication WO 2011/007230, 0.300 g, 0.38 mmol, 1.0 eq) and 1,8-diazabicyclo[5.4.0]undec-7-ene (DBU, 0.076 g, 0.50 mmol, 1.3 eq) in N,N-dimethylacetamide (DMA)(3 ml) 1-iodethyl isopropyl carbonate (step-3, 0.099 g, 0.77 mmol, 2.0 eq) was added at −10° C. and the reaction mixture was stirred at the same temperature for 1 hour then allowed to stir at room temperature for overnight. TLC indicated starting material was consumed and the desired product was observed. The reaction mixture was diluted with water and extracted with CH₂Cl₂ (2×20 ml). The combined organic layers were washed with brine solution, dried over Na₂SO₄, filtered and evaporated under reduced pressure. The residue was purified by silicagel column chromatography using 0.5% methanol:dichloromethane as eluent to give the desired title compound (0.21 g, 60% yield) as an off-white solid. ¹H NMR (300 MHz, CDCl₃): δ ppm 6.82-6.73 (m, 1H), 5.87 (d, 1H, J=7.5 Hz), 4.94-4.83 (m, 1H), 4.72 (s, 1H), 4.58 (s, 1H), 4.52-4.43 (m, 1H), 4.18-4.04 (m, 1H), 3.77-3.64 (m, 1H), 3.50-3.35 (m, 3H), 3.17-3.05 (m, 1H), 2.88 (t, 1H, J=8.1 Hz), 2.70-2.50 (m, 2H), 2.50-2.40 (m, 1H), 2.38-2.23 (m, 2H), 2.0-1.85 (m, 2H), 1.80-1.70 (m, 2H), 1.67 (s, 3H), 1.65-1.62 (m, 2H), 1.57-1.54 (m, 3H), 1.53-1.48 (m, 6H), 1.48-1.44 (m, 2H), 1.43-1.40 (m, 2H), 1.38 (s, 3H), 1.37-1.34 (m, 3H), 1.33-1.28 (m, 6H), 1.27-1.23 (m, 9H), 1.18-1.13 (m, 2H), 1.07-0.97 (m, 2H), 0.97-0.93 (m, 6H), 0.92-0.89 (m, 1H), 0.87 (s, 3H), 0.85-0.81 (m, 9H), 0.82-0.78 (m, 2H); ES MS: [M+H]⁺ 907.5 (100%), [M+Na]⁺ 929.5 (100%); HPLC: 92.6%.

### Example 11

### Preparation of 4-((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxo butanoicacid

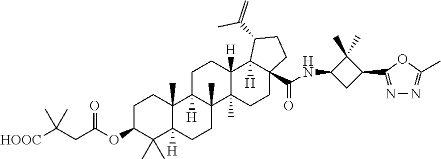

### Step-1: (1R,3aS,5aR,5bR,7aR,9S,11aR,11bR,13aR,13bR)-3a-((1R,3S)-3-(2-acetylhydrazine carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-ylacetate

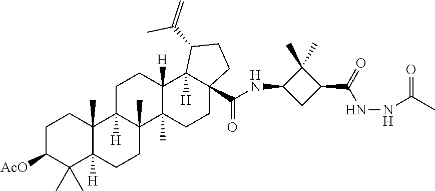

To a stirred solution of (1S,3R)-3-((1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxamido)-2,2-dimethylcyclobutanecarboxylic acid (as described in our earlier publication WO 2011/007230, 2.5 g, 4.01 mmol, 1.0 eq) in DMF (25 ml) HATU (1.98 g, 5.22 mmol, 1.3 eq) and DIPEA (2.58 g, 20.04 mmol, 5.0 eq) were added and stirred at room temperature for 1 hour then acetic hydrazide (0.297 g, 4.01 mmol, 1.0 eq) was added and stirred at room temperature for overnight. After completion of the reaction monitored by TLC, the solvent was evaporated under reduced pressure and the reaction mixture was diluted with water and extracted with ethyl acetate. The combined organic layers were washed with brine solution, dried over Na₂SO₄, filtered, evaporated and purified by silicagel column chromatography by using 1% methanol/dichloromethane as eluent to obtain the title compound (2.2 g, 81% yield) as a white solid. ¹H NMR (300 MHz, CDCl₃): δ ppm 8.38 (brs, 1H), 8.23 (brs, 1H), 5.88 (d, 1H, J=8.1 Hz), 4.72 (s, 1H), 4.58 (s, 1H), 4.50-4.42 (m, 1H), 4.20-4.10 (m, 1H), 3.17-3.05 (m, 1H), 2.58-2.33 (m, 3H), 2.18-2.10 (m, 1H), 2.08-2.04 (m, 6H), 1.98-1.84 (m, 2H), 1.83-1.70 (m, 2H), 1.65-1.54 (m, 5H), 1.53-1.43 (m, 4H), 1.42-1.37 (m, 3H), 1.37-1.30 (m, 6H), 1.30-1.24 (m, 3H), 1.20-1.10 (m, 1H), 1.04-1.0 (m, 1H), 1.98-1.94 (m, 6H), 0.93-0.90 (m, 3H), 0.90-0.87 (m, 1H), 0.85-0.83 (m, 9H), 0.78-0.74 (m, 1H); ES MS: [M+H]⁺ 680.6 (100%).

### Step-2: (1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yl acetate

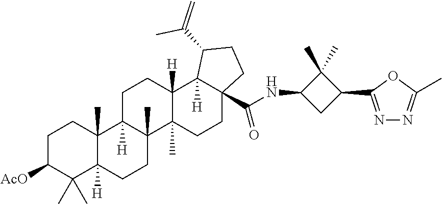

To a stirred solution of (1R,3aS,5aR,5bR,7aR,9S,11aR,11bR,13aR,13bR)-3a-((1R,3S)-3-(2-acetylhydrazine carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-ylacetate (step-1, 2.2 g, 3.23 mmol, 1.0 eq) in CH₂Cl₂ (25 ml) triethylamine (0.98 g, 9.71 mmol, 3.0 eq) and para-toluenesulfonylchloride (0.74 g, 3.88 mmol, 1.2 eq) were added sequentially and stirred at room temperature for overnight. After completion of the reaction monitored by TLC, the reaction mixture was diluted with CH₂Cl₂, washed with water and brine solution, dried over Na₂SO₄, filtered, evaporated and purified by silicagel column chromatography using 1% methanol:dichloromethane as eluent to afford the title compound (2.0 g, 95% yield) as a white solid. H NMR (300 MHz, CDCl₃): δ ppm 5.73 (d, 1H, J=7.5 Hz), 4.73 (s, 1H), 4.59 (s, 1H), 4.50-4.40 (m, 1H), 4.26 (q, 1H, J=8.4 Hz), 3.16-3.05 (m, 2H), 2.70-2.60 (m, 1H), 2.51 (s, 3H), 2.50-2.30 (m, 2H), 2.04 (s, 3H), 1.98-1.88 (m, 2H), 1.80-1.72 (m, 1H), 1.70-1.67 (m, 4H), 1.65-1.60 (m, 2H), 1.60-1.57 (m, 4H), 1.56-1.53 (m, 1H), 1.52-1.40 (m, 5H), 1.38-1.33 (m, 5H), 1.28-1.23 (m, 3H), 1.22-1.10 (m, 2H), 1.10-1.0 (m, 1H), 0.97-0.92 (m, 6H), 0.90-0.87 (m, 1H), 0.87-0.78 (m, 9H), 0.80-0.74 (m, 1H); ES MS: [M+H]⁺ 662.7 (100%).

### Step-3: (1R,3aS,5aR,5bR,7aR,9S,11aR,11bR,13aR,13bR)—N-((1R,3S)-2,2-dimethyl-3-(5-methyl-1,3,4-oxadiazol-2-yl)cyclobutyl)-9-hydroxy-5a,5b,8,8,11a-pentamethyl-1-(prop-1-en-2-yl)icosahydro-1H-cyclopenta[a]chrysene-3a-carboxamide

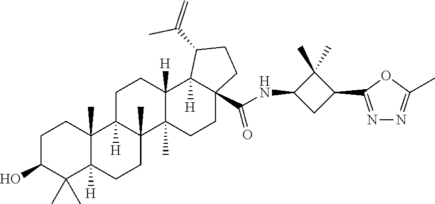

To a stirred solution of (1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yl acetate (step-2, 2.0 g, 3.02 mmol, 1.0 eq) in THF (10 ml) and Methanol (10 ml) potassium carbonate (1.67 g, 12.09 mmol, 4.0 eq) was added and the reaction mixture was stirred at room temperature for 48 hours. TLC indicated starting material was consumed and the desired product was observed. The mixture was filtered through celite pad and washed with dichloromethane. The mixture was concentrated under reduced pressure, diluted with water (100 ml) and extracted with DCM (2×100 ml). The combined organic layers were dried over sodiumsulphate and concentrated under reduced pressure. The residue was purified by silicagel column chromatography using 1% methanol:dichloromethane as eluent to give the desired title compound (1.6 g, 89% yield) as a white solid. ¹H NMR (300 MHz, CDCl₃): δ ppm 5.72 (d, 1H, J=7.8 Hz), 4.73 (s, 1H), 4.58 (s, 1H), 4.26 (q, 1H, J=8.4 Hz), 3.20-3.07 (m, 3H), 2.71-2.60 (m, 1H), 2.51 (s, 3H), 2.50-2.42 (m, 1H), 2.42-2.30 (m, 1H), 2.0-1.87 (m, 2H), 1.80-1.70 (m, 2H), 1.64-1.53 (m, 6H), 1.52-1.40 (m, 6H), 1.39-1.32 (m, 6H), 1.30-1.23 (m, 4H), 1.20-1.12 (m, 1H), 1.0-0.92 (m, 9H), 0.90-0.83 (m, 1H), 0.83-0.78 (m, 6H), 0.74 (s, 3H), 0.70-0.64 (m, 1H).

### Step-4: 4-((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxo butanoicacid

To a stirred solution of (1R,3aS,5aR,5bR,7aR,9S,11aR,11bR,13aR,13bR)—N-((1R,3S)-2,2-dimethyl-3-(5-methyl-1,3,4-oxadiazol-2-yl)cyclobutyl)-9-hydroxy-5a,5b,8,8,11a-pentamethyl-1-(prop-1-en-2-yl)icosahydro-1H-cyclopenta[a]chrysene-3a-carboxamide (step-3, 0.300 g, 0.48 mmol, 1.0 eq) and 2,2-dimethyl succinicanhydride (0.248 g, 1.93 mmol, 4.0 eq) in toluene (3 ml) DMAP (0.118 g, 0.96 mmol, 2.0 eq) was added and the reaction mixture was heated at 90° C. for overnight. TLC indicated starting material was consumed and the desired product was observed. The reaction mixture was concentrated under reduced pressure, cooled to 0° C., acidified to pH=4-5 using 1N citric acid solution and extracted with CH₂Cl₂. The combined organic extracts were washed with water, dried over Na₂SO₄, filtered and evaporated under reduced pressure. The residue was purified by silicagel column chromatography using 2% methanol:dichloromethane as eluent to give the desired title compound (0.200 g, 55% yield) as an off-white solid. ¹H NMR (300 MHz, CDCl₃): δ ppm 5.79 (d, 1H, J=7.8 Hz), 4.72 (s, 1H), 4.58 (s, 1H), 4.52-4.46 (m, 1H), 4.26 (q, 1H, J=8.4 Hz), 3.17-3.07 (m, 2H), 2.72-2.58 (m, 3H), 2.52 (s, 3H), 2.50-2.32 (m, 2H), 2.0-1.88 (m, 2H), 1.80-1.72 (m, 2H), 1.70-1.65 (m, 4H), 1.64-1.53 (m, 4H), 1.52-1.40 (m, 4H), 1.40-1.32 (m, 6H), 1.32-1.23 (m, 9H), 1.20-1.12 (m, 2H), 1.05-1.0 (m, 1H), 0.97-0.90 (m, 6H), 0.90-0.88 (m, 1H), 0.84-0.77 (m, 11H), 0.77-0.72 (m, 1H); ES MS: [M+H]⁺ 748.7 (100%); HPLC: 86.5+9.7% (isomers).

### Example 12

### Preparation of 4-((1R,3aS,5aR,5bR,7aR,9S,11aR,11bR,13aR,13bR)-3a-(4-ethylpiperazine-1-carbon yl)-5a,5b,8,8,11a-pentamethyl-1-(1-methylcyclopropyl)icosahydro-1H-cyclopenta[a]chrysen-9-yloxy)-2,2-dimethyl-4-oxobutanoicacid

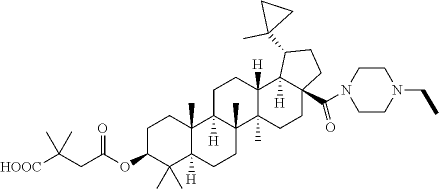

### Step 1: synthesis of (R,3aS,5aR,5bR,7aR,9S,11aR,11bR,13aR,13bR)-benzyl 9-acetoxy-5a,5b,8,8,11a-penta methyl-1-(prop-1-en-2-yl)icosahydro-1H-cyclopenta[a]chrysene-3a-carboxylate

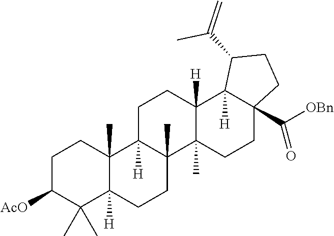

To a stirred solution of (1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxylic acid (20 g, 40.16 mmol, 1.0 eq) in DMF (200 ml) was added potassium carbonate (11.0 g, 80.32 mmol, 2.0 eq) and benzyl bromide (6.8 g, 40.16 mmol, 1.0 eq). The reaction mixture was heated at 70° C. for overnight. TLC indicated starting material was consumed and the desired product was observed. The reaction mixture was cooled to 0° C., diluted with water (500 ml) and the solid was precipitated out. The solids that were collected by filtration dissolved in CH₂Cl₂, dried over Na₂SO₄, filtered, evaporated and purified by silicagel column chromatography by using 2% methanol:dichloromethane as an eluent to gave the desired product (19.0 g, 80% yield) as a white solid. ¹H NMR (300 MHz, CDCl₃): δ ppm 7.38-7.31 (m, 5H), 5.17-5.05 (m, 2H), 4.72 (s, 1H), 4.59 (s, 1H), 4.49-4.42 (m, 1H), 3.07-2.95 (m, 1H), 2.32-2.24 (m, 1H), 2.24-2.12 (m, 1H), 2.04 (s, 3H), 1.95-1.80 (m, 2H), 1.72-1.69 (m, 1H), 1.67 (s, 3H), 1.64-1.58 (m, 3H), 1.50-1.30 (m, 9H), 1.28-1.22 (m, 2H), 1.21-1.15 (m, 1H), 1.15-0.96 (m, 3H), 0.93 (s, 3H), 0.82 (s, 9H), 0.80-0.78 (m, 1H), 0.75 (s, 3H); ES MS: [M+H]⁺ 611.3 (10%); IR (KBr) cm⁻¹: 2967, 2948, 2867, 1741, 1721, 1245, 1127.

### Step 2: (1R,3aS,5aR,5bR,7aR,9S,11aR,11bR,13aR,13bR)-benzyl 9-acetoxy-5a,5b,8,8,11a-penta methyl-1-(1-methylcyclopropyl)icosahydro-1H-cyclopenta[a]chrysene-3a-carboxylate

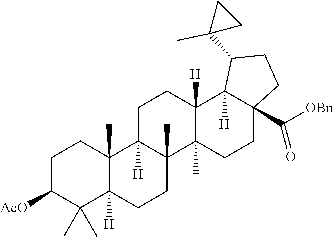

To a flame dried round bottom flask under nitrogen (1R,3aS,5aR,5bR,7aR,9S,11aR,11bR,13aR,13bR)-benzyl 9-acetoxy-5a,5b,8,8,11a-penta methyl-1-(prop-1-en-2-yl)icosahydro-1H-cyclopenta[a]chrysene-3a-carboxylate (step 1) (5.0 g, 8.27 mmol, 1.0 eq) and dry CH₂Cl₂ (100 ml) was added. The reaction mixture was cooled to −25° C., diethylzinc (5.11 g, 41.39 mmol, 5.0 eq, 1.0 M solution in hexane) was added then stirred at the same temperature for 1 hour. Diiodomethane (17.73 g, 66.22 mmol, 8.0 eq) was added and the reaction mixture was allowed to stir at room temperature for overnight. The reaction mixture cooled to 0° C., quenched with saturated NH₄Cl solution, extracted with CH₂Cl₂ (3×50 ml). The combined organic extracts were dried over Na₂SO₄, filtered and evaporated under reduced pressure. The crude residue was purified by silicagel column chromatography by using 2% ethyl acetate:hexane as an eluent gave the desired product (4.93 g, 98% yield) as a white solid. H NMR (300 MHz, CDCl₃): δ ppm 7.38-7.31 (m, 5H), 5.15-5.05 (m, 2H), 4.51-4.41 (m, 1H), 2.28-2.21 (m, 1H), 2.14-2.07 (m, 1H), 2.04 (s, 3H), 1.98-1.90 (m, 1H), 1.90-1.77 (m, 2H), 1.76-1.68 (m, 1H), 1.67-1.60 (m, 3H), 1.58-1.56 (m, 2H), 1.50-1.44 (m, 2H), 1.43-1.25 (m, 8H), 1.24-1.14 (m, 1H), 1.13-0.99 (m, 2H), 0.95 (s, 3H), 0.90 (s, 3H), 0.83 (s, 9H), 0.83-0.78 (m, 1H), 0.75 (s, 3H), 0.41-0.30 (m, 2H), 0.27-0.17 (m, 2H); ES MS: [M+Na]⁺ 625.4 (10%).

### Step 3: (1R,3aS,5aR,5bR,7aR,9S,11aR,11bR,13aR,13bR)-9-acetoxy-5a,5b,8,8,11a-penta methyl-1-(1-methylcyclopropyl)icosahydro-1H-cyclopenta[a]chrysene-3a-carboxylicacid

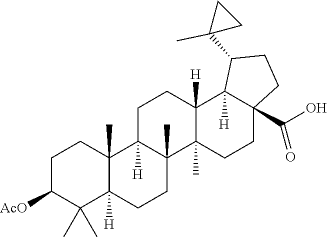

To a solution of (1R,3aS,5aR,5bR,7aR,9S,11aR,11bR,13aR,13bR)-benzyl 9-acetoxy-5a,5b,8,8,11a-penta methyl-1-(1-methylcyclopropyl)icosahydro-1H-cyclopenta[a]chrysene-3a-carboxylate (step 2) (4.3 g, 7.14 mmol, 1.0 eq) in ethyl acetate:ethanol (1:1, 43 ml) was added 10% Pd/C (0.500 g, 0.471 mmol, 0.066 eq) under nitrogen and the reaction mixture was hydrogenated at 50 psi for overnight. TLC indicated starting material was consumed and the desired product was observed. The reaction mixture was filtered through a pad of celite and was washed with ethyl acetate. The filtrate was evaporated under reduced pressure and the residue was purified by silicagel column chromatography by using 4% ethyl acetate:hexane as an eluent gave the desired product (3.4 g, 93% yield) as a white solid. ¹H NMR (300 MHz, CDCl₃): δ ppm 9.95 (brs, 1H), 4.52-4.45 (m, 1H), 2.25-2.18 (m, 1H), 2.14-2.07 (m, 1H), 2.05 (brs, 3H), 1.98-1.85 (m, 3H), 1.74-1.70 (m, 1H), 1.70-1.57 (m, 5H), 1.57-1.30 (m, 10H), 1.30-1.12 (m, 3H), 0.98 (s, 3H), 0.92 (brs, 6H), 0.86 (s, 3H), 0.85 (s, 3H), 0.83 (s, 3H), 0.81-0.78 (m, 1H), 0.42-0.33 (m, 2H), 0.30-0.18 (m, 2H); ES MS: [M−H]⁻ 511.3 (50%); IR cm⁻¹: 3219, 2955, 2871, 1729, 1708, 1268, 979 cm⁻¹.

### Step 4: (1R,3aS,5aR,5bR,7aR,9S,11aR,11bR,13aR,13bR)-3a-(chlorocarbonyl)-5a,5b,8,8,11a-penta methyl-1-(1-methylcyclopropyl)icosahydro-1H-cyclopenta[a]chrysen-9-ylacetate

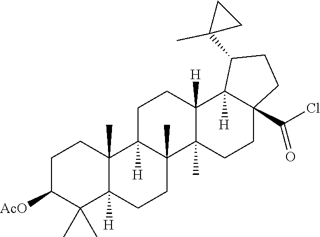

To a stirred solution of (1R,3aS,5aR,5bR,7aR,9S,11aR,11bR,13aR,13bR)-9-acetoxy-5a,5b,8,8,11a-penta methyl-1-(1-methylcyclopropyl)icosahydro-1H-cyclopenta[a]chrysene-3a-carboxylicacid (step 3) (2.0 g, 3.90 mmol, 1.0 eq) in toluene (20 ml) at 0° C. was added thionyl chloride (2.32 g, 19.5 mmol, 5.0 eq). The reaction mixture was allowed to reach room temperature and heated to reflux for 3 hours. After completion of the reaction monitored by TLC, solvent was evaporated in vacuo providing a crude residue that was re-dissolved in toluene (2×10 ml) to remove the excess thionyl chloride. The solution was concentrated in vacuo to produce the desired acid chloride (2.0 g), which is used as such for next step.

### Step 5: (1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl acetate

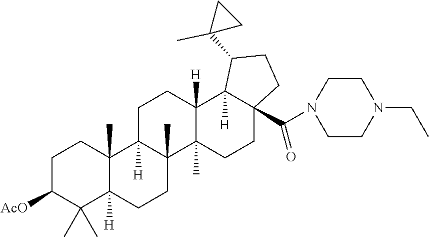

To a stirred solution of ethyl piperazine (0.861 g, 7.54 mmol, 2.0 eq) and triethylamine (1.90 g, 18.8 mmol, 5.0 eq) in CH₂Cl₂ (30 ml) at 0° C. was added a solution of (1R,3aS,5aR,5bR,7aR,9S,11aR,11bR,13aR,13bR)-3a-(chlorocarbonyl)-5a,5b,8,8,8,11a-penta methyl-1-(1-methylcyclopropyl)icosahydro-1H-cyclopenta[a]chrysen-9-ylacetate (step 4) (2.0 g, 3.77 mmol, 1.0 eq) in CH₂Cl₂ (10 ml). The reaction mixture was allowed to stir at room temperature for overnight. The reaction mixture was diluted with water and extracted with CH₂Cl₂ (3×100 ml). The combined organic extracts were dried over Na₂SO₄, filtered and evaporated under reduced pressure. The residue was purified by silicagel column chromatography by using 1% methanol:dichloromethane as an eluent gave the desired product (2.0 g, 87.3% yield) as a white solid. ¹H NMR (300 MHz, CDCl₃): δ ppm 4.48 (t, 1H, J=8.4 Hz), 3.70-3.52 (m, 4H), 2.84-2.72 (m, 1H), 2.48-2.35 (m, 6H), 2.13-2.08 (m, 1H), 2.04 (s, 3H), 2.03-1.98 (m, 1H), 1.98-1.88 (m, 1H), 1.88-1.67 (m, 3H), 1.57-1.44 (m, 4H), 1.42-1.25 (m, 8H), 1.22-1.15 (m, 1H), 1.09 (m, 4H), 1.05-0.98 (m, 1H), 0.97 (s, 3H), 0.93 (s, 3H), 0.90 (s, 3H), 0.86 (s, 3H), 0.83 (brs, 6H), 0.80-0.78 (m, 1H), 0.48-0.40 (m, 1H), 0.38-0.30 (m, 1H), 0.27-0.13 (m, 2H); ES MS: [M+H]⁺ 609.5 (100%).

### Step 6: (4-ethylpiperazin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl) methanone

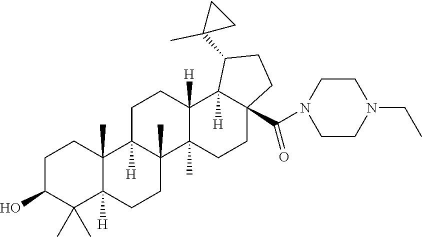

To a stirred solution of (1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl acetate (step 5) (2.0 g, 3.28 mmol, 1.0 eq) in THF (10 ml) and Methanol (10 ml) was added potassium carbonate (3.177 g, 23.0 mmol, 7.0 eq). The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by silicagel column chromatography by using 1% methanol:dichloromethane as an eluent gave the desired product (1.5 g, 80.6% yield) as a white solid. ¹H NMR (300 MHz, CDCl₃): δ ppm 3.67-3.56 (m, 4H), 3.19 (dd, 1H, J=4.8 Hz, 10.2 Hz), 2.84-2.72 (m, 1H), 2.48-2.34 (m, 6H), 2.12-2.0 (m, 2H), 2.0-1.91 (m, 1H), 1.90-1.70 (m, 1H), 1.70-1.60 (m, 6H), 1.58-1.42 (m, 4H), 1.42-1.25 (m, 8H), 1.20-1.12 (m, 1H), 1.09 (t, 3H, J=7.2 Hz), 0.97 (s, 3H), 0.97 (s, 3H), 0.93 (s, 3H), 0.89 (s, 3H), 0.83 (s, 3H), 0.76 (s, 3H), 0.73-0.67 (m, 1H), 0.48-0.40 (m, 1H), 0.40-0.30 (m, 1H), 0.30-0.10 (m, 2H); ES MS: [M+H]⁺ 567.4 (100%).

### Step 7: 4-((1R,3aS,5aR,5bR,7aR,9S,11aR,11bR,13aR,13bR)-3a-(4-ethylpiperazine-1-carbon yl)-5a,5b,8,8,11a-pentamethyl-1-(1-methylcyclopropyl)icosahydro-1H-cyclopenta[a]chrysen-9-yloxy)-2,2-dimethyl-4-oxobutanoicacid

To a stirred solution of (4-ethylpiperazin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl) methanone (step 6) (0.280 g, 0.494 mmol, 1.0 eq) and 2,2-dimethyl succinicanhydride (0.253 g, 1.97 mmol, 4.0 eq) in toluene (5 ml) was added DMAP (0.120 g, 0.988 mmol, 2.0 eq). The reaction mixture was heated at 90° C. for overnight. TLC indicated starting material was consumed and the desired product was observed. The mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by silicagel column chromatography by using 5% methanol:dichloromethane as an eluent gave the desired product (0.120 g, 34.9% yield) as a white solid. ¹H NMR (300 MHz, Pyridine-d5): δ ppm 4.80 (dd, 1H, J=4.5 Hz, 11.1 Hz), 3.78-3.68 (m, 4H), 3.20 (t, 1H, J=9.9 Hz), 2.95 (dd, 2H, J=15.6 Hz, 24.9 Hz), 2.37-2.30 (m, 4H), 2.30-2.20 (m, 4H), 2.20-1.90 (m, 3H), 1.90-1.70 (m, 3H), 1.70-1.60 (m, 2H), 1.58-1.53 (m, 6H), 1.53-1.40 (m, 6H), 1.40-1.32 (m, 3H), 1.32-1.10 (m, 4H), 1.10-1.05 (brs, 6H), 1.04-0.95 (m, 12H), 0.93-0.82 (m, 1H), 0.79 (brs, 3H), 0.62-0.52 (m, 1H), 0.50-0.42 (m, 1H), 0.35-0.22 (m, 2H); ESI MS: [M+H]⁺ 695.5 (100%), [M+Na]⁺ 717.5 (50%); IR (KBr) cm⁻¹: 3436, 2967, 2943, 1728, 1634, 1194, 1011 cm⁻¹; HPLC: 94.60%.

### Example 13

### Preparation of (1R,3S)-3-(((1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylic acid

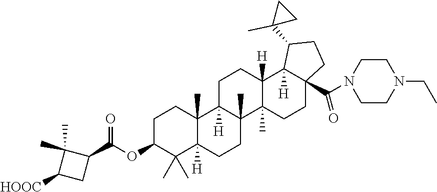

### Step 1: (1S,3R)-3-(benzyloxycarbonyl)-2,2-dimethylcyclobutanecarboxylic 2,4,6-trichlorobenzoic anhydride

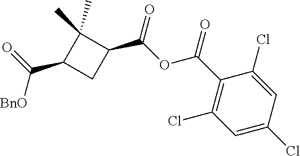

To a stirred solution of (1S,3R)-3-(benzyloxycarbonyl)-2,2-dimethyl cyclobutanecarboxylic acid (0.700 g, 2.67 mmol, 1.0 eq) and triethylamine (0.809 g, 8.01 mmol, 3.0 eq) in THF (14 ml) at 0° C. was added 2,4,6-trichlorobenzoyl chloride (0.782 g, 3.20 mmol, 1.2 eq). The reaction mixture was allowed to stir at room temperature for 4 hours. TLC indicated starting material was consumed and the desired product was observed. The reaction mixture was evaporated under reduced pressure and the crude compound (0.99 g) was used as such for next step.

### Step 2: (1R,3S)-1-benzyl 3-((1R,3aS,5aR,5bR,7aR,9S,11aR,11bR,13aR,13bR)-3a-(4-ethyl piperazine-1-carbonyl)-5a,5b,8,8,11a-pentamethyl-1-(1-methylcyclopropyl)icosahydro-1H-cyclopenta[a]chrysen-9-yl) 2,2-dimethylcyclobutane-1,3-dicarboxylate

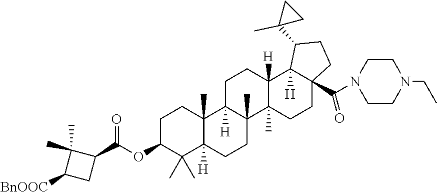

To a stirred solution of (4-ethylpiperazin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl) methanone (0.600 g, 1.05 mmol, 1.0 eq) in toluene (10 ml) was added DMAP (0.258 g, 2.11 mmol, 2.0 eq) and (1S,3R)-3-(benzyloxycarbonyl)-2,2-dimethylcyclobutane carboxylic 2,4,6-trichlorobenzoic anhydride (step 1) (0.991 g, 2.11 mmol, 2.0 eq). The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂ (2×50 ml). The combined organic layers were dried over Na₂SO₄, filtered and evaporated under reduced pressure. The crude residue was purified by silicagel column chromatography by using 1.5% methanol:dichloromethane as an eluent to obtain (0.630 g, 73.4% yield) as a white solid. ¹H NMR (300 MHz, CDCl₃): δ ppm 7.35 (m, 5H), 5.18-5.06 (m, 2H), 4.44 (dd, 1H, J=4.8 Hz, 11.1 Hz), 3.70-3.55 (m, 4H), 2.87-2.72 (m, 3H), 2.70-2.58 (m, 1H), 2.50-2.35 (m, 6H), 2.12-2.0 (m, 3H), 1.97-1.85 (m, 1H), 1.83-1.60 (m, 8H), 1.57-1.43 (m, 4H), 1.40-1.23 (m, 12H), 1.20-1.03 (m, 4H), 1.02-0.82 (m, 19H), 0.82-0.75 (m, 1H), 0.48-0.40 (m, 1H), 0.38-0.30 (m, 1H), 0.28-0.15 (m, 2H); ES MS: [M+H]⁺ 812 (60%), [M+Na]⁺ 834 (10%).

### Step 3: (1R,3S)-3-(((1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylicacid

To a solution of (1R,3S)-1-benzyl 3-((1R,3aS,5aR,5bR,7aR,9S,11aR,11bR,13aR,13bR)-3a-(4-ethyl piperazine-1-carbonyl)-5a,5b,8,8,11a-pentamethyl-1-(1-methylcyclopropyl)icosahydro-1H-cyclopenta[a]chrysen-9-yl) 2,2-dimethylcyclobutane-1,3-dicarboxylate (step 2) (0.630 g, 0.77 mmol, 1.0 eq) in dichloromethane (30 ml) was added palladium (II) acetate (0.0872 g, 0.388 mmol, 0.5 eq), triethyl amine (0.235 g, 2.33 mmol, 3.0 eq) and triethylsilane (0.271 g, 2.33 mmol, 3.0 eq). The reaction mixture was flushed with N₂ and heated to reflux for 48 hours. The mixture was cooled to room temperature, filtered through a pad of celite and was washed with dichloromethane. The filtrate was evaporated under reduced pressure, cooled to 0° C., diluted with water (10 ml), acidified to pH 5.0 with 1N HCl and extracted with CH₂Cl₂ (2×200 ml).

The combined organic layers were dried over Na₂SO₄, filtered and evaporated under reduced pressure. The crude residue was purified by silicagel column chromatography by using 6% methanol:dichloromethane as an eluent gave the desired compound (0.190 g, 33.9% yield) as a white solid. ¹H NMR (300 MHz, Pyridine-d5): δ ppm 4.71 (dd, J=4.5, 11.4 Hz, 1H), 3.80-3.68 (m, 4H), 3.23 (t, J=9.9 Hz, 1H), 3.16-3.04 (m, 2H), 3.04-2.96 (m, 1H), 2.38-2.32 (m, 4H), 2.30-2.20 (m, 4H), 2.15-1.90 (m, 4H), 1.90-1.70 (m, 3H), 1.68-1.62 (m, 2H), 1.56 (s, 3H), 1.54-1.48 (m, 3H), 1.48-1.37 (m, 9H), 1.36-1.25 (m, 3H), 1.14-0.98 (brs, 4H), 1.04 (s, 3H), 1.01 (s, 3H), 0.99 (s, 3H), 0.96 (s, 3H), 0.93 (s, 3H), 0.83 (s, 3H), 0.82-0.79 (m, 1H), 0.63-0.55 (m, 1H), 0.53-0.45 (m, 1H), 0.36-0.25 (m, 2H); ES MS: [M+H]⁺ 721.6 (100%); IR (KBr) cm⁻¹: 3440, 2962, 2943, 1730, 1634, 1467, 1190 cm⁻¹; HPLC: 98.72%.

### Example 14

### Preparation of 4-((1R,3aS,5aR,5bR,7aR,9S,11aR,11bR,13aR,13bS)-3a-((1R,3S)-2,2-dimethyl-3-(piperidine-1-carbonyl)cyclobutylcarbamoyl)-5a,5b,8,8,11a-pentamethyl-1-(2-methyloxiran-2-yl)icosa hydro-1H-cyclopenta[a]chrysen-9-yloxy)-2,2-dimethyl-4-oxobutanoicacid

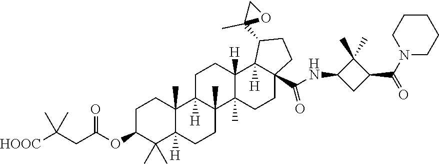

To a stirred solution of 4-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid (0.5 g, 0.643 mmol, 1.0 eq) in CH₂Cl₂ (10 ml) at 0° C. was added meta-chloroperbenzoicacid (0.221.5 g, 1.287 mmol, 2.0 eq). The reaction mixture was allowed to stir at room temperature for 1 hour. TLC indicated starting material was consumed and the desired product was observed. The reaction mixture was quenched with saturated potassium carbonate solution and extracted with CH₂Cl₂ (3×50 ml). The combined organic extracts were dried over Na₂SO₄, filtered and evaporated under reduced pressure. The residue was purified by silicagel column chromatography by using 2% methanol:dichloromethane as an eluent to give the desired product (260 mg, 51% yield) as a white solid. ¹H NMR (300 MHz, CDCl₃): δ ppm 5.97 (d, J=8.2 Hz, 1H), 4.38-4.28 (m, 1H), 4.14-4.02 (m, 1H), 3.76-3.66 (m, 1H), 3.50-3.30 (m, 3H), 2.88 (t, J=8.1 Hz, 1H), 2.68-2.57 (m, 2H), 2.56-2.47 (m, 3H), 2.40-2.32 (m, 1H), 2.30-2.15 (m, 2H), 1.92-1.73 (m, 4H), 1.70-1.48 (m, 9H), 1.42-1.32 (m, 8H), 1.30-1.20 (m, 9H), 1.20-1.05 (m, 9H), 1.0-0.74 (m, 19H), 0.70-0.62 (m, 1H); ES MS: [M+H]⁺ 793.6 (100%), [M+Na]⁺ 815.7 (50%). HPLC: 91.9%.

### Example 15

### Preparation of 4-((1R,3aS,5aR,5bR,7aR,9S,11aR,11bR,13aR,13bS)-1-(1,2-dihydroxypropan-2-yl)-3a-((1R,3S)-2,2-dimethyl-3-(piperidine-1-carbonyl)cyclobutylcarbamoyl)-5a,5b,8,8,11a-penta methylicosahydro-1H-cyclopenta[a]chrysen-9-yloxy)-2,2-dimethyl-4-oxobutanoicacid

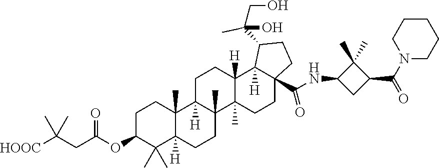

To a stirred solution of 4-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid (0.5 g, 0.643 mmol, 1.0 eq) in Acetone:H₂O (10 ml, 9:1) at 0° C. was added Osmiumtetroxide (1.6 ml, 0.128 mmol, 0.2 eq, 2% solution in toluene) and N-Methyl morpholine-N-oxide (0.452 g, 3.863 mmol, 6.0 eq). The reaction mixture was allowed to stir at room temperature for overnight. TLC indicated starting material was consumed and the desired product was observed. The reaction mixture was quenched with 10% sodiumbisulphite solution and stirred at 0° C. for 30 min. The aqueous layer was extracted with CH₂Cl₂ (3×50 ml). The combined organic layers were dried over Na₂SO₄, filtered and evaporated under reduced pressure. The residue was purified by silicagel column chromatography by using 4.5% methanol:dichloromethane as an eluent to give the desired product (70 mg, 13.4% yield) as a white solid. ¹H NMR (300 MHz, DMSO-d6): δ ppm 12.1 (brs, 1H), 7.37 (d, J=8.1 Hz, 1H), 4.42-4.30 (m, 1H), 4.05-3.92 (m, 1H), 3.65-3.55 (m, 2H), 3.48-3.35 (m, 1H), 3.30-3.20 (m, 2H), 3.20-3.13 (m, 2H), 2.87 (t, J=8.4 Hz, 1H), 2.75-2.67 (m, 2H), 2.30-2.22 (m, 2H), 2.12-2.05 (m, 2H), 2.0-1.80 (m, 2H), 1.70-1.50 (m, 8H), 1.48-1.30 (m, 10H), 1.30-1.10 (m, 17H), 0.99 (s, 3H), 0.93 (s, 3H), 0.85 (s, 3H), 0.80 (s, 3H), 0.78 (s, 3H), 0.77 (s, 3H), 0.75 (s, 3H), 0.73-0.72 (m, 1H); ES MS: [M+H]⁺ 833.6 (100%); HPLC: 90.6%.

### Example 16

### Preparation of 4-((1R,3aS,5aR,5bR,7aR,9S,11aR,11bR,13aR,13bR)-3a-((1R,3S)-3-(5-isobutyl-1,3,4-oxadiazol-2-yl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid

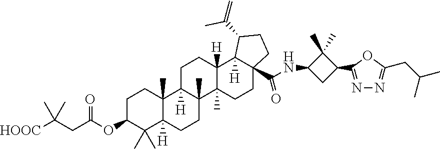

### Step 1: (1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxylicacid

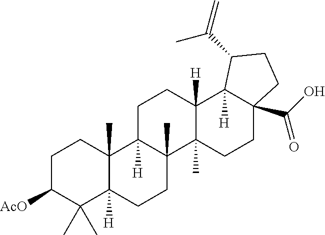

Acetic anhydride (17.08 g, 167.5 mmol, 5.1 eq) was added to a solution of betulinic acid (15.0 g, 32.84 mmol, 1.0 eq), DIPEA (12.73 g, 98.53 mmol, 3.0 eq) and DMAP (0.80 g, 6.56 mmol, 0.2 eq) in THF (225 ml). The mixture was heated at 65-70° C. for overnight. TLC indicated starting material was consumed and the desired product was observed. The mixture was concentrated in vacuo to dryness to yield a white solid. To hydrolyze the mixed anhydride, this solid was suspended into 0.6N hydrochloric acid solution (150 ml) and heated to 100° C. for 6 hours. The suspension was cooled to room temperature and the solid was collected by filtration, washed with water and dried at 50° C. under reduced pressure for 1 hour to obtain the desired product (15 g) as an off-white solid.

### Step 2: (1R,3aS,5aR,5bR,7aR,9S,11aR,11bR,13aR,13bR)-3a-(chlorocarbonyl)-5a,5b,8,8,11a-pentamethyl-1-(prop-1-en-2-yl)icosahydro-1H-cyclopenta[a]chrysen-9-yl acetate

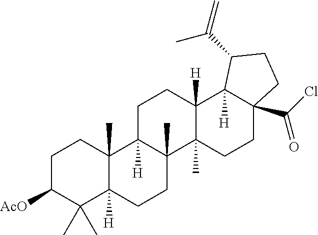

To solution of (1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxylicacid (step 1) (3.5 g, 7.02 mmol, 1.0 eq) in dichloromethane (30 ml) at 0° C. was added oxalyl chloride (4.45 g, 35.14 mmol, 5.0 eq). The reaction mixture was allowed to reach to room temperature and heated to reflux for 3 hours. After completion of the reaction monitored by TLC, solvent was evaporated in vacuo to produce the desired product (3.6 g), which is used as such for next step without further purification.

### Step 3: (1S,3R)-3-amino-2,2-dimethylcyclobutanecarboxylicacid

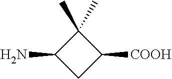

To a stirred solution of (1S,3R)-3-(tert-butoxycarbonylamino)-2,2-dimethylcyclo butanecarboxylic acid (4.0 g, 16.44 mmol, 1.0 eq)) in dichloromethane (25 ml) at 0° C. was added trifluoro acetic acid (15 ml). The reaction mixture was allowed to stir at room temperature for 3 hours. TLC indicated starting material was consumed and the desired product was observed. The reaction mixture was concentrated in vacuo providing a crude residue that was re-dissolved in toluene (20 ml) to remove the excess trifluoroacetic acid. The solution was concentrated in vacuo to produce the desired product (4.5 g), which is used as such for next step without further purification.

### Step 4: (1S,3R)-3-((1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxamido)-2,2-dimethylcyclobutanecarboxylicacid

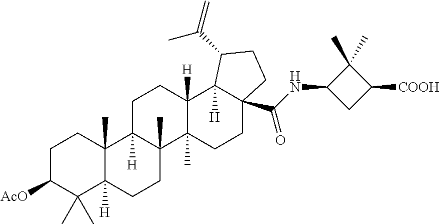

To a stirred solution of (1S,3R)-3-amino-2,2-dimethylcyclobutanecarboxylicacid (step 3) (2.16 g, 8.41 mmol, 1.2 eq) in CH₂Cl₂ (40 ml) at 0° C. was added triethyl amine (5.43 g, 42.0 mmol, 6.0 eq). A solution of (1R,3aS,5aR,5bR,7aR,9S,11aR,11bR,13aR,13bR)-3a-(chlorocarbonyl)-5a,5b,8,8,11a-pentamethyl-1-(prop-1-en-2-yl)icosahydro-1H-cyclopenta[a]chrysen-9-yl acetate (step 2) (3.62 g, 7.0 mmol, 1.0 eq) in CH₂Cl₂ (20 ml) was added and the mixture was allowed to stir at room temperature for overnight. The reaction mixture diluted with CH₂Cl₂ (50 ml), washed with water and brine solution. The combined organic layers were dried over Na₂SO₄, filtered and evaporated under reduced pressure. The crude residue was purified by silicagel column chromatography by using 2% methanol:dichloromethane as an eluent to afford the desired product (1.5 g) as a white solid. ¹H NMR (300 MHz, CDCl₃): δ ppm 5.65 (d, J=7.8 Hz, 1H), 4.73 (s, 1H), 4.59 (s, 1H), 4.51-4.43 (m, 1H), 4.21-4.08 (m, 1H), 3.18-3.06 (m, 1H), 2.73-2.65 (m, 1H), 2.53-2.42 (m, 1H), 2.43-2.30 (m, 1H), 2.07-2.0 (m, 4H), 1.97-1.87 (m, 3H), 1.80-1.72 (m, 2H), 1.67 (brs, 3H), 1.66-1.58 (m, 3H), 1.57-1.50 (m, 2H), 1.50-1.44 (m, 2H), 1.43-1.40 (m, 2H), 1.40-1.30 (m, 6H), 1.28-1.23 (m, 2H), 1.23-1.13 (m, 2H), 1.0-0.90 (m, 10 H), 0.87-0.80 (m, 9H), 0.80-0.75 (m, 1H); ES MS: [M+H]⁺ 624.6 (40%), [M+Na]⁺ 646.5 (20%). [M−H]⁻ 622.5 (100%).

### Step 5: (1R,3aS,5aR,5bR,7aR,9S,11aR,11bR,13aR,13bR)-3a-((1R,3S)-3-(hydrazinecarbonyl)-2,2-dimethylcyclobutylcarbamoyl)-5a,5b,8,8,11a-pentamethyl-1-(prop-1-en-2-yl) icosahydro-1H-cyclopenta[a]chrysen-9-ylacetate

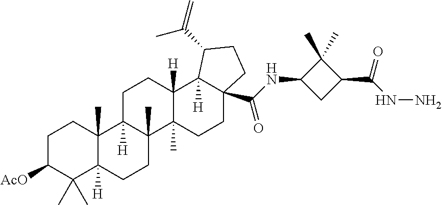

To a stirred solution of (1S,3R)-3-((1R,3aS,5aR,5bR,7aR,9S,11aR,11bR,13aR,13bR)-9-acetoxy-5a,5b,8,8,11a-pentamethyl-1-(prop-1-en-2-yl)icosahydro-1H-cyclopenta[a]chrysene-3a-carboxamido)-2,2-dimethylcyclobutanecarboxylicacid (step 4) (1.5 g, 2.40 mmol, 1.0 eq) in DMF (15 ml) were added HATU (1.19 g, 3.13 mmol, 1.3 eq) and DIPEA (1.86 g, 14.4 mmol, 6.0 eq). The reaction mixture was stirred at room temperature for 45 min., and then Hydrazine hydrate (1.2 g, 24.07 mmol, 10 eq) was added and stirred at room temperature for over night. TLC indicated starting material was consumed and the desired product was observed. The reaction mixture was diluted with ice cooled water and solids that formed were collected by filtration and dried under vacuum. The crude solid was purified by silicagel column chromatography by using 2.5% methanol:dichloromethane as an eluent to give the desired product (1.2 g, 78.4%) as a white solid. ¹H NMR (300 MHz, DMSO-d6): δ ppm 8.78 (s, 1H), 7.56 (d, J=7.5 Hz, 1H), 4.63 (s, 1H), 4.52 (s, 1H), 4.40-4.32 (m, 1H), 4.15 (brs, 1H), 3.95-3.80 (m, 1H), 3.0-2.90 (m, 1H), 2.72-2.58 (m, 1H), 2.40-2.30 (m, 2H), 2.28-2.18 (m, 1H), 2.0 (brs, 3H), 1.98-1.82 (m, 2H), 1.83-1.78 (m, 1H), 1.78-1.67 (m, 1H), 1.67-1.53 (m, 6H), 1.52-1.42 (m, 3H), 1.40-1.20 (m, 10H), 1.20-1.13 (m, 2H), 1.12-1.08 (m, 2H), 1.07-1.0 (m, 1H), 1.0-0.96 (m, 1H), 0.91 (s, 3H), 0.84 (s, 3H), 0.82-0.72 (m, 12H), 0.72-0.68 (m, 1H); ES MS: [M+H]⁺ 638.6 (100%).

### Step 6: (1R,3aS,5aR,5bR,7aR,9S,11aR,11bR,13aR,13bR)-3a-((1R,3S)-2,2-dimethyl-3-(2-(3-methyl butanoyl)hydrazinecarbonyl)cyclobutylcarbamoyl)-5a,5b,8,8,11a-pentamethyl-1-(prop-1-en-2-yl)icosahydro-1H-cyclopenta[a]chrysen-9-ylacetate

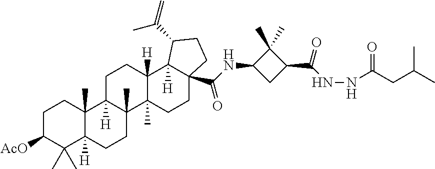

To a stirred solution of (1R,3aS,5aR,5bR,7aR,9S,11aR,11bR,13aR,13bR)-3a-((1R,3S)-3-(hydrazinecarbonyl)-2,2-dimethylcyclobutylcarbamoyl)-5a,5b,8,8,11a-pentamethyl-1-(prop-1-en-2-yl)icosahydro-1H-cyclopenta[a]chrysen-9-ylacetate (step 5) (1.2 g, 1.88 mmol, 1.0 eq) in DMF (12.5 ml) were added HATU (0.930 g, 2.44 mmol, 1.3 eq) and DIPEA (1.45 g, 11.30 mmol, 6.0 eq). The reaction mixture was stirred at room temperature for 1 hour then Isovaleric acid (0.288 g, 2.82 mmol, 1.5 eq) was added stirred at room temperature for overnight. TLC indicated starting material was consumed and the desired product was observed. The reaction mixture was diluted with ice cooled water and solids that formed were collected by filtration and dried under vacuum. The crude solid was purified by silicagel column chromatography by using 2% methanol:dichloromethane as an eluent to give the desired product (1.0 g, 74% yield) as a white solid. ¹H NMR (300 MHz, CDCl₃): δ ppm 8.18 (d, J=2.4 Hz, 2H), 5.92-5.85 (m, 1H), 4.72 (s, 1H), 4.58 (s, 1H), 4.50-4.43 (m, 1H), 4.20-4.10 (m, 1H), 3.15-3.03 (m, 1H), 2.59-2.30 (m, 3H), 2.19-2.08 (m, 3H), 2.04 (brs, 3H), 2.0-1.85 (m, 2H), 1.80-1.70 (m, 1H), 1.70-1.58 (m, 9H), 1.51-1.40 (m, 4H), 1.39-1.20 (m, 10H), 1.20-1.10 (m, 1H), 1.05-0.88 (m, 17H), 0.87-0.79 (m, 9H), 0.78-0.70 (m, 1H); ES MS: [M+H]⁺ 722.7 (100%), [M+Na]⁺ 744.7 (90%).

### Step 7: (1R,3aS,5aR,5bR,7aR,9S,11aR,11bR,13aR,13bR)-3a-((1R,3S)-3-(5-isobutyl-1,3,4-oxadiazol-2-yl)-2,2-dimethylcyclobutylcarbamoyl)-5a,5b,8,8,11a-pentamethyl-1-(prop-1-en-2-yl)icosa hydro-1H-cyclopenta[a]chrysen-9-ylacetate

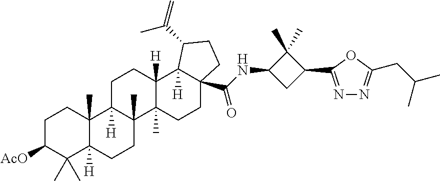

To a stirred solution of (1R,3aS,5aR,5bR,7aR,9S,11aR,11bR,13aR,13bR)-3a-((1R,3S)-2,2-dimethyl-3-(2-(3-methyl butanoyl)hydrazinecarbonyl)cyclobutylcarbamoyl)-5a,5b,8,8,11a-pentamethyl-1-(prop-1-en-2-yl)icosahydro-1H-cyclopenta[a]chrysen-9-ylacetate (step 6) (1.0 g, 1.38 mmol, 1.0 eq) in CH₂Cl₂ (10 ml) at 0° C. was added triethylamine (0.42 g, 4.16 mmol, 3.0 eq) and para-toluenesulfonylchloride (0.34 g, 1.80 mmol, 1.3 eq). The reaction mixture was allowed to stir at room temperature for overnight. TLC indicated starting material was consumed and the desired product was observed. The reaction mixture was diluted with CH₂Cl₂ (50 ml), washed with water (100 ml) and brine solution. The combined organic layers were dried over Na₂SO₄, filtered and evaporated under reduced pressure. The residue was purified by silicagel column chromatography by using 1.5% methanol:dichloromethane as an eluent to give the desired product (0.7 g) as a white solid. ES MS: [M+H]⁺ 704.6 (100%).

### Step 8: (1R,3aS,5aR,5bR,7aR,9S,11aR,11bR,13aR,13bR)-9-hydroxy-N-((1R,3S)-3-(5-iso butyl-1,3,4-oxadiazol-2-yl)-2,2-dimethylcyclobutyl)-5a,5b,8,8,11a-pentamethyl-1-(prop-1-en-2-yl)icosahydro-1H-cyclopenta[a]chrysene-3a-carboxamide

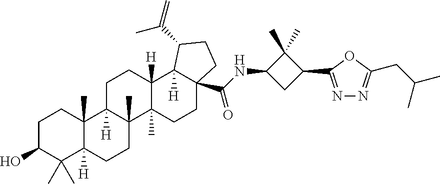

To a stirred solution of (1R,3aS,5aR,5bR,7aR,9S,11aR,11bR,13aR,13bR)-3a-((1R,3S)-3-(5-isobutyl-1,3,4-oxadiazol-2-yl)-2,2-dimethylcyclobutylcarbamoyl)-5a,5b,8,8,11a-pentamethyl-1-(prop-1-en-2-yl)icosa hydro-1H-cyclopenta[a]chrysen-9-ylacetate (step 7) (0.7 g, 0.995 mmol, 1.0 eq) in THF (7 ml) and Methanol (7 ml) was added potassium carbonate (0.961 g, 6.97 mmol, 7.0 eq). The reaction mixture was stirred at room temperature for 48 hours, TLC indicated reaction did not proceed completely. The mixture was filtered through a pad of celite and was evaporated under reduced pressure. To the crude compound in 1,4-dioxane (10 ml) was added 4N NaOH (10 ml) solution and stirred at room temperature for overnight. TLC indicated starting material was consumed and the desired product was observed. The mixture was concentrated under reduced pressure, cooled to 0° C., acidified to pH 5 using 1N citric acid solution and extracted with CH₂Cl₂ (2×100 ml). The combined organic extracts were washed with water, brine solution, dried over Na₂SO₄, filtered and evaporated under reduced pressure. The residue was purified by silicagel column chromatography by using 1% methanol:chloroform as an eluent to give the desired product (0.600 g, 91% yield) as a white solid. ES MS: [M+H]⁺ 662.5 (100%), [M+Na]⁺ 684.5 (30%).

### Step 9: 4-((1R,3aS,5aR,5bR,7aR,9S,11aR,11bR,13aR,13bR)-3a-((1R,3S)-3-(5-isobutyl-1,3,4-oxa diazol-2-yl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoicacid

To a stirred solution of (1R,3aS,5aR,5bR,7aR,9S,11aR,11bR,13aR,13bR)-9-hydroxy-N-((1R,3S)-3-(5-iso butyl-1,3,4-oxadiazol-2-yl)-2,2-dimethylcyclobutyl)-5a,5b,8,8,11a-pentamethyl-1-(prop-1-en-2-yl)icosahydro-1H-cyclopenta[a]chrysene-3a-carboxamide (step 8) (0.500 g, 0.756 mmol, 1.0 eq) and 2,2-dimethyl succinicanhydride (0.38 g, 3.02 mmol, 4.0 eq) in toluene (5 ml) was added DMAP (0.184 g, 1.51 mmol, 2.0 eq). The reaction mixture was heated at 80° C. for overnight. TLC indicated starting material was consumed and the desired product was observed. The mixture was concentrated under reduced pressure, cooled to 0° C., acidified to pH 5 with 1N citric acid solution and extracted with CH₂Cl₂ (2×100 ml). The combined organic extracts were washed with water (100 ml), brine solution (100 ml), dried over Na₂SO₄, filtered and evaporated under reduced pressure. The residue was purified by silicagel column chromatography by using 2% methanol:dichloromethane as an eluent to give the desired product (0.18 g, 30% yield) as a white solid. ¹H NMR (300 MHz, CDCl₃): δ ppm 5.80 (d, J=7.5 Hz, 1H), 4.73 (s, 1H), 4.58 (s, 1H), 4.51-4.43 (m, 1H), 4.32-4.20 (m, 1H), 3.18-3.05 (m, 2H), 2.75-2.60 (m, 4H), 2.58-2.36 (m, 2H), 2.20-2.09 (m, 1H), 2.0-1.83 (m, 3H), 1.80-1.70 (m, 2H), 1.70-1.65 (m, 4H), 1.64-1.52 (m, 4H), 1.50-1.42 (m, 3H), 1.40-1.32 (m, 6H), 1.33-1.22 (m, 9H), 1.20-1.10 (m, 2H), 1.10-0.88 (m, 14H), 0.88-0.76 (m, 12H), 0.76-0.74 (m, 1H); ES MS: [M+H]⁺ 790.7 (100%), [M+Na]⁺ 812.7 (70%); HPLC: 84.02+8.04% (isomers).

### Example 17

### Preparation of (1R,3S)-3-(((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(1-methylcyclopropyl)icosahydro-1H-cyclopenta[a]chrysen-9-yloxy)carbonyl)-2,2-dimethyl cyclobutanecarboxylicacid

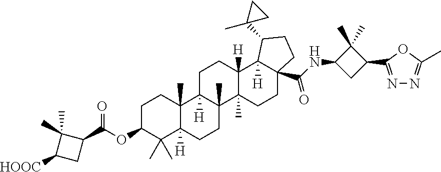

### Step 1: (1R,3aS,5aR,5bR,7aR,9S,11aR,11bR,13aR,13bR)-benzyl-9-acetoxy-5a,5b,8,8,11a-penta methyl-1-(prop-1-en-2-yl)icosahydro-1H-cyclopenta[a]chrysene-3a-carboxylate

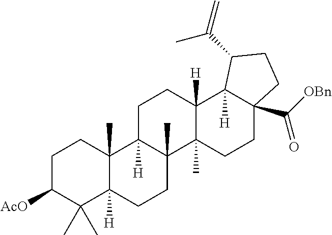

To a stirred solution of (1R,3aS,5aR,5bR,7aR,9S,11aR,1 bR,13aR,13bR)-9-acetoxy-5a,5b,8,8,11a-pentamethyl-1-(prop-1-en-2-yl)icosahydro-1H-cyclopenta[a]chrysene-3a-carboxylic acid (20 g, 40.16 mmol, 1.0 eq) in DMF (200 ml) was added potassium carbonate (11.0 g, 80.32 mmol, 2.0 eq) and benzyl bromide (6.8 g, 40.16 mmol, 1.0 eq). The reaction mixture was heated at 70° C. for overnight. TLC indicated starting material was consumed and the desired product was observed. The reaction mixture was cooled to 0° C., diluted with water (500 ml) and the solid was precipitated out. The solids that were collected by filtration dissolved in CH₂Cl₂, dried over Na₂SO₄, filtered, evaporated and purified by silicagel column chromatography by using 2% methanol:dichloromethane as an eluent gave the desired product (19.0 g, 80% yield) as a white solid. ¹H NMR (300 MHz, CDCl₃): δ ppm 7.38-7.31 (m, 5H), 5.17-5.05 (m, 2H), 4.72 (s, 1H), 4.59 (s, 1H), 4.49-4.42 (m, 1H), 3.07-2.95 (m, 1H), 2.32-2.24 (m, 1H), 2.24-2.12 (m, 1H), 2.04 (s, 3H), 1.95-1.80 (m, 2H), 1.72-1.69 (m, 1H), 1.67 (s, 3H), 1.64-1.58 (m, 3H), 1.50-1.30 (m, 9H), 1.28-1.22 (m, 2H), 1.21-1.15 (m, 1H), 1.15-0.96 (m, 3H), 0.93 (s, 3H), 0.82 (s, 9H), 0.80-0.78 (m, 1H), 0.75 (s, 3H); ES MS: [M+H]⁺ 611.3 (10%); IR (KBr) cm⁻¹: 2967, 2948, 2867, 1741, 1721, 1245, 1127.

### Step 2: (1R,3aS,5aR,5bR,7aR,9S,11aR,11bR,13aR,13bR)-benzyl-9-acetoxy-5a,5b,8,8,11a-penta methyl-1-(1-methylcyclopropyl)icosahydro-1H-cyclopenta[a]chrysene-3a-carboxylate

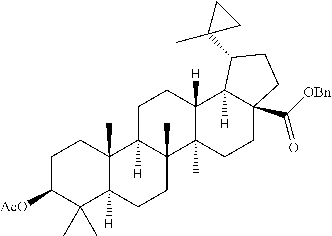

To a flame dried round bottom flask under nitrogen (1R,3aS,5aR,5bR,7aR,9S,11aR,11bR,13aR,13bR)-benzyl-9-acetoxy-5a,5b,8,8,11a-penta methyl-1-(prop-1-en-2-yl)icosahydro-1H-cyclopenta[a]chrysene-3a-carboxylate (step 1) (5.0 g, 8.27 mmol, 1.0 eq) and dry CH₂Cl₂ (100 ml) was added. The reaction mixture was cooled to −25° C., diethylzinc (5.11 g, 41.39 mmol, 5.0 eq, 1.0 M solution in hexane) was added then stirred at the same temperature for 1 hour. Diiodomethane (17.73 g, 66.22 mmol, 8.0 eq) was added and the reaction mixture was allowed to stir at room temperature for overnight. The reaction mixture cooled to 0° C., quenched with saturated NH₄Cl solution, extracted with CH₂Cl₂ (3×50 ml). The combined organic extracts were dried over Na₂SO₄, filtered and evaporated under reduced pressure. The crude residue was purified by silicagel column chromatography by using 2% ethyl acetate:hexane as an eluent gave the desired product (4.93 g, 98% yield) as a white solid. H NMR (300 MHz, CDCl₃): δ ppm 7.38-7.31 (m, 5H), 5.15-5.05 (m, 2H), 4.51-4.41 (m, 1H), 2.28-2.21 (m, 1H), 2.14-2.07 (m, 1H), 2.04 (s, 3H), 1.98-1.90 (m, 1H), 1.90-1.77 (m, 2H), 1.76-1.68 (m, 1H), 1.67-1.60 (m, 3H), 1.58-1.56 (m, 2H), 1.50-1.44 (m, 2H), 1.43-1.25 (m, 8H), 1.24-1.14 (m, 1H), 1.13-0.99 (m, 2H), 0.95 (s, 3H), 0.90 (s, 3H), 0.83 (s, 9H), 0.83-0.78 (m, 1H), 0.75 (s, 3H), 0.41-0.30 (m, 2H), 0.27-0.17 (m, 2H); ES MS: [M+Na]⁺ 625.4 (10%).

### Step 3: (1R,3aS,5aR,5bR,7aR,9S,11aR,11bR,13aR,13bR)-9-acetoxy-5a,5b,8,8,11a-pentamethyl-1-(1-methylcyclopropyl)icosahydro-1H-cyclopenta[a]chrysene-3a-carboxylicacid

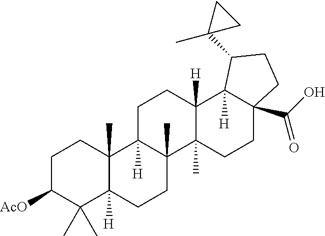

To a solution of (1R,3aS,5aR,5bR,7aR,9S,11aR,11bR,13aR,13bR)-benzyl-9-acetoxy-5a,5b,8,8,11a-penta methyl-1-(1-methylcyclopropyl)icosahydro-1H-cyclopenta[a]chrysene-3a-carboxylate (step 2) (4.3 g, 7.14 mmol, 1.0 eq) in ethyl acetate:ethanol (1:1, 43 ml) was added 10% Pd/C (0.500 g, 0.471 mmol, 0.066 eq) under nitrogen and the reaction mixture was hydrogenated at 50 psi for overnight. TLC indicated starting material was consumed and the desired product was observed. The reaction mixture was filtered through a pad of celite and was washed with ethyl acetate. The filtrate was evaporated under reduced pressure and the residue was purified by silicagel column chromatography by using 4% ethyl acetate:hexane as an eluent gave the desired product (3.4 g, 93% yield) as a white solid. ¹H NMR (300 MHz, CDCl₃): δ ppm 9.95 (brs, 1H), 4.52-4.45 (m, 1H), 2.25-2.18 (m, 1H), 2.14-2.07 (m, 1H), 2.05 (s, 3H), 1.98-1.85 (m, 3H), 1.70-1.57 (m, 5H), 1.57-1.30 (m, 10H), 1.30-1.12 (m, 3H), 1.08-1.0 (m, 1H), 0.98 (s, 3H), 0.92 (brs, 6H), 0.86 (s, 3H), 0.85 (s, 3H), 0.83 (s, 3H), 0.81-0.78 (m, 1H), 0.42-0.33 (m, 2H), 0.30-0.18 (m, 2H); ES MS: [M−H]⁻ 511.3 (50%); IR cm⁻¹: 3219, 2955, 2871, 1729, 1708, 1268, 979 cm⁻¹.

### Step 4: (1R,3aS,5aR,5bR,7aR,9S,11aR,11bR,13aR,13bR)-3a-(chlorocarbonyl)-5a,5b,8,8,11a-penta methyl-1-(1-methylcyclopropyl)icosahydro-1H-cyclopenta[a]chrysen-9-ylacetate

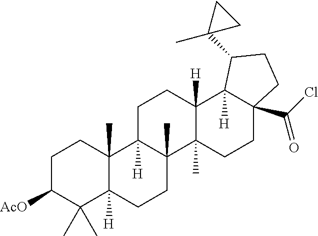

To a solution of (1R,3aS,5aR,5bR,7aR,9S,11aR,11bR,13aR,13bR)-9-acetoxy-5a,5b,8,8,11a-pentamethyl-1-(1-methylcyclopropyl)icosahydro-1H-cyclopenta[a]chrysene-3a-carboxylicacid (step 3) (1.7 g, 3.32 mmol, 1.0 eq) in toluene (15 ml) at 0° C. was added thionyl chloride (1.97 g, 16.6 mmol, 5.0 eq). The reaction mixture was allowed to reach to room temperature and heated to reflux for 3 hours. After completion of the reaction monitored by TLC, solvent was evaporated in vacuo providing a crude residue that was re-dissolved in toluene (2×10 ml) to remove the excess thionyl chloride. The solution was concentrated in vacuo to produce the desired product (1.76 g), which is used as such for next step.

### Step 5: (1S,3R)-3-amino-2,2-dimethylcyclobutanecarboxylicacidhydrochloride

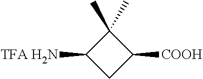

To a solution of (1S,3R)-3-(tert-butoxycarbonylamino)-2,2-dimethylcyclobutane carboxylic acid (7.0 g, 28.8 mmol, 1.0 eq) in dichloromethane (50 ml) at 0° C. was added trifluoroacetic acid (50 ml). The reaction mixture was allowed to stir at room temperature for 4 hours. TLC indicated starting material was consumed and the desired product was observed. The reaction mixture was evaporated under reduced pressure and the crude compound (7.5 g) was used as such for the next step.

### Step 6: (1S,3R)-3-((1R,3aS,5aR,5bR,7aR,9S,11aR,11bR,13aR,13bR)-9-acetoxy-5a,5b,8,8,11a-penta methyl-1-(1-methylcyclopropyl)icosahydro-1H-cyclopenta[a]chrysene-3a-carboxamido)-2,2-dimethylcyclobutanecarboxylicacid

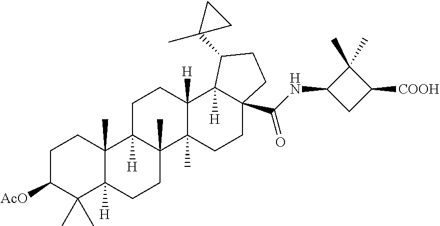

To a stirred solution of (1S,3R)-3-amino-2,2-dimethylcyclobutanecarboxylicacidhydrochloride (step 5) (0.95 g, 3.97 mmol, 1.2 eq) and triethylamine (2.0 g, 19.88 mmol, 6.0 eq) in CH₂Cl₂ (20 ml) at 0° C. was added a solution of (1R,3aS,5aR,5bR,7aR,9S,11aR,11bR,13aR,13bR)-3a-(chlorocarbonyl)-5a,5b,8,8,11a-penta methyl-1-(1-methylcyclopropyl)icosahydro-1H-cyclopenta[a]chrysen-9-ylacetate (step 4) (1.76 g, 3.31 mmol, 1.0 eq) in CH₂Cl₂ (15 ml). The reaction mixture was allowed to stir at room temperature for overnight. The reaction mixture was diluted with CH₂Cl₂, washed with water (2×100 ml) and brine solution (100 ml). The combined organic layers were dried over Na₂SO₄, filtered and evaporated under reduced pressure. The crude residue was purified by silicagel column chromatography by using 1.5% methanol/dichloromethane as an eluent to afford desired product (1.5 g, 75% yield) as a white solid. ¹H NMR (300 MHz, CDCl₃): δ ppm 5.60 (d, J=7.8 Hz, 1H), 4.52-4.44 (m, 1H), 4.17-4.05 (m, 1H), 2.71-2.63 (m, 1H), 2.43-2.27 (m, 2H), 2.05 (s, 3H), 2.0-1.36 (m, 16H), 1.35-1.30 (m, 5H), 1.27-1.24 (m, 2H), 1.22-1.10 (m, 2H), 1.04-0.76 (m, 24H), 0.45-0.38 (m, 1H), 0.38-0.30 (m, 1H), 0.28-0.15 (m, 2H); ES MS: [M+H]⁺ 638.5 (100%), [M+Na]⁺ 660.4 (50%).

### Step 7: (1R,3aS,5aR,5bR,7aR,9S,11aR,11bR,13aR,13bR)-3a-((1R,3S)-3-(2-acetylhydrazine carbonyl)-2,2-dimethylcyclobutylcarbamoyl)-5a,5b,8,8,11a-pentamethyl-1-(1-methyl cyclopropyl)icosahydro-1H-cyclopenta[a]chrysen-9-ylacetate

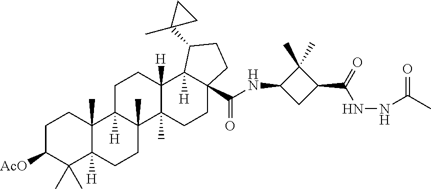

To a stirred solution of (1S,3R)-3-((1R,3aS,5aR,5bR,7aR,9S,11aR,11bR,13aR,13bR)-9-acetoxy-5a,5b,8,8,11a-penta methyl-1-(1-methylcyclopropyl)icosahydro-1H-cyclopenta[a]chrysene-3a-carboxamido)-2,2-dimethylcyclobutanecarboxylicacid (step 6) (1.5 g, 2.35 mmol, 1.0 eq) in DMF (15 ml), HATU (1.16 g, 3.06 mmol, 1.3 eq) and DIPEA (1.82 g, 14.12 mmol, 6.0 eq) were added then stirred at room temperature for 45 minutes. Acetic hydrazide (0.209 g, 2.82 mmol, 1.2 eq) was added and the reaction mixture was stirred at room temperature for overnight. TLC indicated starting material was consumed and the desired product was observed. The reaction mixture was diluted with ice cooled water and solids that formed were collected by filtration, washed with water (100 ml) and dried under vacuum. The crude solid was purified by silicagel column chromatography by using 2% methanol/dichloromethane as an eluent to obtain desired product (1.3 g, 79% yield) as a white solid. ¹H NMR (300 MHz, CDCl₃): δ ppm 8.36 (d, J=4.8 Hz, 1H), 8.19 (d, J=4.8 Hz, 1H), 5.85 (d, J=8.1 Hz, 1H), 4.52-4.42 (m, 1H), 4.17-4.05 (m, 1H), 2.57-2.48 (m, 1H), 2.42-2.27 (m, 2H), 2.18-2.08 (m, 1H), 2.06 (s, 3H), 2.04 (s, 3H), 2.02-1.92 (m, 1H), 1.90-1.70 (m, 4H), 1.62-1.54 (m, 2H), 1.54-1.35 (m, 8H), 1.35-1.27 (m, 6H), 1.27-1.22 (m, 1H), 1.20-1.10 (m, 2H), 1.07-1.0 (m, 1H), 0.97 (s, 3H), 0.96-0.90 (m, 9H), 0.89-0.87 (m, 1H), 0.87-0.82 (m, 9H), 0.80-0.78 (m, 1H), 0.47-0.38 (m, 1H), 0.38-0.30 (m, 1H), 0.28-0.15 (m, 2H); ES MS: [M+H]⁺ 694.5 (60%), [M+Na]⁺ 716.5 (100%).

### Step 8: (1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(1-methylcyclopropyl)icosahydro-1H-cyclopenta[a]chrysen-9-ylacetate

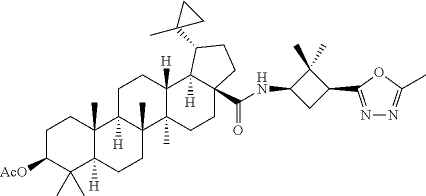

To a stirred solution of (1R,3aS,5aR,5bR,7aR,9S,11aR,11bR,13aR,13bR)-3a-((1R,3S)-3-(2-acetylhydrazine carbonyl)-2,2-dimethylcyclobutylcarbamoyl)-5a,5b,8,8,11a-pentamethyl-1-(1-methyl cyclopropyl)icosahydro-1H-cyclopenta[a]chrysen-9-ylacetate (step 7) (1.3 g, 1.87 mmol, 1.0 eq) and triethylamine (0.568 g, 5.62 mmol, 3.0 eq) in CH₂Cl₂ (15 ml) was added para-toluenesulfonylchloride (0.464 g, 2.43 mmol, 1.3 eq). The reaction mixture was stirred at room temperature for overnight. TLC indicated starting material was consumed and the desired product was observed. The reaction mixture was washed with water (50 ml), dried over Na₂SO₄, filtered and evaporated under reduced pressure. The crude residue was purified by silicagel column chromatography by using 25% ethyl acetate:hexane as eluent to afford desired product (0.75 g, 59% yield) as a white solid. ¹H NMR (300 MHz, CDCl₃): δ ppm 5.69 (d, J=7.8 Hz, 1H), 4.52-4.43 (m, 1H), 4.30-4.17 (m, 1H), 3.09 (dd, 1H, J=9.9, 8.4 Hz), 2.68-2.57 (m, 1H), 2.51 (s, 3H), 2.40-2.17 (m, 2H), 2.04 (s, 3H), 2.0-1.83 (m, 2H), 1.80-1.66 (m, 3H), 1.65-1.54 (m, 7H), 1.53-1.38 (m, 6H), 1.37-1.30 (m, 6H), 1.28-1.10 (m, 4H), 1.10-1.0 (m, 1H), 0.97 (s, 3H), 0.91 (s, 3H), 0.90 (s, 3H), 0.87-0.80 (m, 9H), 0.80-0.75 (m, 1H), 0.46-0.38 (m, 1H), 0.38-0.29 (m, 1H), 0.28-0.15 (m, 2H); ES MS: [M+H]⁺ 676.5 (90%), [M+Na]⁺ 698.5 (100%).

### Step 9: (1R,3aS,5aR,5bR,7aR,9S,11aR,11bR,13aR,13bR)—N-((1R,3S)-2,2-dimethyl-3-(5-methyl-1,3,4-oxadiazol-2-yl)cyclobutyl)-9-hydroxy-5a,5b,8,8,11a-pentamethyl-1-(1-methylcyclopropyl)icosahydro-1H-cyclopenta[a]chrysene-3a-carboxamide

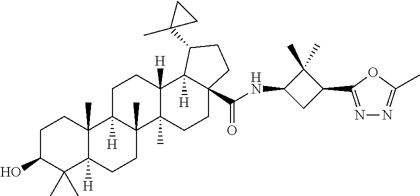

To a stirred solution of (1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(1-methylcyclopropyl)icosahydro-1H-cyclopenta[a]chrysen-9-ylacetate (step 8) (0.75 g, 1.11 mmol, 1.0 eq) in THF (7.5 ml) and Methanol (7.5 ml) was added potassium carbonate (1.07 g, 7.77 mmol, 7.0 eq). The reaction mixture was stirred at room temperature for 48 hours. TLC indicated starting material was consumed and the desired product was observed. The mixture was filtered through a pad of celite, washed with EtOAc (50 ml) and evaporated under reduced pressure. The crude residue was purified by silicagel column chromatography by using 1.5% methanol:dichloromethane as an eluent to give the desired product (0.5 g, 71.4% yield) as a white solid. ¹H NMR (300 MHz, CDCl₃): δ ppm 5.70 (d, J=7.5 Hz, 1H), 4.30-4.15 (m, 1H), 3.24-3.17 (m, 1H), 3.09 (dd, J=9.9, 8.1 Hz, 1H), 2.68-2.57 (m, 1H), 2.51 (s, 3H), 2.43-2.28 (m, 2H), 2.02-1.83 (m, 2H), 1.82-1.70 (m, 3H), 1.60-1.54 (m, 2H), 1.53-1.40 (m, 6H), 1.40-1.32 (m, 7H), 1.33-1.28 (m, 2H), 1.28-1.22 (m, 2H), 1.20-1.12 (m, 2H), 0.98 (s, 3H), 0.96 (s, 3H), 0.93 (s, 3H), 0.91 (s, 3H), 0.82 (s, 3H), 0.79 (s, 3H), 0.75 (s, 3H), 0.73-0.67 (m, 1H), 0.47-0.38 (m, 1H), 0.48-0.39 (m, 1H), 0.27-0.15 (m, 2H); ES MS: [M+H]⁺ 634.4 (60%), [M+Na]⁺ 656.4 (100%).

### Step 10: (1S,3R)-3-(benzyloxycarbonyl)-2,2-dimethylcyclobutanecarboxylic2,4,6-trichlorobenzoic anhydride

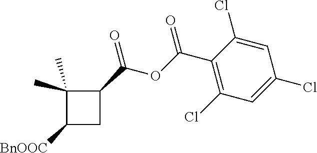

To a stirred solution of (1S,3R)-3-(benzyloxycarbonyl)-2,2-dimethylcyclobutane carboxylic acid (0.27 g, 1.03 mmol, 1.0 eq) and triethylamine (0.312 g, 3.09 mmol, 3.0 eq) in THF (3.0 ml) at 0° C. was added 2,4,6-trichlorobenzoyl chloride (0.301 g, 1.23 mmol, 1.2 eq). The reaction mixture was allowed to stir at room temperature for 3 hours. TLC indicated starting material was consumed and the desired product was observed. The reaction mixture was evaporated under reduced pressure obtained the crude compound (0.48 g) used as such for next step.

### Step 11: (1R,3S)-1-benzyl3-((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-penta methyl-1-(1-methylcyclopropyl)icosahydro-1H-cyclopenta[a]chrysen-9-yl)2,2-dimethyl cyclobutane-1,3-dicarboxylate

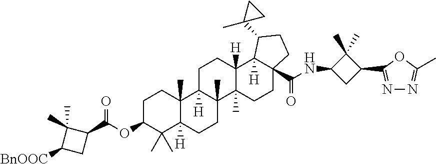

To a stirred solution of (1R,3aS,5aR,5bR,7aR,9S,11aR,11bR,13aR,13bR)—N-((1R,3S)-2,2-dimethyl-3-(5-methyl-1,3,4-oxadiazol-2-yl)cyclobutyl)-9-hydroxy-5a,5b,8,8,11a-pentamethyl-1-(1-methylcyclopropyl)icosahydro-1H-cyclopenta[a]chrysene-3a-carboxamide (step 9) (0.31 g, 0.489 mmol, 1.0 eq) in toluene (5 ml) was added DMAP (0.298 g, 2.44 mmol, 5.0 eq) and (1S,3R)-3-(benzyloxycarbonyl)-2,2-dimethylcyclobutanecarboxylic2,4,6-trichlorobenzoic anhydride (step 10) (0.458 g, 0.979 mmol, 2.0 eq) dissolved in toluene (5 ml). The reaction mixture was heated to reflux for overnight. TLC indicated starting material was consumed and the desired product was observed. The reaction mixture was cooled to room temperature diluted with CH₂Cl₂ (50 ml), washed with water (2×50 ml) and brine solution (2×50 ml). The combined organic layers were dried over Na₂SO₄, filtered and evaporated under reduced pressure. The crude residue was purified by silicagel column chromatography by using 0.5% methanol:dichloromethane as an eluent to obtain desired product (0.34 g, 79% yield) as a white solid. ¹H NMR (300 MHz, CDCl₃): δ ppm 7.40-7.32 (m, 5H), 5.69 (d, J=7.8 Hz, 1H), 5.12 (dd, J=16.2, 12.3 Hz, 2H), 4.44 (dd, J=11.1, 4.8 Hz, 1H), 4.28-4.17 (m, 1H), 3.09 (dd, J=9.6, 8.1 Hz, 1H), 2.86-2.73 (m, 2H), 2.70-2.58 (m, 2H), 2.52 (s, 3H), 2.42-2.28 (m, 2H), 2.08-1.90 (m, 2H), 1.90-1.67 (m, 4H), 1.66-1.61 (m, 4H), 1.58-1.54 (m, 3H), 1.53-1.40 (m, 6H), 1.37-1.30 (m, 9H), 1.29-1.22 (m, 2H), 1.22-1.07 (m, 3H), 0.97 (s, 3H), 0.96 (s, 3H), 0.91 (s, 3H), 0.89 (s, 3H), 0.84 (s, 3H), 0.82 (s, 3H), 0.79 (s, 3H), 0.78-0.75 (m, 1H), 0.45-0.38 (m, 1H), 0.38-0.29 (m, 1H), 0.28-0.15 (m, 2H); ES MS: [M+H]⁺ 878.6 (20%).

### Step 12: (1R,3S)-3-(((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(1-methylcyclopropyl)icosahydro-1H-cyclopenta[a]chrysen-9-yloxy) carbonyl)-2,2-dimethyl cyclobutanecarboxylicacid

To a solution of (1R,3S)-1-benzyl3-((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-penta methyl-1-(1-methylcyclopropyl)icosahydro-1H-cyclopenta[a]chrysen-9-yl)2,2-dimethyl cyclobutane-1,3-dicarboxylate (step 11) (0.34 g, 0.387 mmol, 1.0 eq) in dichloromethane (8 ml) was added palladium (II) acetate (0.0260 g, 0.116 mmol, 0.30 eq), triethyl amine (0.156 g, 1.55 mmol, 4.0 eq) and triethylsilane (0.09 g, 0.775 mmol, 2.0 eq). The mixture was flushed with N₂ and was heated to reflux for 48 hours. The reaction mixture was cooled to room temperature, filtered through a pad of celite and was washed with dichloromethane (50 ml). The combined organic layers were washed with water (50 ml), brine solution (50 ml) dried over Na₂SO₄, filtered and evaporated under reduced pressure. The crude residue was purified by silicagel column chromatography (eluent: 3% methanol:dichloromethane) followed by recrystallization over acetonitrile gave the desired product (0.090 g, 30% yield) as a white solid. ¹H NMR (300 MHz, CDCl₃): δ ppm 6.13 (d, J=7.5 Hz, 1H), 4.50 (t, J=8.1 Hz, 1H), 4.28-4.15 (m, 1H), 3.14-3.06 (m, 1H), 2.90-2.76 (m, 2H), 2.73-2.57 (m, 2H), 2.51 (s, 3H), 2.40-2.28 (m, 1H), 2.18-1.54 (m, 14H), 1.50-0.42 (m, 4H), 1.40-1.32 (m, 9H), 1.30-1.13 (m, 5H), 1.08-1.0 (m, 6H), 0.89 (brs, 6H), 0.85 (s, 3H), 0.83 (s, 3H), 0.81 (s, 3H), 0.79 (s, 3H), 0.48-0.38 (m, 1H), 0.38-0.30 (m, 1H), 0.28-0.14 (m, 2H); ES MS: [M+H]⁺ 788.5 (100%), [M+Na]⁺ 810.6 (100%); IR (KBr) cm⁻¹: 3411, 2953, 2870, 1720, 1566, 1192; HPLC: 90.8+6.3% (isomers).

### Example 18

### Preparation of 2-((1R,3S)-3-(((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclopropyl)acetic acid

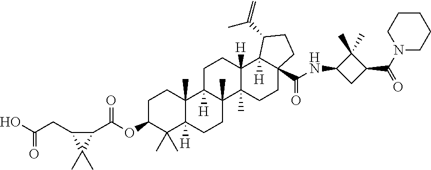

### Step 1: (1S,3R)-3-(2-methoxy-2-oxoethyl)-2,2-dimethylcyclopropanecarboxylicacid

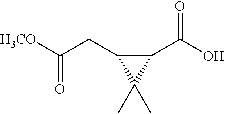

To a solution of Jones reagent (12 ml) in acetone (60 ml) at 0° C. was added methyl 2-((1R,3S)-3-(hydroxymethyl)-2,2-dimethylcyclopropyl)acetate (3.00 g, 17.4 mmol, 1.0 eq) in acetone (30 ml). The reaction mixture was allowed to stir at room temperature for overnight. TLC indicated starting material was consumed and the desired product was observed. The reaction mixture was quenched with isopropanol (25 ml) and solvent was evaporated under reduced pressure. The mixture was diluted with saturated brine solution and extracted with CH₂Cl₂ (3×50 ml). The combined organic layers were dried over Na₂SO₄, filtered and evaporated under reduced pressure. The residue was purified by silicagel column chromatography by using 1% methanol:dichloromethane as an eluent to give the desired product (1.2 g, 46% yield) as a pale yellow oil. ¹HNMR (300 MHz, CDCl₃): δ ppm 10.5 (brs, 1H), 3.68 (s, 3H), 2.76 (d, J=7.2 Hz, 2H), 1.61-1.43 (m, 2H), 1.23 (s, 3H), 1.21 (s, 3H).

### Step 2: 2,4,6-trichlorobenzoic (1S,3R)-3-(2-methoxy-2-oxoethyl)-2,2-dimethylcyclopropanecarboxylicanhydride

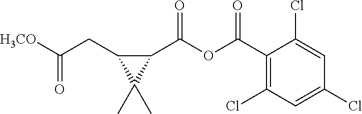 [SMILES: COC(=O)C[C@@H]1[C@H](C(=O)OC(=O)c2c(Cl)cc(Cl)cc2Cl)C1(C)C]

To a stirred solution of (1S,3R)-3-(2-methoxy-2-oxoethyl)-2,2-dimethylcyclopropanecarboxylic acid (step 1) (0.250 g, 1.34 mmol, 1.0 eq) and triethylamine (0.407 g, 4.03 mmol, 3.0 eq) in THF (8 ml) at 0° C. was added 2,4,6-trichlorobenzoyl chloride (0.393 g, 1.61 mmol, 1.2 eq). The reaction mixture was allowed to stir at room temperature for 3 hours. TLC indicated starting material was consumed and the desired product was observed. The reaction mixture was evaporated under reduced pressure to obtain the crude product (0.424 g), which was used as such for next step.

### Step 3: (1S,3R)-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yl) 3-(2-methoxy-2-oxoethyl)-2,2-dimethylcyclo propanecarboxylate

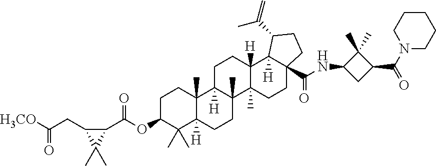

To a stirred solution of (1R,3aS,5aR,5bR,7aR,9S,11aR,11bR,13aR,13bR)—N-((1R,3S)-2,2-dimethyl-3-(piperidine-1-carbonyl)cyclobutyl)-9-hydroxy-5a,5b,8,8,11a-pentamethyl-1-(prop-1-en-2-yl)icosahydro-1H-cyclopenta[a]chrysene-3a-carboxamide (0.5 g, 0.771 mmol, 1.0 eq) in toluene (15 ml) was added DMAP (0.188 g, 1.54 mmol, 2.0 eq) and 2,4,6-trichlorobenzoic (1S,3R)-3-(2-methoxy-2-oxoethyl)-2,2-dimethylcyclopropanecarboxylicanhydride (step 2) (0.424 g, 1.08 mmol, 1.4 eq) in toluene (15 ml). The reaction mixture was heated at 90° C. for overnight. TLC indicated starting material was consumed and the desired product was observed. The reaction mixture was evaporated under reduced pressure, diluted with water (20 ml) and the aqueous layer was extracted with CH₂Cl₂ (3×50 ml). The combined organic layers were dried over Na₂SO₄, filtered and evaporated under reduced pressure. The residue was purified by silicagel column chromatography by using 1% methanol:dichloromethane as an eluent to obtain desired product (0.500 g, 79% yield) as a white solid. ¹H NMR (300 MHz, CDCl₃): δ ppm 5.89 (d, J=7.5 Hz, 1H), 4.72 (s, 1H), 4.58 (s, 1H), 4.48-4.39 (m, 1H), 4.18-4.05 (m, 1H), 3.70-3.65 (m, 4H), 3.50-3.35 (m, 3H), 3.20-3.05 (m, 1H), 2.88 (t, J=8.1 Hz, 1H), 2.76 (d, J=7.5 Hz, 2H), 2.60-2.40 (m, 1H), 2.40-2.20 (m, 2H), 2.03-1.85 (m, 3H), 1.70-1.63 (m, 7H), 1.62-1.50 (m, 6H), 1.50-1.40 (m, 4H), 1.40-1.38 (m, 5H), 1.38-1.30 (m, 4H), 1.28-1.22 (m, 3H), 1.19 (s, 3H), 1.18 (s, 3H), 1.15-1.08 (m, 1H), 1.05-1.0 (m, 1H), 0.99-0.90 (m, 8H), 0.87 (s, 3H), 0.83 (brs, 9H), 0.80-0.74 (m, 1H); ES MS: [M+H]⁺ 817.7 (100%), [M+Na]⁺ 839.8 (30%).

### Step 4: 2-((1R,3S)-3-(((1R,3aS,5aR,5bR,7aR,9S,11aR,11bR,13aR,13bR)-3a-((1R,3S)-2,2-di methyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclopropyl)acetic acid

To a stirred solution of (1S,3R)-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1 H-cyclopenta[a]chrysen-9-yl) 3-(2-methoxy-2-oxoethyl)-2,2-dimethylcyclo propanecarboxylate (step 3) (0.500 g, 0.612 mmol, 1.0 eq) in 1,4-dioxane (18 ml) at 0° C. was added aq. 2N NaOH solution (3.0 ml). The reaction mixture was allowed to stir at room temperature for overnight. TLC indicated starting material was consumed and the desired product was observed. The reaction mixture was diluted with water (30 ml), cooled to 0° C., acidified with 1N HCl to pH 5.0. The aqueous layer was extracted with CH₂Cl₂ (3×50 ml).

The combined organic layers were dried over Na₂SO₄, filtered and evaporated under reduced pressure. The residue was purified by silicagel column chromatography by using 20% ethyl acetate:hexane as an eluent to obtain desired product (0.170 g, 34.6% yield) as a white solid. ¹H NMR (300 MHz, CDCl₃): δ ppm 5.89 (d, J=8.7 Hz, 1H), 4.72 (s, 1H), 4.58 (s, 1H), 4.50-4.42 (m, 1H), 4.18-4.05 (m, 1H), 3.78-3.67 (m, 1H), 3.50-3.32 (m, 3H), 3.18-3.05 (m, 1H), 2.88 (t, J=8.1 Hz, 1H), 2.80 (d, J=7.5 Hz, 2H), 2.58-2.42 (m, 1H), 2.40-2.12 (m, 5H), 2.0-1.82 (m, 3H), 1.80-1.72 (m, 2H), 1.70-1.67 (m, 5H), 1.52-1.40 (m, 7H), 1.40-1.33 (m, 7H), 1.32-1.28 (m, 2H), 1.28-1.23 (m, 3H), 1.21 (s, 3H), 1.18 (s, 3H), 1.17-1.10 (m, 2H), 1.05-1.0 (m, 1H), 1.21 (s, 3H), 1.18 (s, 3H), 1.18-1.10 (m, 1H), 0.87 (s, 3H), 0.83 (s, 9H), 0.79-0.74 (m, 1H); ES MS: [M+H]⁺ 803.6 (100%), [M+Na]⁺ 825.6 (50%); HPLC: 94.2%

### Example 19

### Preparation of 2-((1R,3S)-3-(((1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yloxy)carbonyl)-2,2-dimethylcyclopropyl)aceticacid

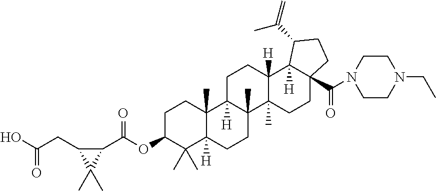

### Step 1: (1R,3aS,5aR,5bR,7aR,9S,11aR,11bR,13aR,13bR)-9-acetoxy-5a,5b,8,8,11a-pentamethyl1-(prop-1-en-2-yl)icosahydro-1H-cyclopenta[a]chrysene-3a-carboxylicacid

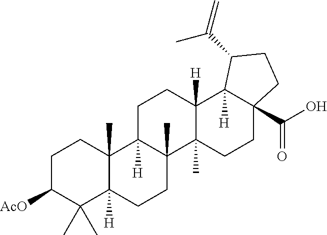

Acetic anhydride (17.08 g, 167.5 mmol, 5.1 eq) was added to a solution of Betulinic acid (15.0 g, 32.84 mmol, 1.0 eq), DIPEA (12.73 g, 98.53 mmol, 3.0 eq) and DMAP (0.80 g, 6.56 mmol, 0.2 eq) in THF (225 ml). The mixture was heated at 65-70° C. for overnight. TLC indicated starting material was consumed and the desired product was observed. The mixture was concentrated in vacuo to dryness to yield a white solid. To hydrolyze the mixed anhydride, this solid was suspended into 0.6N hydrochloric acid solution (150 ml) and heated to 100° C. for 6 hours. The suspension was cooled to room temperature and the solid was collected by filtration, washed with water and dried at 50° C. under reduced pressure for 1 hour to obtain desired product (15 g) as an off-white solid. ¹H NMR (300 MHz, CDCl₃): δ ppm 4.74 (s, 1H), 4.61 (s, 1H), 4.49-4.44 (m, 1H), 3.04-2.96 (m, 1H), 2.32-2.15 (m, 2H), 2.05 (s, 3H), 2.02-1.93 (m, 2H), 1.65-1.35 (m, 17H), 1.32-1.16 (m, 3H), 1.10-1.0 (m, 1H), 1.0-0.9 (m, 7H), 0.87-0.82 (m, 9H), 0.79-0.75 (m, 1H); ES MS: [M−H]⁻ 497.0 (100%).

### Step 2: (1R,3aS,5aR,5bR,7aR,9S,11aR,1 bR,13aR,13bR)-3a-(chlorocarbonyl)-5a,5b,8,8,11a-penta methyl-1-(prop-1-en-2-yl)icosahydro-1H-cyclopenta[a]chrysen-9-ylacetate

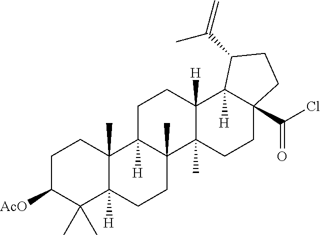

To a stirred solution of (1R,3aS,5aR,5bR,7aR,9S,11aR,11bR,13aR,13bR)-9-acetoxy-5a,5b,8,8,11a-pentamethyl1-(prop-1-en-2-yl)icosahydro-1H-cyclopenta[a]chrysene-3a-carboxylicacid (step 1) (3.5 g, 7.02 mmol, 1.0 eq) in CH₂Cl₂ (30 ml) at 0° C. was added oxalyl chloride (2.229 g, 17.5 mmol, 2.5 eq). The reaction mixture was allowed to stir at room temperature for overnight. After completion of the reaction monitored by TLC, the mixture was concentrated in vacuo providing a crude residue that was re-dissolved in CH₂Cl₂ to remove the excess oxalyl chloride. The solution was concentrated in vacuo to obtain the desired compound (3.5 g), which is used as such for next step without further purification.

### Step 3: (1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-ylacetate

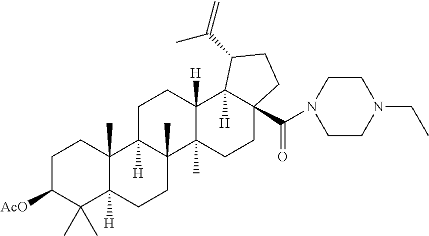

To a stirred solution of ethyl piperazine (1.54 g, 13.5 mmol, 2.0 eq) and triethylamine (4.79 g, 47.4 mmol, 7.0 eq) in CH₂Cl₂ (40 ml) at 0° C. was added a solution of (1R,3aS,5aR,5bR,7aR,9S,11aR,11bR,13aR,13bR)-3a-(chlorocarbonyl)-5a,5b,8,8,11a-penta methyl-1-(prop-1-en-2-yl)icosahydro-1H-cyclopenta[a]chrysen-9-ylacetate (step 2) (3.5 g, 6.78 mmol, 1.0 eq) in CH₂Cl₂ (15 ml). The reaction mixture was allowed to stir at room temperature for overnight. The reaction mixture was quenched with water and extracted with CH₂Cl₂ (3×50 ml). The combined organic layers were dried over Na₂SO₄, filtered and evaporated under reduced pressure. The residue was purified by silicagel column chromatography by using 1% methanol:dichloromethane as an eluent to obtain desired product (2.8 g, 70% yield) as a white solid. ¹H NMR (300 MHz, CDCl₃): δ ppm 4.72 (s, 1H), 4.58 (s, 1H), 4.50-4.43 (m, 1H), 3.70-3.60 (m, 4H), 3.03-2.82 (m, 2H), 2.50-2.38 (m, 6H), 2.15-2.05 (m, 1H), 2.07 (s, 3H), 2.02-1.94 (m, 1H), 1.92-1.80 (m, 1H), 1.78-1.70 (m, 1H), 1.68 (s, 3H), 1.67-1.48 (m, 6H), 1.45-1.32 (m, 7H), 1.30-1.23 (m, 2H), 1.20-1.08 (m, 4H), 1.05-0.97 (m, 1H), 0.96-0.93 (m, 6H), 0.93-0.88 (m, 1H), 0.87-0.80 (m, 9H), 0.80-0.75 (m, 1H); ES MS: [M+H]⁺ 595.6 (100%).

### Step 4: (4-ethylpiperazin-1-yl)((1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysen-3a-yl)methanone

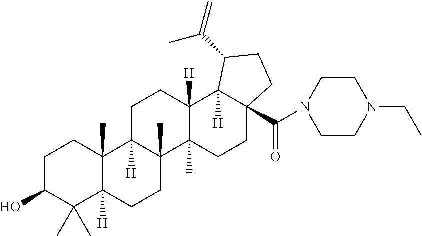

To a stirred solution of (1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-ylacetate (step 3) (2.8 g, 4.71 mmol, 1.0 eq) in THF (20 ml) and Methanol (20 ml) was added potassium carbonate (4.55 g, 32.97 mmol, 7.0 eq). The reaction mixture was stirred at room temperature for 48 hours. TLC indicated starting material was consumed and the desired product was observed. The mixture was filtered through a pad of celite, washed with CH₂Cl₂ and the filtrate was concentrated under reduced pressure. The crude residue was purified by silicagel column chromatography by using 2% methanol:dichloromethane as an eluent gave the desired product (1.7 g, 65% yield) as a white solid. ¹H NMR (300 MHz, CDCl₃): δ ppm 4.72 (s, 1H), 4.58 (s, 1H), 3.80-3.60 (m, 4H), 3.22-3.15 (m, 1H), 3.02-2.80 (m, 2H), 2.60-2.40 (m, 6H), 2.14-2.05 (m, 1H), 2.02-1.80 (m, 2H), 1.78-1.73 (m, 1H), 1.68 (s, 3H), 1.64-1.50 (m, 6H), 1.47-1.30 (m, 7H), 1.30-1.22 (m, 2H), 1.20-1.10 (m, 4H), 1.0-0.88 (m, 11H), 0.82 (s, 3H), 0.75 (s, 3H), 0.70-0.65 (m, 1H); ES MS: [M+H]⁺ 553.6 (100%).

### Step 5: (1S,3R)-((1R,3aS,5aR,5bR,7aR,9S,11aR,11bR,13aR,13bR)-3a-(4-ethylpiperazine-1-carbon yl)-5a,5b,8,8,11a-pentamethyl-1-(prop-1-en-2-yl)icosahydro-1H-cyclopenta[a]chrysen-9-yl) 3-(2-methoxy-2-oxoethyl)-2,2-dimethylcyclopropanecarboxylate

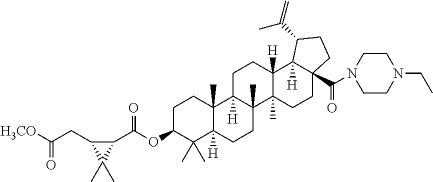

To a stirred solution of 2,4,6-trichlorobenzoic (1S,3R)-3-(2-methoxy-2-oxoethyl)-2,2-dimethylcyclopropanecarboxylic anhydride (0.497 g, 1.26 mmol, 1.4 eq) in toluene (15 ml) was added (4-ethylpiperazin-1-yl)((1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysen-3a-yl)methanone (step 4) (0.500 g, 0.904 mmol, 1.0 eq) and DMAP (0.220 g, 1.808 mmol, 2.0 eq). The reaction mixture was heated at 90° C. for overnight. TLC indicated starting material was consumed and the desired product was observed. The reaction mixture was evaporated under reduced pressure, diluted with water (20 ml) and extracted with CH₂Cl₂ (3×50 ml). The combined organic layers were dried over Na₂SO₄, filtered and evaporated under reduced pressure. The crude residue was purified by silicagel column chromatography by using 1% methanol:dichloromethane as an eluent to obtain desired product (0.500 g, 76% yield) as a white solid.

### Step 6: 2-((1R,3S)-3-(((1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yloxy)carbonyl)-2,2-dimethylcyclopropyl)aceticacid

To a stirred solution of (1S,3R)-((1R,3aS,5aR,5bR,7aR,9S,11aR,11bR,13aR,13bR)-3a-(4-ethylpiperazine-1-carbon yl)-5a,5b,8,8,11a-pentamethyl-1-(prop-1-en-2-yl)icosahydro-1H-cyclopenta[a]chrysen-9-yl) 3-(2-methoxy-2-oxoethyl)-2,2-dimethylcyclopropanecarboxylate (step 5) (0.500 g, 0.693 mmol, 1.0 eq) in 1,4-dioxane (18 ml) at 0° C. was added aq. 2N NaOH solution (3.46 ml). The reaction mixture was allowed to stir at room temperature for overnight. TLC indicated starting material was consumed and the desired product was observed. The reaction mixture was evaporated under reduced pressure, diluted with water (50 ml), cooled to 0° C., acidified with 1N HCl to pH 5. The aqueous layer was extracted with CH₂Cl₂ (3×50 ml). The combined organic layers were dried over Na₂SO₄, filtered and evaporated under reduced pressure. The residue was purified by silicagel column chromatography by using 2.5% methanol:dichloromethane as an eluent gave the desired product (0.300 g, 61% yield) as a white solid. ¹H NMR (300 MHz, CDCl₃): δ ppm 4.72 (s, 1H), 4.58 (s, 1H), 4.57-4.51 (m, 1H), 3.90-3.40 (m, 4H), 3.02-2.92 (m, 1H), 2.90-2.68 (m, 4H), 2.60-2.40 (m, 6H), 2.40-2.13 (m, 4H), 2.12-2.0 (m, 2H), 2.0-1.78 (m, 3H), 1.67-1.50 (m, 6H), 1.48-1.30 (m, 9H), 1.28-1.25 (m, 1H), 1.20 (s, 3H), 1.16 (s, 3H), 1.11 (t, J=7.2 Hz, 3H), 1.02-0.97 (m, 1H), 0.95 (s, 3H), 0.93 (s, 3H), 0.83 (brs, 9H), 0.80-0.75 (m, 1H); ES MS: [M+H]⁺ 707.8 (100%), [M+Na]⁺ 729.9 (20%); HPLC: 99.7%.

### Example 20

### Preparation of (1S,3R)-3-(((1R,3aS,5aR,5bR,7aR,9S,11aR,11bR,13aR,13bR)-3a-((1S,3R)-3-(4-ethylpiperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclobutane carboxylic acid

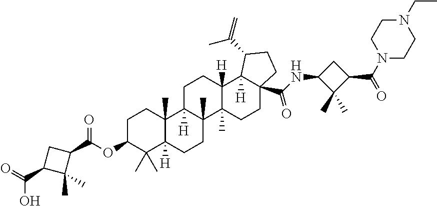

### Step 1: Synthesis of (1R,3aS,5aR,5bR,7aR,9S,11aR,11bR,13aR,13bR)-3a-((1S,3R)-3-(4-ethylpiperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-ylacetate

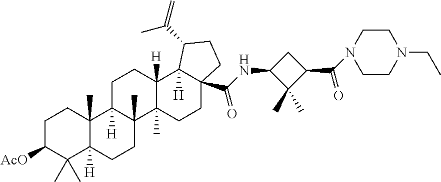

To a stirred solution of N-((1S,3R)-3-(4-ethylpiperazine-1-carbonyl)-2,2-dimethylcyclobutyl)-2,2,2-trifluoroacetamide (1.63 g, 5.80 mmol, 1.0 eq) in CH₂Cl₂ (20 ml) at 0° C. was added Et₃N (3.51 g, 34.84 mmol, 6.0 eq) and a solution of (1R,3aS,5aR,5bR,7aR,9S,11aR,11bR,13aR,13bR)-3a-(chlorocarbonyl)-5a,5b,8,8,11a-pentamethyl-1-(prop-1-en-2-yl)icosahydro-1H-cyclopenta[a]chrysen-9-yl acetate (3.0 g, 5.80 mmol, 1.0 eq) in CH₂Cl₂ (20 ml). The reaction mixture was allowed to stir at room temperature for overnight. TLC indicated starting material was consumed and the desired product was observed. The reaction mixture diluted with water, organic layer was separated and the aqueous layer was extracted with CH₂Cl₂ (3×25 ml). The combined organic layers were dried over Na₂SO₄, filtered and evaporated under reduced pressure. The crude residue was purified by silicagel column chromatography by using 2% methanol/dichloromethane as eluent to afford desired compound (1.4 g, 33% yield) as an off-white solid. ¹H NMR (300 MHz, CDCl₃): δ ppm 5.90 (d, 1H, J=7.8 Hz,), 4.73 (s, 1H), 4.58 (s, 1H), 4.50-4.42 (m, 1H), 4.11 (q, 1H, J=8.1 Hz), 3.84-3.74 (m, 1H), 3.60-3.42 (m, 3H), 3.17-3.06 (m, 1H), 2.88 (t, 1H, J=7.8 Hz), 2.57-2.27 (m, 8H), 2.04 (s, 3H), 1.99-1.72 (m, 3H), 1.74 (s, 3H), 1.65-1.60 (m, 2H), 1.59-1.53 (m, 6H), 1.53-1.41 (m, 4H), 1.41-1.35 (m, 6H), 1.34-1.20 (m, 5H), 1.20-1.14 (m, 1H), 1.09 (t, 3H, J=7.2 Hz), 1.05-0.98 (m, 1H), 0.95 (s, 3H), 0.92 (s, 3H), 0.90-0.85 (m, 1H), 0.83 (brs, 9H), 0.79-0.74 (m, 1H); ES MS: [M+H]⁺ 720.5 (100%).

### Step 2: Synthesis of (1R,3aS,5aR,5bR,7aR,9S,11aR,11bR,13aR,13bR)—N-((1S,3R)-3-(4-ethylpiperazine-1-carbonyl)-2,2-dimethylcyclobutyl)-9-hydroxy-5a,5b,8,8,11a-pentamethyl-1-(prop-1-en-2-yl)icosahydro-1H-cyclopenta[a]chrysene-3a-carboxamide

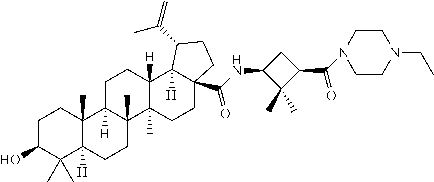

To a stirred solution of (1R,3aS,5aR,5bR,7aR,9S,11aR,11bR,13aR,13bR)-3a-((1S,3R)-3-(4-ethylpiperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-ylacetate (step 1, 1.4 g, 1.94 mmol, 1.0 eq) in THF (15 ml) and Methanol (15 ml) was added potassium carbonate (1.87 g, 13.6 mmol, 7.0 eq). The reaction mixture was stirred at room temperature for 48 hours. TLC indicated starting material was consumed and the desired product was observed. The reaction mixture was filtered through a pad of celite and the filterate was evaporated under reduced pressure. The crude residue was purified by silicagel column chromatography by using 4% methanol:dichloromethane as an eluent to give the desired product (1.0 g, 75% yield) as an off-white solid. ¹H NMR (300 MHz, CDCl₃): δ ppm 5.91 (d, J=9.0 Hz, 1H), 4.73 (s, 1H), 4.58 (s, 1H), 4.11 (q, J=8.4 Hz, 1H), 3.83-3.74 (m, 1H), 3.60-3.43 (m, 3H), 3.22-3.05 (m, 2H), 2.89 (t, J=7.8 Hz, 1H), 2.55-2.29 (m, 8H), 2.0-1.88 (m, 2H), 1.80-1.70 (m, 2H), 1.67 (s, 3H), 1.64-1.54 (m, 6H), 1.54-1.33 (m, 10H), 1.30-1.22 (m, 4H), 1.19 (s, 3H), 1.09 (t, 3H, J=7.2 Hz), 1.06-0.87 (m, 8H), 0.83 (s, 3H), 0.80 (s, 3H), 0.74 (s, 3H), 0.70-0.65 (m, 1H); ES MS: [M+H]⁺ 678.6 (100%).

### Step 3: Synthesis of (1S,3R)-1-tert-butyl 3-((1R,3aS,5aR,5bR,7aR,9S,11aR,1 bR,13aR,13bR)-3a-((1S,3R)-3-(4-ethylpiperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yl) 2,2-dimethylcyclobutane-1,3-dicarboxylate

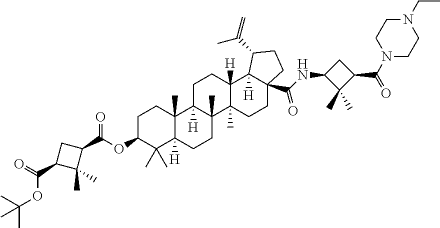

To a stirred solution of (1R,3aS,5aR,5bR,7aR,9S,11aR,11bR,13aR,13bR)—N-((1S,3R)-3-(4-ethylpiperazine-1-carbonyl)-2,2-dimethylcyclobutyl)-9-hydroxy-5a,5b,8,8,11a-pentamethyl-1-(prop-1-en-2-yl)icosahydro-1H-cyclopenta[a]chrysene-3a-carboxamide (step 2, 1.0 g, 1.47 mmol, 1.0 eq) in toluene (20 ml) was added DMAP (0.90 g, 7.38 mmol, 5.0 eq) and (1S,3R)-3-(benzyloxycarbonyl)-2,2-dimethylcyclobutane carboxylic 2,4,6-trichlorobenzoic anhydride (1.23 g, 2.95 mmol, 2.0 eq). The reaction mixture was heated to reflux for overnight. TLC indicated starting material was consumed and the desired product was observed. The reaction mixture was evaporated under reduced pressure, diluted with water and the aqueous layer was extracted with CH₂Cl₂ (3×15 ml). The combined organic layers were dried over Na₂SO₄, filtered and evaporated under reduced pressure. The crude residue was purified by silicagel column chromatography by using 3% methanol:dichloromethane as an eluent to obtain desired product (1.0 g, 76% yield) as a white solid. ES MS: [M+H]⁺ 888.7 (100%).

### Step 4: Synthesis of (1S,3R)-3-(((1R,3aS,5aR,5bR,7aR,9S,11aR,11bR,13aR,13bR)-3a-((1S,3R)-3-(4-ethylpiperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclobutane carboxylic acid

To compound (1S,3R)-1-tert-butyl 3-((1R,3aS,5aR,5bR,7aR,9S,11aR,11bR,13aR,13bR)-3a-((1S,3R)-3-(4-ethylpiperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yl) 2,2-dimethylcyclobutane-1,3-dicarboxylate (step 3, 0.500 g, 0.56 mmol, 1.0 eq) in round bottom flask at 0° C. was added 3N HCl/1,4-dioxane (5 ml). The reaction mixture was allowed to stir at room temperature for overnight. TLC indicated starting material was consumed and the desired product was observed. The reaction mixture was diluted with water and extracted with CH₂Cl₂ (3×20 ml). The combined organic layers were dried over Na₂SO₄, filtered and evaporated under reduced pressure. The crude residue was purified by silicagel column chromatography by using 4% methanol:dichloromethane as an eluent to obtain desired product (0.230 g) as a white solid. ¹H NMR (300 MHz, DMSO-d6): 8 ppm 12.12 (brs, 1H), 7.54 (d, J=6.9 Hz, 1H), 4.65 (s, 1H), 4.52 (s, 1H), 4.38-4.29 (m, 1H), 3.94-3.83 (m, 1H), 3.60-3.50 (m, 1H), 3.44-3.37 (m, 3H), 3.02-2.86 (m, 2H), 2.85-2.70 (m, 2H), 2.64-2.58 (m, 1H), 2.43-2.38 (m, 1H), 2.37-2.15 (m, 8H), 1.94-1.82 (m, 3H), 1.76-1.66 (m, 1H), 1.65-1.60 (m, 4H), 1.60-1.50 (m, 4H), 1.45-1.23 (m, 18H), 1.14-0.95 (m, 3H), 0.99 (t, 3H, J=7.2 Hz), 0.90 (s, 3H), 0.88 (s, 3H), 0.85-0.76 (m, 12H), 0.68 (s, 3H); ES MS: [M+H]⁺ 832.7 (100%); HPLC: 90.0%.

### Example 21

### Preparation of (1R,3S)-3-(((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclobutane carboxylicacid

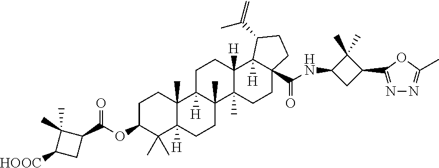

### Step 1: Synthesis of (1S,3R)-3-(benzyloxycarbonyl)-2,2-dimethylcyclobutanecarboxylic 2,4,6-trichlorobenzoic anhydride

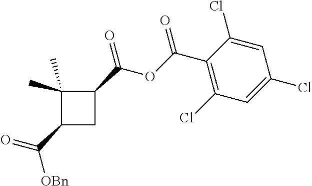

To a stirred solution of (1S,3R)-3-(benzyloxycarbonyl)-2,2-dimethylcyclobutane carboxylicacid (0.90 g, 3.43 mmol, 1.0 eq) in THF (10 ml) at 0° C. under nitrogen was added triethyl amine (1.43 ml, 10.30 mmol, 3.0 eq) and 2,4,6-trichlorobenzoyl chloride (1.0 g, 4.12 mmol, 1.2 eq). The reaction mixture was allowed to stir at room temperature for 4 hours. TLC indicated starting material was consumed and the desired product was observed. The reaction mixture was evaporated under reduced pressure to obtain the crude compound (1.6 g), which is used as such for next step without further purification.

### Step 2: Synthesis of (1R,3S)-1-benzyl3-((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yl)2,2-dimethylcyclobutane-1,3-dicarboxylate

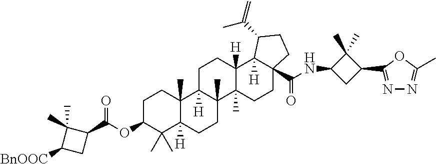

To a stirred solution of (1R,3aS,5aR,5bR,7aR,9S,11aR,11bR,13aR,13bR)—N-((1R,3S)-2,2-dimethyl-3-(5-methyl-1,3,4-oxadiazol-2-yl)cyclobutyl)-9-hydroxy-5a,5b,8,8,11a-pentamethyl-1-(prop-1-en-2-yl)icosahydro-1H-cyclopenta[a]chrysene-3a-carboxamide (1.0 g, 1.61 mmol, 1.0 eq) in toluene (25 ml) at 0° C. was added DMAP (0.985 g, 8.07 mmol, 5.0 eq) and (1S,3R)-3-(benzyloxycarbonyl)-2,2-dimethylcyclobutanecarboxylic 2,4,6-trichlorobenzoic anhydride (step 1) (1.5 g, 3.23 mmol, 2.0 eq) in toluene (10 ml). The reaction mixture was slowly warmed to room temperature and heated to reflux for overnight. TLC indicated starting material was consumed and the desired product was observed. The reaction mixture was diluted with CH₂Cl₂ (50 ml), washed with water (2×100 ml) and brine solution (50 ml). The combined organic layers were dried over Na₂SO₄, filtered and evaporated under reduced pressure. The crude residue was purified by silicagel column chromatography by using 2% methanol:dichloromethane as an eluent to obtain the desired product (0.98 g, 70% yield) as a white solid. ¹H NMR (300 MHz, CDCl₃): δ ppm 7.40-7.30 (m, 5H), 5.72 (d, J=7.8 Hz, 1H), 5.18-5.05 (m, 2H), 4.72 (s, 1H), 4.58 (s, 1H), 4.48-4.38 (m, 1H), 4.30-4.18 (m, 1H), 3.16-3.05 (m, 2H), 2.85-2.72 (m, 2H), 2.70-2.58 (m, 2H), 2.51 (s, 3H), 2.48-2.30 (m, 2H), 1.67 (s, 3H), 1.36 (s, 3H), 1.33 (s, 3H), 0.95 (s, 6H), 0.91 (s, 3H), 0.82 (s, 6H), 0.81 (s, 6H), 2.08-0.74 (m, 24H); ES MS: [M+H]⁺ 864.5 (100%), [M+Na]⁺ 886.6 (30%).

### Step 3: Synthesis of (1R,3S)-3-(((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclobutane carboxylic acid

To a stirred solution of (1R,3S)-1-benzyl 3-((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yl) 2,2-dimethylcyclobutane-1,3-dicarboxylate (step 2) (1.0 g, 1.15 mmol, 1.0 eq) in dichloromethane (20 ml) was added palladium (II) acetate (0.078 g, 0.347 mmol, 0.3 eq), triethyl amine (0.48 ml, 3.47 mmol, 3.0 eq) and triethylsilane (0.269 g, 2.31 mmol, 2.0 eq). The mixture was flushed with nitrogen and heated to reflux for overnight. TLC indicated starting material was not consumed, again excess palladium (II) acetate (0.052 g, 0.23 mmol, 0.2 eq) and triethyl amine (0.32 ml, 2.3 mmol, 2.0 eq) was added and refluxed for 24 hours. The reaction mixture was cooled to room temperature, filtered through a pad of celite and washed with dichloromethane (100 ml). The filtrate was washed with water (2×100 ml), brine solution, dried over Na₂SO₄, filtered and evaporated under reduced pressure. The crude residue was purified by silicagel column chromatography (eluent: 3% methanol:dichloromethane) followed by recrystallization over methyl-tert-butyl ether gave the desired product (0.3 g, 34% yield) as a white solid. H NMR (300 MHz, CDCl₃): δ ppm 6.21 (d, J=7.5 Hz, 1H), 4.73 (s, 1H), 4.58 (s, 1H), 4.55-4.45 (m, 1H), 4.30-4.20 (m, 1H), 3.23-3.07 (m, 2H), 2.91-2.82 (m, 1H), 2.82-2.74 (m, 1H), 2.74-2.55 (m, 2H), 2.51 (s, 3H), 2.50-2.36 (m, 1H), 1.68 (s, 3H), 1.37 (s, 3H), 1.36 (s, 3H), 1.05 (brs, 6H), 0.89 (s, 3H), 0.84 (s, 3H), 0.83 (s, 3H), 0.81 (brs, 6H), 2.23-0.72 (m, 25H); ES MS: [M+H]⁺ 774.5 (50%), [M+Na]⁺ 796.5 (100%); IR (KBr) cm⁻¹: 3409, 2953, 2869, 1729, 1193; HPLC: 91.6%.

### Example 22

### Preparation of 4-(((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-2,2-bis(methyl-d3)-4-oxobutanoic acid

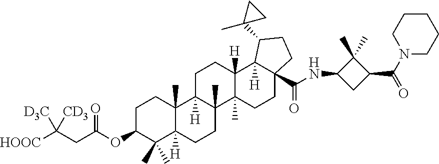

To a stirred solution of (1R,3aS,5aR,5bR,7aR,9S,11aR,11bR,13aR,13bR)—N-((1R,3S)-2,2-dimethyl-3-(piperidine-1-carbonyl)cyclobutyl)-9-hydroxy-5a,5b,8,8,11a-pentamethyl-1-(1-methyl cyclopropyl)icosahydro-1H-cyclopenta[a]chrysene-3a-carboxamide (0.500 g, 0.754 mmol, 1.0 eq) in toluene (12 ml) was added 2,2-dimethylsuccinicanhydride (D₂-CH₃)(0.404 g, 3.02 mmol, 4.0 eq) and DMAP (0.184 g, 1.51 mmol, 2.0 eq). The reaction mixture was heated to reflux for 48 hours. TLC indicated starting material was consumed and the desired product was observed. The mixture was concentrated under reduced pressure, diluted with water (50 ml) and extracted with CH₂Cl₂ (3×50 ml). The combined organic extracts were dried over Na₂SO₄, filtered and evaporated under reduced pressure. The crude residue was purified by silicagel column chromatography by using 2% methanol:dichloromethane as an eluent to obtain the desired product (0.300 g, 50% yield) as a white solid. ¹H NMR (300 MHz, CDCl₃): δ ppm 9.7 (s, 1H), 5.94 (d, J=9.0 Hz, 1H), 4.55-4.45 (m, 1H), 4.13-4.03 (m, 1H), 3.77-3.70 (m, 1H), 3.50-3.30 (m, 3H), 2.874 (t, J=8.1 Hz, 1H), 0.97 (s, 3H), 0.92 (s, 3H), 0.89 (s, 3H), 0.85 (s, 3H), 0.83 (s, 9H), 2.70-0.75 (m, 38H), 0.47-0.38 (m, 1H), 0.38-0.30 (m, 1H), 0.28-0.14 (m, 2H); ES MS: [M−H]⁻ 795.5 (90%); IR (KBr) cm⁻¹: 3400, 2947, 2868, 1733, 1619, 1194, 980; HPLC: 88.5+6.8% (isomers).

### Example 23

### Preparation of (1R,3S)-3-((((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

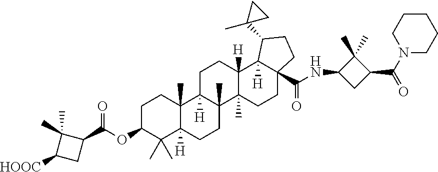

### Step 1: Synthesis of (1R,3S)-1-benzyl3-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(1-methylcyclopropyl)icosahydro-1H-cyclopenta[a]chrysen-9-yl) 2,2-dimethylcyclobutane-1,3-dicarboxylate

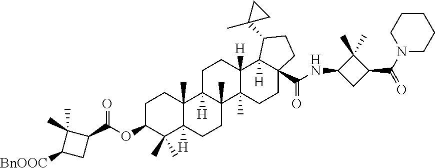

To a stirred solution of (1R,3aS,5aR,5bR,7aR,9S,11aR,11bR,13aR,13bR)—N-((1R,3S)-2,2-dimethyl-3-(piperidine-1-carbonyl)cyclobutyl)-9-hydroxy-5a,5b,8,8,11a-pentamethyl-1-(1-methyl cyclopropyl)icosahydro-1H-cyclopenta[a]chrysene-3a-carboxamide (0.450 g, 0.693 mmol, 1.0 eq) in toluene (10 ml) was added DMAP (0.169 g, 1.38 mmol, 2.0 eq) and (1S,3R)-3-(benzyloxycarbonyl)-2,2-dimethylcyclobutanecarboxylic2,4,6-trichlorobenzoicanhydride (0.648 g, 1.38 mmol, 2.0 eq). The reaction mixture was heated at 90° C. for overnight. TLC indicated starting material was consumed and the desired product was observed. The reaction mixture was evaporated under reduced pressure, diluted with water (30 ml) and extracted with CH₂Cl₂ (2×100 ml). The combined organic layers were dried over Na₂SO₄, filtered and evaporated under reduced pressure. The crude solid (0.500 g) was used as such for next step without further purification. ES MS: [M+H]⁺ 907.5 (50%); IR (KBr) cm⁻¹: 3408, 2953, 2866, 1729, 1736, 1623, 1459, 1186, 1022.

### Step 2: Synthesis of (1R,3S)-3-(((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(1-methylcyclopropyl)icosahydro-1H-cyclopenta[a]chrysen-9-yloxy)carbonyl)-2,2-dimethylcyclobutane carboxylic acid

To a solution of (1R,3S)-1-benzyl3-((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(1-methylcyclopropyl)icosahydro-1H-cyclopenta[a]chrysen-9-yl)2,2-dimethylcyclobutane-1,3-dicarboxylate (step 1) (0.500 g, 0.55 mmol, 1.0 eq) in dichloromethane (15 ml) was added palladium (II) acetate (0.062 g, 0.279 mmol, 0.5 eq), triethyl amine (0.23 ml, 1.67 mmol, 3.0 eq) and triethylsilane (0.26 ml, 1.67 mmol, 3.0 eq). The reaction mixture was flushed with N₂ and heated to reflux for 48 hours. TLC indicated starting material was consumed and the desired product was observed. The reaction mixture was cooled to room temperature, filtered through a pad of celite and was washed with dichloromethane. The filtrate was diluted with water (100 ml), organic layer was separated and the aqueous layer was extracted with dichloromethane (2×100 ml). The combined organic layers were dried over Na₂SO₄, filtered and evaporated under reduced pressure. The crude residue was purified by silicagel column chromatography by using 6% methanol:dichloromethane as an eluent to obtain the desired product (0.050 g) as a white solid. ¹H NMR (300 MHz, CDCl₃): δ ppm 6.21 (d, J=7.5 Hz, 1H), 4.57-4.48 (m, 1H), 4.15-4.03 (m, 1H), 3.78-3.68 (m, 1H), 3.50-3.30 (m, 3H), 2.92-2.77 (m, 3H), 2.77-2.60 (m, 2H), 2.40-2.30 (m, 1H), 2.23-0.74 (m, 38H), 1.04 (s, 3H), 1.02 (s, 3H), 0.89 (s, 6H), 0.85 (s, 3H), 0.83 (s, 6H), 0.81 (s, 3H), 0.47-0.38 (m, 1H), 0.38-0.27 (m, 1H), 0.28-0.13 (m, 2H); ES MS: [M+H]⁺ 817.4 (100%), [M+Na]⁺ 839.5 (50%); HPLC: 95.5%.

### Example 24

### Preparation of 4-(((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(prop-1-en-2-yl)icosahydro-1H-cyclopenta[a]chrysen-9-yl)oxy)-2,2-bis(methyl-d3)-4-oxobutanoic acid

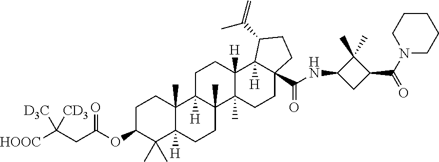

To a stirred solution of (1R,3aS,5aR,5bR,7aR,9S,11aR,11bR,13aR,13bR)—N-((1R,3S)-2,2-dimethyl-3-(piperidine-1-carbonyl)cyclobutyl)-9-hydroxy-5a,5b,8,8,11a-pentamethyl-1-(prop-1-en-2-yl)icosahydro-1H-cyclopenta[a]chrysene-3a-carboxamide (0.500 g, 0.77 mmol, 1.0 eq) in toluene (5 ml) was added 2,2-dimethylsuccinicanhydride (D₂-CH₃) (0.413 g, 3.08 mmol, 4.0 eq) and DMAP (0.188 g, 1.54 mmol, 2.0 eq). The reaction mixture was heated at 90° C. for overnight. TLC indicated starting material was consumed and the desired product was observed. The reaction mixture was evaporated under reduced pressure, cooled to 0° C., acidified with 1N HCl to pH 5 and extracted with CH₂Cl₂ (2×50 ml). The combined organic extracts were washed with water, dried over Na₂SO₄, filtered and evaporated under reduced pressure. The crude residue was purified by silicagel column chromatography by using 1% methanol:dichloromethane as an eluent to obtain the desired product (0.130 g, 21.5% yield) as a white solid. ¹H NMR (300 MHz, CDCl₃): δ ppm 5.99 (d, J=7.8 Hz, 1H), 4.72 (s, 1H), 4.57 (s, 1H), 4.53-4.43 (m, 1H), 4.11 (q, J=8.4 Hz, 1H), 3.78-3.64 (m, 1H), 3.50-3.30 (m, 3H), 3.18-3.06 (m, 1H), 2.88 (t, J=8.4 Hz, 1H), 2.70-2.54 (m, 2H), 2.53-2.37 (m, 2H), 2.28-2.17 (m, 1H), 1.67 (s, 3H), 0.95 (s, 3H), 0.92 (s, 3H), 0.87 (s, 3H), 0.82 (s, 6H), 0.78 (s, 3H), 2.05-0.72 (m, 32H); ES MS: [M+H]⁺ 783.5 (100%), [M+Na]⁺ 805.6 (50%); HPLC: 90.2%+6.9% (isomers).

### Example 25

### Preparation of 4-(((1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yl)oxy)-2,2-bis(methyl-d3)-4-oxobutanoic acid

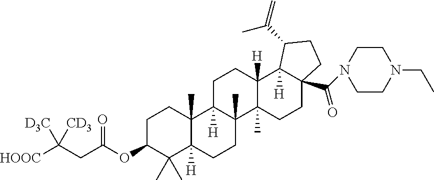

### Step 1: Synthesis of (1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yl acetate

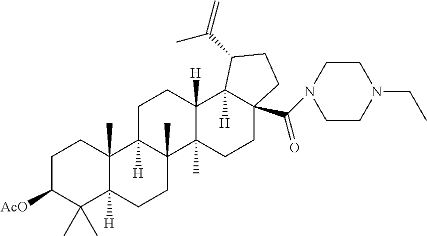

To a stirred solution of Ethyl piperazine (11.06 g, 96.8 mmol, 2.0 eq) and triethylamine (24.46 g, 242 mmol, 5.0 eq) in CH₂Cl₂ (150 ml) at 0° C. was added a solution of (1R,3aS,5aR,5bR,7aR,9S,11aR,11bR,13aR,13bR)-3a-(chlorocarbonyl)-5a,5b,8,8,11a-penta methyl-1-(prop-1-en-2-yl)icosahydro-1H-cyclopenta[a]chrysen-9-yl acetate (25 g, 48.4 mmol, 1.0 eq) in CH₂Cl₂ (150 ml). The reaction mixture was allowed to stir at room temperature for overnight. TLC indicated starting material was consumed and the desired product was observed. The reaction mixture was quenched with water and extracted with CH₂Cl₂ (3×200 ml). The combined organic layers were dried over Na₂SO₄, filtered and evaporated under reduced pressure. The crude residue was purified by silicagel column chromatography by using 2% methanol:dichloromethane as an eluent to obtain the desired product (26 g, 90% yield) as a white solid. H NMR (300 MHz, CDCl₃): δ ppm 4.72 (s, 1H), 4.57 (s, 1H), 4.50-4.42 (m, 1H), 3.68-3.56 (m, 4H), 3.03-2.82 (m, 2H), 2.48-2.32 (m, 6H), 2.04 (s, 3H), 1.68 (s, 3H), 1.10 (t, J=7.2 Hz, 3H), 0.95 (s, 3H), 0.94 (s, 3H), 0.84 (s, 3H), 0.836 (s, 3H), 0.83 (s, 3H), 2.15-0.75 (m, 23H); ES MS: [M+H]⁺ 595.4 (100%).

### Step 2: Synthesis of (4-ethylpiperazin-1-yl)((1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysen-3a-yl)methanone

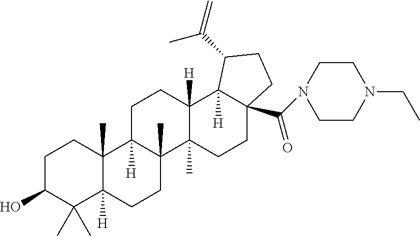

To a stirred solution of (1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yl acetate (step 1) (26 g, 43.7 mmol, 1.0 eq) in THF (200 ml) and Methanol (200 ml) was added potassium carbonate (42.28 g, 306.3 mmol, 7.0 eq). The reaction mixture was stirred at room temperature for 48 hours. TLC indicated starting material was consumed and the desired product was observed. The mixture was filtered through a pad of celite and the filtrate was evaporated under reduced pressure. The crude residue was diluted with water and extracted with CH₂Cl₂ (3×100 ml). The combined organic layers were dried over Na₂SO₄, filtered and evaporated under reduced pressure. The crude solid was recrystallized over acetonitrile gave the desired product (24 g, 99% yield) as a white solid. ¹H NMR (300 MHz, CDCl₃): δ ppm 4.72 (s, 1H), 4.57 (s, 1H), 3.67-3.56 (m, 4H), 3.22-3.13 (m, 1H), 3.02-2.82 (m, 2H), 2.47-2.33 (m, 6H), 1.68 (s, 3H), 1.09 (t, J=7.2 Hz, 3H), 0.95 (s, 6H), 0.94 (s, 3H), 0.82 (s, 3H), 0.75 (s, 3H), 2.15-0.65 (m, 23H); ES MS: [M+H]⁺ 553.3 (100%).

### Step 3: Synthesis of 4-(((1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yl)oxy)-2,2-bis(methyl-d3)-4-oxobutanoic acid

To a stirred solution of (4-ethylpiperazin-1-yl)((1R,3aS,5aR,5bR,7aR,9S,11aR,11bR,13aR,13bR)-9-hydroxy-5a,5b,8,8,11a-pentamethyl-1-(prop-1-en-2-yl)icosahydro-1H-cyclopenta[a]chrysen-3a-yl)methanone (step 2) (0.800 g, 1.44 mmol, 1.0 eq) in toluene (15 ml) was added 2,2-dimethyl succinicanhydride (D₂-CH₃) (0.775 g, 5.78 mmol, 4.0 eq) and DMAP (0.353 g, 2.89 mmol, 2.0 eq). The reaction mixture was heated at 90° C. for overnight. TLC indicated starting material was consumed and the desired product was observed. The reaction mixture was evaporated under reduced pressure, cooled to 0° C., acidified with 1N HCl to pH 5 and extracted with CH₂Cl₂ (2×50 ml). The combined organic extracts were washed with water, dried over Na₂SO₄, filtered and evaporated under reduced pressure. The crude residue was purified by silicagel column chromatography by using 5% methanol:dichloromethanes as an eluent to obtain the desired product (0.570 g, 57.4% yield) as a white solid. ¹H NMR (300 MHz, CDCl₃): δ ppm 4.92 (s, 1H), 4.77 (s, 1H), 4.80-4.79 (m, 1H), 3.85-3.75 (m, 4H), 3.47-3.35 (m, 1H), 3.35-3.25 (m, 1H), 3.02-2.87 (m, 2H), 1.78 (s, 3H), 0.98 (s, 3H), 0.93 (s, 3H), 0.75 (s, 3H), 2.48-0.78 (m, 38H); ES MS: [M+H]⁺ 687.4 (100%), [M+Na]⁺ 709 (70%); IR (KBr) cm⁻¹: 3435, 2943, 2871, 1728, 1634, 1196, 981; HPLC: 90.79+8.34% (isomers).

### Example 26

### Preparation of 5-(((1R,3aS,5aR,5bR,7aR,9S,11aR,11bR,13aR,13bR)-3a-(4-ethylpiperazine-1-carbonyl)-5a,5b,8,8,8,11a-pentamethyl-1-(1-methylcyclopropyl)icosahydro-1H-cyclopenta[a]chrysen-9-yl)oxy)-3,3-dimethyl-5-oxopentanoic acid

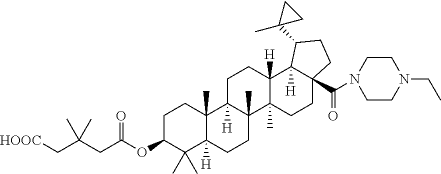

### Step 1: Synthesis of (1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl acetate

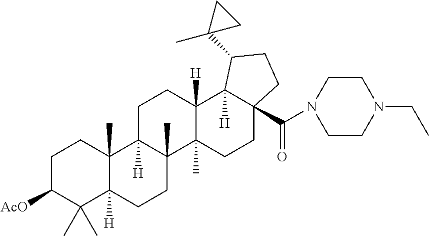

To a stirred solution of Ethyl piperazine (0.400 g, 3.50 mmol, 1.2 eq) and triethylamine (2.0 ml, 14.59 mmol, 5.0 eq) in CH₂Cl₂ (10 ml) at 0° C. was added a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-yl acetate (1.55 g, 2.91 mmol, 1.0 eq) in CH₂Cl₂ (10 ml). The reaction mixture was allowed to stir at room temperature for overnight. TLC indicated starting material was consumed and the desired product was observed. The reaction mixture was washed with water (20 ml) and brine solution (10 ml). The organic layer was dried over Na₂SO₄, filtered and evaporated under reduced pressure. The crude residue was purified by silicagel column chromatography by using 1.5% methanol:dichloromethane as an eluent to obtain the desired product (1.5 g) as a white solid. ¹H NMR (300 MHz, CDCl₃): δ ppm 4.52-4.42 (m, 1H), 3.68-3.52 (m, 4H), 2.77 (t, J=10.5 Hz, 1H), 2.47-2.32 (m, 6H), 2.04 (s, 3H), 1.09 (t, J=7.2 Hz, 3H), 0.97 (s, 3H), 0.93 (s, 3H), 0.90 (s, 3H), 0.86 (s, 3H), 0.84 (s, 6H), 2.08-0.78 (m, 24H), 0.48-0.39 (m, 1H), 0.40-0.30 (m, 1H), 0.28-0.14 (m, 2H); ES MS: [M+H]⁺ 609.4 (100%).

### Step 2: Synthesis of (4-ethylpiperazin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl) methanone

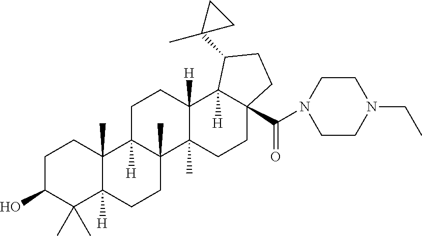

To a stirred solution of (1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl acetate (step 1) (1.5 g, 2.46 mmol, 1.0 eq) in THF (15 ml) and Methanol (15 ml) was added potassium carbonate (2.38 g, 17.26 mmol, 7.0 eq). The reaction mixture was stirred at room temperature for 48 hours. TLC indicated starting material was consumed and the desired product was observed. The mixture was filtered through a pad of celite and the filtrate was evaporated under reduced pressure. The crude solid was purified by silicagel column chromatography by using 2% methanol:dichloromethane as an eluent to obtain the desired product (1.05 g, 75% yield) as a white solid. ¹H NMR (300 MHz, CDCl₃): δ ppm 3.65-3.55 (m, 4H), 3.19 (q, J=5.4 Hz, 1H), 2.85-2.72 (m, 1H), 2.50-2.30 (m, 6H), 1.09 (t, J=7.2 Hz, 3H), 0.977 (s, 3H), 0.97 (s, 3H), 0.93 (s, 3H), 0.89 (s, 3H), 0.84 (s, 3H), 0.76 (s, 3H), 2.13-0.65 (m, 24H), 0.49-0.40 (m, 1H), 0.38-0.28 (m, 1H), 0.29-0.12 (m, 2H); ES MS: [M+H]⁺ 567.3 (100%).

### Step 3: Synthesis of 5-((1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yloxy)-3,3-dimethyl-5-oxopentanoic acid

To a stirred solution of (4-ethylpiperazin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl)methanone (step 2) (0.500 g, 0.88 mmol, 1.0 eq) in toluene (5 ml) was added 3,3-dimethylglutaric anhydride (0.500 g, 3.53 mmol, 4.0 eq) and DMAP (0.21 g, 1.76 mmol, 2.0 eq). The reaction mixture was refluxed for overnight. TLC indicated starting material was consumed and the desired product was observed. The reaction mixture was evaporated under reduced pressure, diluted with CH₂Cl₂ (100 ml), washed the organic layer with water (2×100 ml) and brine solution (2×50 ml). The combined organic extracts were dried over Na₂SO₄, filtered and evaporated under reduced pressure. The crude residue was purified by silicagel column chromatography (eluent: 1.5% methanol:dichloromethane), followed by recrystallization over acetonitrile gave the desired product (0.350 g, 56% yield) as a white solid. ¹H NMR (300 MHz, pyridine-d5): δ ppm 4.82-4.72 (m, 1H), 3.78-3.68 (m, 4H), 3.27-3.16 (m, 1H), 2.87-2.72 (m, 4H), 2.38-2.16 (m, 8H), 1.01 (s, 3H), 0.98 (s, 3H), 0.97 (s, 3H), 0.93 (s, 3H), 0.82 (s, 3H), 2.14-0.83 (m, 34H), 0.62-0.53 (m, 1H), 0.52-0.43 (m, 1H), 0.35-0.022 (m, 2H); ES MS: [M+H]⁺ 709.4 (100%); HPLC: 99.5%.

### Example 27

### Preparation of 2,2-dimethyl-4-oxo-4-(((1R,3aS,5aR,5bR,7aR,9S,11aR,1bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yl)oxy)butanoic acid

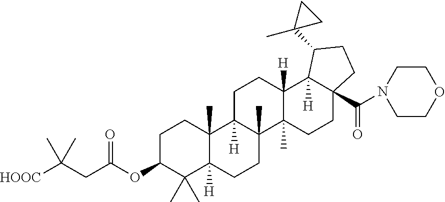

### Step 1: Synthesis of (1R,3aS,5aR,5bR,7aR,9S,11aR,11bR,13aR,13bR)-5a,5b,8,8,11a-pentamethyl-1-(1-methyl cyclopropyl)-3a-(morpholine-4-carbonyl)icosahydro-1H-cyclopenta[a]chrysen-9-yl acetate

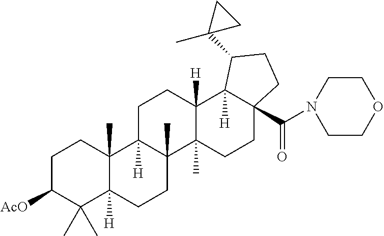

To a stirred solution of Morpholine (0.305 g, 3.50 mmol, 1.2 eq) and triethylamine (2.02 ml, 14.62 mmol, 5.0 eq) in CH₂Cl₂ (10 ml) at 0° C. was added a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-yl acetate (1.55 g, 2.92 mmol, 1.0 eq) in CH₂Cl₂ (10 ml). The reaction mixture was allowed to stir at room temperature for overnight. TLC indicated starting material was consumed and the desired product was observed. The reaction mixture was diluted with CH₂Cl₂ (50 ml), washed with water (2×50 ml) and brine solution (50 ml). The organic layer was dried over Na₂SO₄, filtered and evaporated under reduced pressure. The crude residue was purified by silicagel column chromatography by using 10% ethyl acetate:hexanes as an eluent to obtain the desired product (1.65 g, 97% yield) as a white solid. ¹H NMR (300 MHz, CDCl₃): δ ppm 4.53-4.42 (m, 1H), 3.70-3.50 (m, 8H), 2.83-2.72 (m, 1H), 2.04 (s, 3H), 0.97 (s, 3H), 0.93 (s, 3H), 0.90 (s, 3H), 0.88 (s, 3H), 0.86 (s, 3H), 0.84 (s, 3H), 2.07-0.78 (m, 24H), 0.48-0.40 (m, 1H), 0.40-0.32 (m, 1H), 0.28-0.15 (m, 2H); ES MS: [M+H]⁺ 582.3 (100%).

### Step 2: Synthesis of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl) (morpholino)methanone

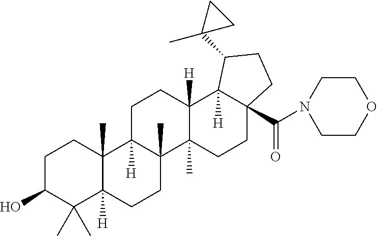

To a stirred solution of (1R,3aS,5aR,5bR,7aR,9S,11aR,11bR,13aR,13bR)-5a,5b,8,8,11a-penta methyl-1-(1-methylcyclopropyl)-3a-(morpholine-4-carbonyl)icosahydro-1H-cyclopenta[a]chrysen-9-yl acetate (step 1) (1.65 g, 2.83 mmol, 1.0 eq) in THF (12 ml) and Methanol (12 ml) was added potassium carbonate (2.74 g, 19.87 mmol, 7.0 eq). The reaction mixture was stirred at room temperature for 48 hours. TLC indicated starting material was consumed and the desired product was observed. The mixture was filtered through a pad of celite and the filtrate was evaporated under reduced pressure. The crude solid was purified by silicagel column chromatography by using 1% methanol:dichloromethanes as an eluent to obtain the desired product (1.5 g, 96% yield) as a white solid. ¹H NMR (300 MHz, CDCl₃): δ ppm 3.67-3.55 (m, 8H), 3.23-3.15 (m, 1H), 2.83-2.70 (m, 1H), 0.98 (s, 3H), 0.97 (s, 3H), 0.93 (s, 3H), 0.899 (s, 3H), 0.839 (s, 3H), 0.76 (s, 3H), 2.10-0.65 (m, 24H), 0.50-0.40 (m, 1H), 0.39-0.30 (m, 1H), 0.28-0.15 (m, 2H); ES MS: [M+H]⁺ 540.3 (100%), [M+K]⁺ 578.3 (50%).

### Step 3: Synthesis of 2,2-dimethyl-4-oxo-4-((1R,3aS,5aR,5bR,7aR,9S,11aR,11bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yloxy)butanoic acid

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl)(morpholino) methanone (step 2) (0.600 g, 1.11 mmol, 1.0 eq) in toluene (6 ml) was added 2,2-dimethyl succinicanhydride (0.569 g, 4.40 mmol, 4.0 eq) and DMAP (0.27 g, 2.20 mmol, 2.0 eq). The reaction mixture was refluxed for overnight. TLC indicated starting material was consumed and the desired product was observed. The reaction mixture was diluted with CH₂Cl₂ (50 ml), washed the organic layer with water (2×50 ml) and brine solution (50 ml). The combined organic extracts were dried over Na₂SO₄, filtered and evaporated under reduced pressure. The crude solid was purified by silicagel column chromatography (eluent: 2% methanol:dichloromethane) followed by recrystallization over CH₃CN gave the desired product (0.35 g, 47% yield) as a white solid. ¹H NMR (300 MHz, CDCl₃): δ ppm 4.55-4.46 (m, 1H), 3.67-3.53 (m, 8H), 2.82-2.72 (m, 1H), 2.72-2.53 (m, 2H), 2.10-2.0 (m, 2H), 0.97 (s, 3H), 0.92 (s, 3H), 0.90 (s, 3H), 0.85 (s, 3H), 0.83 (s, 3H), 0.81 (s, 3H), 1.95-0.75 (m, 28H), 0.48-0.40 (m, 1H), 0.39-0.31 (m, 1H), 0.28-0.15 (m, 2H); ES MS: [M+H]⁺ 668.4 (100%), [M+Na]⁺ 690.4 (60%); HPLC: 94.2%.

### Example 28

### Preparation of (1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yl)oxy)carbonyl)cyclobutane-1-carboxylic acid

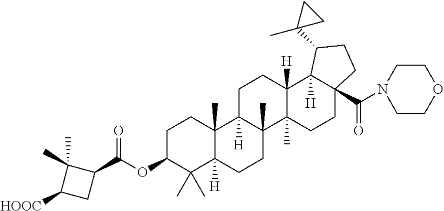

### Step 1: Synthesis of (1R,3S)-1-benzyl 3-((1R,3aS,5aR,5bR,7aR,9S,11aR,11bR,13aR,13bR)-5a,5b,8,8,11a-penta methyl-1-(1-methylcyclopropyl)-3a-(morpholine-4-carbonyl)icosahydro-1H-cyclopenta[a]chrysen-9-yl) 2,2-dimethylcyclobutane-1,3-dicarboxylate

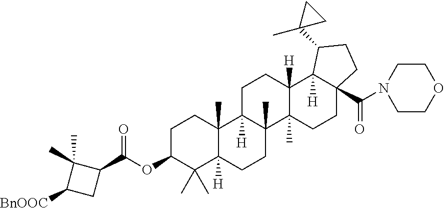

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl)(morpholino)methanone (0.8 g, 1.48 mmol, 1.0 eq) in toluene (10 ml) was added DMAP (0.9 g, 7.42 mmol, 5.0 eq) and (1S,3R)-3-(benzyloxycarbonyl)-2,2-dimethylcyclobutanecarboxylic 2,4,6-trichlorobenzoic anhydride (1.34 g, 2.96 mmol, 2.0 eq) in toluene (10 ml). The reaction mixture was heated to reflux for overnight. TLC indicated starting material was consumed and the desired product was observed. The reaction mixture was diluted with CH₂Cl₂ (50 ml) and washed with water (2×100 ml) and brine solution (100 ml). The organic layer was dried over Na₂SO₄, filtered and evaporated under reduced pressure. The crude solid was purified by silicagel column chromatography by using 15% ethyl acetate:hexanes as an eluent to obtain the desired product (0.95 g, 81% yield) as an off-white solid. ¹H NMR (300 MHz, CDCl₃): δ ppm 7.37-7.30 (m, 5H), 5.17-5.07 (m, 2H), 4.44 (dd, J=4.8, 11.1 Hz, 1H), 3.70-3.53 (m, 8H), 2.86-2.57 (m, 4H), 0.97 (brs, 6H), 0.93 (s, 3H), 0.89 (s, 3H), 0.85 (s, 3H), 0.84 (s, 3H), 0.83 (s, 3H), 2.10-0.75 (m, 28H), 0.48-0.40 (m, 1H), 0.38-0.30 (m, 1H), 0.28-0.15 (m, 2H); ES MS: [M+H]⁺ 784.5 (100%).

### Step 2: Synthesis of (1R,3S)-2,2-dimethyl-3-(((1R,3aS,5aR,5bR,7aR,9S,11aR,11bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yloxy)carbonyl)cyclobutanecarboxylic acid

To a solution of (1R,3S)-1-benzyl 3-((1R,3aS,5aR,5bR,7aR,9S,11aR,11bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yl) 2,2-dimethylcyclobutane-1,3-dicarboxylate (step 1) (0.95 g, 1.21 mmol, 1.0 eq) in dichloromethane (20 ml) was added palladium (II) acetate (0.136 g, 0.60 mmol, 0.5 eq), triethyl amine (0.5 ml, 3.63 mmol, 3.0 eq) and triethylsilane (0.38 ml, 2.42 mmol, 2.0 eq). The reaction mixture was heated to reflux for overnight. TLC indicated starting material was consumed and the desired product was observed. The reaction mixture was cooled to room temperature, filtered through a pad of celite and washed with dichloromethane (100 ml). The filtrate was washed with water (2×100 ml) and brine solution (100 ml). The organic layer was dried over Na₂SO₄, filtered and evaporated under reduced pressure. The crude solid was purified by silicagel column chromatography (eluent: 6% methanol:dichloromethane), followed by recrystallization over CH₃CN gave the desired product (0.45 g, 53% yield) as a white solid. ¹H NMR (300 MHz, CDCl₃): δ 4.46 (dd, J=4.8, 11.1 Hz, 1H), 3.68-3.55 (m, 8H), 2.87-2.72 (m, 3H), 2.59 (q, J=11.1 Hz, 1H), 1.07 (s, 3H), 0.97 (s, 3H), 0.93 (s, 3H), 0.90 (s, 3H), 0.86 (s, 3H), 0.85 (s, 3H), 2.12-0.77 (m, 31H), 0.48-0.40 (m, 1H), 0.40-0.30 (m, 1H), 0.27-0.15 (m, 2H); ES MS: [M+H]⁺ 694.4 (100%), [M+Na]⁺ 716.3 (60%); HPLC: 94.9%.

### Example 29

### Preparation of (1R,3S)-3-((((1R,3aS,5aR,5bR,7aR,9S,11aR,11bR,13aR,13bR)-3a-(4-methoxypiperidine-1-carbonyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylic acid

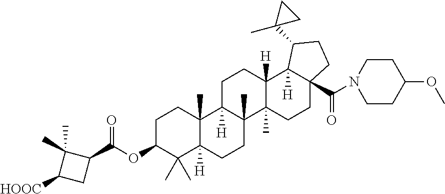

### Step 1: Synthesis of tert-butyl 4-oxopiperidine-1-carboxylate

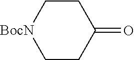 [SMILES: CC(C)(C)OC(=O)N1CCC(=O)CC1]

To a stirred solution of piperidin-4-one hydrochloride (13.0 g, 84.62 mmol, 1.0 eq) in 1,4-dioxane:water (4:1, 130 ml) at 0° C. was added triethyl amine (21.36 g, 211.5 mmol, 2.5 eq) and di-tert-butyldicarbonate (27.67 g, 126.9 mmol, 1.5 eq). The reaction mixture was allowed to stir at room temperature for overnight. TLC indicated starting material was consumed and the desired product was observed. The reaction mixture was evaporated under reduced pressure, diluted with CH₂Cl₂ (350 ml), washed with water (2×50 ml). The organic layer was dried over Na₂SO₄, filtered and evaporated under reduced pressure. The crude residue was purified by silicagel column chromatography by using 1% methanol:dichloromethanes as an eluent to obtain the desired product (16.1 g, 95% yield) as a white solid.

### Step 2: Synthesis of tert-butyl 4-hydroxypiperidine-1-carboxylate

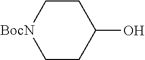 [SMILES: CC(C)(C)OC(=O)N1CCC(O)CC1]

To a stirred solution of tert-butyl 4-oxopiperidine-1-carboxylate (step 1) (13.0 g, 65.32 mmol, 1.0 eq) in methanol (130 ml) at 0° C. was added sodium borohydride (0.741 g, 19.59 mmol, 0.3 eq). The reaction mixture was allowed to stir at room temperature for overnight. TLC indicated starting material was consumed and the desired product was observed. The reaction mixture was evaporated under reduced pressure, diluted with water (200 ml) and extracted with CH₂Cl₂ (2×200 ml). The combined organic layers were dried over Na₂SO₄, filtered and evaporated under reduced pressure to obtain the desired product (13.0 g) as a white solid. ¹H NMR (300 MHz, CDCl₃): δ ppm 3.90-3.78 (m, 3H), 3.07-2.97 (m, 2H), 1.92-1.80 (m, 2H), 1.52-1.40 (m, 11H); ES MS: [M+Na]⁺ 224.1 (100%).

### Step 3: Synthesis of tert-butyl 4-methoxypiperidine-1-carboxylate

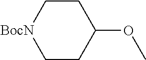

To a stirred solution of NaH (0.716 g, 29.85 mmol, 3.0 eq, 60% dispersion in mineral oil) in THF (10 ml) at 0° C. was added tert-butyl 4-hydroxypiperidine-1-carboxylate (step 2) (2.0 g, 9.95 mmol, 1.0 eq) in THF (10 m). The reaction mixture was stirred at 0° C. for 10 minutes. then Methyliodide (1.0 ml, 19.90 mmol, 2.0 eq) was added. The reaction mixture was allowed to stir at room temperature for overnight. TLC indicated starting material was consumed and the desired product was observed. The reaction mixture was quenched with saturated NH₄Cl solution (10 ml), diluted with water (10 ml) and extracted with CH₂Cl₂ (3×20 ml). The combined organic layers were dried over Na₂SO₄, filtered and evaporated under reduced pressure. The crude residue was washed with n-hexanes and dried under vacuum to obtain the desired product (2.0 g) as colorless oil. ¹H NMR (300 MHz, CDCl₃): δ ppm 3.82-3.70 (m, 2H), 3.37 (s, 3H), 3.36-3.30 (m, 1H), 3.12-3.0 (m, 2H), 1.90-1.80 (m, 2H), 1.58-1.50 (m, 2H), 1.45 (s, 9H); ES MS: [M+Na]⁺ 238.1 (70%).

### Step 4: Synthesis of 4-methoxypiperidine, TFA

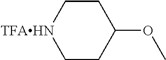

To a stirred solution of tert-butyl 4-methoxypiperidine-1-carboxylate (step 3) (2.0 g, 9.30 mmol, 1.0 eq) in CH₂Cl₂ (10 ml) at 0° C. was added trifluoroacetic acid (10 ml). The reaction mixture was allowed to stir at room temperature for 4 hours. TLC indicated starting material was consumed and the desired product was observed. The reaction mixture was evaporated under reduced pressure, washed with n-hexanes (30 ml) and dried under vacuum to obtain the desired product (5.0 g) as colorless oil. ¹H NMR (300 MHz, CDCl₃): δ ppm 7.93 (brs, 1H), 3.64-3.57 (m, 1H), 3.37 (s, 3H), 3.36-3.32 (m, 2H), 3.30-3.20 (m, 2H), 2.10-1.90 (m, 4H); ES MS: [M+H]⁺ 116.0 (100%).

### Step 5: Synthesis of (1R,3aS,5aR,5bR,7aR,9S,11aR,11bR,13aR,13bR)-3a-(4-methoxypiperidine-1-carbonyl)-5a,5b,8,8,11a-pentamethyl-1-(1-methylcyclopropyl)icosahydro-1H-cyclopenta[a]chrysen-9-yl acetate

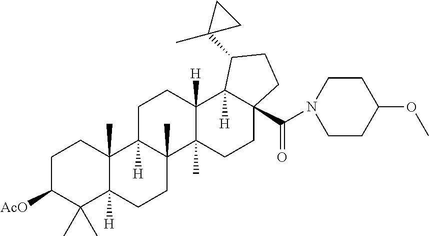

To a stirred solution of 4-methoxypiperidine, TFA (step 4) (1.41 g, 12.26 mmol, 5.0 eq) and triethylamine (2.39 ml, 17.16 mmol, 7.0 eq) in CH₂Cl₂ (15 ml) at 0° C. was added a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-yl acetate (1.3 g, 2.45 mmol, 1.0 eq) in CH₂Cl₂ (15 ml). The reaction mixture was allowed to stir at room temperature for overnight. TLC indicated starting material was consumed and the desired product was observed. The reaction mixture was diluted with water and extracted with CH₂Cl₂ (3×15 ml). The combined organic layers were dried over Na₂SO₄, filtered and evaporated under reduced pressure. The crude residue was purified by silicagel column chromatography by using 0.5% methanol:dichloromethanes as an eluent to obtain the desired product (1.1 g, 73.8% yield) as a white solid. ¹H NMR (300 MHz, CDCl₃): δ ppm 4.52-4.45 (m, 1H), 4.08-3.85 (m, 2H), 3.45-3.39 (m, 1H), 3.36 (s, 3H), 3.23-3.0 (m, 2H), 2.82-2.70 (m, 1H), 2.04 (s, 3H), 0.96 (s, 3H), 0.92 (s, 3H), 0.898 (s, 3H), 0.85 (s, 3H), 0.84 (s, 3H), 0.83 (s, 3H), 2.13-0.77 (m, 28H), 0.48-0.40 (m, 1H), 0.38-0.27 (m, 1H), 0.28-0.12 (m, 2H); ES MS: [M+H]⁺ 610.4 (100%).

### Step 6: Synthesis of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl) (4-methoxypiperidin-1-yl) methanone

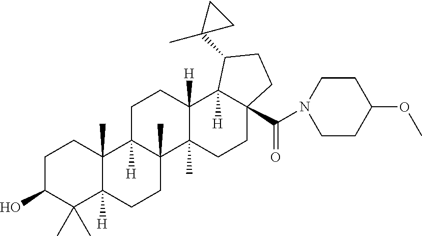

To a stirred solution of (1R,3aS,5aR,5bR,7aR,9S,11aR,11bR,13aR,13bR)-3a-(4-methoxy piperidine-1-carbonyl)-5a,5b,8,8,11a-pentamethyl-1-(1-methylcyclopropyl)icosahydro-1H-cyclopenta[a]chrysen-9-yl acetate (step 5) (1.1 g, 1.80 mmol, 1.0 eq) in 1,4-dioxane (11 ml) was added aqueous 4N NaOH solution (0.55 ml). The reaction mixture was refluxed for 36 hours. TLC indicated starting material was consumed and the desired product was observed. The reaction mixture was diluted with water and the aqueous layer was extracted with CH₂Cl₂ (3×20 ml). The combined organic layers were dried over Na₂SO₄, filtered and evaporated under reduced pressure. The crude residue was purified by silicagel column chromatography by using 2% methanol:dichloromethanes as an eluent to obtain the desired product (0.750 g, 73.5% yield) as an off-white solid. ¹H NMR (300 MHz, DMSO-d6): δ ppm 3.92-3.73 (m, 2H), 3.23 (s, 3H), 3.17-2.93 (m, 3H), 2.82-2.70 (m, 1H), 0.93 (s, 3H), 0.87 (s, 6H), 0.83 (s, 3H), 0.779 (s, 3H), 0.65 (s, 3H), 2.10-0.60 (m, 29H), 0.36-0.27 (m, 2H), 0.22-0.13 (m, 2H); ES MS: [M+H]⁺ 568.3 (100%), [M+Na]⁺ 590.3 (20%); IR (KBr) cm⁻¹: 3480, 2993, 2948, 2867, 1619, 1454, 1090.

### Step 7: Synthesis of (1R,3S)-1-benzyl 3-((1R,3aS,5aR,5bR,7aR,9S,11aR,11bR,13aR,13bR)-3a-(4-methoxypiperidine-1-carbonyl)-5a,5b,8,8,11a-pentamethyl-1-(1-methylcyclopropyl)icosahydro-1H-cyclopenta[a]chrysen-9-yl) 2,2-dimethylcyclobutane-1,3-dicarboxylate

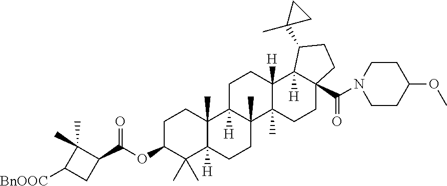

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl)(4-methoxypiperidin-1-yl)methanone (step 6) (0.400 g, 0.705 mmol, 1.0 eq) in toluene (10 ml) was added DMAP (0.430 g, 3.52 mmol, 5.0 eq) and (1S,3R)-3-(benzyloxycarbonyl)-2,2-dimethylcyclobutane carboxylic 2,4,6-trichlorobenzoic anhydride (0.660 g, 1.41 mmol, 2.0 eq). The reaction mixture was heated to reflux for overnight. TLC indicated starting material was consumed and the desired product was observed. The reaction mixture was evaporated under reduced pressure, diluted with water (20 ml) and extracted with CH₂Cl₂ (3×15 ml). The combined organic layers were dried over Na₂SO₄, filtered and evaporated under reduced pressure. The crude residue was purified by silicagel column chromatography by using 2% methanol:dichloromethanes as an eluent to obtain the desired product (0.410 g, 71% yield) as an off-white solid. ¹H NMR (300 MHz, CDCl₃): δ ppm 7.40-7.33 (m, 5H), 5.18-5.07 (m, 2H), 4.44 (dd, J=4.8, 10.8 Hz, 1H), 4.10-3.87 (m, 2H), 3.47-3.37 (m, 1H), 3.36 (s, 3H), 3.23-3.0 (m, 2H), 2.87-2.60 (m, 4H), 0.96 (brs, 6H), 0.92 (s, 3H), 0.89 (s, 3H), 0.85 (s, 3H), 0.84 (s, 3H), 0.83 (s, 3H), 2.13-0.75 (m, 32H), 0.47-0.30 (m, 2H), 0.27-0.15 (m, 2H); ES MS: [M+H]⁺ 812.5 (100%).

### Step 8: Synthesis of (1R,3S)-3-(((1R,3aS,5aR,5bR,7aR,9S,11aR,11bR,13aR,13bR)-3a-(4-methoxypiperidine-1-carbonyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylicacid

To a solution of (1R,3S)-1-benzyl 3-((1R,3aS,5aR,5bR,7aR,9S,11aR,11bR,13aR,13bR)-3a-(4-methoxypiperidine-1-carbonyl)-5a,5b,8,8,11a-pentamethyl-1-(1-methylcyclopropyl)icosahydro-1H-cyclopenta[a]chrysen-9-yl) 2,2-dimethylcyclobutane-1,3-dicarboxylate (step 7) (0.410 g, 0.50 mmol, 1.0 eq) in dichloromethane (10 ml) was added palladium (II) acetate (0.056 g, 0.252 mmol, 0.5 eq), triethyl amine (0.24 ml, 1.76 mmol, 3.5 eq) and triethylsilane (0.24 ml, 1.51 mmol, 3.0 eq). The reaction mixture was heated to reflux under nitrogen atmosphere for 48 hours. TLC indicated starting material was consumed and the desired product was observed. The reaction mixture was cooled to room temperature, filtered through a pad of celite and washed with dichloromethane. The filtrate was dried over Na₂SO₄, filtered and evaporated under reduced pressure. The crude residue was purified by silicagel column chromatography by using 1% methanol:dichloromethanes as an eluent to obtain the desired product (0.280 g, 76.9% yield) as a white solid. ¹H NMR (300 MHz, CDCl₃): δ ppm 4.52-4.42 (m, 1H), 4.10-3.85 (m, 2H), 3.47-3.40 (m, 1H), 3.36 (s, 3H), 3.23-3.0 (m, 2H), 2.90-2.72 (m, 3H), 2.67-2.52 (m, 1H), 1.08 (s, 3H), 0.97 (s, 3H), 0.92 (s, 3H), 0.89 (s, 3H), 0.86 (s, 9H), 2.14-0.77 (m, 32H), 0.48-0.40 (m, 1H), 0.38-0.30 (m, 1H), 0.28-0.13 (m, 2H); ES MS: [M+H]⁺ 722.4 (100%); IR (KBr) cm⁻¹: 3437, 2947, 2869, 1732, 1630, 1411, 1189; HPLC: 96.5%.

### Example 30

### Preparation of 3-(1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl)oxy)carbonyl)-3-(methyl-d3)butanoic-4,4,4-d3 acid

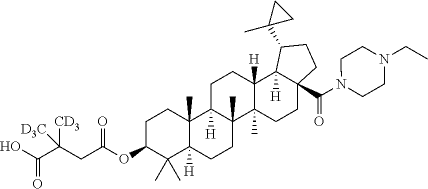

### Step 1: Synthesis of (1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl) icosa hydro-1H-cyclopenta[a]chrysen-9-yl acetate

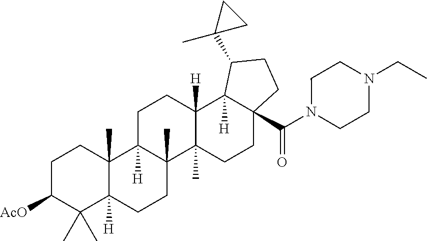

To a stirred solution of ethyl piperazine (0.358 mL, 3.77 mmol, 2.0 eq) and triethylamine (1.3 mL, 9.4 mmol, 5.0 eq) in CH₂Cl₂ (10 ml) at 0° C. was added a solution of (1R,3aS,5aR,5bR,7aR,9S,11aR,11bR,13aR,13bR)-3a-(chlorocarbonyl)-5a,5b,8,8,8,11a-pentamethyl-1-(1-methylcyclopropyl)icosahydro-1H-cyclopenta[a]chrysen-9-yl acetate (1.0 g, 1.88 mmol, 1.0 eq) in DCM (10 ml). The reaction mixture was allowed to stir at room temperature for overnight. The reaction mixture was diluted with water and extracted with CH₂Cl₂ (3×100 ml). The combined organic extracts were dried over Na₂SO₄, filtered and evaporated under reduced pressure. The residue was purified by column chromatography by using 30% ethyl acetate and hexane as an eluent to obtain the compound (1.1 g, 95.0%) as a white solid. H¹ NMR (DMSO-d₆, 300 MHz): δ 4.36 (t, 1H), 3.45 (bs, 4H), 2.29 (bs, 4H), 2.12-2.04 (m, 2H), 1.99 (s, 3H), 1.94-1.86 (m, 2H), 1.69-1.48 (m, 9H), 1.45-1.06 (m, 14H), 1.04-0.98 (m, 6H), 0.95-0.79 (m, 15H), 0.34 (t, 2H) and 0.19 (t, 2H); Mass: [M]⁺ 609.37 (100%), [M+Na]⁺ 631.36 (75%).

### Step 2: Synthesis of (4-ethylpiperazin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl) methanone

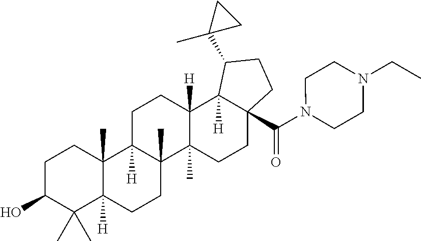

To a stirred solution of (1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl) icosa hydro-1H-cyclopenta[a]chrysen-9-yl acetate (step 1) (1.1 g, 1.80 mmol, 1.0 eq) in THF (10 ml) in Methanol (10 ml) was added potassium carbonate (1.74 g, 12.6 mmol, 7.0 eq). The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by column chromatography by using 50% ethyl acetate and hexane as an eluent gave the compound (0.9 g, 87.0%) as a white solid. H¹ NMR (DMSO-d₆, 300 MHz): δ 4.28 (t, 1H), 3.45 (bs, 4H), 3.01 (t, 1H), 2.78 (d, 1H), 2.30-2.25 (m, 11H), 2.09 (d, 1H), 1.94-1.88 (m, 2H), 1.61-1.43 (m, 9H), 1.35-1.23 (m, 8H), 1.20-1.09 (m, 2H), 1.00-0.93 (m, 3H), 0.87 (s, 6H), 0.83 (s, 3H), 0.77 (m, 3H), 0.65 (s, 3H) 0.31 (bd, 2H) and 0.19 (bd, 2H); Mass: [M]⁺ 567.43 (100%), [M+Na]⁺ 589.39 (60%).

### Step 3: Synthesis of 3-(1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl)oxy)carbonyl)-3-(methyl-d3)butanoic-4,4,4-d3 acid

To a stirred solution of (4-ethylpiperazin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysen-3a-yl)methanone (step 2) (0.9 g, 1.59 mmol, 1.0 eq) and 3,3-dimethyldihydrofuran-2,5-dione (0.9 mL, 6.36 mmol, 4.0 eq) in toluene (18 ml) was added DMAP (0.387 g, 3.18 mmol, 2.0 eq). The reaction mixture was heated at 90° C. for overnight. TLC indicated starting material was consumed and the desired product was observed. The mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by column chromatography by using 5% methanol and DCM as an eluent gave the compound (0.520 g, 45.0%) as a white solid. H¹ NMR (CDCl₃, 300 MHz): δ 4.54 (t, 1H), 3.50 (bs, 4H), 3.08 (bs, 2H), 2.81-2.67 (m, 5H), 1.99 (s, 3H), 1.94-1.86 (m, 2H), 1.69-1.48 (m, 7H), 1.45-1.06 (m, 14H), 1.04-0.98 (m, 6H), 0.95-0.79 (m, 15H), 0.34 (t, 2H) and 0.19 (t, 2H); Mass: [M]⁺ 700.37 (100%); HPLC: 93.10%.

### Example 31

### Preparation of 2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)oxy)butanoic acid

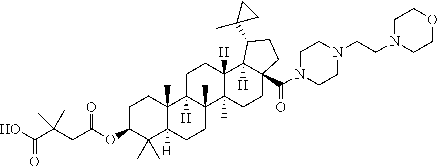

### Step 1: Synthesis of tert-butyl piperazine-1-carboxylate

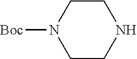 [SMILES: CC(C)(C)OC(=O)N1CCNCC1]

The stirred solution of piperazine (20 g, 1.0 eq) in DCM (400 mL) was stirred at 0° C. under nitrogen atmosphere. Boc anhydride (23.2 mL, 0.5 eq) was dissolved in DCM (400 mL) then added drop wise to said solution and the reaction mixture was maintained at same temperature for 3 hours. After completion of the reaction (monitored by TLC), the reaction mixture was obtained solid which solid was filtered and washed with DCM. The filtrate was concentrated under reduced presser and dissolved in water (300 mL) cooled to 0° C. then added solid K₂CO₃ portion wise (300 g) and extracted with DCM. The organic layer was dried with Na₂SO₄ and filtered then concentrated and dissolved in diisopropyl ether and filtered and washed with diisopropyl ether organic layer was concentrated offered crude semisolid compound which was purified by column chromatography to obtain the semisolid product (10.0 g, 23.0%). H¹ NMR (CD₃OD, 300 MHz): δ 3.40 (t, 4H), 2.77 (t, 4H) and 1.46 (s, 9H); Mass: [M]⁺ 187.14 (30%), [M+Na]⁺ 209.14 (50%).

### Step 2: Synthesis of 4-(2-bromoethyl)morpholine

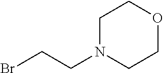

To the stirred solution of morpholine (20 g, 1.0 eq) in Acetone (200 mL) K₂CO₃ (63 g, 2.0 eq) was added followed by dibromoethane (39.4 mL, 2.0 eq) then the reaction mixture was refluxed for 24 hours. After completion of the reaction (monitored by TLC), the reaction mixture was filtered and filtrate was concentrated and extracted with DCM, washed with water, dried with Na₂SO₄, filtered and concentrated then crud was purified by column to obtain the semisolid product (3.0 g, 7.0%). H¹ NMR (CD₃OD, 300 MHz): δ 3.74 (t, 4H), 3.45 (t, 2H), 2.81 (t, 2H), and 2.53 (t, 4H); Mass: [M]⁺ 195.56 (100%).

### Step 3: Synthesis of tert-butyl 4-(2-morpholinoethyl)piperazine-1-carboxylate

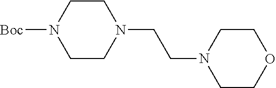 [SMILES: CC(C)(C)OC(=O)N1CCN(CCN2CCOCC2)CC1]

To the stirred solution of 4-(2-bromoethyl)morpholine (step 2) (3.5 g, 1.0 eq) in acetonitrile (40 mL) K₂CO₃ (7.5 g, 3.0 eq) was added followed by tert-butyl piperazine-1-carboxylate (step 1) (3.4 g, 1.0 eq) then the reaction mixture was stirred at room temperature for 12 hours. After completion of the reaction (monitored by TLC), the reaction mixture was filtered and filtrate was concentrated, extracted with DCM and washed with water, dried with Na₂SO₄, filtered, concentrated and purified by column to obtain the solid compound (5.0 g, 92.0%). H¹ NMR (CD₃OD, 300 MHz): δ 3.70 (t, 4H), 3.44 (t, 4H), 12.55-2.45 (m, 12H) and 1.45 (s, 9H); Mass: [M]⁺ 300.14 (100%), [M+Na]⁺ 322.12 (80%).

### Step 4: Synthesis of (1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl acetate

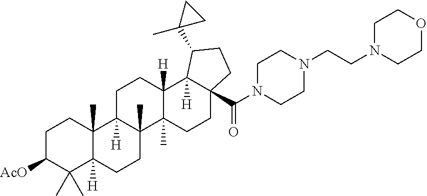

To a stirred solution of 4-(2-(piperazin-1-yl)ethyl)morpholine (0.86 g, 7.54 mmol, 1.9 eq) and triethylamine (1.90 g, 18.8 mmol, 5.0 eq) in CH₂Cl₂ (30 ml) (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-yl acetate (1.1 g, 3.77 mmol, 1.0 eq) in CH₂Cl₂(10 ml) was added at 0° C. The reaction mixture was allowed to stir at room temperature for overnight. The reaction mixture was diluted with water and extracted with CH₂Cl₂. The combined organic extracts were dried over Na₂SO₄, filtered and evaporated under reduced pressure. The residue was purified by column chromatography by using 8% methanol:DCM as an eluent to give the desired product (1.1 g, 70.0%) as a white solid. H¹ NMR (DMSO-D₆, 300 MHz): δ 4.40-4.35 (q, 1H), 3.55 (t, 4H), 3.45 (bs, 4H), 2.76 (s, 1H), 2.57 (m, 1H), 2.39-2.10 (m, 13H), 1.96 (m, 3H), 1.91 (m, 2H), 1.69-1.05 (s, 16H), 0.95 (s, 3H), 0.87 (s, 3H), 0.84 (s, 3H), 0.82 (m, 12H), 0.34-0.29 (t, 2H) and 0.19-0.18 (q, 2H); Mass: [M]⁺ 694.39 (100%).

### Step 5: Synthesis of (1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)(4-(2-morpholinoethyl)piperazin-1-yl)methanone

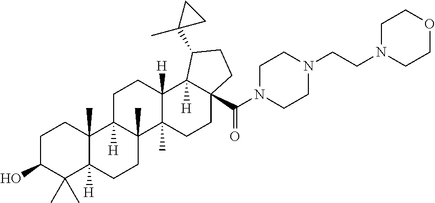

To a stirred solution of (1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl acetate (step 4) (1.1 g, 3.28 mmol, 1.0 eq) in THF (10 ml) and Methanol (10 ml) potassium carbonate (2.0 g, 23.0 mmol, 7.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by column chromatography by using 1% methanol:dichloromethane as an eluent to give the desired product (0.85 g, 82.0%) as a white solid. H¹ NMR (CDCl₃, 300 MHz): δ 4.28-4.26 (d, 1H), 3.55 (t, 4H), 3.44 (bs, 4H), 3.17 (d, 1H), 2.98 (q, 1H), 2.78-2.63 (m, 2H), 2.35-2.31 (m, 12H), 2.05 (bd, 1H), 1.95-1.87 (m, 3H), 1.61-1.25 (m, 19H), 0.93 (s, 3H), 0.86 (s, 9H), 0.77 (s, 3H), 0.65 (m, 3H), 0.34-0.29 (t, 2H) and 0.19-0.14 (q, 2H); Mass: [M]⁺ 652.51 (100%).

### Step 6: Synthesis of 2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)oxy)butanoic acid

To a stirred solution of (1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-morpholinoethyl)piperazin-1-yl)methanone (step 5) (0.30 g, 0.494 mmol, 1.0 eq) and 2,2-dimethyl succinicanhydride (0.24 g, 1.97 mmol, 4.0 eq) in toluene (5 ml) DMAP (0.120 g, 0.988 mmol, 2.0 eq) was added. The reaction mixture was heated at 90° C. for overnight. TLC indicated starting material was consumed and the desired product was observed. The mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by column chromatography by using 5% methanol:dichloromethane as an eluent to give the desired product (0.220 g, 61.0%) as a white solid. H¹ NMR (CDCl₃, 300 MHz): δ 4.40-4.35 (q, 1H), 3.57-3.54 (m, 4H), 3.45 (t, 4H), 2.42-2.3 5 (m, 14H), 2.05 (bs, 1H), 1.87 (m, 2H), 1.65-1.23 (m, 18H), 1.16 (m, 8H), 1.10-0.94 (m, 2H), 0.87 (s, 3H), 0.84-0.78 (m, 16H), 0.32 (t, 1H) and 0.19 (t, 2H); Mass: [M]⁻ 780.49 (100%); HPLC: 98.84%.

### Example 32

### Preparation of (1R,3S)-2,2-dimethyl-3-(1R,3aS,5aR,5bR,7aR,9S,11aR,11bR,13aR,13bR)-5a,5b,8,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)oxy)carbonyl)cyclobutane-1-carboxylic acid

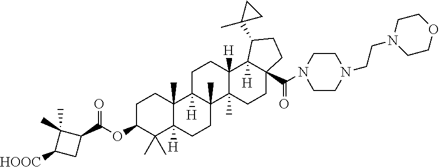

### Step 1: Synthesis of 1-benzyl 3-((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)(1R,3S)-2,2-dimethylcyclobutane-1,3-di carboxylate

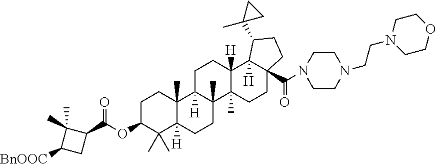

To a stirred solution of (1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-morpholinoethyl)piperazin-1-yl)methanone (0.550 g, 1.05 mmol, 1.0 eq) in toluene (11 ml) DMAP (0.206 g, 2.11 mmol, 2.0 eq) and (1S,3R)-3-(benzyloxycarbonyl)-2,2-dimethylcyclobutane carboxylic 2,4,6-trichlorobenzoic anhydride (0.792 g, 2.11 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂. The combined organic layers were dried over Na₂SO₄, filtered and evaporated under reduced pressure. The crude residue was purified by column chromatography by using 8.0% methanol:DCM as an eluent to obtain the desired product (0.650 g, 85.0% yield) as a white solid. H¹ NMR (DMSO-d₆, 300 MHz): −δ 7.35 (m, 5H), 5.10-5.03 (q, 2H), 4.36 (t, 3H), 4.13 (d, 1H), 3.63-3.60 (t, 4H), 3.50 (bs, 4H), 2.96-2.80 (m, 3.0), 2.63 (s, 2H), 2.95 (bs, 9H), 2.36-2.27 (m, 2H), 2.08-1.65 (m, 4H), 1.65-1.25 (m, 25H), 0.94 (s, 3H), 0.90 (s, 3H), 0.87-0.80 (m, 20H), 0.34 0.30, (m, 2H) and 0.19-0.16 (m, 2H); Mass: [M]⁻ 896.67 (100%).

### Step 2: Synthesis of (1R,3S)-2,2-dimethyl-3-(1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)oxy)carbonyl)cyclobutane-1-carboxylic acid

To a solution of 1-benzyl 3-((1R,3aS,5aR,5bR,7aR,9S,11aR,11bR,13aR,13bR)-5a,5b,8,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)(1R,3S)-2,2-dimethylcyclobutane-1,3-dicarboxylate (step 1) (0.650 g, 0.77 mmol, 1.0 eq) in MeOH (3 mL) and ethyl acetate (10 mL) palladium carbon (0.150 g, 0.388 mmol, 0.5 eq) was added. The reaction mixture was in hydrogen atmosphere at room temperature for overnight. Completion of the reaction mixture was filtered through a pad of celite and washed with MeOH and DCM. The filtrate was evaporated under reduced pressure, the crude residue was purified by column chromatography by using 12% methanol:DCM as an eluent to obtain the desired compound (0.050 g, 8.0%) as a white solid. H¹ NMR (DMSO-d₆, 300 MHz): δ 12.15 (bs, 1H), 4.34 (m, 1H), 3.53 (q, 4H), 3.45 (m, 4H), 2.79-2.76 (m, 3H), 2.76 (m, 19H), 2.39 (m, 2H), 1.89 (m, 3H), 1.53-1.12 (m, 17H), 0.94 (m, 3H), 0.90-0.82 (m, 18H), 0.32, (t, 2H) and 0.17 (t, 2H); Mass: [M]⁻ 806.57 (40%); HPLC: 93.63%.

### Example 33

### Preparation of 2,2-dimethyl-4-oxo-4-(1R,3aS,5aR,5bR,7aR,9S,11aR,11bR,13aR,13bR)-5a,5b,8,8,11a-pentamethyl-1-(1-methylcyclopropyl)-3a-(4-(3-morpholinopropyl) piperazine-1-carbonyl)icosahydro-1H-cyclopenta[a]chrysen-9-yl)oxy)butanoic acid

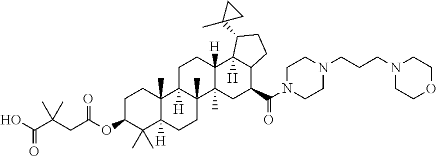

### Step 1: Synthesis of 4-(3-bromopropyl)morpholine

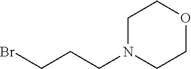

To the stirred solution of morpholine (20 g, 1.0 eq) in Acetone (200 mL) K₂CO₃ (126 g, 4.0 eq) was added followed by 1,3-dibromopropane (92.6 g, 4.0 eq) then the reaction mixture was refluxed for 24 hours. After completion of the reaction (monitored by TLC), the reaction mixture was filtered and filtrate was concentrated, extracted with DCM and washed with water. The organic layer was dried with Na₂SO₄, filtered and filtrated was concentrated which crud was purified by column to obtain the semisolid desired product (8.0 g, 16.0%). H¹ NMR (DMSO-d₆, 300 MHz): δ 4.32 (t, 2H), 3.79 (t, 2H), 3.57 (t, 5H), 2.48-2.34 (m, 4H) and 1.99-1.90 (m, 1H); Mass: [M]⁺ 209.45 (100%).

### Step 2: Synthesis of tert-butyl 4-(3-morpholinopropyl)piperazine-1-carboxylate

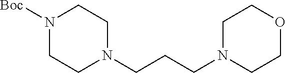 [SMILES: CC(C)(C)OC(=O)N1CCN(CCCN2CCOCC2)CC1]

To the stirred solution of 4-(3-bromopropyl)morpholine (step 1) (4.0 g, 1.2 eq) in acetonitrile (60 mL) K₂CO₃ (7.8 g, 3.5 eq) was added followed by tert-butyl piperazine-1-carboxylate (3.0 g, 1.0 eq) then the reaction mixture was stirred at room temperature for 12 hours. After completion of the reaction (monitored by TLC), the reaction mixture was filtered and filtrate was concentrated, extracted with DCM and washed with water. The organic layer was dried with Na₂SO₄, filtered and concentrated which crud was purified by column to obtain the solid desired product (3.5 g, 69.0%). H¹ NMR (CDCl₃, 300 MHz): δ 3.73 (t, 4H), 3.70 (t, 4H), 2.46-2.36 (m, 12H), 1.75-1.65 (m, 2H), and 1.45 (s, 9H); Mass: [M]⁺ 313.13 (100%).

### Step 3: Synthesis of (1R,3aS,5aR,5bR,7aR,9S,11aR,1bR,13aR,13bR)-5a,5b,8,8,11a-pentamethyl-1-(1-methylcyclopropyl)-3a-(4-(3-morpholinopropyl)piperazine-1-carbonyl)icosahydro-1H-cyclopenta[a]chrysen-9-yl acetate

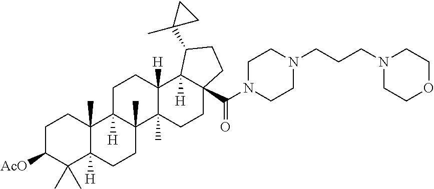

To a stirred solution of 4-(3-(piperazin-1-yl)propyl)morpholine (1.2 g, 5.8 mmol, 2.0 eq) and triethylamine (2. mL, 14.5 mmol, 5.0 eq) in CH₂Cl₂ (20 ml) at 0° C. was added a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-yl acetate (1.5 g, 2.9 mmol, 1.0 eq) in CH₂Cl₂(20 ml). The reaction mixture was allowed to stir at room temperature for overnight. The reaction mixture was diluted with water and extracted with CH₂Cl₂. The combined organic extracts were dried over Na₂SO₄, filtered and evaporated under reduced pressure. The residue was purified by column chromatography by using 8% methanol:DCM as an eluent to obtain the desired product (0.8 g, 40.0%) as a white solid. H¹ NMR (DMSO-D₆, 300 MHz): δ 4.47-4.42 (q, 1H), 4.22 (d, 1H), 3.72 (t, 4H), 3.62 (bs, 4H), 3.23 (q, 1H), 2.81 (q, 1H), 2.52-2.39 (m, 11H), 2.20 (d, 1H), 2.02 (s, 5H), 1.76-1.17 (m, 22H), 0.95 (s, 3H), 0.93-0.83 (m, 15H), 0.39-0.34 (q, 2H) and 0.24-0.20 (q, 2H); Mass: [M]⁺ 707.51 (100%).

### Step 4: Synthesis of (1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)(4-(3-morpholinopropyl)piperazin-1-yl)methanone

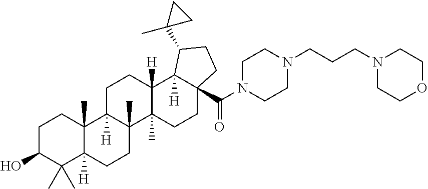

To a stirred solution of (1R,3aS,5aR,5bR,7aR,9S,11aR,11bR,13aR,13bR)-5a,5b,8,8,11a-pentamethyl-1-(1-methylcyclopropyl)-3a-(4-(3-morpholinopropyl)piperazine-1-carbonyl)icosahydro-1H-cyclopenta[a]chrysen-9-yl acetate (step 3) (0.8 g, 1.13 mmol, 1.0 eq) in THF (7 ml) and Methanol (7 ml) potassium carbonate (1.0 g, 7.9.0 mmol, 7.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by column chromatography by using 1% methanol:dichloromethane as an eluent to obtain the desired product (0.700 g, 93.0%) as a white solid. H¹ NMR (CDCl₃, 300 MHz): δ 4.47-4.42 (q, 1H), 4.22 (d, 1H), 3.72 (t, 4H), 3.62 (bs, 4H), 3.23 (q, 1H), 2.81 (q, 1H), 2.52-2.39 (m, 11H), 2.20 (d, 1H), 2.02 (s, 5H), 1.76-1.17 (m, 20H), 0.95 (s, 3H), 0.93-0.83 (m, 15H), 0.39-0.34 (q, 2H) and 0.24-0.20 (q, 2H); Mass: [M]⁺ 666.42 (100%), [M+Na]⁺ 688.37 (35%).

### Step 5: Synthesis of 2,2-dimethyl-4-oxo-4-(1R,3aS,5aR,5bR,7aR,9S,11aR,11bR,13aR,13bR)-5a,5b,8,8,11a-pentamethyl-1-(1-methylcyclopropyl)-3a-(4-(3-morpholinopropyl) piperazine-1-carbonyl)icosahydro-1H-cyclopenta[a]chrysen-9-yl)oxy)butanoic acid

To a stirred solution of (1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(3-morpholinopropyl)piperazin-1-yl)methanone (step 4) (0.100 g, 0.150 mmol, 1.0 eq) and 2,2-dimethyl succinicanhydride (0.076 g, 0.60 mmol, 4.0 eq) in toluene (10 ml) DMAP (0.036 g, 0.30 mmol, 2.0 eq) was added. The reaction mixture was heated at 90° C. for overnight. TLC indicated starting material was consumed and the desired product was observed. The reaction mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by column chromatography by using 5% methanol:dichloromethane as an eluent to obtain the desired product (0.020 g, 16.0%) as a white solid. H¹ NMR (CD₃OD, 300 MHz): δ 4.49-4.43 (m, 1H), 3.74-3.68 (m, 4H), 3.64 (bs, 4H), 3.42 (m 1H), 2.77-2.73 (m, 2H), 2.66-2.45 (m, 10H), 2.19-1.97 (m, 3H), 1.82-1.37 (m, 16H), 1.29-1.17 (m, 14H), 0.95 (s, 4H), 0.93-0.82 (m, 15H), 0.37 (t, 2H) and 0.24 (t, 2H); Mass: [M]⁺ 794.60 (100%); HPLC: 90.16%.

### Example 34

### Preparation of (1R,3S)-2,2-dimethyl-3-(1R,3aS,5aR,5bR,7aR,9S,11aR,11bR,13aR,13bR)-5a,5b,8,8,8,11a-pentamethyl-1-(1-methylcyclopropyl)-3a-(4-(3-morpholinopropyl) piperazine-1-carbonyl)icosahydro-1H-cyclopenta[a]chrysen-9-yl)oxy)carbonyl)cyclobutane-1-carboxylic acid

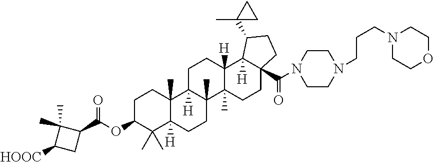

### Step 1: Synthesis of 1-benzyl 3-((1R,3aS,5aR,5bR,7aR,9S,11aR,11bR,13aR,13bR)-5a,5b,8,8,11a-pentamethyl-1-(1-methylcyclopropyl)-3a-(4-(3-opolinopropyl)piperazine-1-carbonyl)icosahydro-1H-cyclopenta[a]chrysen-9-yl)(1R,3S)-2,2-dimethylcyclobutane-1,3-dicarboxylate

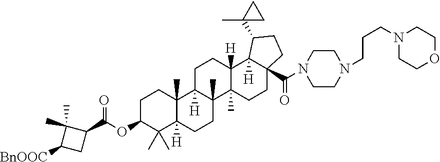

To a stirred solution of (1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(3-morpholinopropyl)piperazin-1-yl)methanone (0.400 g, 0.01 mmol, 1.0 eq) in toluene (20 ml) DMAP (0.128 g, 1.20 mmol, 2.0 eq) and (1S,3R)-3-(benzyloxycarbonyl)-2,2-dimethylcyclobutane carboxylic 2,4,6-trichlorobenzoic anhydride (0.6 g, 1.32 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂. The combined organic layers were dried over Na₂SO₄, filtered and evaporated under reduced pressure. The crude residue was purified by column chromatography by using 5% methanol:dichloromethane as an eluent to obtain (0.400 g, 80.0%) as a white solid. H¹ NMR (CD₃OD, 300 MHz): −δ 7.33 (m, 5H), 5.13-5.10 (q, 2H), 4.41-4.31 (m, 2H), 3.47-3.24 (m, 4H), 2.29-2.27 (m, 4H), 2.37 (d, 2H), 2.08-1.90 (m, 5H), 1.23-1.05 (m, 10H), 1.05-0.87 (m, 30H), 0.83-0.35 (m, 20H), 0.25, (m, 2H) and 0.19 (m, 2H); Mass: [M]⁺ 910.67 (100%).

### Step 2: Synthesis of (1R,3S)-2,2-dimethyl-3-(1R,3aS,5aR,5bR,7aR,9S,11aR,11bR,13aR,13bR)-5a,5b,8,8,11a-pentamethyl-1-(1-methylcyclopropyl)-3a-(4-(3-morpholinopropyl) piperazine-1-carbonyl)icosahydro-1H-cyclopenta[a]chrysen-9-yl)oxy)carbonyl)cyclobutane-1-carboxylic acid

To a solution of 1-benzyl 3-((1R,3aS,5aR,5bR,7aR,9S,11aR,11bR,13aR,13bR)-5a,5b,8,8,8,11a-pentamethyl-1-(1-methylcyclopropyl)-3a-(4-(3-morpholinopropyl)piperazine-1-carbonyl)icosahydro-1H-cyclopenta[a]chrysen-9-yl)(1R,3S)-2,2-dimethylcyclobutane-1,3-dicarboxylate (step 1) (0.400 g, 0.44 mmol, 1.0 eq) in the EtOAc (30 mL) and MeOH (10 mL) then 10% palladium carbon (0.200 g) was added. The reaction mixture was in hydrogen atmosphere at room temperature, overnight. Completion of the reaction mixture was filtered through a pad of celite and was washed with MeOH and DCM. The filtrate was evaporated under reduced pressure, the crude residue was purified by column chromatography by using 10% methanol:DCM as an eluent to obtain the desired compound (0.050 g, 13.0%) as a white solid. H¹ NMR (CDCl₃, 300 MHz): δ 4.37 (t, 1H), 3.58 (bs, 4H), 3.47 (bs, 4H), 2.82-2.76 (m, 3H), 2.39-2.34 (m, 15H), 2.09 (m, 2H), 1.92-1.86 (m, 4H), 1.52-1.40 (m, 9H), 1.39-1.26 (m, 10H), 1.17-1.02 (m, 4H), 0.94-0.82 (m, 20H), 0.34, (t, 2H) and 0.20 (t, 2H); Mass: [M]⁺ 820.62 (100%); HPLC: 90.43%.

### Example 35

### Preparation of 4-(1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

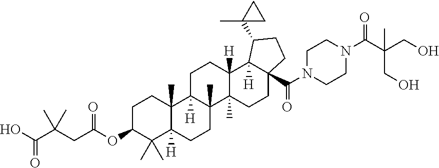

### Step 1: Synthesis of tert-butyl 4-(3-hydroxy-2-(hydroxymethyl)-2-methylpropanoyl)piperazine-1-carboxylate

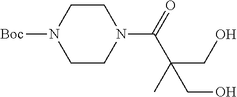 [SMILES: CC(C)(C)OC(=O)N1CCN(C(=O)C(C)(CO)CO)CC1]

EDCI, HCl (6.16 g, 32.25 mmol, 3.0 eq) and diisopropylethylamine (9.30 ml, 53.75 mmol, 5 eq) were added to a solution of tert-butyl piperazine-1-carboxylate (2.0 g, 10.75 mmol, 1.0 ew) and 3-hydroxy-2-(hydroxymethyl)-2-methylpropanoic acid (1.5 g, 11.82 mmol, 1.1 eq) then HOBt (1.74 g, 12.9 mmole, 1.2 eq) was added and stirred for overnight at room temperature under nitrogen. The solvent was removed in vacuo and the residue purified by column chromatography on silica gel eluting with 5% methanol and DCM to obtain the title compound as a white solid (1.6 g. 50.0%). H¹ NMR (CDCl₃, 300 MHz): δ 4.54 (t, 2H), 3.53 (d, 8H), 3.31 (t, 4H), 1.40 (s, 9H) and 1.08 (s, 3H); Mass: [M]⁺ 302.17 (80%), [M+Na]⁺ 325.15 (100%).

### Step 2: Synthesis of (1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl acetate

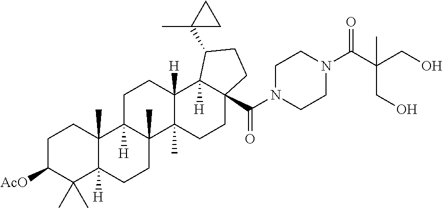

To a stirred solution of 3-hydroxy-2-(hydroxymethyl)-2-methyl-1-(piperazin-1-yl)propan-1-one (0.914 g, 4.52 mmol, 2.0 eq) and triethylamine (1.57 mL, 11.3 mmol, 5.0 eq) in CH₂Cl₂ (14 ml)(1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-yl acetate (1.2 g, 2.26 mmol, 1.0 eq) in CH₂Cl₂(10 ml) was added at 0° C. The reaction mixture was allowed to stir at room temperature for overnight. The reaction mixture was diluted with water and extracted with CH₂Cl₂. The combined organic extracts were dried over Na₂SO₄, filtered and evaporated under reduced pressure. The residue was purified by column chromatography by using 5% methanol:DCM as an eluent to obtain the desired product (0.900 g, 57.0%) as a white solid. H¹ NMR (DMSO-D₆, 300 MHz): δ 4.61-4.53 (m, 3H), 4.38 (m, 1H), 3.65-3.48 (m, 15H), 2.97 (s, 1H), 2.11-1.87 (m, 6H), 1.60-1.23 (m, 16H), 1.10 (d, 6H), 0.95 (s, 3H), 0.88-0.79 (m, 13H), 0.36-0.29 (t, 2H) and 0.20-0.12 (q, 2H); Mass: [M]⁺ 697.53 (25%), [M+Na]⁺ 719.45 (40%).

### Step 3: Synthesis of 3-hydroxy-1-(4-(1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)piperazin-1-yl)-2-(hydroxymethyl)-2-methylpropan-1-one

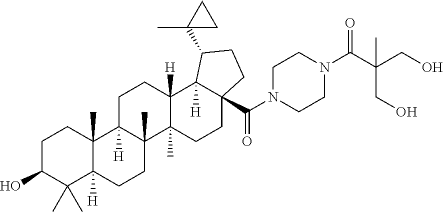

To a stirred solution of (1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl acetate (step 2) (0.9 g, 1.29 mmol, 1.0 eq) in THF (10 ml) and Methanol (10 ml) potassium carbonate (1.247 g, 9.03 mmol, 7.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by column chromatography by using 7% methanol:DCM as an eluent to obtain the desired product (0.430 g, 57.0%) as a white solid. H¹ NMR (CDCl₃, 300 MHz): δ 4.28 (d, 1H), 3.38 (bs, 4H), 3.07-2.94 (m, 2H), 2.80-2.76 (m, 1H), 2.59 (m, 4H), 2.09 (d, 1H), 1.95-1.87 (m, 2H), 1.61-1.53 (m, 12H), 1.43-1.23 (m, 9H), 1.10 (m, 3H), 0.93 (s, 3H), 0.89-0.87 (m, 13H), 0.78 (s, 3H), 0.65 (s, 4H), 0.31 (m, 2H) and 0.19 (m, 2H); Mass: [M]⁺ 655.36 (80%), [M+Na]⁺ 677.33 (100%).

### Step 4: Synthesis of 4-(1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

To a stirred solution of 3-hydroxy-1-(4-((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)piperazin-1-yl)-2-(hydroxymethyl)-2-methylpropan-1-one (step 3) (0.200 g, 0.305 mmol, 1.0 eq) and 2,2-dimethyl succinicanhydride (0.156 g, 1.22 mmol, 4.0 eq) in toluene (5 ml) DMAP (0.075 g, 0.61 mmol, 2.0 eq) was added. The reaction mixture was heated at 90° C. for 6 hours. TLC indicated starting material was consumed and the desired product was observed. The reaction mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by column chromatography by using 2% methanol:DCM as an eluent gave the desired product (0.050 g, 21.0%) as a white solid. H¹ NMR (DMSO-d₆, 300 MHz): δ 11.79 (s, 1H), 4.27 (d, 1H), 3.47-3.44 (m, 4H), 2.97 (bs, 4H), 2.08-1.90 (m, 4H), 1.69-1.35 (m, 22H), 1.14 (s, 9H), 0.94 (s, 3H), 0.87-0.78 (m, 19H), 0.65 (s, 3H), 0.34 (t, 2H) and 0.19 (t, 2H); Mass: [M]⁻ 783.49 (40%); HPLC: 93.31%.

### Example 36

### Preparation of (1R,3S)-3-(1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)oxy) carbonyl)-2,2-dimethylcyclobutane-1-carboxylic acid

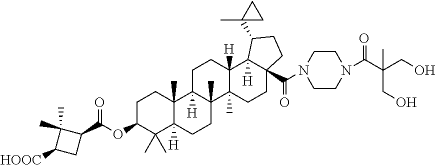

### Step 1: Synthesis of 1-benzyl 3-((1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)(1R,3S)-2,2-dimethylcyclobutane-1,3-dicarboxylate

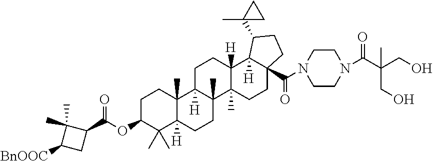

To a stirred solution of 3-hydroxy-1-(4-((1R,3aS,5aR,5bR,7aR,9S,11aR,1 bR,13aR,13bR)-9-hydroxy-5a,5b,8,8,11a-pentamethyl-1-(1-methylcyclopropyl)icosahydro-1H-cyclopenta[a]chrysene-3a-carbonyl)piperazin-1-yl)-2-(hydroxymethyl)-2-methylpropan-1-one (0.230 g, 0.351 mmol, 1.0 eq) in toluene (5 ml) DMAP (0.085 g, 0.702 mmol, 2.0 eq) and (1S,3R)-3-(benzyloxycarbonyl)-2,2-dimethylcyclobutane carboxylic 2,4,6-trichlorobenzoic anhydride (0.329 g, 0.702 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂. The combined organic layers were dried over Na₂SO₄, filtered and evaporated under reduced pressure. The crude residue was purified by column chromatography by using 30% EtOAC:Hexane as an eluent to obtain the desired product (0.300 g, 95.0% yield) as a white solid. H¹ NMR (DMSO-d₆, 300 MHz): −δ 7.38-7.33 (m, 5H), 5.14-5.03 (q, 2H), 4.33 (bs, 1H), 3.40-3.38 (m, 4H), 2.95-2.80 (m, 4H), 2.04-1.93 (m, 8H), 1.58-1.42 (m, 13H), 1.30-1.10 (m, 16H), 0.94 (s, 3H), 0.87-0.74 (m, 22H), 0.35, (t, 2H) and 0.21 (t, 2H); Mass: [M]⁻ 898.65 (100%).

### Step 2: Synthesis of (R,3S)-3-(1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)oxy) carbonyl)-2,2-dimethylcyclobutane-1-carboxylic acid

To a solution of 1-benzyl 3-((1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)(1R,3S)-2,2-dimethylcyclobutane-1,3-dicarboxylate (step 1) (0.030 g, 0.334 mmol, 1.0 eq) in EtOAc (6 ml) and methanol (2 mL) palladium Carbon (0.070 g, 0.668 mmole, 2 eq) was added and set hydrogen atmosphere for overnight. The TLC indication starting material was disappeared. The reaction mixture was filtered through celite and washed with methanol and DCM the filtration was concentrated and the crude residue was purified by column chromatography by using 5% methanol:DCM as an eluent to obtain the desired compound (135 g, 50.0%) as a white solid. H¹ NMR (DMSO-d₆, 300 MHz): δ 12.08 (s, 1H), 4.37 (d, 1H), 3.50-3.41 (m, 6H), 3.18-3.13 (m, 1H), 2.82-2.70 (m, 4H), 1.46-1.18 (m, 15H), 1.00 (s, 4H), 0.95-0.82 (m, 25H), 0.32, (t, 2H) and 0.19 (t, 2H); Mass: [M]⁺ 808.65 (100%); HPLC: 89.05%.

### Example 37

### Preparation of 4-(1R,3aS,5aR,5bR,7aR,9S,11aR,11bR,13aR,13bR)-3a-(4-(tert-butoxy carbonyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

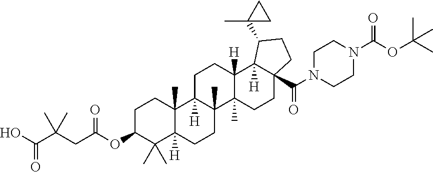

### Step 1: Synthesis of tert-butyl 4-((1R,3aS,5aR,5bR,7aR,9S,11aR,11bR,13aR,13bR)-9-acetoxy-5a,5b,8,8,11a-pentamethyl-1-(1-methylcyclopropyl)icosahydro-1H-cyclopenta[a]chrysene-3a-carbonyl)piperazine-1-carboxylate

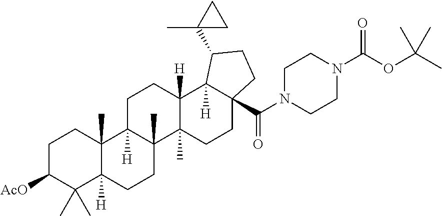

To a stirred solution of tert-butyl piperazine-1-carboxylate (0.900 g, 4.83 mmol, 2.0 eq) and triethylamine (1.2 mL, 11.3 mmol, 4.0 eq) in CH₂Cl₂ (10 ml), a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-yl acetate (1.2 g, 2.26 mmol, 1.0 eq) in CH₂Cl₂ (10 ml) was added at 0° C. The reaction mixture was allowed to stir at room temperature for overnight. The reaction mixture was diluted with water and extracted with CH₂Cl₂. The combined organic extracts were dried over Na₂SO₄, filtered and evaporated under reduced pressure. The residue was purified by column chromatography by using 2% methanol:DCM as an eluent to obtain the desired product (1.3 g, 84.0%) as a white solid. H¹ NMR (CDCl₃, 300 MHz): δ 4.51-4.45 (t, 1H), 3.54 (bs, 4H), 3.38 (bs, 4H), 2.78 (t, 1H), 2.07 (s, 5H), 2.04-1.59 (m, 9H), 1.52-1.46 (m, 12H), 1.41-1.33 (m, 9H), 1.28-0.97 (m, 3H), 0.92 (s, 3H), 0.90 (s, 6H), 0.83 (m, 6H), 0.78 (s, 1H), 0.45-0.31 (m, 2H) and 0.24-0.22 (m, 2H); Mass: [M]⁺ 681.42 (100%), [M+Na]⁺ 703.41 (50%).

### Step 2: Synthesis of tert-butyl 4-((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)piperazine-1-carboxylate

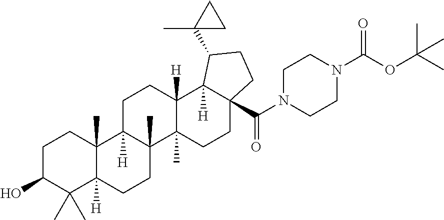

To a stirred solution of tert-butyl 4-((1R,3aS,5aR,5bR,7aR,9S,11aR,11bR,13aR,13bR)-9-acetoxy-5a,5b,8,8,11a-pentamethyl-1-(1-methylcyclopropyl)icosahydro-1H-cyclopenta[a]chrysene-3a-carbonyl)piperazine-1-carboxylate (step 1) (1.3 g, 1.91 mmol, 1.0 eq) in THF (6.5 ml) and Methanol (6.5 ml) potassium carbonate (1.8 g, 13.37 mmol, 7.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by column chromatography by using 30% EtOAc:Hexane as an eluent to obtain the desired product (1.1 g, 88.0%) as a white solid. H¹ NMR (DMSO-d₆, 300 MHz): δ 4.26 (d, 1H), 3.46 (bs, 4H), 3.24 (bs, 4H), 3.01-2.94 (m, 1H), 2.77 (t, 1H), 2.08-1.86 (m, 4H), 1.61-1.45 (m, 9H), 1.39 (m, 8H), 1.31-1.06 (m, 12H), 0.93 (s, 3H), 0.87 (s, 9H), 0.78 (m, 3H), 0.65 (s, 3H), 0.32 (m, 2H) and 0.19 (m, 2H); Mass: [M]639.44 (100%), [M+Na]⁺ 661.45 (40%).

### Step 3: Synthesis of 4-(1R,3aS,5aR,5bR,7aR,9S,11aR,11bR,13aR,13bR)-3a-(4-(tert-butoxy carbonyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

To a stirred solution of tert-butyl 4-(1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)piperazine-1-carboxylate (step 2) (0.400 g, 0.626 mmol, 1.0 eq) and 2,2-dimethyl succinicanhydride (0.321 g, 2.5 mmol, 4.0 eq) in toluene (8 ml) DMAP (0.152 g, 1.25 mmol, 2.0 eq) was added. The reaction mixture was heated at 90° C. for 6 hours. TLC indicated starting material was consumed and the desired product was observed. The mixture was concentrated under reduced pressure, cooled to 0° C., acidified to pH=5 with 1N HCl and extracted with CH₂Cl₂.

The combined organic extracts were washed with water, dried over Na₂SO₄, filtered and evaporated under reduced pressure. The crude residue was purified by column chromatography by using 10% methanol:DCM as an eluent to obtain the desired product (0.390 g, 81.0%) as a white solid. H¹ NMR (DMSO-d₆, 300 MHz): δ 12.19 (s, 1H), 4.40 (t, 1H), 3.56 (bs, 4H), 3.45 (bs, 4H), 3.24 (bs, 3H), 2.76 (m, 1H), 2.53 (m, 3H), 2.08-1.86 (m, 3H), 1.66-1.50 (m, 8H), 1.45 (s, 10H), 1.39 (m, 3H), 1.33 (s, 3H), 1.16 (m, 7H), 0.94 (s, 3H), 0.87 (s, 3H). 0.81-0.77 (m, 13H), 0.34 (q, 2H) and 0.22-0.13 (m, 2H); Mass: [M]⁺ 767.49 (70%); HPLC: 89.30%.

### Example 38

### Preparation of (1R,3S)-3-(1R,3aS,5aR,5bR,7aR,9S,11aR,11bR,13aR,13bR)-3a-(4-(tert-butoxycarbonyl)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

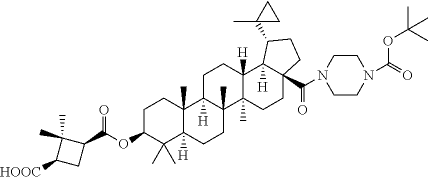

### Step 1: Synthesis of 1-benzyl 3-(1R,3aS,5aR,5bR,7aR,9S,11aR,11bR,13aR,13bR)-3a-(4-(tert-butoxycarbonyl)piperazine-1-carbonyl)-5a,5b,8,8,11a-pentamethyl-1-(1-methylcyclopropyl)icosahydro-1H-cyclopenta[a]chrysen-9-yl)(1R,3S)-2,2-dimethylcyclobutane-1,3-di carboxylate

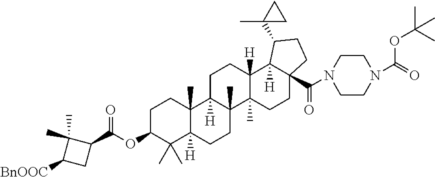

To a stirred solution of tert-butyl 4-((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)piperazine-1-carboxylate (0.650 g, 1.01 mmol, 1.0 eq) in toluene (15 ml) DMAP (0.250 g, 2.03 mmol, 2.0 eq) and (1S,3R)-3-(benzyloxycarbonyl)-2,2-dimethylcyclobutane carboxylic 2,4,6-trichlorobenzoic anhydride (0.950 g, 2.03 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂. The combined organic layers were dried over Na₂SO₄, filtered and evaporated under reduced pressure. The crude residue was purified by column chromatography by using 30% EtOAc and hexane as an eluent to obtain (0.850 g, 94.0%) as a white solid. H¹ NMR (CDCl₃, 300 MHz): −δ 7.34 (m, 5H), 5.16-5.11 (m, 2H), 4.47-4.42 (m, 1H), 3.54 (m, 4H), 3.37 (m, 4H), 2.95-2.37 (m, 4H), 2.07 (m, 1H), 2.05-1.86 (m, 3H), 1.71-1.52 (m, 9H), 1.46 (m, 10H), 1.40-1.25 (m, 10H), 1.23-1.11 (m 5H), 0.99-0.79 (m, 20H), 0.45-0.32, (m, 2H) and 0.24-0.18 (m, 2H); Mass: [M]⁺ 883.56 (70%), [M+Na]⁺ 905.57 (100%).

### Step 2: Synthesis of (1R,3S)-3-(1R,3aS,5aR,5bR,7aR,9S,11aR,11bR,13aR,13bR)-3a-(4-(tert-butoxycarbonyl)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

To a solution of 1-benzyl 3-((1R,3aS,5aR,5bR,7aR,9S,11aR,11bR,13aR,13bR)-3a-(4-(tert-butoxycarbonyl)piperazine-1-carbonyl)-5a,5b,8,8,11a-pentamethyl-1-(1-methylcyclopropyl)icosahydro-1H-cyclopenta[a]chrysen-9-yl)(1R,3S)-2,2-dimethylcyclobutane-1,3-dicarboxylate (step 1) (0.850 g, 0.96 mmol, 1.0 eq) in Ethylacetate (15 ml) palladium Carbon (0.070 g, 0.668 mmole, 2 eq) was added and set hydrogen atmosphere for overnight. Observed TLC starting material was disappear, the reaction mixture was filtered through celite filtrate was concentrated and crude residue was purified by column chromatography by using 5% methanol:DCM as an eluent to obtain the desired compound (0.415 g, 54.0%) as a white solid. H¹ NMR (DMSO-d₆, 300 MHz): δ 12.13 (s, 1H), 4.37-4.32 (t, 1H), 3.45 (bs, 4H), 3.24 (bs, 4H), 2.82 (t, 2H), 2.35-2.22 (m, 2H), 2.08-1.86 (m, 6H), 1.66-1.45 (m, 8H), 1.46 (m, 11H), 1.40-1.25 (m, 9H), 1.16-1.12 (m 4H), 0.95-0.81 (m, 20H), 0.34, (m, 2H) and 0.19 (m, 2H); Mass: [M]⁺ 793.48 (50%), [M+Na]⁺ 815.47 (100%); HPLC: 89.11%.

### Example 39

### Preparation of 4-(((1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

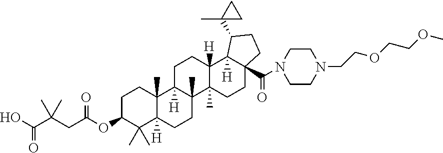

### Step 1: Synthesis of tert-butyl 4-(2-hydroxyethyl)piperazine-1-carboxylate

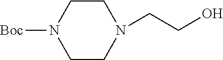 [SMILES: CC(C)(C)OC(=O)N1CCN(CCO)CC1]

To a stirred solution of 1-(2-Hydroxyethyl)piperazine (8.0 g, 61.45 mmol, 1.0 eq) in 1,4-dioxane (80 ml) di-tert-butyldicarbonate (15.93 g, 73.7 mmol, 1.2 eq) was added at 0° C. The reaction mixture was allowed to stir at room temperature for 3 hours. TLC indicated starting material was consumed and the desired product was observed. The reaction mixture was evaporated under reduced pressure to get the desired compound (11.0 g, 77.7% yield) as light green color syrup solid. This was used directly for next step without further purification. H NMR (300 MHz, CDCl₃): δ ppm 3.65-3.61 (m, 2H), 3.46-3.43 (m, 4H), 2.55 (t, J=5.4 Hz, 2H), 2.47-2.44 (m, 4H), 1.45 (s, 9H); ES Mass: [M+Na]⁺ 253.09 (100%).

### Step 2: Synthesis of tert-butyl 4-(2-(2-methoxyethoxyl)ethyl)piperazine-1-carboxylate

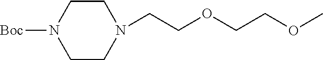 [SMILES: COCCOCCN1CCN(C(=O)OC(C)(C)C)CC1]

To a stirred solution of tert-butyl 4-(2-hydroxyethyl) piperazine-1-carboxylate (step 1) (6.0 g, 26.08 mmol, 1.0 eq) in THF (40 ml) sodium hydride (2.5 g, 104.34 mmol, 4.0 eq) was added at 0° C. and stirred for 1 hour at 0° C. followed by 2-methoxyethyl 4-methylbenzenesulfonate (9.0 g, 39.13 mmol) in THF (20 mL) slowly to the reaction mixture at 0° C. Then reaction mixture was refluxed for 3 hours. TLC indicated starting material was consumed and the desired product was observed. The reaction mixture was quenched with ice cold water at 0° C. The reaction mixture was extracted with EtOAc (2×200 mL). The combined organic layers were dried over Na₂SO₄, filtered and evaporated under reduced pressure to obtain the desired product (4.0 g) as syrup. ¹H NMR (300 MHz, CDCl₃): δ ppm 3.65-3.53 (m, 6H), 3.48-3.36 (m, 4H), 3.38 (s, 3H), 2.6 (t, J=6.0 Hz, 2H), 2.47-2.44 (m, 4H), 1.46 (s, 9H); ES Mass: [M+1]⁺ 289.15 (100%).

### Step 3: Synthesis of I-(2-(2-methoxyethoxyl)ethyl)piperazine hydrochloride

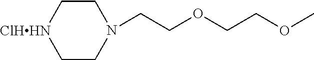

To a stirred solution of tert-butyl 4-(2-(2-methoxyethoxyl)ethyl)piperazine-1-carboxylate (step 2) (2.2 g, 7.62 mmol, 1.0 eq) in CH₂Cl₂ (5 ml) 4M HCl in dioxane (18 mL) was added. The reaction mixture was stirred at room temperature for 3 hours. TLC indicated starting material was consumed and the desired product was observed. The reaction mixture was concentrated under reduced pressure to get the residue (1.71 gr) and used directly for next step without further purification.

### Step 4: Synthesis of (1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl acetate

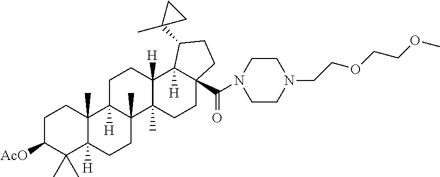

To a stirred solution of 1-(2-(2-methoxyethoxyl)ethyl)piperazine hydrochloride (step 3) (1.69 g, 7.54 mmol, 2.0 eq) and triethylamine (2.6 mL, 18.85 mmol, 5.0 eq) in CH₂Cl₂ (30 ml) a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-ylacetate (2.0 g, 3.77 mmol, 1.0 eq) in CH₂Cl₂(10 ml) was added at 0° C. The reaction mixture was allowed to stir at room temperature for overnight. The reaction mixture was diluted with water and extracted with CH₂Cl₂ (3×100 ml). The combined organic extracts were dried over Na₂SO₄, filtered and evaporated under reduced pressure. The residue was purified by silica gel column chromatography by using 4% methanol:dichloromethane as an eluent gave the desired product (1.9 g, 73.4% yield) as off-white solid. ¹H NMR (300 MHz, DMSO): δ ppm 4.4-4.35 (m, 1H), 3.49-3.41 (m, 8H), 3.23 (s, 3H), 2.79-2.74 (m, 1H), 2.44 (s, 3H), 2.33 (bs, 4H), 2.1-2.0 (m, 1H), 1.97 (s, 3H), 1.94-1.92 (m, 2H), 1.78-1.18 (m, 19H), 1.09-1.02 (m, 4H), 0.98 (s, 3H), 0.95 (s, 3H), 0.87-0.79 (m, 11H), 0.32-0.3 (m, 2H), 0.19-0.17 (m, 2H); ES Mass: [M]⁺ 683.44.

### Step 5: Synthesis of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)(4-(2-(2-methoxyethoxyl)ethyl)piperazin-1-yl)methanone

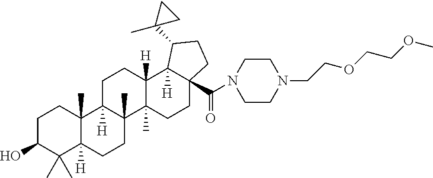

To a stirred solution of (1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl acetate (step 4) (1.9 g, 2.78 mmol, 1.0 eq) in THF (10 ml) and Methanol (10 ml) potassium carbonate (2.7 g, 19.5 mmol, 7.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by silicagel column chromatography by using 4% methanol:dichloromethane as an eluent gave the desired product (1.52 g, 85.43% yield) as a white solid. ¹H NMR (300 MHz, DMSO): δ ppm 4.28-4.27 (m, 1H), 3.48-3.41 (m, 8H), 3.23 (s, 3H), 2.98-2.97 (m, 1H), 2.77 (bs, 1H), 2.44-2.42 (m, 3H), 2.32-2.12 (bs, 4H), 2.09-1.87 (m, 3H), 1.61-1.44 (m, 10H), 1.34-1.23 (m, 10H), 1.12-1.08 (m, 2H), 0.93 (s, 3H), 0.86 (s, 9H), 0.83 (s, 3H), 0.77 (s, 3H), 0.31-0.29 (m, 2H), 0.19-0.17 (m, 2H); ES Mass: [M+H]⁺ 641.45 (100%).

### Step 6: Synthesis of 4-(((1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-(2-methoxyethoxyl)ethyl)piperazin-1-yl)methanone (step 5) (0.45 g, 0.7 mmol, 1.0 eq) and 2,2-dimethyl succinicanhydride (0.36 g, 2.8 mmol, 4.0 eq) in toluene (5 ml) DMAP (0.171 g, 1.4 mmol, 2.0 eq) was added. The reaction mixture was heated at 90° C. for overnight. TLC indicated starting material was consumed and the desired product was observed. The mixture was concentrated under reduced pressure, cooled to 0° C., acidified to pH=6 with 0.5N HCl and extracted with CH₂Cl₂. The combined organic extracts were washed with water, dried over Na₂SO₄, filtered and evaporated under reduced pressure. The crude residue was purified by silica gel column chromatography by using 5% methanol:dichloromethane as an eluent gave the desired product (0.2 g, 37.2% yield) as a white solid. ¹H NMR (300 MHz, DMSO): δ ppm 12.2 (bs, 1H), 4.38 (t, J=5.1 Hz, 1H), 3.48-3.41 (m, 10H), 3.23 (s, 3H), 3.11-2.92 (m, 3H), 2.41-2.31 (m, 5H), 2.09-1.87 (m, 4H), 1.66-1.32 (m, 16H), 1.29-1.21 (m, 2H), 1.16-1.15 (m, 6H), 1.14-1.07 (m, 2H), 1.02-0.94 (m, 4H), 0.87-0.78 (m, 15H), 0.33-0.28 (m, 2H), 0.19-0.17 (m, 2H); ES Mass: [M+H]⁺ 770.4 (100%); HPLC purity: 92.8+6.3%.

### Example 40

### Preparation of (1R,3S)-3-((((1R,3aS,5aR,5bR,7aR,9S,11aR,11bR,13aR,13bR)-3a-(4-(2-(2-methoxyethoxy)ethyl)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

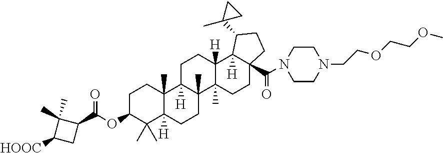

### Step 1: Synthesis of (1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl (1S,3R)-2,2-dimethyl-3-(2-phenylacetoxy)cyclobutane-1-carboxylate

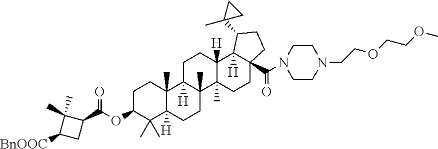

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-(2-methoxyethoxyl)ethyl)piperazin-1-yl)methanone (0.600 g, 0.937 mmol, 1.0 eq) in toluene (10 ml) DMAP (0.316 g, 1.87 mmol, 2.0 eq) and (1S,3R)-3-(benzyloxycarbonyl)-2,2-dimethylcyclobutane carboxylic 2,4,6-trichlorobenzoic anhydride (0.88 g, 1.87 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂ (2×50 ml). The combined organic layers were dried over Na₂SO₄, filtered and evaporated under reduced pressure. The crude residue was purified by silica gel column chromatography by using 3% methanol:dichloromethane as an eluent to obtain the desired product (0.60 g, 72.4% yield) as a white solid. ¹H NMR (300 MHz, DMSO): δ ppm 7.36 (m, 5H), 5.08 (q, J=7.2 Hz, 2H), 4.34 (t, J=7.8 Hz, 1H), 3.56-3.51 (m, 8H), 3.49-3.42 (m, 2H), 3.23 (s, 3H), 2.96-2.8 (m, 2H), 2.73-2.66 (m, 3H), 2.55-2.34 (m, 2H), 2.08-1.9 (m, 5H), 1.65-1.01 (m, 21H), 0.94 (s, 3H), 0.87 (s, 3H), 0.84-0.8 (m, 20H), 0.32-0.3 (m, 2H), 0.18-0.15 (m, 2H); ES Mass: [M]⁺ 885.62 (100%).

### Step 2: Synthesis of (1R,3S)-3-((((1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

To a solution of (1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl (1S,3R)-2,2-dimethyl-3-(2-phenylacetoxyl)cyclobutane-1-carboxylate (step 1) (0.6 g, 0.678 mmol, 1.0 eq) in EtOAc:MeOH (1:1, 8 mL) 10% Pd/C (60 mg) was added. Hydrogen gas was bubbled through the reaction mixture via a balloon for 2 hours. The resultant mixture was filtered through celite and washed with methanol and the filtrate was concentrated and the crude residue was purified by silicagel column chromatography by using 5% methanol:dichloromethane as an eluent gave the desired compound (0.29 g, 53.9% yield) as a white solid. ¹H NMR (300 MHz, DMSO): δ ppm 12.16 (bs, 1H), 4.35 (t, J=7.8 Hz, 1H), 3.59-3.41 (m, 8H), 3.24 (s, 3H), 2.97-2.9 (m, 2H), 2.82-2.73 (m, 5H), 2.41-2.22 (m, 5H), 1.92-1.86 (m, 5H), 1.66-1.34 (m, 15H), 1.3-1.21 (m, 5H), 1.19-1.09 (m, 3H), 0.95-0.82 (m, 21H), 0.39-0.29 (m, 2H), 0.26-0.16 (m, 2H); ES Mass: [M+H]⁺ 796.43 (100%); HPLC purity: 97.8%.

### Example 41

### Preparation of 4-(((1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

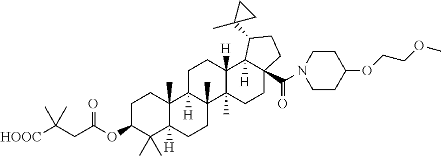

### Step 1: Synthesis of tert-Butyl 4-oxopiperidine-1-carboxylate

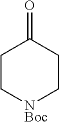 [SMILES: CC(C)(C)OC(=O)N1CCC(=O)CC1]

To a stirred solution of piperidin-4-onehydrochloride (13 g, 95.94 mmol, 1.0 eq) in 1,4-dioxane:water (4:1, 130 ml) triethylamine (24.2 g, 239.8 mmol, 2.5 eq) and di-tert-butyldicarbonate (31.4 g, 143.9 mmol, 1.5 eq) were added at 0° C. The reaction mixture was allowed to stir at room temperature for overnight. TLC indicated starting material was consumed and the desired product was observed. The reaction mixture was evaporated under reduced pressure, diluted with CH₂Cl₂ (350 ml), washed with water (2×50 ml). The organic layer was dried over Na₂SO₄, filtered, evaporated under reduced pressure and purified by silicagel column chromatography by using 1% methanol:dichloromethane as an eluent to obtain the desired product (16.1 g, 84.3% yield) as a white solid. ¹H NMR (300 MHz, CDCl₃): 8 ppm (m, 4H), 2.46-2.42 (m, 4H), 1.49 (s, 9H); ES Mass: [M+1]⁺ 200.

### Step 2: Synthesis of tert-butyl 4-hydroxypiperidine-1-carboxylate

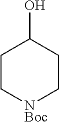 [SMILES: CC(C)(C)OC(=O)N1CCC(O)CC1]

To a stirred solution of tert-Butyl 4-oxopiperidine-1-carboxylate (step 1) (13 g, 65.3 mmol, 1.0 eq) in Methanol (130 ml) sodiumborohydride (0.741 g, 19.59 mmol, 0.3 eq) was added at 0° C. The reaction mixture was allowed to stir at room temperature for overnight. TLC indicated starting material was consumed and the desired product was observed. The reaction mixture was evaporated under reduced pressure, diluted with water (200 ml) and extracted with CH₂Cl₂ (2×200 ml). The combined organic layers were dried over Na₂SO₄, filtered and evaporated under reduced pressure to obtain the desired product (13.0 g, 99%) as a white solid. ¹H NMR (300 MHz, CDCl₃+D₂O): δ ppm (m, 3H), 3.06-2.98 (m, 2H), 1.88-1.83 (m, 2H), 1.53-1.46 (m, 2H), 1.45 (s, 9H); ES Mass: [M+Na]⁺ 224.07.

### Step 3: Synthesis of 2-methoxyethyl 4-methylbenzenesulfonate

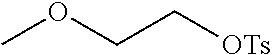

To a stirred solution of 2-methoxyethanol (10 g, 131.4 mmol, 1.0 eq) in CH₂Cl₂ (100 ml) at 0° C. was added triethyl amine (39.84 g, 394.4 mmol, 3.0 eq), 4-dimethylaminopyridine (4.81 g, 39.44 mmol, 0.3 eq) and para-toluenesulphonylchloride (27.5 g, 144.6 mmol, 1.1 eq). The reaction mixture was allowed to stir at room temperature for overnight. TLC indicated starting material was consumed and the desired product was observed. The reaction mixture was diluted with water (200 ml), organic layer was separated and the aqueous layer was extracted with CH₂Cl₂ (2×200 ml). The combined organic layers were dried over Na₂SO₄, filtered and evaporated under reduced pressure. The crude residue was purified by silicagel column chromatography by using dichloromethane as an eluent to obtain the desired product (12.0 g, 39.6% yield) as a brown liquid. ¹H NMR (300 MHz, CDCl₃): δ ppm (d, J=8.1 Hz, 2H), 7.35 (d, J=8.1 Hz, 2H), 4.17-4.14 (m, 2H), 3.59-3.56 (m, 2H), 3.31 (s, 3H), 2.45 (s, 3H); ES Mass: [M+Na]⁺ 252.96.

### Step 4: Synthesis of tert-butyl 4-(2-methoxyethoxyl)piperidine-1-carboxylate

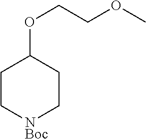 [SMILES: COCCOC1CCN(C(=O)OC(C)(C)C)CC1]

To a suspension of sodium hydride (0.228 g, 9.95 mmol, 4.0 eq, 60% dispersion in mineral oil) in THF (10 ml) tert-butyl 4-hydroxypiperidine-1-carboxylate (step 2)(0.500 g, 2.48 mmol, 1.0 eq) was added at 0° C. The reaction mixture was stirred at 0° C. for 30 minutes, 2-methoxyethyl 4-methylbenzenesulfonate (step 3) (0.858 g, 3.73 mmol, 1.5 eq) in THF (5 ml) was added and the reaction was allowed to reach to room temperature and heated to reflux for overnight. TLC indicated starting material was consumed and the desired product was observed. The reaction mixture was quenched with saturated NH₄Cl solution (20 ml) and extracted with CH₂Cl₂ (2×20 ml). The combined organic layers were dried over Na₂SO₄, filtered and evaporated under reduced pressure. The crude residue was purified by silicagel column chromatography by using methanol:dichloromethane as an eluent to obtain the desired product (0.340 g, 58.9% yield) as a brown liquid. ¹H NMR (300 MHz, CDCl₃): 6 ppm (m, 2H), 3.68-3.39 (m, 5H), 3.39 (s, 3H), 3.07-2.98 (m, 2H), 1.86-1.84 (m, 2H), 1.58-1.49 (m, 2H), 1.45 (s, 9H); ES Mass: [M+Na]⁺ 282.03.

### Step 5: Synthesis of 4-(2-methoxyethoxyl)piperidine hydrochloride

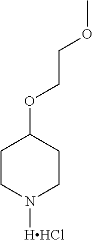

To a stirred solution of tert-butyl 4-(2-methoxyethoxyl)piperidine-1-carboxylate (step 4) (3.0 g, 7.32 mmol, 1.0 eq) in CH₂Cl₂ (9 ml) 4M HCl in dioxane (20 mL) was added. The reaction mixture was stirred at room temperature for 3 hours. TLC indicated starting material was consumed and the desired product was observed. The reaction mixture was concentrated under reduced pressure to get the residue (2.3 gr) and used directly for next step without further purification.

### Step 6: Synthesis of (1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl acetate

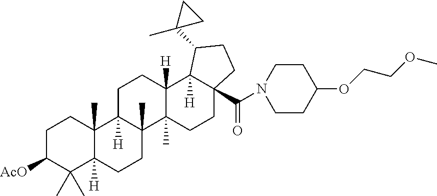

To a stirred solution of 4-(2-methoxyethoxyl)piperidine hydrochloride (step 5)(1.47 g, 7.52 mmol, 2.0 eq) and triethylamine (1.9 g, 18.82 mmol, 5.0 eq) in CH₂Cl₂ (30 ml) a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-ylacetate (2.0 g, 3.76 mmol, 1.0 eq) in CH₂Cl₂(10 ml) was added at 0° C. The reaction mixture was allowed to stir at room temperature for overnight. The reaction mixture was diluted with water and extracted with CH₂Cl₂ (3×100 ml). The combined organic extracts were dried over Na₂SO₄, filtered and evaporated under reduced pressure. The residue was purified by silicagel column chromatography by using 20% EtOAc:hexane as an eluent gave the desired product (2.1 g, 85% yield) as off-white solid. ¹H NMR (300 MHz, DMSO): δ ppm 4.4-4.35 (m, 1H), 3.84-3.78 (m, 2H), 3.55-3.48 (m, 3H), 3.43-3.39 (m, 2H), 3.23 (s, 3H), 3.13-2.94 (m, 2H), 2.1-2.02 (m, 1H), 1.99 (s, 3H), 1.96-1.73 (m, 5H), 1.68-1.08 (m, 23H), 0.95 (s, 3H), 0.9-0.76 (m, 15H), 0.34-0.29 (m, 2H), 0.19-0.15 (m, 2H); ES Mass: [M+H]⁺ 654.42.

### Step 7: Synthesis of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)(4-(2-methoxyethoxyl)piperidin-1-yl)methanone

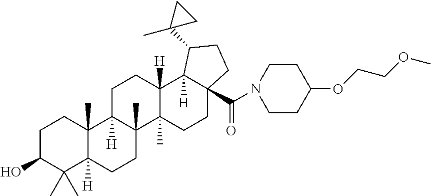

To a stirred solution of (1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl acetate (step 6) (2.1 g, 3.2 mmol, 1.0 eq) in THF (10 ml) and Methanol (10 ml) potassium carbonate (3.09 g, 22.4 mmol, 7.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by silicagel column chromatography by using 20% EtOAc:hexane as an eluent gave the desired product (1.8 g, 91% yield) as a white solid. ¹H NMR (300 MHz, DMSO): δ ppm 4.29-4.27 (m, 1H), 3.94-3.68 (m, 2H), 3.57-3.46 (m, 3H), 3.43-3.39 (m, 2H), 3.23 (s, 3H), 3.11-2.92 (m, 2H), 2.1-2.03 (m, 1H), 1.96-1.73 (m, 5H), 1.68-1.41 (m, 9H), 1.37-1.04 (m, 14H), 0.93 (s, 3H), 0.9-0.74 (m, 12H), 0.68-0.62 (m, 3H), 0.34-0.29 (m, 2H), 0.19-0.14 (m, 2H); ES Mass: [M+H]⁺ 612.43.

### Step 8: Synthesis of 4-(((1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-methoxyethoxyl)piperidin-1-yl)methanone (step 7) (0.6 g, 0.98 mmol, 1.0 eq) and 2,2-dimethyl succinicanhydride (0.5 g, 3.9 mmol, 4.0 eq) in toluene (12 ml) DMAP (0.239 g, 1.95 mmol, 2.0 eq) was added. The reaction mixture was heated at 90° C. for overnight. TLC indicated starting material was consumed and the desired product was observed. The mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by silicagel column chromatography by using 30% EtOAc:hexane as an eluent gave the desired product (0.3 g, 41.1% yield) as a white solid. ¹H NMR (300 MHz, DMSO): δ ppm 12.1 (bs, 1H), 4.38-4.36 (m, 1H), 3.54-3.5 (m, 3H), 3.47-3.43 (m, 2H), 3.23 (s, 3H), 3.07-2.99 (m, 2H), 2.81-2.76 (m, 1H), 2.5-2.41 (m, 4H), 2.06-2.04 (m, 2H), 1.92-1.79 (m, 13H), 1.62-1.33 (m, 10H), 1.15 (m, 12H), 0.95 (s, 3H), 0.87-0.78 (m, 12H), 0.37-0.28 (m, 2H), 0.22-0.13 (m, 2H); ES Mass: [M]⁺ 740.4; HPLC: 88.08+8.9%.

### Example 42

### Preparation of (1R,3S)-3-((((1R,3aS,5aR,5bR,7aR,9S,11aR,11bR,13aR,13bR)-3a-(4-(2-methoxyethoxyl)piperidine-1-carbonyl)-5a,5b,8,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

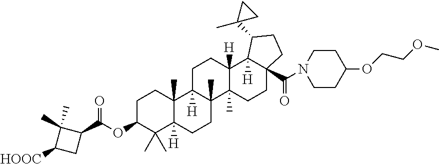

### Step 1: Synthesis of (1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl (1S,3R)-2,2-dimethyl-3-(2-phenylacetoxy)cyclobutane-1-carboxylate

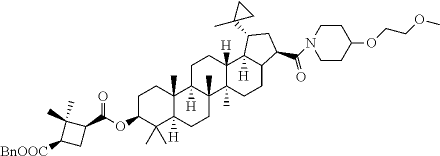

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-methoxyethoxyl)piperidin-1-yl)methanone (0.600 g, 0.98 mmol, 1.0 eq) in toluene (10 ml) DMAP (0.239 g, 1.95 mmol, 2.0 eq) and (1S,3R)-3-(benzyloxycarbonyl)-2,2-dimethylcyclobutane carboxylic 2,4,6-trichlorobenzoic anhydride (0.991 g, 2.11 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂ (2×50 ml). The combined organic layers were dried over Na₂SO₄, filtered and evaporated under reduced pressure. The crude residue was purified by silicagel column chromatography by using 1.5% methanol:dichloromethane as an eluent to obtain (0.8 g, 95% yield) as a white solid. ¹H NMR (300 MHz, CDCl₃): δ ppm 7.41-7.26 (m, 5H), 51.8-5.06 (m, 2H), 4.49-4.38 (m, 1H), 4.15-3.94 (m, 2H), 3.66-3.59 (m, 2H), 3.56-3.48 (m, 3H), 3.39 (s, 3H), 3.16-3.05 (m, 1H), 2.98-2.58 (m, 5H), 2.42-2.31 (m, 1H), 2.12-1.97 (m, 3H), 1.96-1.83 (m, 2H), 1.72-1.58 (m, 5H), 1.56-1.42 (m, 6H), 1.39-1.22 (m, 11H), 1.16-1.08 (m, 3H), 1.01 (s, 3H), 0.95-0.76 (m, 19H), 0.48-0.39 (m, 1H), 0.36-0.29 (m, 1H), 0.26-0.16 (m, 2H); ES Mass: [M+H]⁺ 857.46.

### Step 2: Synthesis of (1R,3S)-3-((((1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid:

To a solution of (1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl (1S,3R)-2,2-dimethyl-3-(2-phenylacetoxyl)cyclobutane-1-carboxylate (step 1) (0.8 g, 0.93 mmol, 1.0 eq) in EtOAc:MeOH (1:1, 8 mL) 10% Pd/C (80 mg) was added. Hydrogen gas was bubbled through the reaction mixture via a balloon for 2 hours. The resultant mixture was filtered through celite and washed with methanol and the filtrate was concentrated and the crude residue was purified by silicagel column chromatography by using 6% methanol:dichloromethane as an eluent gave the desired compound (0.35 g, 48.9% yield) as a white solid. ¹H NMR (300 MHz, CDCl₃): δ ppm 4.49-4.44 (m, 1H), 4.12-3.96 (m, 2H), 3.63-3.62 (m, 2H), 3.56-3.53 (m, 2H), 3.39 (s, 3H), 3.12-3.05 (m, 1H), 3.02-2.76 (m, 3H), 2.64-2.53 (m, 1H), 2.4-2.35 (m, 1H), 2.05-2.0 (m, 3H), 1.91-1.88 (m, 3H), 1.73-1.54 (m, 5H), 1.53-1.23 (m, 18H), 1.15-1.04 (m, 6H), 1.04-0.78 (m, 19H), 0.43-0.39 (m, 1H), 0.38-0.29 (m, 1H), 0.26-0.14 (m, 2H); ES Mass: [M+H]⁺ 767.54; HPLC: 98.32%.

### Example 43

### Preparation of 4-(((1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

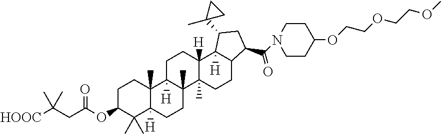

### Step 1: Synthesis of 2-(2-methoxyethoxy) ethyl 4-methylbenzenesulfonate

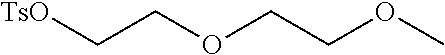 [SMILES: COCCOCCOS(=O)(=O)c1ccc(C)cc1]

To a stirred solution of 2-(2-Methoxyethoxyl)ethanol (15 g, 125.0 mmol, 1.0 eq) in CH₂Cl₂ (150 ml) triethyl amine (52 mL, 375.0 mmol, 3.0 eq), 4-dimethylaminopyridine (3.05 g, 25.0 mmol, 0.3 eq) and para-toluenesulphonylchloride (26.2 g, 137.5 mmol, 1.1 eq) were added at 0° C. The reaction mixture was allowed to stir at room temperature for overnight. TLC indicated starting material was consumed and the desired product was observed. The reaction mixture was diluted with water (200 ml), organic layer was separated and the aqueous layer was extracted with CH₂Cl₂ (2×300 ml). The combined organic layers were dried over Na₂SO₄, filtered and evaporated under reduced pressure. The crude residue was purified by silicagel column chromatography by using dichloromethane as an eluent to obtain the desired product (20.0 g, 58.4% yield) as a brown liquid. ¹H NMR (300 MHz, CDCl₃): □□□□□□□d, J=8.4 Hz, 2H), 7.34 (d, J=8.1 Hz, 2H), 4.19-4.15 (m, 2H), 3.71-3.68 (m, 2H), 3.59-3.57 (m, 2H), 3.48-3.46 (m, 2H), 3.35 (s, 3H), 2.45 (s, 3H); ES Mass: [M]⁺ 275.

### Step 2: Synthesis of tert-butyl 4-(2-(2-methoxyethoxyl)ethoxy) piperidine-1-carboxylate

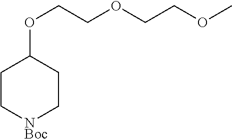 [SMILES: COCCOCCOC1CCN(C(=O)OC(C)(C)C)CC1]

To a suspension of sodium hydride (1.79 g, 74.62 mmol, 3.0 eq, 60% dispersion in mineral oil) in THF (20 ml) tert-butyl 4-hydroxypiperidine-1-carboxylate (5.0 g, 24.87 mmol, 1.0 eq) in THF (20 mL) was added at 0° C. The reaction mixture was stirred at 0° C. for 30 minutes, 2-(2-methoxyethoxy) ethyl 4-methylbenzenesulfonate (step 1) (10.24 g, 37.31 mmol, 1.5 eq) in THF (35 ml) was added and the reaction mixture was allowed to reach to room temperature and heated to reflux for overnight. TLC indicated starting material was consumed and the desired product was observed. The reaction mixture was quenched with saturated NH₄Cl solution (20 ml) and extracted with EtOAc (2×50 ml). The combined organic layers were dried over Na₂SO₄, filtered and evaporated under reduced pressure. The crude residue was purified by silicagel column chromatography by using methanol:dichloromethane as an eluent to obtain the desired product (5.0 g, 66.3% yield) as a brown liquid. ¹H NMR (300 MHz, CDCl₃): δ ppm (m, 2H), 3.68-3.53 (m, 6H), 3.58-3.53 (m, 2H), 3.52-3.44 (m, 1H), 3.39 (s, 3H), 1.85-1.81 (m, 2H), 1.57-1.48 (m, 2H), 1.45 (s, 9H); ES Mass: [M+Na]⁺ 326.13.

### Step 3: Synthesis of 4-(2-(2-methoxyethoxyl)ethoxy)piperidine hydrochloride

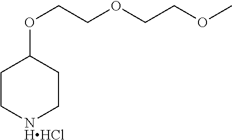

To a stirred solution of tert-butyl 4-(2-methoxyethoxyl)piperidine-1-carboxylate (step 2) (3 g, 9.88 mmol, 1.0 eq) in CH₂C2 (9 ml) 4M HCl in dioxane (24 mL) was added. The reaction mixture was stirred at room temperature for 3 hours. TLC indicated starting material was consumed and the desired product was observed. The reaction mixture was concentrated under reduced pressure to get the residue and used directly for next step without further purification.

### Step 4: Synthesis of (1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl acetate

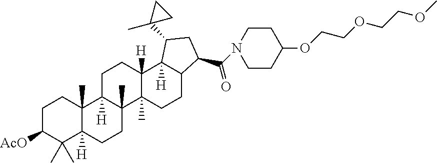

To a stirred solution of 4-(2-(2-methoxyethoxyl)ethoxy)piperidine hydrochloride (step 3) (1.86 g, 7.75 mmol, 2.0 eq) and triethylamine (1.95 g, 19.39 mmol, 5.0 eq) in CH₂Cl₂ (30 ml) a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-ylacetate (2.06 g, 3.87 mmol, 1.0 eq) in CH₂Cl₂(10 ml) was added at 0° C. The reaction mixture was allowed to stir at room temperature for overnight. The reaction mixture was diluted with water and extracted with CH₂Cl₂ (3×100 ml). The combined organic extracts were dried over Na₂SO₄, filtered and evaporated under reduced pressure. The residue was purified by silicagel column chromatography by using 25% EtOAc:n-Hexane as an eluent gave the desired product (2.1 g, 77.5% yield) as white solid. ¹H NMR (300 MHz, CDCl₃): δ ppm 4.48 (t, J=8.4 Hz, 1H), 4.08-3.89 (m, 2H), 3.65 (s, 6H), 3.56-3.53 (m, 3H), 3.38 (s, 3H), 3.14-3.09 (m, 2H), 2.82-2.72 (m, 1H), 2.09-1.98 (m, 7H), 1.96-1.65 (m, 9H), 1.56-1.28 (m, 15H), 1.18-1.05 (m, 2H), 0.96-0.82 (m, 16H), 0.46-0.4 (m, 1H), 0.39-0.3 (m, 1H), 0.26-0.15 (m, 2H); ES Mass: [M+Na]⁺ 721.47.

### Step 5: Synthesis of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)(4-(2-(2-methoxyethoxyl)ethoxy)piperidin-1-yl)methanone

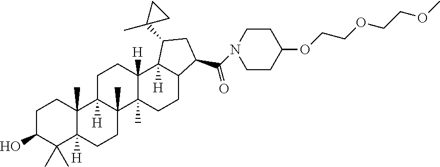

To a stirred solution of (1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl acetate (step 4) (2.1 gr, 3.0 mmol, 1.0 eq) in THF (10 ml) and Methanol (10 ml) potassium carbonate (2.08 g, 15.0 mmol, 7.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by silicagel column chromatography by using 25% EtOAc:n-Hexane as an eluent gave the desired product (1.8 g, 91.4% yield) as a white solid. ¹H NMR (300 MHz, CDCl₃): δ ppm 4.2-4.05 (m, 1H), 3.95-3.89 (m, 1H), 3.65 (s, 6H), 3.58-3.49 (m, 3H), 3.38 (s, 3H), 3.22-3.0 (m, 3H), 2.82-2.74 (m, 1H), 2.13-1.91 (m, 2H), 1.9-1.8 (m, 4H), 1.74-1.54 (m, 7H), 1.52-1.41 (m, 14H), 1.3-1.1 (m, 4H), 0.97-0.68 (m, 16H), 0.49-0.42 (m, 1H), 0.41-0.29 (m, 1H), 0.28-0.15 (m, 2H); ES Mass: [M]⁺ 656.46.

### Step 6: Synthesis of 4-(((1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-(2-methoxyethoxyl)ethoxy)piperidin-1-yl)methanone (step 5) (0.350 g, 0.533 mmol, 1.0 eq) and 2,2-dimethyl succinicanhydride (0.273 g, 2.13 mmol, 4.0 eq) in toluene (5 ml) DMAP (0.130 g, 1.07 mmol, 2.0 eq) was added. The reaction mixture was heated at 90° C. for overnight. TLC indicated starting material was consumed and the desired product was observed. The mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by silicagel column chromatography by using 1% methanol:dichloromethane as an eluent gave the desired product (0.120 g, 28.7% yield) as a white solid. ¹H NMR (300 MHz, CDCl₃): δ ppm 12.1 (bs, 1H), 4.4-4.35 (m, 1H), 3.91-3.78 (m, 2H), 3.56-3.48 (m, 7H), 3.47-3.4 (m, 2H), 3.23 (s, 3H), 3.12-3.04 (m, 2H), 2.8-2.72 (m, 1H), 2.64-2.53 (m, 1H), 2.2-1.7 (m, 6H), 1.68-1.4 (m, 9H), 1.39-1.23 (m, 9H), 1.2-1.07 (m, 11H), 0.95 (s, 3H), 0.87-0.78 (m, 15H), 0.39-0.29 (m, 2H), 0.24-0.12 (m, 2H); ES Mass: [M]⁺ 784.57; HPLC: 87.88+9.4%.

### Example 44

### Preparation of (1R,3S)-3-((((1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

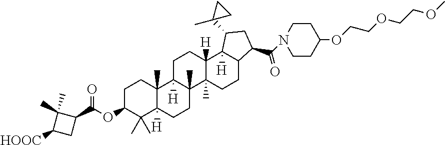

### Step 1: Synthesis of (1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl (1S,3R)-2,2-dimethyl-3-(2-phenylacetoxy)cyclobutane-1-carboxylate

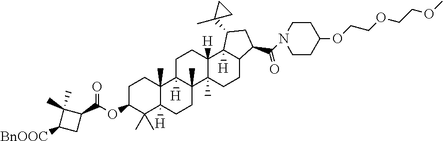

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-(2-methoxyethoxyl)ethoxy)piperidin-1-yl)methanone (0.700 g, 1.06 mmol, 1.0 eq) in toluene (10 ml) DMAP (0.261 g, 2.13 mmol, 2.0 eq) and (1S,3R)-3-(benzyloxycarbonyl)-2,2-dimethylcyclobutane carboxylic 2,4,6-trichlorobenzoic anhydride (1.0 g, 2.13 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂ (2×50 ml). The combined organic layers were dried over Na₂SO₄, filtered and evaporated under reduced pressure. The crude residue was purified by silicagel column chromatography by using 25% EtOAc:n-hexane as an eluent to obtain the desired product (0.6 g, 62.5% yield) as a white solid. ¹H NMR (300 MHz, CDCl₃): δ ppm 7.41-7.29 (m, 5H), 5.14-5.11 (m, 2H), 4.49-4.42 (m, 1H), 4.11-3.92 (m, 2H), 3.69-3.65 (m, 6H), 3.59-3.53 (m, 3H), 3.38 (s, 3H), 3.18-3.1 (m, 2H), 2.92-2.58 (m, 3H), 2.41-2.34 (m, 1H), 2.09-1.85 (m, 4H), 1.62-1.52 (m, 4H), 1.5-1.44 (m, 5H), 1.38-1.28 (m, 12H), 1.25-1.02 (m, 7H), 0.99-0.8 (m, 21H), 0.47-0.41 (m, 1H), 0.39-0.28 (m, 1H), 0.27-0.17 (m, 2H); ES Mass: [M]⁺ 900.55.

### Step 2: Synthesis of (1R,3S)-3-((((1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

To a solution of (1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl (1S,3R)-2,2-dimethyl-3-(2-phenylacetoxyl)cyclobutane-1-carboxylate (step 1) (0.6 g, 0.67 mmol, 1.0 eq) in EtOAc:MeOH (2:1, 15 mL) 10% Pd/C (60 mg) was added. Hydrogen gas was bubbled through the reaction mixture via a balloon for 2 hours. The resultant mixture was filtered through celite and washed with methanol and the filtrate was concentrated and the crude residue was purified by silicagel column chromatography by using 1% methanol:dichloromethane as an eluent gave the desired compound (0.25 g, 46.4% yield) as a white solid. ¹H NMR (300 MHz, CDCl₃): δ ppm 12.16 (bs, 1H), 4.34 (t, J=6.6 Hz, 1H), 3.92-3.84 (m, 2H), 3.58-3.46 (m, 5H), 3.44-3.4 (m, 3H), 3.23 (s, 3H), 3.12-3.0 (m, 3H), 2.82-2.71 (m, 3H), 2.12-2.02 (m, 2H), 2.09-1.76 (m, 8H), 1.68-1.45 (m, 9H), 1.41-1.08 (m, 14H), 0.95-0.82 (m, 21H), 0.37-0.26 (m, 2H), 0.24-0.14 (m, 2H); ES Mass: [M+Na]⁺ 833.44; HPLC: 95.4%.

### Example 45

### Preparation of 4-(((1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

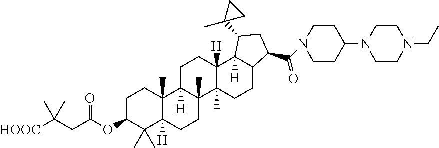

### Step 1: Synthesis of tert-butyl 4-(4-ethylpiperazin-1-yl)piperidine-1-carboxylate

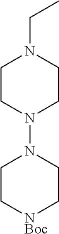 [SMILES: CCN1CCN(N2CCN(C(=O)OC(C)(C)C)CC2)CC1]

To a stirred solution of tert-Butyl 4-oxopiperidine-1-carboxylate (5 g, 25.09 mmol, 1.0 eq) in dichloroethane (100 ml) N-ethylpiperazine (2.86 g, 25.09 mmol, 1.0 eq) was added at 0° C. The reaction mixture was allowed to stir at room temperature for 30 minutes. The reaction mixture was brought to 0° C., then added AcOH (2.15 mL, 37.64 mmol, 1.5 eq) and followed by NaBH(OAc)₃ (7.98 g, 37.64 mmol, 1.5 eq). The reaction mixture was allowed to stir at room temperature for overnight. TLC indicated starting material was consumed and the desired product was observed. The reaction mixture was quenched with sat.NaHCO₃ and extracted with CH₂Cl₂ (2×200 ml). The combined organic layers were washed with water, brine and dried over Na₂SO₄, filtered and evaporated under reduced pressure. The crude residue was purified by silicagel column chromatography by using 5% MeOH:Dichloromethane as an eluent to obtain the desired product (2.7 g, 36.2% yield) as a brown liquid. ¹H NMR (300 MHz, CDCl₃): δ ppm (m, 2H), 2.75-2.4 (m, 12H), 1.92-1.88 (m, 2H), 1.45 (s, 9H), 1.38-1.31 (m, 2H), 1.3 (t, J=4.2 Hz, 3H). ES Mass: [M+Na]⁺ 320.2.

### Step 2: Synthesis of l-ethyl-4-(piperidin-4-yl)piperazine hydrochloride

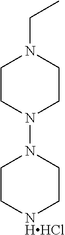

To a stirred solution of tert-butyl 4-(2-methoxyethoxyl)piperidine-1-carboxylate (step 1) (3.0 g, 10.08 mmol, 1.0 eq) in CH₂Cl₂ (9 ml) 4M HCl in dioxane (24 mL) was added. The reaction mixture was stirred at room temperature for 3 hours. TLC indicated starting material was consumed and the desired product was observed. The reaction mixture was concentrated under reduced pressure to get the residue and used directly for next step without further purification.

### Step 3: Synthesis of (1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl acetate

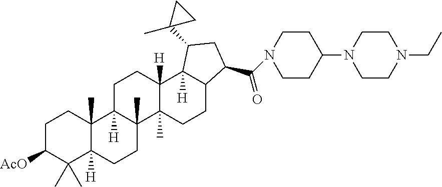

To a stirred solution of 1-ethyl-4-(piperidin-4-yl) piperazine hydrochloride (step 2) (1.76 g, 7.53 mmol, 2.0 eq) and triethylamine (1.9 g, 18.82 mmol, 5.0 eq) in CH₂Cl₂ (30 ml), a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-ylacetate (2.0 g, 3.76 mmol, 1.0 eq) in CH₂Cl₂(10 ml) was added at 0° C. The reaction mixture was allowed to stir at room temperature for overnight. The reaction mixture was diluted with water and extracted with CH₂Cl₂ (3×100 ml). The combined organic extracts were dried over Na₂SO₄, filtered and evaporated under reduced pressure. The residue was purified by silicagel column chromatography by using 3% methanol:dichloromethane as an eluent gave the desired product (2.0 g, 76.9% yield) as off-white solid. ¹H NMR (300 MHz, CDCl₃): δ ppm 4.52-4.46 (m, 3H), 2.81-2.3 (m, 13H), 2.12-1.95 (m, 3H), 2.05 (s, 3H), 1.96-1.85 (m, 2H), 1.76-1.58 (m, 10H), 1.53-1.24 (m, 12H), 1.18-1.06 (m, 4H), 0.97-0.84 (m, 19H), 0.44-0.42 (m, 1H), 0.34-0.32 (m, 1H), 0.24-0.18 (m, 2H); ES Mass: [M]⁺ 692.51.

### Step 4: Synthesis of (4-(4-ethylpiperazin-1-yl)piperidin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)methanone

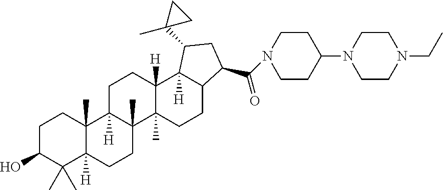

To a stirred solution of (1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl acetate (step 3) (2.0 g, 2.88 mmol, 1.0 eq) in THF (10 ml) and Methanol (10 ml) potassium carbonate (2.79 g, 20.2 mmol, 7.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by silicagel column chromatography by using 4% methanol:dichloromethane as an eluent gave the desired product (1.8 g, 95.7% yield) as a white solid. ¹H NMR (300 MHz, CDCl3): δ ppm 4.51-4.22 (m, 2H), 3.23-3.17 (m, 1H), 2.78-2.36 (m, 13H), 2.12-1.98 (m, 3H), 1.96-1.85 (m, 2H), 1.78-1.22 (m, 22H), 1.15-1.11 (m, 4H), 0.97 (s, 6H), 0.92 (s, 3H), 0.89 (s, 3H), 0.84 (s, 3H), 0.76 (s, 3H), 0.73-0.68 (m, 1H), 0.45-0.41 (m, 1H), 0.38-0.29 (m, 1H), 0.2-0.14 (m, 2H); ES Mass: [M]⁺ 650.49.

### Step 5: Synthesis of 4-(((1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

To a stirred solution of (4-(4-ethylpiperazin-1-yl)piperidin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)methanone (step 4) (0.7 g, 1.08 mmol, 1.0 eq) and 2,2-dimethyl succinicanhydride (0.551 g, 4.3 mmol, 4.0 eq) in toluene (14 ml) DMAP (0.262 g, 2.15 mmol, 2.0 eq) was added. The reaction mixture was heated at 90° C. for overnight. TLC indicated starting material was consumed and the desired product was observed. The mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by silicagel column chromatography by using 5% methanol:dichloromethane as an eluent gave the desired product (0.3 g, 35.8% yield) as a white solid. ¹H NMR (300 MHz, CD₃OD): δ ppm 4.48-4.44 (m, 3H), 2.82-2.51 (m, 14H), 2.22-2.13 (m, 2H), 2.09-191 (m, 4H), 1.82-1.28 (m, 21H), 1.23 (s, 6H), 1.18-1.11 (m, 4H), 1.03 (s, 3H), 0.96-0.82 (m, 17H), 0.42-0.34 (m, 2H), 0.24-0.19 (m, 2H); ES Mass: [M+H]⁺ 779.55; HPLC: 88%+6%.

### Example 46

### Preparation of (1R,3S)-3-((((1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

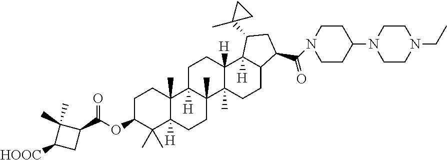

### Step 1: Synthesis of (1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl (1S,3R)-2,2-dimethyl-3-(2-phenylacetoxy)cyclobutane-1-carboxylate

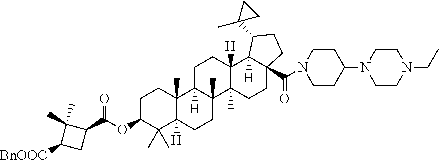

To a stirred solution of (4-(4-ethylpiperazin-1-yl)piperidin-1-yl)((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)methanone (0.700 g, 1.07 mmol, 1.0 eq) in toluene (10 ml) DMAP (0.263 g, 2.15 mmol, 2.0 eq) and (1S,3R)-3-(benzyloxycarbonyl)-2,2-dimethylcyclobutane carboxylic 2,4,6-trichlorobenzoic anhydride (1.01 g, 2.15 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂ (2×50 ml). The combined organic layers were dried over Na₂SO₄, filtered and evaporated under reduced pressure. The crude residue was purified by silicagel column chromatography by using 1.5% methanol:dichloromethane as an eluent to obtain the desired product (0.7 g, 72.7% yield) as a brown color solid. ¹H NMR (300 MHz, CDCl₃): δ ppm 7.39-7.29 (m, 5H), 5.19-5.08 (m, 2H), 4.51-4.32 (m, 3H), 2.84-2.56 (m, 15H), 2.13-1.96 (m, 4H), 1.96-1.83 (m, 3H), 1.73-1.57 (m, 6H), 1.53-1.24 (m, 18H), 1.23-1.08 (m, 5H), 1.1-0.76 (m, 22H), 0.45-0.42 (m, 1H), 0.35-0.32 (m, 1H), 0.24-0.17 (m, 2H); ES Mass: [M+H]⁺ 895.67.

### Step 2: Synthesis of (1R,3S)-3-((((1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid

To a solution of (1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl (1S,3R)-2,2-dimethyl-3-(2-phenylacetoxyl)cyclobutane-1-carboxylate (step 1) (0.7 g, 0.783 mmol, 1.0 eq) in EtOAc:MeOH (2:1, 10 mL) 10% Pd/C (70 mg) was added. Hydrogen gas was bubbled through the reaction mixture via a balloon for 2 hours. The resultant mixture was filtered through celite and washed with methanol and the filtrate was concentrated and the crude residue was purified by silicagel column chromatography by using 1% methanol:dichloromethane as an eluent gave the desired compound (0.3 g, 47.7% yield) as a brown color solid. ¹H NMR (300 MHz, CD₃OD): δ ppm 4.44-4.39 (m, 3H), 2.83-2.47 (m, 15H), 2.22-2.17 (m, 2H), 2.07-1.94 (m, 5H), 1.82-1.61 (m, 7H), 1.6-1.27 (m, 17H), 1.25-1.1 (m, 5H), 1.11 (s, 3H), 1.03 (s, 3H), 0.95-0.84 (m, 16H), 0.39-0.34 (m, 2H), 0.24-0.22 (m, 2H); ES Mass: [M+H]⁺ 805.64; HPLC: 94.5%.

### Example 47

### Preparation of 2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxyl)piperidine-1-carbonyl)icosahydro-1H-cyclopenta[a]chrysen-9-yl)oxy)butanoic acid

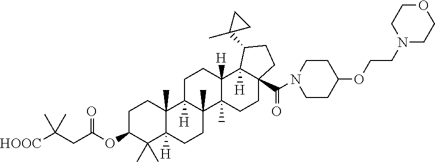

### Step 1: Synthesis of 4-(2-bromoethyl)morpholine

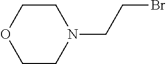

To a stirred solution of morpholine (15 g, 172.17 mmol, 1.0 eq) in acetone (150 ml) K₂CO₃ (47.5 g, 344.35 mmol, 2 eq) and 1,2-dibromoethane (29.7 mL, 344.35 mmol, 2 eq) were added at 0° C. The reaction mixture was refluxed for 12 hours. TLC indicated starting material was consumed and the desired product was observed. The reaction mixture was filtered through celite and the filtrate was concentrated under reduced pressure to get the residue (4.8 g, 14.4%) and used directly for next step without further purification. ¹H NMR (300 MHz, CDCl₃): δ ppm 3.74-3.71 (m, 4H), 3.43 (t, J=7.2 Hz, 2H), 2.78 (t, J=7.2 Hz, 2H), 2.53-2.5 (m, 4H).

### Step 2: Synthesis of tert-butyl 4-(2-morpholinoethoxyl)piperidine-1-carboxylate

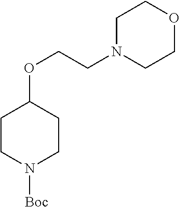 [SMILES: CC(C)(C)OC(=O)N1CCC(OCCN2CCOCC2)CC1]

To a suspension of sodium hydride (1.9 g, 79.49 mmol, 4.0 eq, 60% dispersion in mineral oil) in THF (20 ml) tert-butyl 4-hydroxypiperidine-1-carboxylate (5.0 g, 19.87 mmol, 1.0 eq) in THF (20 mL) was added at 0° C. The reaction mixture was stirred at 0° C. for 30 minutes., 4-(2-bromoethyl)morpholine (step 1) (4.6 g, 23.84 mmol, 1.2 eq) in THF (40 ml) was added and the reaction was allowed to reach to room temperature and heated to reflux for overnight. TLC indicated starting material was consumed and the desired product was observed. The reaction mixture was quenched with saturated NH₄Cl solution (20 ml) and extracted with EtOAc (2×50 ml). The combined organic layers were dried over Na₂SO₄, filtered and evaporated under reduced pressure. The crude residue was purified by silicagel column chromatography by using methanol:dichloromethane as an eluent to obtain the desired product (1.8 g, 28.8% yield) as a yellow liquid. ¹H NMR (300 MHz, CDCl₃): δ ppm 3.81-3.68 (m, 6H), 3.63-3.56 (m, 2H), 3.48-3.4 (m, 1H), 3.11-3.03 (m, 2H), 2.59 (t, J=6.0 Hz, 2H), 2.53-2.49 (m, 4H), 1.88-1.79 (m, 2H), 1.58-1.42 (m, 2H), 1.43 (s, 9H); ES Mass: [M+H]⁺ 315.12.

### Step 3: Synthesis of 4-(2-(piperidin-4-yloxy)ethyl)morpholine hydrochloride

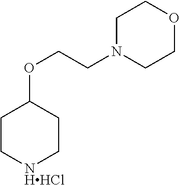

To a stirred solution of tert-butyl 4-(2-morpholinoethoxyl)piperidine-1-carboxylate (step 2) (1.8 g, 5.72 mmol, 1.0 eq) in CH₂Cl₂ (9 ml) 4M HCl in dioxane (24 mL) was added. The reaction mixture was stirred at room temperature for 3 hours. TLC indicated starting material was consumed and the desired product was observed. The reaction mixture was concentrated under reduced pressure to get the residue (1.43 gr) and used directly for next step without further purification.

### Step 4: Synthesis of (1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxyl)piperidine-1-carbonyl)icosahydro-1H-cyclopenta[a]chrysen-9-yl acetate

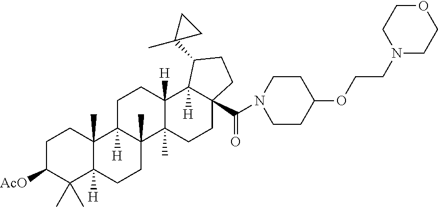

To a stirred solution of 4-(2-(piperidin-4-yloxy)ethyl)morpholine hydrochloride (step 3) (1.4 g, 5.76 mmol, 1.7 eq) and triethylamine (1.71 g, 16.94 mmol, 5.0 eq) in CH₂Cl₂ (30 ml), a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-ylacetate (1.8 g, 3.38 mmol, 1.0 eq) in CH₂Cl₂ (10 ml) was added at 0° C. The reaction mixture was allowed to stir at room temperature for overnight. The reaction mixture was diluted with water and extracted with CH₂Cl₂ (3×100 ml). The combined organic extracts were dried over Na₂SO₄, filtered and evaporated under reduced pressure. The residue was purified by silicagel column chromatography by using 4% methanol:dichloromethane as an eluent gave the desired product (2.1 g, 87.5% yield) as brown color solid. ¹H NMR (300 MHz, CDCl₃): δ ppm 4.51-4.46 (m, 1H), 4.06-3.88 (m, 2H), 3.74-3.69 (m, 4H), 3.66-3.58 (m, 2H), 3.53-3.44 (m, 1H), 3.22-3.03 (m, 2H), 2.61 (t, J=6.0 Hz, 2H), 2.54-2.48 (m, 4H), 2.12-1.98 (m, 6H), 1.96-1.58 (m, 10H), 1.54-1.23 (m, 12H), 1.23-1.01 (m, 3H), 0.96 (s, 3H), 0.94-0.78 (m, 16H), 0.45-0.42 (m, 1H), 0.38-0.29 (m, 1H), 0.26-0.13 (m, 2H); ES Mass: [M+H]⁺ 710.46.

### Step 5: Synthesis of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3aH-cyclopenta[a]chrysen-3a-yl)(4-(2-morpholinoethoxyl)piperidin-1-yl)methanone

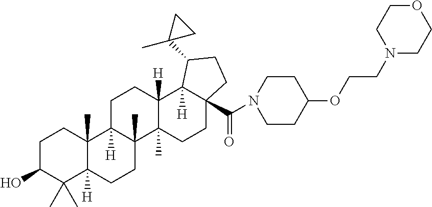

To a stirred solution of (1R,3aS,5aR,5bR,7aR,9S,11aR,1 bR,13aR,13bR)-5a,5b,8,8,11a-pentamethyl-1-(1-methylcyclopropyl)-3a-(4-(2-morpholinoethoxy)piperidine-1-carbonyl)icosahydro-1H-cyclopenta[a]chrysen-9-yl acetate (step 4) (2.1 g, 2.96 mmol, 1.0 eq) in THF (10 ml) and Methanol (10 ml) potassium carbonate (2.04 g, 14.8 mmol, 5.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by silicagel column chromatography by using 4% methanol:dichloromethane as an eluent gave the desired product (2.0 g, 96.4% yield) as a white solid. ¹H NMR (300 MHz, CDCl₃): δ ppm 3.91-3.73 (m, 3H), 3.58-3.43 (m, 7H), 3.18-2.96 (m, 2H), 2.45-2.38 (m, 6H), 2.11-2.02 (m, 1H), 1.98-1.71 (m, 6H), 1.64-1.04 (m, 18H), 0.94 (s, 3H), 0.9-0.74 (m, 15H), 0.68-0.61 (m, 4H), 0.34-0.29 (m, 2H), 0.19-0.15 (m, 2H); ES Mass: [M+H]⁺ 668.5.

### Step 6: Synthesis of 2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxyl)piperidine-1-carbonyl)icosahydro-1H-cyclopenta[a]chrysen-9-yl)oxy)butanoic acid

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-morpholinoethoxyl)piperidin-1-yl)methanone (step 5) (0.7 g, 1.05 mmol, 1.0 eq) and 2,2-dimethyl succinicanhydride (0.537 g, 4.19 mmol, 4.0 eq) in toluene (15 ml) DMAP (0.252 g, 2.09 mmol, 2.0 eq) was added. The reaction mixture was heated at 90° C. for overnight. TLC indicated starting material was consumed and the desired product was observed. The mixture was concentrated under reduced pressure, cooled to 0° C., acidified to pH=6-7 with 1N HCl and extracted with CH₂Cl₂. The combined organic extracts were washed with water, dried over Na₂SO₄, filtered and evaporated under reduced pressure. The crude residue was purified by silicagel column chromatography by using 5% methanol: dichloromethane as an eluent gave the desired product (0.3 g, 36% yield) as a white solid. ¹H NMR (300 MHz, CDCl₃): δ ppm 4.48-4.41 (m, 1H), 3.98-3.72 (m, 7H), 3.56-3.48 (m, 1H), 3.21-3.15 (m, 2H), 2.94-2.72 (m, 4H), 2.67-2.54 (m, 4H), 2.12-1.98 (m, 3H), 1.94-1.58 (m, 10H), 1.54-1.26 (m, 21H), 1.18-1.08 (m, 2H), 0.96-0.82 (m, 18H), 0.43-0.42 (m, 1H), 0.34-0.32 (m, 1H), 0.24-0.17 (m, 2H); ES Mass: [M+H]⁺ 796.59; HPLC: 89%+8%.

### Example 48

### Preparation of (1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxyl)piperidine-1-carbonyl)icosahydro-1H-cyclopenta[a]chrysen-9-yl)oxy)carbonyl)cyclobutane-1-carboxylic acid

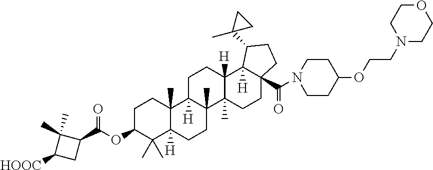

### Step 1: Synthesis of (1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxyl)piperidine-1-carbonyl)icosahydro-1H-cyclopenta[a]chrysen-9-yl (1S,3R)-2,2-dimethyl-3-(2-phenylacetoxy)cyclobutane-1-carboxylate

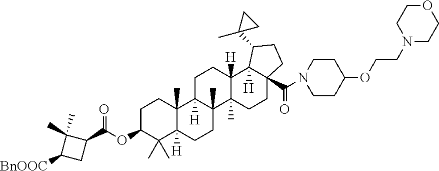

To a stirred solution of ((1R,3aS,5aR,5bR,7aR,9S,11aR,11bR,13aR,13bR)-9-hydroxy-5a,5b,8,8,11a-pentamethyl-1-(1-methylcyclopropyl)icosahydro-3 aH-cyclopenta[a]chrysen-3a-yl)(4-(2-morpholinoethoxyl)piperidin-1-yl)methanone (0.700 g, 1.05 mmol, 1.0 eq) in toluene (10 ml) DMAP (0.258 g, 2.09 mmol, 2.0 eq) and (1S,3R)-3-(benzyloxycarbonyl)-2,2-dimethylcyclobutane carboxylic 2,4,6-trichlorobenzoic anhydride (0.99 g, 2.09 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂ (2×50 ml). The combined organic layers were dried over Na₂SO₄, filtered and evaporated under reduced pressure. The crude residue was purified by silicagel column chromatography by using 4% methanol:dichloromethane as an eluent to obtain desired product (0.630 g, 65.9% yield) as a white solid. ¹H NMR (300 MHz, CDCl₃): δ ppm 7.38-7.31 (m, 5H), 5.16-5.06 (m, 2H), 4.48-4.41 (m, 1H), 4.07-3.86 (m, 2H), 3.78-3.71 (m, 4H), 3.66-3.61 (m, 2H), 3.54-3.43 (m, 1H), 3.18-3.08 (m, 2H), 2.78-2.71 (m, 3H), 2.68-2.52 (m, 6H), 2.42-2.32 (m, 1H), 2.12-1.96 (m, 4H), 1.95-1.76 (m, 6H), 1.53-1.22 (m, 17H), 1.17-1.06 (m, 2H), 0.96-0.74 (m, 24H), 0.45-0.42 (m, 1H), 0.35-0.31 (m, 1H), 0.23-0.16 (m, 2H); ES Mass: [M+H]⁺ 912.56.

### Step 2: Synthesis of (1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxyl)piperidine-1-carbonyl)icosahydro-1H-cyclopenta[a]chrysen-9-yl)oxy)carbonyl)cyclobutane-1-carboxylic acid

To a solution of (1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxy)piperidine-1-carbonyl)icosahydro-1H-cyclopenta[a]chrysen-9-yl (1S,3R)-2,2-dimethyl-3-(2-phenylacetoxyl)cyclobutane-1-carboxylate (step 1) (0.7 g, 0.768 mmol, 1.0 eq) in EtOAc:MeOH (1:1, 10 mL) 10% Pd/C (70 mg) was added. Hydrogen gas was bubbled through the reaction mixture via a balloon for 2 hours. The resultant mixture was filtered through celite and washed with methanol and the filtrate was concentrated and the crude residue was purified by silicagel column chromatography by using 5% methanol:dichloromethane as an eluent gave the desired compound (0.3 g, 47.6% yield) as a white color solid. ¹H NMR (300 MHz, CDCl₃): δ ppm 4.47-4.43 (m, 1H), 4.1-3.84 (m, 2H), 3.78-3.72 (m, 4H), 3.68-3.61 (m, 2H), 3.54-3.46 (m, 1H), 3.22-3.18 (m, 2H), 2.82-2.73 (m, 3H), 2.7-2.52 (m, 6H), 2.06-1.98 (m, 3H), 1.96-1.76 (m, 4H), 1.74-1.59 (m, 6H), 1.54-1.25 (m, 18H), 1.18-0.78 (m, 25H), 0.43-0.42 (m, 1H), 0.33-0.32 (m, 1H), 0.24-0.18 (m, 2H); ES Mass: [M+H]⁺ 822.54; HPLC: 91%.

### Example 49

### Preparation of 2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)oxy)butanoic acid

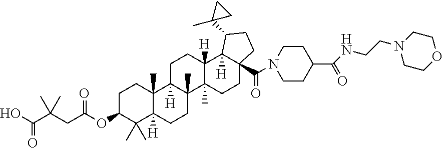

### Step 1: Synthesis of 1-(tert-butyl) 4-ethylpiperidine-1,4-dicarboxylate

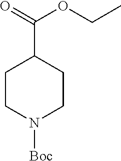 [SMILES: CCOC(=O)C1CCN(C(=O)OC(C)(C)C)CC1]

To a stirred solution of Ethyl 4-piperidinecarboxylate (10.0 g, 63.61 mmol, 1.0 eq) in CH₂Cl₂ (100 ml) Et₃N (12.8 g, 127.21 mmol, 2.0 eq) and (Boc)₂O (16.6 gr, 76.33 mmol) were added. The reaction mixture was stirred at room temperature for overnight. TLC indicated starting material was consumed and the desired product was observed. The reaction mixture was diluted with water. The aqueous layer was extracted with CH₂Cl₂ (2×250 ml). The combined organic layers were dried over Na₂SO₄, filtered and evaporated under reduced pressure. The crude residue (15 gr, 92% yield) was used for next step without further purification.

### Step 2: Synthesis of 1-(tert-butoxycarbonyl)piperidine-4-carboxylic acid

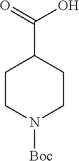 [SMILES: CC(C)(C)OC(=O)N1CCC(C(=O)O)CC1]

To a stirred solution of 1-(tert-butyl) 4-ethyl piperidine-1,4-dicarboxylate (step 1)(15.0 g, 58.29 mmol, 1.0 eq) in THF:MeOH (1:1, 150 ml) 2N NaOH solution (150 mL) was added. The reaction mixture was stirred at room temperature for overnight. TLC indicated starting material was consumed and the desired product was observed. The reaction mixture was washed with EtOAc (150 mL). The aqueous layer was acidified with 1N HCl at 0° C. and extracted with CH₂Cl₂ (2×250 ml). The combined organic layers were dried over Na₂SO₄, filtered and evaporated under reduced pressure. The crude residue (65 gr, 45% yield) was used for next step without further purification. ¹H NMR (300 MHz, CDCl₃+D₂O): δ ppm 4.05-4.01 (m, 2H), 2.89-2.82 (m, 2H), 2.53-2.46 (m, 1H), 1.94-1.89 (m, 2H), 1.71-1.58 (m, 2H), 1.46 (s, 9H); ES Mass: [M−H]⁺ 228.05.

### Step 3: Synthesis of tert-butyl 4-((2-morpholinoethyl)carbamoyl)piperidine-1-carboxylate

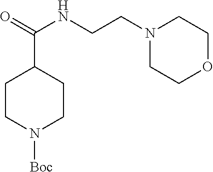 [SMILES: CC(C)(C)OC(=O)N1CCC(C(=O)NCCN2CCOCC2)CC1]

To a stirred solution of 1-(tert-butoxycarbonyl)piperidine-4-carboxylic acid (step 2) (6.0 g, 26.16 mmol, 1.0 eq) in DMF (60 ml) EDCI (9.9 gr, 52.33 mmol), HOBt (7.7 gr, 52.33 mmol), 4-(2-Aminoethyl)morpholine (3.41 gr, 26.16 mmol) and DIPEA (16.9 gr, 130.8 mmol) were added. The reaction mixture was stirred at room temperature for overnight. TLC indicated starting material was consumed and the desired product was observed. The reaction mixture was diluted with water. The aqueous layer was extracted with EtOAc (2×250 ml). The combined organic layers was washed with aq.NaHCO₃, water, brine and were dried over Na₂SO₄, filtered and evaporated under reduced pressure. The crude residue (3.0 gr, 33.7% yield) was used for next step without further purification. ¹H NMR (300 MHz, CDCl₃): δ ppm 6.05 (bs, 1H), 4.15-4.14 (m, 2H), 3.73-3.7 (m, 4H), 3.38-3.33 (m, 2H), 2.79-2.72 (m, 2H), 2.51-2.45 (m, 6H), 2.29-2.21 (m, 1H), 1.83-1.79 (m, 2H), 1.69-1.67 (m, 2H), 1.46 (s, 9H); ES Mass: [M+H]⁺ 342.15.

### Step 4: Synthesis of N-(2-morpholinoethyl)piperidine-4-carboxamide hydrochloride

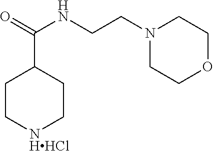

To a stirred solution of tert-butyl 4-((2-morpholinoethyl)carbamoyl)piperidine-1-carboxylate (step 3) (3.0 g, 8.78 mmol, 1.0 eq) in CH₂Cl₂ (9 ml) 4M HCl in dioxane (20 mL) was added. The reaction mixture was stirred at room temperature for 3 hours. TLC indicated starting material was consumed and the desired product was observed. The reaction mixture was concentrated under reduced pressure to get the residue (2.44 gr) and used directly for next step without further purification.

### Step 5: Synthesis of (1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl acetate

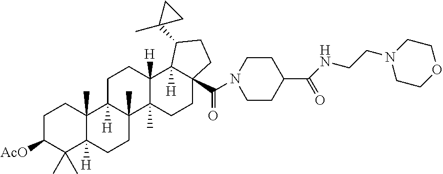

To a stirred solution of N-(2-morpholinoethyl)piperidine-4-carboxamide hydrochloride (step 4) (2.15 g, 7.75 mmol, 2.0 eq) and triethylamine (1.95 g, 19.39 mmol, 5.0 eq) in CH₂Cl₂ (30 ml), a solution of (1R,3aS,5aR,5bR,7aR,9S,11aR,11bR,13aR,13bR)-3a-(chlorocarbonyl)-5a,5b,8,8,11a-pentamethyl-1-(1-methylcyclopropyl)icosahydro-1H-cyclopenta[a]chrysen-9-ylacetate (2.06 g, 3.87 mmol, 1.0 eq) in CH₂Cl₂(10 ml) was added at 0° C. The reaction mixture was allowed to stir at room temperature for overnight. The reaction mixture was diluted with water and extracted with CH₂Cl₂ (3×100 ml). The combined organic extracts were dried over Na₂SO₄, filtered and evaporated under reduced pressure. The residue was purified by silicagel column chromatography by using 1% methanol:dichloromethane as an eluent gave the desired product (2.5 g, 87.7% yield) as an off white solid. ¹H NMR (300 MHz, CDCl₃): δ ppm 6.05 (s, 1H), 4.52-4.33 (m, 3H), 3.74-3.68 (m, 4H), 3.41-3.3 (m, 2H), 2.83-2.68 (m, 2H), 2.51-2.44 (m, 6H), 2.38-2.26 (m, 1H), 2.05 (s, 3H), 2.12-1.97 (m, 2H), 1.91-1.78 (m, 4H), 1.71-1.25 (m, 20H), 1.18-1.1 (m, 2H), 0.97 (s, 3H), 0.92 (s, 3H), 0.88 (s, 3H), 0.86-0.78 (m, 10H), 0.48-0.41 (m, 1H), 0.38-0.31 (m, 1H), 0.26-0.15 (m, 2H); ES Mass: [M]⁺ 736.45.

### Step 6: Synthesis of 1-((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)-N-(2-morpholinoethyl)piperidine-4-carboxamide

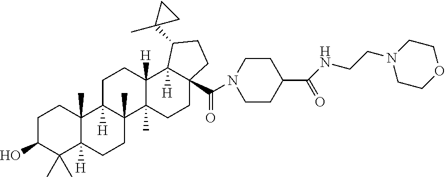

To a stirred solution of (1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl acetate (step 5) (3.0 g, 4.07 mmol, 1.0 eq) in THF (30 ml) and Methanol (30 ml) potassium carbonate (5.0 g, 36.63 mmol, 9.0 eq) was added. The reaction mixture was stirred at room temperature for 48 hours. TLC indicated starting material was consumed and the desired product was observed. The reaction mixture was filtered through a pad of celite and washed with CH₂Cl₂. The filtrate was evaporated under reduced pressure and the crude was purified by silicagel column chromatography by using 1% methanol:dichloromethane as an eluent gave the desired product (2.0 g, 70.9% yield) as a white solid. ¹H NMR (300 MHz, CDCl₃): δ ppm 6.09 (bs, 1H), 4.5-4.28 (m, 2H), 3.76-3.68 (m, 4H), 3.41-3.32 (m, 2H), 3.24-3.16 (m, 1H), 2.83-2.68 (m, 2H), 2.53-2.43 (m, 6H), 2.38-2.26 (m, 1H), 2.12-1.94 (m, 2H), 1.98-1.77 (m, 4H), 1.75-1.24 (m, 21H), 1.18-1.09 (m, 2H), 1.1-0.88 (m, 12H), 0.83 (s, 3H), 0.78 (s, 3H), 0.73-0.68 (m, 1H), 0.48-0.41 (m, 1H), 0.36-0.29 (m, 1H), 0.26-0.14 (m, 2H); ES Mass: [M+H]⁺ 695.49.

### Step 7: Synthesis of 2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)oxy)butanoic acid

To a stirred solution of 1-((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)-N-(2-morpholinoethyl)piperidine-4-carboxamide (step 6)(0.6 g, 0.864 mmol, 1.0 eq) and 2,2-dimethyl succinicanhydride (0.442 g, 3.45 mmol, 4.0 eq) in toluene (12 ml) DMAP (0.211 g, 1.72 mmol, 2.0 eq) was added. The reaction mixture was heated at 90° C. for overnight. TLC indicated starting material was consumed and the desired product was observed. The mixture was concentrated under reduced pressure, cooled to 0° C., acidified to pH=5 with 1N HCl and extracted with CH₂Cl₂. The combined organic extracts were washed with water, dried over Na₂SO₄, filtered and evaporated under reduced pressure. The crude residue was purified by silicagel column chromatography by using 5% methanol:dichloromethane as an eluent gave the desired product (0.5 g, 70.4% yield) as a white solid. ¹H NMR (300 MHz, DMSO): δ ppm 12.2 (bs, 1H), 7.78 (t, J=5.1 Hz, 1H), 4.39-4.35 (m, 1H), 4.26-4.12 (m, 2H), 3.56-3.53 (m, 4H), 3.17-3.11 (m, 2H), 2.81-2.73 (m, 3H), 2.35-2.29 (m, 7H), 2.12-1.88 (m, 3H), 1.68-1.23 (m, 22H), 1.18-1.06 (m, 10H), 1.02-0.94 (m, 4H), 0.89-0.76 (m, 15H), 0.32-0.29 (m, 2H), 0.19-0.18 (m, 2H); ES Mass: [M+H]⁺ 823.41; HPLC purity: 90+8.6%.

### Example 50

### Preparation of (1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)oxy)carbonyl)cyclobutane-1-carboxylic acid

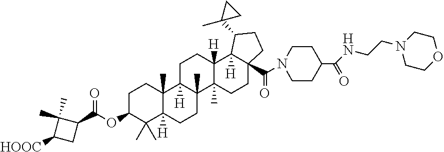

### Step 1: Synthesis of 1-benzyl 3-((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)(1R,3S)-2,2-dimethylcyclobutane-1,3-dicarboxylate

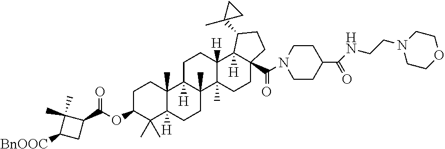

To a stirred solution of 1-((1R,3aS,5aR,5bR,7aR,9S,11aR,11bR,13aR,13bR)-9-hydroxy-5a,5b,8,8,8,11a-pentamethyl-1-(1-methylcyclopropyl)icosahydro-1H-cyclopenta[a]chrysene-3a-carbonyl)-N-(2-morpholinoethyl)piperidine-4-carboxamide (0.700 g, 1.0 mmol, 1.0 eq) in toluene (10 ml) DMAP (0.246 g, 2.01 mmol, 2.0 eq) and (1S,3R)-3-(benzyloxycarbonyl)-2,2-dimethylcyclobutane carboxylic 2,4,6-trichlorobenzoic anhydride (0.94 g, 2.01 mmol, 2.0 eq) were added. The reaction mixture was heated to 90° C. for overnight. TLC indicated starting material was consumed and the desired product was observed. The reaction mixture was evaporated under reduced pressure and diluted with water. The aqueous layer was extracted with CH₂Cl₂ (2×50 ml). The combined organic layers were dried over Na₂SO₄, filtered and evaporated under reduced pressure. The crude residue was purified by silicagel column chromatography by using 1.5% methanol:dichloromethane as an eluent to obtain the desired product (0.8 g, 84.6% yield) as a white solid. ¹H NMR (300 MHz, DMSO): δ ppm 7.79-7.76 (bs, 1H), 7.38-7.3 (m, 5H), 5.08 (ABq, J=12.6 Hz, 2H), 4.37-4.31 (m, 1H), 4.19-4.14 (m, 2H), 3.55-3.52 (m, 4H), 3.15-3.13 (m, 2H), 2.96-2.81 (m, 4H), 2.41-2.2 (m, 9H), 2.13-1.88 (m, 6H), 1.68-1.08 (m, 28H), 0.92 (s, 3H), 0.9-0.73 (m, 16H), 0.36-0.27 (m, 2H), 0.22-0.13 (m, 2H); ES Mass: [M+H]⁺ 939.5.

### Step 2: Synthesis of (1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)oxy)carbonyl)cyclobutane-1-carboxylic acid

To a solution of 1-benzyl 3-((1R,3aS,5aR,5bR,7aR,9S,11aR,11bR,13aR,13bR)-5a,5b,8,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)(1R,3S)-2,2-dimethylcyclobutane-1,3-dicarboxylate (step 1) (0.8 g, 0.85 mmol, 1.0 eq) in EtOAc:MeOH (1:1, 8 mL) 10% Pd/C (80 mg) was added.

Hydrogen gas was bubbled through the reaction mixture via a balloon for 2 hours. The resultant mixture was filtered through celite and washed with methanol and the filtrate was concentrated and the crude residue was purified by silicagel column chromatography by using 6% methanol:dichloromethane as an eluent gave the desired compound (0.5 g, 69.2% yield) as a white solid. ¹H NMR (300 MHz, DMSO): δ ppm 12.18 (bs, 1H), 7.77 (bs, 1H), 4.38-4.2 (m, 1H), 4.19-4.12 (m, 2H), 3.56-3.52 (m, 4H), 3.17-3.11 (m, 2H), 2.83-2.73 (m, 4H), 2.35-2.28 (m, 9H), 2.09-1.83 (m, 6H), 1.71-1.22 (m, 25H), 1.14-1.05 (m, 3H), 0.95-0.78 (m, 19H), 0.38-0.27 (m, 2H), 0.21-0.14 (m, 2H); ES Mass: [M+H]⁺ 849.58; HPLC purity: 93%.

### Example 51

### Preparation of 4-(((1R,3aS,5aR,5bR,7aR,9S,11aR,11bR,13aR)-3a-(((1S,3R)-2,2-dimethyl-3-(morpholine-4-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

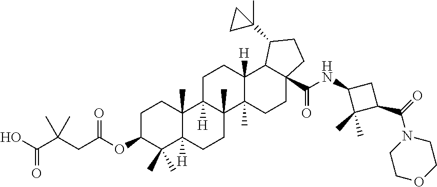

### Step 1: Synthesis of Synthesis of (1S,3R)-3-acetyl 2,2-dimethylcyclobutane carboxylic acid

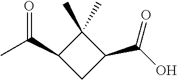

To a stirred solution of (1S)-(−)-Verbenone (50 g, 332.8 mmol) in CCl₄:CAN:H₂O (3.4 Lit), NaIO₄ (284.5 g, 1331.5 mmol) followed by RuCl₃.H₂O (catalytic) were added at 0° C. and allowed to stir at room temperature for 12 hours. After completion of the reaction as monitored by TLC, the reaction mixture was diluted with EtOAc (1000 ml), filtered through celite bed and the organic layer was separated, the aqueous layer extracted with ethyl acetate (2×500 ml), combined organic layers were dried with Na₂SO₄ and concentrated under reduced pressure. The resulting crude solid material was washed with hexane and dried under vacuum. Wt: 42.8 g; Yield: 76%. ¹H NMR (300 MHz, CDCl₃): δ 2.91-2.77 (m, 2H), 2.63 (q, 1H, J=9 Hz), 2.07 (s, 3H), 1.86-1.95 (m, 1H), 1.40 (s, 3H), 0.97 (s, 3H); Mass: [M+1]⁺ 171 (10%), [M+Na]⁺ 193 (72%).

### Step 2: Synthesis of benzyl ((1S,3R)-3-acetyl-2,2-dimethylcyclobutyl)carbamate

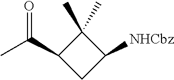

To a stirred solution of (1S,3R)-3-acetyl 2,2-dimethylcyclobutanoic acid (step 1) (16 g, 47.05 mmol, 1.0 eq.) in Toluene (160 ml) were added triethylamine (6.17 g, 61.17 mmol, 1.3 eq.) and DPPA (diphenylphosphoryl azide)(16.82 g, 61.17 mmol, 1.3 eq.). The reaction mixture was refluxed under nitrogen atmosphere for 1 hour, and then a solution of benzyl alcohol (6.65 g, 61.17 mmol, 1.3 eq.) was added. The whole reaction mixture was refluxed for overnight. TLC shows starting material was consumed and the desired product was observed. The reaction mixture was cooled to room temperature, quenched with water and extracted with dichloromethane (3×300 mL). The combined organic extracts were washed with water, dried over Na₂SO₄, filtered and evaporated under reduced pressure. The crude residue was purified by silica gel column chromatography using 8% Ethyl acetate in hexane as eluent gave the desired product. Wt.: 26 g, Yield: 95%. ¹H NMR (300 MHz, CDCl₃): δ 7.37-7.32 (m, 5H), 5.14-5.01 (m, 2H), 4.79 (d, 1H, J=7.2 Hz), 3.92 (q, 1H, J=9.0 Hz), 2.74 (t, 1H, J=9.0 Hz), 2.15-2.10 (m, 2H), 2.07 (s, 3H), 1.40 (s, 3H), 0.82 (s, 3H); Mass: [M+1]⁺ 276 (100%), [M+Na]⁺ 298 (66%).

### Step 3: Synthesis of (1R,3S)-3-((benzyloxy)carbonyl)amino)-2,2-dimethylcyclobutane-1-carboxylic acid

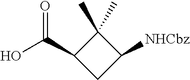

To a stirred solution of benzyl ((1S,3R)-3-acetyl-2,2-dimethylcyclobutyl)carbamate (step 2) (20 g, 72.72 mmol, 1.0 eq.) in 1,4-dioxane (2000 mL) at 0° C. was slowly added a freshly prepared NaOBr solution [prepared by adding bromine (46.52 g, 1163.6 mmol, 4.0 eq.) to a stirred solution of sodium hydroxide (46.52 g, 1163.6 mmol, 16.0 eq.) and water (1200 ml) at 0° C., then stirred for 10 minutes]. The reaction mixture was allowed stir at room temperature for overnight. TLC indicted starting material was consumed and the desired product was observed. The reaction mixture was extracted with methyl t-butyl ether (1500 mL) to remove certain impurities. The separated aqueous layer was cooled to 0° C., acidified to pH 4-5 with 1N HCl and extracted with dichloromethane (5×500 ml). The combined organic layer were dried over Na2SO4, filtered evaporated under reduced pressure gave the crude 20 g of compound. Which was purified by column chromatography 10% ethyl acetate in hexane gave the desired product. Wt: 13 g; Yield: 64.54%. ¹H NMR (300 MHz, CDCl₃): δ 7.35 (m, 5H), 5.15-5.02 (m, 2H), 4.87 (d, 1H), 3.99-3.91 (m, 1H), 2.70-2.60 (m, 1H), 2.42-2.34 (m, 1H), 2.11-1.97 (m, 1H), 1.32 (s, 3H), 0.98 (s, 3H); Mass: [M+1]⁺ 279 (100%), [M+Na]⁺ 301 (50%).

### Step 4: Synthesis of benzyl (1S,3R)-2,2-dimethyl-3-(morpholine-4-carbonyl)cyclobutyl)carbamate

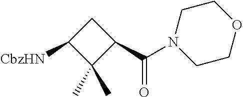

To a stirred solution of (1R,3S)-3-(((benzyloxy)carbonyl)amino)-2,2-dimethylcyclobutane-1-carboxylic acid (step 3) (10 g 36.08 mmol, 1.0 eq.) and morpholine (6.6 ml, 2.0 eq.) in dichloromethane (100 mL) was added HOBt (7.28 g, 54 mmol, 1.5 eq.) followed by EDCI (6.63 g 34.66 mmol, 2.0 eq.) at 0° C. The reaction mixture was allowed to stir at room temperature for overnight under nitrogen. TLC indicated starting material was consumed and the desired product was observed. The reaction mixture was washed with water, brine solution, dried over Na₂SO₄, filtered and evaporated under reduced pressure. The residue was purified by silica gel chromatography using 1.5% MeOH:CH₂Cl₂ as eluent to gave the desired product an off white solid. Wt.: 10 g; Yield 74.3%. ¹H NMR (300 MHz, CDCl₃): δ 7.41-7.33 (m, 5H), 5.18 (d, 1H, J=8.7 Hz), 5.09 (q, 2H, J=12.0 Hz), 3.95 (q, 1H, J=9.0 Hz), 3.79-3.38 (m, 8H), 2.82 (t, 1H, J=8.4 Hz), 2.50-2.35 (m, 1H), 2.35-2.20 (m, 1H), 1.34 (s, 3H), 0.89 (s, 3H, J=7.2 Hz); Mass: [M+1]⁺ 347 (10%), [M+Na]⁺ 369 (100%).

### Step 5: Synthesis of ((1R,3S)-3-amino-2,2-dimethylcyclobutyl)(morpholino)methanone

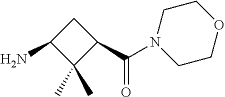

To a stirred solution of benzyl (1S,3R)-2,2-dimethyl-3-(morpholine-4-carbonyl)cyclobutyl)carbamate (step 4) (10 g 26.8 mmol) in ethanol (80 mL) 10% Pd/C (2 g, catalytic) was added at room temperature and the reaction mixture stirred under H₂ gas atmosphere at room temperature for about 18 hours. After completion of the reaction (monitored by TLC) the reaction mixture was filtered through a celite bed, the obtained filtrate was concentrated under reduced pressure to afford free amine was liquid which proceed to next step without characterization. Wt.: 5.51 g; Yield: 90%. ¹H NMR (300 MHz, CDCl₃): δ 7.41-7.33 (m, 5H), 5.18 (d, 1H, J=8.7 Hz), 5.09 (q, 2H, J=12.0 Hz), 3.95 (q, 1H, J=9.0 Hz), 3.79-3.38 (m, 8H), 2.82 (t, 1H, J=8.4 Hz), 2.50-2.35 (m, 1H), 2.35-2.20 (m, 1H), 1.34 (s, 3H), 0.89 (s, 3H, J=7.2 Hz); Mass: [M+1]⁺ 347 (10%), [M+Na]⁺ 346 (100%).

### Step 6: Synthesis of (1R,3aS,5aR,5bR,7aR,9S,11aR,11bR,13aR)—N-((1S,3R)-2,2-dimethyl-3-(morpholine-4-carbonyl)cyclobutyl)-9-hydroxy-5a,5b,8,8,11a-pentamethyl-1-(prop-1-en-2-yl)icosahydro-3aH-cyclopenta[a]chrysene-3a-carboxamide

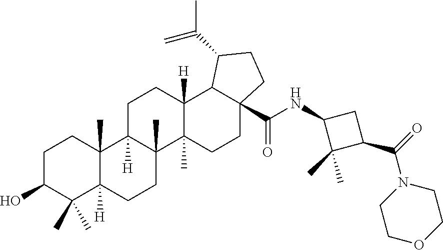

To a stirred solution of (1R,3aS,5aR,5bR,7aR,9S,11aR,11bR,13aR)-9-hydroxy-5a,5b,8,8,11a-pentamethyl-1-(prop-1-en-2-yl)icosahydro-3 aH-cyclopenta[a]chrysene-3a-carboxylic acid (20.0 g, 43.85 mmol, 1 eq.), in DCM:DMF (1:1, 14 v) was added EDCI (8.18 g, 94.27 mmol, 2.15 eq.), HOBt (8.87 g, 1.5 eq.) triethyl amine (22.1 g, 219 mmol, 5.0 eq.) at 0-5° C. The reaction mixture was stirred for 1 h at 0-5° C. temperature. ((1R,3S)-3-amino-2,2-dimethylcyclobutyl)(morpholino)methanone (step 5) in DCM:DMF (1:1, 6v) was added to above solution at 0-5° C. and allowed stir reaction mixture at room temperature for 16 hours. After completion of the reaction (monitored by TLC) reaction mixture was diluted with Dichloromethane washed with 1N HCl solution, water, brine solution and dried over Na₂SO₄. The solvent was evaporated and crude compound was purified by silica gel chromatography (100-200 mesh, elution 2% methanol in dichloromethane). Wt.: 14 g; Yield: 49.26%. ¹H NMR (300 MHz, CDCl₃): δ 5.89 (d, 1H, J=8.1 Hz), 4.73 (s, 1H), 4.58 (s, 1H), 4.11-4.73 (m, 1H, J=8.1 Hz), 3.43-3.80 (m, 8H), 3.10-3.17 (m, 2H), 2.84-2.86 (t, 1H, J=7.8 Hz), 2.31-2.45 (m, 3H), 1.85-1.96 (m, 2H), 1.50-1.78 (m, 14H), 1.31-1.49 (m, 8H), 1.24-1.28 (m, 3H), 1.12-1.16 (m, 3H), 0.97 (s, 3H), 0.95 (s, 3H), 0.84 (s, 3H), 0.80 (s, 3H), 0.74 (s, 3H), 0.67 (m, 1H); Mass: [M+1]⁺ 651 (100%), [M+2]⁺ 652 (40%); HPLC purity: 94.13%.

### Step 7: Synthesis of (1R,3aS,5aR,5bR,7aR,9S,11aR,11bR,13aR)—N-((1S,3R)-2,2-dimethyl-3-(morpholine-4-carbonyl)cyclobutyl)-9-hydroxy-5a,5b,8,8,11a-pentamethyl-1-(1-methylcyclopropyl-)icosahydro-3aH-cyclopenta[a]chrysene-3a-carboxamide)

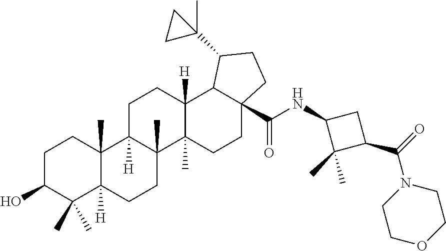

To a stirred solution of (1R,3aS,5aR,5bR,7aR,9S,11aR,11bR,13aR)—N-((1S,3R)-2,2-dimethyl-3-(morpholine-4-carbonyl)cyclobutyl)-9-hydroxy-5a,5b,8,8,11a-pentamethyl-1-(prop-1-en-2-yl)-icosahydro-3 aH-cyclopenta[a]chrysene-3a-carboxamide (step 6) (1.5 g, 2.30 mmol, 1.0 eq.) in dry dichloromethane (12 v) cooled to −25 to −30° C. were added 1.5 Molar Diethyl zinc in toluene (9.26 mL, 13.84 mmol, 6 eq.) and stirred for 1 hour at this temperature. Diiodomethane (4.9 g, 1.49 mL, 18.4 mmol, 8 eq.) was added slowly to the above solution, stirred for 1 h at −25 to −30° C. and then reaction mixture allowed to warm slowly to room temperature and stirred for 12-14 hours. Completion of reaction was monitored by TLC chromatography, the reaction mixture was quenched with saturated ammonium chloride solution and followed by 1N HCl solution. The reaction mixture diluted with dichloromethane, washed with water, brine and dried with Na₂SO₄. The solvent was evaporated and without purification of crude compound proceed to next step. Wt.: 1.3 g; Yield: 84.96%. ¹H NMR (300 MHz, CDCl₃): δ 5.86 (d, 1H, J=8.1 Hz), 4.08-4.11 (m, 1H), 3.57-3.76 (m, 6H), 3.42-3.45 (m, 2H), 3.12-3.23 (m, 2H), 2.84 (t, 1H, J=8.1 Hz), 2.29-2.34 (m, 3H), 1.83-1.90 (m, 2H), 1.89-1.09 (m, 25H), 0.97 (s, 3H), 0.96 (s, 3H), 0.92 (s, 3H), 0.89 (s, 3H), 0.82, 0.75, 0.70 (s, 9H), 0.39-0.47 (m, 1H), 0.28-0.37 (m, 1H), 0.14-0.27 (m, 2H); Mass: [M+1]⁺ 793 (80%), [M+Na]⁺, 815 (100%); HPLC: 93.13%.

### Step 8: Synthesis of 4-(((1R,3aS,5aR,5bR,7aR,9S,11aR,11bR,13aR)-3a-(((1S,3R)-2,2-dimethyl-3-(morpholine-4-carbonyl)cyclobutyl)carbamoyl)-5a,5b,8,8,11a-pentamethyl-1-(1-methylcyclopro-pyl)icosahydro-1H-cyclopenta[a]chrysen-9-yl)oxy)-2,2-dimethyl-4-oxobutanoic acid

To a stirred solution of (1R,3aS,5aR,5bR,7aR,9S,11aR,11bR,13aR)—N-((1S,3R)-2,2-dimethyl-3-(morpholine-4-carbonyl)cyclobutyl)-9-hydroxy-5a,5b,8,8,11a-pentamethyl-1-(1-methylcyclopro-pyl)icosahydro-3 aH-cyclopenta[a]chrysene-3a-carboxamide) (step 7) (0.25 g, mmol) 2,2-dimethyl succinic anhydride (0.192 g, 1.50 mmol, 4 eq.) and DMAP (g, mmol) in pyridine (10 v) were added at room temperature then the reaction mixture was refluxed for 12-14 hours. Completion of reaction was monitored by TLC chromatography, the reaction mixture was diluted with ethyl acetate, washed with water, 1N HCl solution, water, brine and dried with Na₂SO₄. The solvent was evaporated and purified by silica gel chromatography (100-200 mesh, elution 2% methanol in dichloromethane). Wt: 0.120 mg; Yield: 40%. ¹H NMR (300 MHz, CDCl₃): δ 5.89 (d, 1H, J=8.1 Hz), 4.43-4.52 (m, 1H), 4.08-4.11 (m, 1H), 3.65-3.80 (m, 4H), 3.50-3.65 (m, 4H), 3.42-3.45 (m, 2H), 2.80-2.88 (m, 1H), 2.65 (t, 1H, J=9 Hz)), 2.60 (m, 1H), 2.28-2.34 (m, 4H), 1.90-1.94 (m 2H), 1.30-1.75 (m, 32H), 0.96 (s, 3H), 0.91 (s, 3H), 0.89 (s, 3H), 0.81, 0.82, 0.83 (s, 9H), 0.31-0.33 (m, 1H), 0.21-0.24 (m, 1H), 0.18-0.24 (m, 2H); Mass: [M+1]⁺ 793 (80%), [M+Na]⁺, 815 (100%); HPLC: 90.13%.

### Example 52

### Preparation of 5-(((1R,3aS,5aR,5bR,7aR,9S,11aR,11bR,13aR)-3a-(((1S,3R)-2,2-dimethyl-3-(morpholine-4-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid

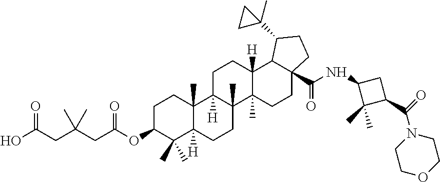

To a stirred solution of (1R,3aS,5aR,5bR,7aR,9S,11aR,11bR,13aR)—N-((1S,3R)-2,2-dimethyl-3-(morpholine-4-carbonyl)cyclobutyl)-9-hydroxy-5a,5b,8,8,11a-pentamethyl-1-(1-methylcyclopropyl)icosahydro-3 aH-cyclopenta[a]chrysene-3a-carboxamide) (0.25 g, 0.376 mmol) 3,3-dimethyl glutaric anhydride (0.5 mL, 1.504 mmol, 4 eq.) and DMAP (0.1 g, 0.752 mmol, 2 eq) in pyridine (10 v) were added at room temperature then the reaction mixture was refluxed for 12-14 hours. Completion of reaction mixture was monitored by TLC chromatography, the reaction mixture was diluted with ethyl acetate, washed with water, 1N HCl, water, brine and dried with Na₂SO₄. The solvent was evaporated and purified by silica gel chromatography (100-200 mesh, elution 2% methanol in dichloromethane). Wt: 0.149 g; Yield: 50%. ¹H NMR (300 MHz, CDCl₃): δ 5.88 (d, 1H, J=8.1 Hz), 4.49-4.52 (m, 1H), 4.08-4.11 (m, 1H), 3.65-3.80 (m, 4H), 3.50-3.65 (m, 2H), 3.43-3.45 (m, 2H), 2.80-2.88 (m, 1H), 2.42-2.46 (m, 4H), 2.31-2.34 (m, 3H), 1.90-1.94 (m, 2H), 1.30-1.75 (m, 23H), 1.22-1.26 (m, 2H), 1.14 (s. 2H), 1.01-1.05 (m, 2H), 0.97 (s, 3H), 0.92 (s, 3H), 0.90 (s, 3H), 0.85, 0.84, 0.82, 0.78 (s, 12H), 0.76 (m, 1H), 0.40-0.41 (m, 1H), 0.31-0.33 (m, 1H), 0.16-0.29 (m, 2H); Mass: [M+1]⁺ 807 (100%), [M+Na]⁺ 830 (80%); HPLC: 92.46%.

### Example 53

### Preparation of (1R,3S)-3-((((1R,3aS,5aR,5bR,7aR,9S,11aR,11bR,13aR)-3a-(((1S,3R)-2,2-dimethyl-3-(morpho-line-4-carbonyl)cyclobutyl)carbamoyl)-5a,5b,8,8,11a-pentamethyl-1-(prop-1-en-2-yl)icosa-hydro-1H-cyclopenta[a]chrysen-9-yl)oxy)carbonyl)-2,2-dimethylcyclobutane-1-carboxylic acid

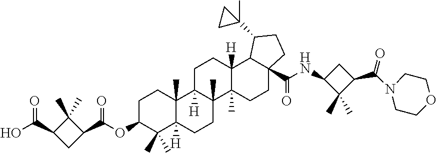

### Step 1: Synthesis of 1-benzyl 3-((1R,3aS,-5aR,5bR,7aR,9S,11aR,11bR,13aR)-3a-(((1S,3R)-3-(4-ethylpiperazine-1-car-bonyl)-2,2-dimethylcy-clobutyl)carbamoyl)-5a,5b,8,8,11a-pentamethyl-1-(prop-1-en-2-yl)-ico-sahydro-1H-cyclopenta-[a]chrysen-9-yl)(1R,3S)-2,2-dimethylcyclobutane-1,3-dicarboxylate

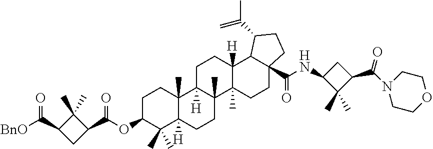

To a stirred solution of (1R,3aS,5aR,5bR,7aR,9S,11aR,11bR,13aR)—N-((1S,3R)-3-(4-ethylpiperaz-ine-1-carbonyl)-2,2-dimethylcyclobutyl)-9-hydroxy-5a,5b,8,8,11a-pentamethyl-1-(prop-1-en-2-yl)icosahydro-3 aH-cyclopenta[a]chrysene-3a-carboxamide (0.250 g. 0.37 mmol) in pyridine (5 ml), DMAP (94 mg, 0.74 mmol, 2 eq.) was added followed by the crude reaction mixture (1.06 g, 0.22 mmol, 6 eq.)(anhydride formation). The reaction mixture was stirred at reflux temperature for about 12 hours. After completion of the reaction (monitored by TLC), the mixture was cooled to room temperature, diluted with ethyl acetate, washed with dil. HCl, water, followed by brine and dried over Na₂SO₄, and concentrated under reduced pressure. The crude reaction mixture was purified by column chromatography using ethyl acetate and hexane mixture as eluent to afford the desired product (150 mg, 48.85%). ¹H NMR (300 MHz, CDCl₃): δ 7.32-7.38 (m, 5H), δ 5.96 (d, 1H, J=8.1 Hz), 4.48 (s, 1H), 4.10-4.16 (m, 2H), 3.54-3.74 (m, 8H), 3.44 (t, 2H, J=6.6 Hz), 2.75-2.80 (m, 4H), 2.23-2.39 (m, 5H), 1.90-2.11 (m, 5H), 1.5-1.76 (m, 1H), 1.22-1.42 (m, 17H), 1.04 (m, 3H), 0.99 (m, 3H), 0.24-0.95 (m, 21H), 0.37-0.44 (m, 1H), 0.80-0.47 (m, 1H), 0.17-0.30 (m, 2H); Mass: [M+1]⁺ 895.28 (100%), [M+Na]⁺ 918 (20%).

### Step 2: Synthesis of (1R,3S)-3-((((1R,3aS,5aR,5bR,7aR,9S,11aR,11bR,13aR)-3a-(((1S,3R)-2,2-dimethyl-3-(morpho-line-4-carbonyl)cyclobutyl)carbamoyl)-5a,5b,8,8,11a-pentamethyl-1-(prop-1-en-2-yl) icosa-hydro-1H-cyclopenta[a]chrysen-9-yl)oxy)carbonyl)-2,2-dimethylcyclobutane-1-carboxylic acid

To a stirred solution of 1-benzyl 3-((1R,3aS,-5aR,5bR,7aR,9 S,11aR,11bR,13aR)-3a-(((1S,3R)-3-(4-ethylpiperazine-1-carbonyl)-2,2-dimethylcy-clobutyl)carbamoyl)-5a,5b,8,8,11a-pentamethyl-1-(prop-1-en-2-yl)icosahydro-1H-cyclopenta-[a]chrysen-9-yl)(1R,3S)-2,2-dimethylcyclobutane-1,3-dicarboxylate (150 mg, 0.13 mmol), in dry DCM (4 ml), triethyl amine (0.036 mL. 0.6 mmol, 2.0 eq.) and Et₃SiH (0.022 mL, 0.19 mmol, 1.2 eq.) were added at 0° C. then Pd(OAc)₂ (12.5 mg, catalytic) was added under nitrogen atmosphere and the reaction mixture was stirred at reflux temperature over night. After completion of the reaction (monitored by TLC), diluted with DCM and concentrate under reduced pressure then purified by column chromatography to get the title compound as a white solid, Wt: 0.04 g; Yield: 32.84%. ¹H NMR (300 MHz, CDCl₃): δ 5.96 (d, 1H, J=8.1 Hz), 4.49 (s, 1H), 4.10-4.15 (m, 2H), 3.55-3.73 (m, 8H), 3.44 (t, 2H, J=6.6 Hz), 2.76-2.80 (m, 4H), 2.24-2.40 (m, 5H), 1.90-2.12 (m, 5H), 1.49-1.76 (m, 1H), 1.21-1.42 (m, 17H), 1.05 (m, 3H), 0.98 (m, 3H), 0.23-0.94 (m, 21H), 0.38-0.45 (m, 1H), 0.79-0.48 (m, 1H), 0.15-0.29 (m, 2H); Mass: [M+1]⁺ 819.18 (100%), [M+Na]⁺ 842 (10%); HPLC Purity: 92.91%.

### Example 54

### Preparation of 1-benzyl 3-((1R,3aS,5aR,5bR,7aR,9S,11aR,11bR,13aR)-3a-(((1S,3R)-3-(4-ethylpiperazine-1-car-bonyl)-2,2-dimethylcyclobutyl)carbamoyl)-5a,5b,8,8,11a-pentamethyl-1-(prop-1-en-2-yl)-ico-sahydro-1H-cyclopenta[a]chrysen-9-yl)(1R,3S)-2,2-dimethylcyclobutane-1,3-dicarboxylate

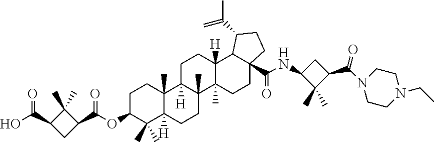

### Step 1: Synthesis of benzyl ((1S,3R)-3-(4-ethylpiperazine-1-carbonyl)-2,2-dimethylcyclobutyl)-carbamate

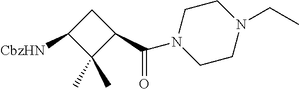

To a stirred solution of (1R,3S)-3-(((benzyloxy)carbonyl)amino)-2,2-dimethylcyclobutane-1-carboxylic acid (10 g 198.5 mmol, 1.0 eq.) and ethyl piperazine (8.22 g, 397 mmol, 2.0 eq) in dichloromethane (100 mL) was added HOBt (7.3 g, 1.5 eq.) followed by EDCI (13.18 g, 2.0 eq) at 0° C. The reaction mixture was allowed to stir at room temperature for overnight under nitrogen. TLC indicated starting material was consumed and the desired product was observed. The reaction mixture was washed with water, brine solution, dried over Na₂SO₄, filtered and evaporated under reduced pressure. The residue was purified by silica gel chromatography using 1.5% MeOH:CH₂Cl₂ as eluent to provide the desired product an off white solid. Wt: 6 g; Yield: 44.70%; ¹H NMR (300 MHz, CDCl₃): δ 7.41-7.33 (m, 5H), 5.12 (d, 2H, J=12 Hz), 5.04 (d, 1H, J=12 Hz), 3.89 (q, 1H, J=9.0 Hz), 3.81 (b, 1H), 3.56 (m, 3H), 2.81 (t, 1H, J=8.4 Hz), 2.69-2.42 (m, 4H), 2.22-2.42 (m, 4H), 1.34 (s, 3H), 1.71 (t, 3H) 0.87 (s, 3H); Mass: [M+1]⁺ 347 (100%), [M+Na]⁺ 370 (75%).

### Step 2: Synthesis of ((1R,3S)-3-amino-2,2-dimethylcyclobutyl)(4-ethylpiperazin-1-yl)methanone

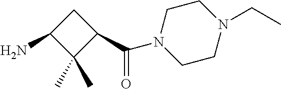

To a stirred solution of benzyl ((1S,3R)-3-(4-ethylpiperazine-1-carbonyl)-2,2-dimethylcyclobutyl)-carbamate (step 1) (10 g 26.8 mmol) in ethanol (80 mL) 10% Pd/C (2 g, catalytic) was added at room temperature and the reaction mixture stirred under H₂ gas atmosphere at room temperature for about 18 hours. After completion of the reaction (monitored by TLC) the reaction mixture was filtered through a celite bed, the obtained filtrate was concentrated under reduced pressure to afford free amine was liquid which proceed to next step without characterization. Wt: 5.0 g; Yield: 80%.

### Step 3: Synthesis of (1R,3aS,5aR,5bR,7aR,9S,11aR,11bR,13aR)—N-((1S,3R)-3-(4-ethylpiperazine-1-carbonyl)-2,2-di-methylcyclobutyl)-9-hydroxy-5a,5b,8,8,11a-pentamethyl-1-(prop-1-en-2-yl)icosahydro-3aH-cyclopenta[a]chrysene-3a-carboxamide

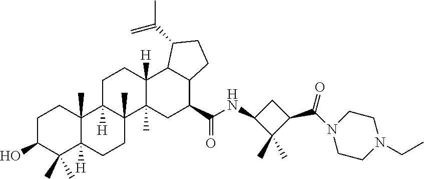

To a stirred solution of (1R,3aS,5aR,5bR,7aR,9S,11aR,11bR,13aR)-9-hydroxy-5a,5b,8,8,11a-pentamethyl-1-(prop-1-en-2-yl)icosahydro-3 aH-cyclopenta[a]chrysene-3a-carboxylic acid (2.6 g, 5 mmol, 1 eq.), in DCM:DMF (1:1, 14 v) was added EDCI (2 g, 10 mmol, 2.15 eq.), HOBt (1.0 g, 7.5 mmol, 1.5 eq.), triethyl amine (2.8 g, 20 mmol, 4.0 eq.) at 0-5° C. The reaction mixture was stirred for 1 hour at 0-5° C. temperature. ((1R,3S)-3-amino-2,2-dimethylcyclobutyl)(4-ethylpiperazin-1-yl) methanone (Step 2) in DCM:DMF (1:1, 6v) was added to above solution at 0-5° C. and allowed to stir reaction mixture at room temperature for 16 hours. After completion of the reaction (monitored by TLC) reaction mixture was diluted with dichloromethane washed with 1N HCl solution, water, brine solution and dried over Na₂SO₄. The solvent was evaporated and crude compound was purified by silica gel chromatography (100-200 mesh, elution 2% methanol in dichloromethane). Wt: 1.4 g; Yield: 36.26%.

### Step 4: Synthesis of 1-benzyl 3-((1R,3aS,-5aR,5bR,7aR,9S,11aR,11bR,13aR)-3a-(((1S,3R)-3-(4-ethylpiperazine-1-car-bonyl)-2,2-dimethylcy-clobutyl)carbamoyl)-5a,5b,8,8,11a-pentamethyl-1-(prop-1-en-2-yl)-ico-sahydro-1H-cyclopenta-[a]chrysen-9-yl)(1R,3S)-2,2-dimethylcyclobutane-1,3-dicarboxylate

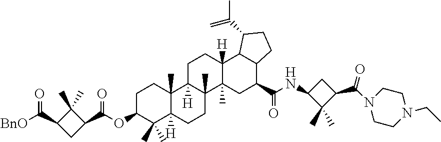

To a stirred solution of (1R,3aS,5aR,5bR,7aR,9S,11aR,11bR,13aR)—N-((1S,3R)-3-(4-ethylpiperaz-ine-1-carbonyl)-2,2-dimethylcyclobutyl)-9-hydroxy-5a,5b,8,8,11a-pentamethyl-1-(prop-1-en-2-yl)icosahydro-3 aH-cyclopenta[a]chrysene-3a-carboxamide (step 3) in pyridine (0.2 g) in pyridine (2 ml), DMAP (75 mg) was added followed by the crude reaction mixture (anhydride formation). The reaction mixture was stirred at reflux temperature for about 12 hours. After completion of the reaction (monitored by TLC), the mixture was cooled to room temperature, diluted with ethyl acetate, washed with dil. HCl, water, followed by brine and dried over Na₂SO₄, and concentrated under reduced pressure. The crude reaction mixture was purified by column chromatography using ethyl acetate and hexane mixture as eluent to afford the desired product (120 mg, 44.44%). ¹H NMR (300 MHz, CDCl₃): δ 7.32-7.38 (m, 5H), δ 6.19 (d, 1H, J=8.1 Hz), 4.72 (s, 1H), 4.56 (s, 1H) 4.47 (t, 1H, J=8.7 Hz), 4.10-4.22 (m, 1H), 3.73-3.86 (m, 1H), 3.43-3.63 (m, 3H), 3.08-3.15 (m, 1H), 2.76-2.88 (m, 3H), 2.18-2.62 (m, 12H), 1.97-2.03 (m, 5H), 1.76-1.90 (m, 23H), 1.53-1.61 (m, 8H), 1.45-1.52 (m, 5H), 1.32-1.37 (m, 5H), 1.24-1.27 (m, 6H), 1.05-1.13 (m, 4H), 0.96 (m, 4H), 0.90 (m, 4H), 0.82-0.84 (m, 14H); Mass: [M+1]⁺ 832.75 (100%), [M+Na]⁺ 855 (10%).

### Step 5: Synthesis of 1-benzyl 3-((1R,3aS,5aR,5bR,7aR,9S,11aR,11bR,13aR)-3a-(((1S,3R)-3-(4-ethylpiperazine-1-car-bonyl)-2,2-dimethylcyclobutyl)carbamoyl)-5a,5b,8,8,11a-pentamethyl-1-(prop-1-en-2-yl)-ico-sahydro-1H-cyclopenta[a]chrysen-9-yl)(1R,3S)-2,2-dimethylcyclobutane-1,3-dicarboxylate

To a stirred solution of 1-benzyl 3-((1R,3aS,-5aR,5bR,7aR,9 S,11aR,11bR,13aR)-3a-(((1S,3R)-3-(4-ethylpiperazine-1-carbonyl)-2,2-dimethylcy-clobutyl)carbamoyl)-5a,5b,8,8,11a-pentamethyl-1-(prop-1-en-2-yl)icosahydro-1H-cyclopenta-[a]chrysen-9-yl)(1R,3S)-2,2-dimethylcyclobutane-1,3-dicarboxylate (step 4) (120 mg), in dry DCM (3 ml), triethyl amine (0.036 mL. 0.26 mmol, 2.0 eq.) and Et₃SiH (0.018 mL, 0.15 mmol, 1.2 eq. were added at 0° C. then Pd(OAc)₂(10 mg) was added under nitrogen atmosphere and the reaction mixture was stirred at reflux temperature over night.

After completion of the reaction (monitored by TLC), diluted with DCM and concentrate under reduced pressure then purified by column chromatography to get the title compound as a white solid. ¹H NMR (300 MHz, CDCl₃): δ 6.19 (d, 1H, J=8.1 Hz), 4.73 (s, 1H), 4.58 (s, 1H) 4.47 (t, 1H, J=8.7 Hz), 4.10-4.21 (m, 1H), 3.74-3.87 (m, 1H), 3.43-3.62 (m, 3H), 3.09-3.16 (m, 1H), 2.75-2.86 (m, 3H), 2.19-2.64 (m, 12H), 1.98-2.04 (m, 5H), 1.75-1.89 (m, 23H), 1.54-1.62 (m, 8H), 1.44-1.50 (m, 5H), 1.32-1.37 (m, 5H), 1.25-1.26 (m, 6H), 1.05-1.13 (m, 4H), 0.96 (m, 4H), 0.90 (m, 4H), 0.82-0.84 (m, 14H); Mass: [M+1]⁺ 832.75 (100%), [M+Na]⁺ 855 (10%); HPLC Purity: 86.65%.

### Example 55

### Preparation of 4-(((1R,3aS,5aR,5bR,7aR,9S,11aR,11bR,13aR)-3a-(((1S,3R)-2,2-dimethyl-3-(piperidine-1-carbonyl)cyclobutyl)carbamoyl)-5a,5b,8,8,11a-pentamethyl-1-(1-methylcyclopro-pyl)icosahydro-1H-cyclopenta[a]chrysen-9-yl)oxy)-2,2-dimethyl-4-oxobutanoic acid

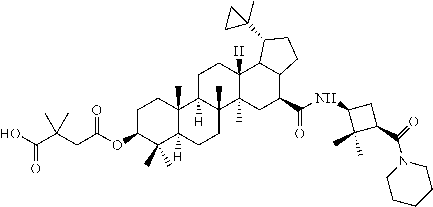

### Step 1: Synthesis of (1R,3aS,5aR,5bR,7aR,9S,11aR,11bR,13aR)—N-((1R,3S)-2,2-dimethyl-3-(piperidine-1-carbonyl)cyclobutyl)-9-hydroxy-5a,5b,8,8,11a-pentamethyl-1-(1-methylcyclopropyl)icosahydro-3aH-cyclopenta[a]chrysene-3a-carboxamide

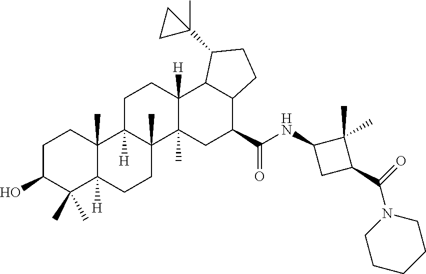

To a stirred solution of (1R,3aS,5aR,5bR,7aR,9S,11aR,11bR,13aR)—N-((1R,3S)-2,2-dimethyl-3-(piperidine-1-carbonyl)cyclobutyl)-9-hydroxy-5a,5b,8,8,11a-pentamethyl-1-(prop-1-en-2-yl)icosahydro-3 aH-cyclopenta[a]chrysene-3a-carboxamide (1.5 g, 2.30 mmol, 1.0 eq.) in dry dichloromethane (12 v) cooled to −25 to −30° C. were added 1.5 Molar Diethyl zinc in toluene (9.26 mL, 13.84 mmol, 6 eq.) and stirred for 1 hour at this temperature. Diiodomethane (4.9 g, 1.49 mL, 18.4 mmol, 8 eq.) was added slowly to the above solution, stirred for 1 hour at −25 to −30° C. and then reaction mixture allowed to warm slowly to room temperature and stirred for 12-14 hour. Completion of reaction was monitored by TLC chromatography, the reaction mixture was quenched with saturated ammonium chloride solution and followed by 1N HCl solution. The reaction mixture diluted with dichloromethane, washed with water, brine and dried with Na₂SO₄. The solvent was evaporated and purified compound by column chromatography 15% Ethyl acetate in Hexane to provide two compounds. Wt.: 1.0 g; Yield: 65.35% (Major) and Wt.: 0.3 g; Yield: 19% (Minor). ¹H NMR (300 MHz, CDCl₃): Minor compound: δ 5.90 (d, 1H, J=8.1 Hz), 4.06-4.13 (m, 1H), 3.42-3.52 (m, 1H), 3.31-3.40 (m, 2H), 3.13-3.22 (m, 1H), 2.87 (t, 1H, J=8.1 Hz), 2.23-2.40 (m, 3H), 1.89-1.98 (m, 2H), 1.10-1.88 (m, 25H), 0.97 (s, 3H), 0.96 (s, 3H), 0.92 (s, 3H), 0.89 (s, 3H), 0.82, 0.75, 0.70 (s, 9H), 0.39-0.47 (m, 1H), 0.22-0.46 (m, 1H), 0.12-0.23 (m, 2H); Mass: [M+1]⁺ 663 (100%), [M+Na]⁺, 686 (10%); HPLC: 92.13%.

### Step 2: Synthesis of 4-(((1R,3aS,5aR,5bR,7aR,9S,11aR,11bR,13aR)-3a-(((1S,3R)-2,2-dimethyl-3-(piperidine-1-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid

To a stirred solution of above minor compound [(1R,3aS,5aR,5bR,7aR,9S,11aR,11bR,13aR)—N-((1R,3S)-2,2-dimethyl-3-(piperidi-carbonyl)cyclobutyl)-9-hydroxy-5a,5b,8,8,11a-pentamethyl-1-(1-methylcyclopropyl)icosahydro-3 aH-cyclopenta[a]chrysene-3a-carboxamide)] (0.25 g, mmol) 2,2-dimethylsuccinic anhydride (0.192 g, 1.50 mmol, 4 eq.) and DMAP (0.10 g, mmol) in pyridine (10 v) were added at room temperature then the reaction mixture was refluxed for 12-14 hours. Completion of reaction was monitored by TLC chromatography, the reaction mixture was diluted with ethyl acetate, washed with water, 1N HCl solution, water, brine and dried with Na₂SO₄. The solvent was evaporated and purified by silica gel chromatography (100-200 mesh, elution 2% methanol in dichloromethane). Wt: 0.120 mg; Yield: 40%. ¹H NMR (300 MHz, CDCl₃): δ 5.96 (d, 1H, J=8.1 Hz), 4.46-4.54 (m, 1H), 4.06-4.09 (m, 1H), 3.62-3.73 (m, 1H), 3.42-3.53 (m, 1H), 3.32-3.41 (m, 2H), 2.80-2.91 (t, 1H), 2.51-2.72 (m, 3H), 2.22-2.40 (m, 4H), 1.90-1.96 (m 2H), 1.20-1.80 (m, 32H), 0.96 (s, 3H), 0.91 (s, 3H), 0.89 (s, 3H), 0.81, 0.82, 0.83 (s, 9H), 0.49-0.39 (m, 1H), 0.29-0.34 (m, 1H), 0.14-0.28 (m, 2H); Mass: [M+1]⁺ 791 (100%), [M+Na]⁺, 815 (10%); HPLC: 89.51%.

### Example 56

### Preparation of 4-((1R,3aS,5aR,5bR,7aR,9S,11aR,11bR,13aR,13bR)-3a-(4-cyano-4-phenylpiperidine-1-carbonyl)-5a,5b,8,8,11a-pentamethyl-1-(1-methylcyclopropyl)icosahydro-1H-cyclopenta[a]chrysen-9-yloxy)-2,2-dimethyl-4-oxobutanoic acid

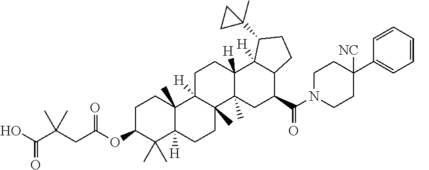 [SMILES: CC(C)(CC(=O)O[C@H]1CC[C@]2(C)[C@H]3CC[C@@H]4[C@@H]5C(CC[C@H]5C5(C)CC5)[C@@H](C(=O)N5CCC(C#N)(c6ccccc6)CC5)C[C@@]4(C)[C@]3(C)CC[C@H]2C1(C)C)C(=O)O]

### Step 1: synthesis of (1R,3aS,5aR,5bR,7aR,9S,11aR,11bR,13aR,13bR)-9-acetoxy-5a,5b,8,8,11a-pentamethyl-1-(1-methylcyclopropyl)icosahydro-1H-cyclopenta[a]chrysene-3a-carboxylic acid

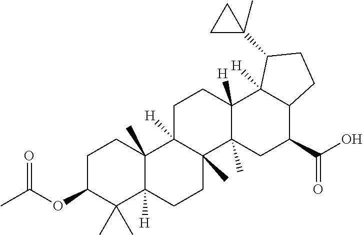 [SMILES: CC(=O)O[C@H]1CC[C@]2(C)[C@H]3CC[C@@H]4[C@@H]5C(CC[C@H]5C5(C)CC5)[C@@H](C(=O)O)C[C@@]4(C)[C@]3(C)CC[C@H]2C1(C)C]

To a stirred solution of (1R,3aS,5aR,5bR,7aR,9S,11aR,11bR,13aR,13bR)-benzyl 9-acetoxy-5a,5b,8,8,11a-pentamethyl-1-(1-methylcyclopropyl)icosahydro-1H-cyclopenta[a]chrysene-3a-carboxylate (6.5 g, 10.7 mmol) in Ethyl acetate:Ethanol (300+200 ml) 10% Pd/C (2 g) was added and stirred the reaction under H₂ atmosphere (60 psi) for about 12 hours and completion of the reaction monitored by TLC the reaction mixture was filtered washed with ethanol, the solvent was evaporated and purified by silica gel column (100-200 mesh, elution 15% EtOAc in hexane to afford the title compound as an off white solid (5.0 g). ¹H NMR (300 MHz, CDCl₃): δ 0.22-0.25 (m, 2H), 0.26-0.27 (m, 2H), 0.08-0.91 (m, 11H), 1.12-1.28 (m, 3H), 1.30-1.33 (m, 10H), 1.50-1.58 (m, 2H), 2.01 (s, 3H), 2.03-2.04 (m, 1H), 2.11-2.13 (m, 1H), 3.01-3.13 (m, 1H), 4.36-4.37 (m, 1H); Mass: 512 [M+1]⁺ 513 (100%).

### Step 2: Synthesis of (1R,3aS,5aR,5bR,7aR,9S,11aR,11bR,13aR,13bR)-3a-(4-cyano-4-phenylpiperidine-1-carbonyl)-5a,5b,8,8,11a-pentamethyl-1-(1-methylcyclopropyl)icosahydro-1H-cyclopenta[a]chrysen-9-yl acetate

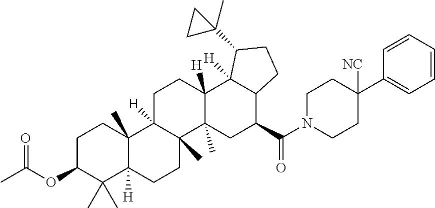 [SMILES: CC(=O)O[C@H]1CC[C@]2(C)[C@H]3CC[C@@H]4[C@@H]5C(CC[C@H]5C5(C)CC5)[C@@H](C(=O)N5CCC(C#N)(c6ccccc6)CC5)C[C@@]4(C)[C@]3(C)CC[C@H]2C1(C)C]

To a stirred solution of (1R,3aS,5aR,5bR,7aR,9S,11aR,11bR,13aR,13bR)-9-acetoxy-5a,5b,8,8,11a-pentamethyl-1-(1-methylcyclopropyl)icosahydro-1H-cyclopenta[a]chrysene-3a-carboxylic acid (step 1) (4.0 g, 7.54 mmol)(Prepared as described in J. Med. Chem. 2009. 52, 3248-3258) in DCM (30 ml) Oxolyl chloride (3 ml, 23.6 mmol) in DCM (50 ml) was added at 0° C. and stirred at room temperature for about 3 hours and completion of the reaction monitored by TLC then the solvent was evaporated under nitrogen atmosphere and dissolved in DCM (20 ml), which was added to the above stirred solution of 4-phenylpiperidine-4-carbonitrile (1.4 g, 7.54 mmol in 10 ml DCM and triehylamine (2.2 ml, 15.08 mmol) at 0° C., and allowed to stir at room temperature for about 16 hours. After completion of the reaction (monitored by TLC), the reaction mixture was diluted with DCM and washed with water, bicarbonate solution and brine solution and dried over Na₂SO₄, the solvent was evaporated and purified by silica gel column (100-200 mesh, elution 30% EtOAc in hexane) to afford the title compound as an off white solid. Wt: 3.2 g: ¹H NMR (300 MHz, CDCl₃): δ 0.22-0.25 (m, 2H), 0.26-0.27 (m, 2H), 0.79-0.93 (m, 8H), 1.07-1.26 (m, 10H), 1.29-1.56 (m, 12H), 169-1.99 (m, 8H), 2.09 (s, 3H), 2.23-3.45 (m, 6H), 3.82-4.13 (m, 5H), 4.20-4.24 (m, 1H), 4.61-4.75 (m, 1H), 4.77 (s, 1H), 4.80 (s, 1H), 5.31-5.35 (m, 1H), 7.22-7.60 (m, 3H), 7.70-7.88 (m, 2H), 10.50 (s, 1H); Mass: 679[M+1]⁺ 680 (100%).

### Step 3: Synthesis of 1-((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)-4-phenylpiperidine-4-carbonitrile

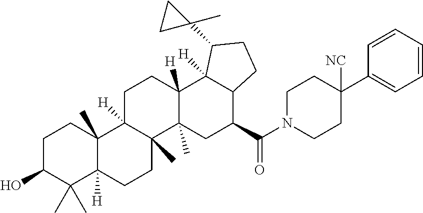 [SMILES: CC1([C@@H]2CCC3[C@H]2[C@H]2CC[C@@H]4[C@@]5(C)CC[C@H](O)C(C)(C)[C@@H]5CC[C@@]4(C)[C@]2(C)C[C@@H]3C(=O)N2CCC(C#N)(c3ccccc3)CC2)CC1]

To a stirred solution of (1R,3aS,5aR,5bR,7aR,9S,11aR,11bR,13aR,13bR)-3a-(4-cyano-4-phenylpiperidine-1-carbonyl)-5a,5b,8,8,11a-pentamethyl-1-(1-methylcyclopropyl)icosahydro-1H-cyclopenta[a]chrysen-9-yl acetate (step 2) (step 4, 3.0 g, 4.41 mmol) in MeOH:THF (20 ml) potassium carbonate (4.46 g, 30.8 mmol) was added at 0° C. and allowed to stir at room temperature for about 16 hours. After completion of the reaction (monitored by TLC), the volatile was evaporated and the aqueous layer was extracted with ethyl acetate and the organic layer was washed with water, brine and dried over Na₂SO₄ then the solvent was evaporated and the resulting crude was purified by silica gel column (100-200 mesh, elution 5% MeOH/DCM) to afford the title compound as an off white solid (2.1 g). ¹H NMR (300 MHz, CDCl₃): δ 0.22-0.25 (m, 2H), 0.26-0.27 (m, 2H), 0.79-0.93 (m, 8H), 1.07-1.26 (m, 10H), 1.29-1.56 (m, 12H), 169-1.99 (m, 8H), 2.23-3.45 (m, 6H), 3.82-4.13 (m, 5H), 4.20-4.24 (m, 1H), 4.61-4.75 (m, 1H), 4.77 (s, 1H), 4.80 (s, 1H), 5.31-5.35 (m, 1H), 7.22-7.60 (m, 3H), 7.70-7.88 (m, 2H), 10.50 (s, 1H); Mass: 638 [M+1]⁺ 639 (100%).

### Step 4: Synthesis of 4-((1R,3aS,5aR,5bR,7aR,9S,11aR,11bR,13aR,13bR)-3a-(4-cyano-4-phenylpiperidine-1-carbonyl)-5a,5b,8,8,11a-pentamethyl-1-(1-methylcyclopropyl)icosahydro-1H-cyclopenta[a]chrysen-9-yloxy)-2,2-dimethyl-4-oxobutanoic acid

2,2-Dimethylsuccinic anhydride (0.60 g, 3.73 mmol) was added to a stirred solution of 1-((1R,3aS,5aR,5bR,7aR,9S,11aR,11bR,13aR,13bR)-9-hydroxy-5a,5b,8,8,11a-pentamethyl-1-(1-methylcyclopropyl)icosahydro-1H-cyclopenta[a]chrysene-3a-carbonyl)-4-phenylpiperidine-4-carbonitrile (step 3) (2.0 g, 2.94 mmol) and DMAP (1.14 g, 8.80 mmol) in Toluene (30 ml) at room temperature and refluxed for about 24 hours. After completion of the reaction (monitored by TLC), the reaction mixture was diluted with EtOAc, washed with water, 1N HCl, water, brine and dried over Na₂SO₄, the solvent was evaporated and purified by silica gel column (100-200 mesh, elution 5% MeOH/DCM to afford the title compound as an off white solid. Wt: 0.2 g: ¹H NMR (300 MHz, CDCl₃): δ 0.22-0.25 (m, 2H), 0.26-0.27 (m, 2H), 0.79-0.93 (m, 8H), 0.93 (m, 6H), 1.07-1.26 (m, 10H), 1.29-1.56 (m, 12H), 169-1.99 (m, 8H), 2.09-2.18 (m, 4H), 2.23-3.45 (m, 6H), 3.82-4.13 (m, 5H), 4.20-4.24 (m, 1H), 4.61-4.75 (m, 1H), 4.77 (s, 1H), 4.80 (s, 1H), 5.31-5.35 (m, 1H), 7.22-7.60 (m, 3H), 7.70-7.88 (m, 2H), 10.50 (s, 1H); Mass: 766[M+1]⁺ 767 (100%); HPLC Purity: 87.6%.

The following compounds were prepared according to the synthetic routes described in the examples above.

| Example | Structure/Observed Molecular | |
|---|---|---|
| No. | Weight | IUPAC NAme |
| 57 | 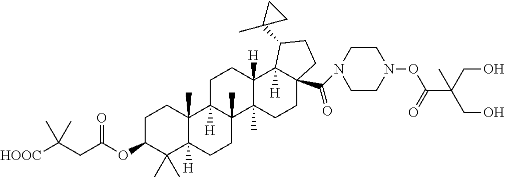 Molecular Weight: 799.10 | 4-(((1R,3aS,5aR,5bR,7aR,9S,11aR,11bR, 13aR,13bR)-3a-(4-((3-hydroxy-2- (hydroxymethyl)-2-methylpropanoyl)oxy) piperazine-1-carbonyl)-5a,5b,8,8,11a- pentamethyl-1-(1-methylcyclopropyl) icosahydro-1H-cyclopenta[a]chrysen-9-yl) oxy)-2,2-dimethyl-4-oxobutanoic acid |
| 58 | 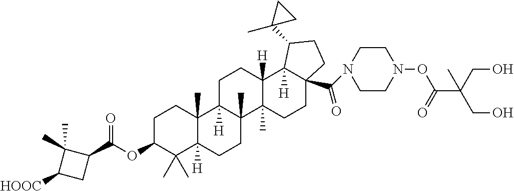 Molecular Weight: 825.14 | (1R,3S)-3- ((((1R,3aS,5aR,5bR,7aR,9S,11aR,11bR, 13aR,13bR)-3a-(4-((3-hydroxy-2- (hydroxymethyl)-2-methylpropanoyl)oxy) piperazine-1-carbonyl)-5a,5b,8,8,11a- pentamethyl-1-(1-methylcyclopropyl) icosahydro-1H-cyclopenta[a]chrysen-9-yl) oxy)carbonyl)-2,2-dimethylcyclobutane-1- carboxylic acid |
| 59 | 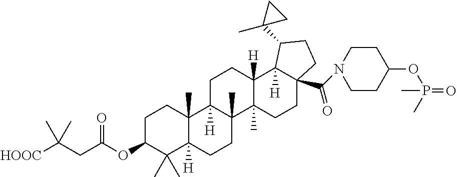 Molecular Weight: 758.03 | 4-(((1R,3aS,5aR,5bR,7aR,9S,11aR,11bR, 13aR,13bR)-3a-(4-((dimethylphosphoryl)oxy) piperidine-1-carbonyl)-5a,5b,8,8,11a- pentamethyl-1-(1-methylcyclopropyl) icosahydro-1H-cyclopenta[a]chrysen-9- yl)oxy)-2,2-dimethyl-4-oxobutanoic acid |
| 60 | 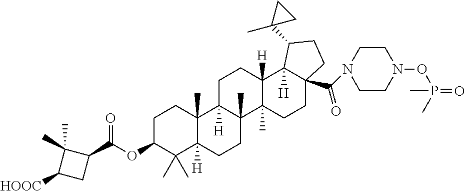 Molecular Weight: 784.07 | (1R,3S)-3- ((((1R,3aS,5aR,5bR,7aR,9S,11aR,11bR, 13aR,13bR)-3a-(4-((dimethylphosphoryl) oxy)piperidine-1-carbonyl)-5a,5b,8,8,11a- pentamethyl-1-(1-methylcyclopropyl) icosahydro-1H-cyclopenta[a]chrysen-9- yl)oxy)carbonyl)-2,2-dimethylcyclobutane- 1-carboxylic acid |
| 61 | 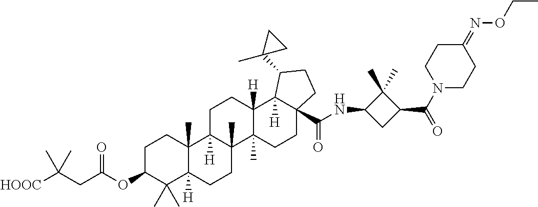 Molecular Weight: 848.22 | 4-(((1R,3aS,5aR,5bR,7aR,9S,11aR,11bR, 13aR,13bR)-3a-(((1R,3S)-3-(4-(ethoxyimino) piperidine-1-carbonyl)-2,2-dimethylcyclobutyl) carbamoyl)-5a,5b,8,8,11a-pentamethyl- 1-(1-methylcyclopropyl)icosahydro-1H- cyclopenta[a]chrysen-9-yl)oxy)-2,2-dimethyl- 4-oxobutanoic acid |
| 62 | 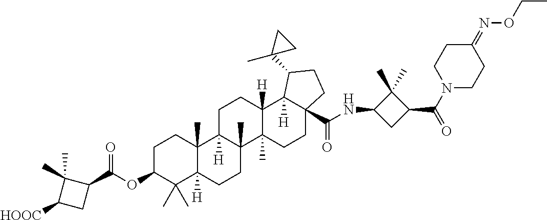 Molecular Weight: 874.26 | (1R,3S)-3- ((((1R,3aS,5aR,5bR,7aR,9S,11aR,11bR, 13aR,13bR)-3a-(((1R,3S)-3-(4-(ethoxyimino) piperidine-1-carbonyl)-2,2- dimethylcyclobutyl)carbamoyl)-5a,5b,8,8, 11a-pentamethyl-1-(1-methylcyclopropyl) icosahydro-1H-cyclopenta[a]chrysen-9-yl) oxy)carbonyl)-2,2-dimethylcyclobutane-1- carboxylic acid |
| 63 | 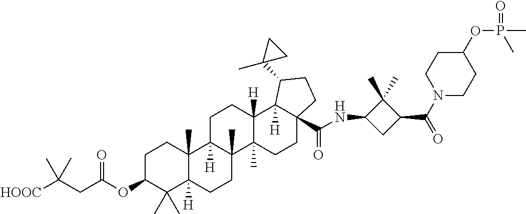 Molecular Weight: 883.20 | 4-(((1R,3aS,5aR,5bR,7aR,9S,11aR,11bR, 13aR,13bR)-3a-(((1R,3S)-3-(4- ((dimethylphosphoryl)oxy)piperidine-1- carbonyl)-2,2-dimethylcyclobutyl) carbamoyl)-5a,5b,8,8,11a-pentamethyl-1- (1-methylcyclopropyl)icosahydro-1H- cyclopenta[a]chrysen-9-yl)oxy)-2,2- dimethyl-4-oxobutanoic acid |
| 64 | 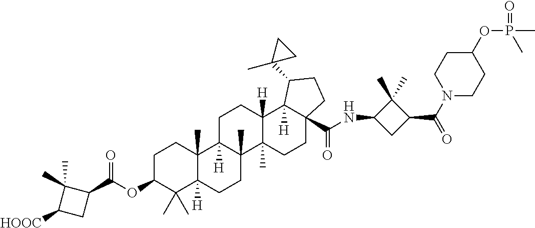 Molecular Weight: 909.24 | (1R,3S)-3- ((((1R,3aS,5aR,5bR,7aR,9S,11aR,11bR, 13aR,13bR)-3a-(((1R,3S)-3-(4- ((dimethylphosphoryl)oxy)piperidine-1- carbonyl)-2,2-dimethylcyclobutyl) carbamoyl)-5a,5b,8,8,11a-pentamethyl-1- (1-methylcyclopropyl)icosahydro-1H- cyclopenta[a]chrysen-9-yl)oxy)carbonyl)- 2,2-dimethylcyclobutane-1-carboxylic acid |
| 65 | 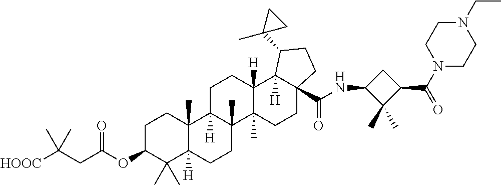 Molecular Weight: 820.21 | 4-(((1R,3aS,5aR,5bR,7aR,9S,11aR,11bR, 13aR,13bR)-3a-(((1S,3R)-3-(4- ethylpiperazine-1-carbonyl)-2,2- dimethylcyclobutyl)carbamoyl)-5a,5b,8, 8,11a-pentamethyl-1-(1- methylcyclopropyl)icosahydro-1H- cyclopenta[a]chrysen-9-yl)oxy)-2,2- dimethyl-4-oxobutanoic acid |
| 66 | 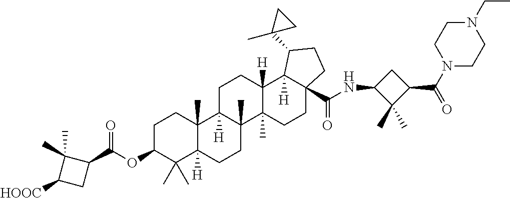 Molecular Weight: 846.25 | (1R,3S)-3- ((((1R,3aS,5aR,5bR,7aR,9S,11aR,11bR, 13aR,13bR)-3a-(((1S,3R)-3-(4- ethylpiperazine-1-carbonyl)-2,2- dimethylcyclobutyl)carbamoyl)-5a,5b,8, 8,11a-pentamethyl- 1-(1-methylcyclopropyl)icosahydro-1H- cyclopenta[a]chrysen-9-yl)oxy)carbonyl)- 2,2-dimethylcyclobutane-1-carboxylic acid |

### Pharmacological Activity

The compounds described herein can be tested for their antiviral activity following procedures known to a person of ordinary skill in the art. For example, the following protocols can be employed for testing the compounds. These protocols are illustrative and do not limit to the scope of the invention.

### Example 67

### Evaluation of Compounds Antiviral Activity

MT2 cells were infected with HIV-1 strain 92HT599 (10 TCID 50/30000 cells). The infected cells were plated at the concentration of −30 000 cells per well in 96 well plate. Test compound was added to the micro plate in defined format with the final concentration of DMSO (vehicle) is not more than 1%. Incubation was carried out in CO2 incubator for ˜96 hours for viral infection. At the end of incubation period an aliquot from each well was taken for p24 estimation. The quantitation of p24 is an index for antiviral activity of the compound. Percent inhibition was calculated with reference to control values (vehicle controls).

p-24 estimation was carried out using Advance biosciences kit as per the procedure detailed by supplier.

Results

TABLE 1

| | Antiviral activity | | | Antiviral activity | |
|---|---|---|---|---|---|
| Compound | % inhibition | | Compound | % inhibition | |
| No | 1000 nM | 100 nM | No | 1000 nM | 100 nM |
| 1 | 100 | 95 | 49 | 98 | 99 |
| 2 | 98 | 77 | 50 | 99 | 99 |
| 3 | 97 | 97 | 42 | 99 | 94 |
| 4 | 98 | 98 | 41 | 99 | 97 |
| 5 | 92 | 49 | 31 | 99 | 99 |
| 6 | 92 | 85 | 33 | 99 | 100 |
| 7 | 97 | 98 | 34 | 100 | 100 |
| 8 | 100 | 100 | 32 | 99 | 99 |
| 9 | 99 | 64 | 43 | 99 | 99 |
| 11 | 100 | 100 | 44 | 100 | 97 |
| 12 | 100 | 100 | 45 | 100 | 100 |
| 13 | 99 | 83 | 46 | 100 | 100 |
| 14 | 100 | 100 | 47 | 99 | 100 |
| 15 | 97 | 93 | 48 | 99 | 97 |
| 16 | 100 | 100 | 35 | 84 | 26 |
| 17 | 100 | 100 | 36 | 99 | 33 |
| 18 | 100 | 100 | 37 | 99 | 30 |
| 19 | 95 | 91 | 38 | 99 | 68 |
| 24 | 100 | 100 | | 100 | 99 |
| 21 | 100 | 100 | 51 | 100 | 100 |
| 56 | 99 | 76 | 52 | 100 | 100 |
| 25 | 100 | 100 | 54 | 100 | 100 |
| 22 | 100 | 100 | 28 | 99 | 98 |
| 26 | 99 | 94 | 30 | 99 | 97 |
| 23 | 100 | 100 | 39 | 99 | 97 |
| 27 | 88 | 94 | 40 | 99 | 100 |
| 29 | 99 | 95 | | | |

### Example 68

### Evaluation of Compounds Cyto-Toxicity

For cyto-toxicity assay the same amount of MT2 cells as in antiviral assay without virus was added to the 96 well plates. The cyto-toxicity was measured using MTT reagent in parallel with p24 estimation. The percent viability is calculated in comparison with vehicle control.

Results

TABLE 2

| | Cytotoxicity | | | Cytotoxicity | |
|---|---|---|---|---|---|
| Compound | % viability | | Compound | % viability | |
| No | 1000 nM | 100 nM | No | 1000 nM | 100 nM |
| 1 | 92 | 96 | 39 | 88 | 83 |
| 2 | 95 | 96 | 40 | 81 | 79 |
| 3 | 99 | 100 | 49 | 88 | 85 |
| 4 | 86 | 82 | 50 | 96 | 86 |
| 5 | 98 | 95 | 42 | 84 | 86 |
| 6 | 87 | 94 | 41 | 86 | 86 |
| 7 | 96 | 99 | 31 | 95 | 100 |
| 8 | 97 | 90 | 33 | 87 | 100 |
| 9 | 88 | 82 | 34 | 91 | 100 |
| 11 | 98 | 96 | 32 | 95 | 100 |
| 12 | 99 | 100 | 43 | 100 | 100 |
| 13 | 79 | 86 | 44 | 86 | 93 |
| 14 | 88 | 86 | 45 | 92 | 100 |
| 15 | 95 | 95 | 46 | 90 | 100 |
| 16 | 79 | 81 | 47 | 96 | 100 |
| 17 | 89 | 77 | 48 | 100 | 100 |
| 18 | 77 | 82 | 35 | 81 | 70 |
| 19 | 100 | 100 | 36 | 90 | 77 |
| 24 | 99 | 83 | 37 | 72 | 73 |
| 21 | 96 | 94 | 38 | 83 | 85 |
| 56 | 80 | 83 | 51 | 83 | 85 |
| 25 | 84 | 100 | 52 | 85 | 95 |
| 22 | 87 | 100 | 54 | 100 | 100 |
| 26 | 99 | 100 | 29 | 77 | 81 |
| 23 | 93 | 100 | 28 | 78 | 81 |
| 27 | 94 | 100 | 30 | 82 | 83 |

### Example 69

### Evaluation of Compounds Single Dose Oral Pharmacokinetic Study

The test item was administered through oral route to animals (rat/mice) at 30 mg/kg dose in a suitable vehicle at 10 ml/kg dose volume. Blood samples (˜50 uL at each time point) were collected from retro-orbital plexus using K3 EDTA as anticoagulant in eppendorf tubes at defined time intervals under light ether anesthesia. The samples were centrifuged at 3500×g to separate plasma and stored at −80° C. until analysis.

Sample analysis: Test samples were analyzed using LC-MS-MS after developing fit-for-purpose method for each of test compound.

Results

TABLE 3

| | Rat oral PK @30 mg/kg | | | |
|---|---|---|---|---|
| Compound | Cmax | AUC 0-t | AUC 0-inf | t½ |
| No | μg/mL | μg · hr/mL | μg · hr/mL | hrs |
| 1 | 1.217 | 3.121 | 3.227 | 1.535 |
| 4 | 5.92 | 22.268 | 22.646 | 4.436 |
| 11 | 1.968 | 8.054 | 8.164 | 3.657 |

TABLE 4

| | Mice oral PK @30 mg/kg | | | |
|---|---|---|---|---|
| Compound | Cmax | AUC 0-t | AUC 0-inf | t½ |
| No | μg/mL | μg · hr/mL | μg · hr/mL | hrs |
| 1 | 8.794 | 35.942 | 36.674 | 6.231 |
| 4 | 9.777 | 137.796 | 155.394 | 14.508 |
| 11 | 11.349 | 114.968 | 137.623 | 3.657 |

TABLE 5

| | Mice oral PK @30 mg/kg | | | |
|---|---|---|---|---|
| Compound | Cmax | AUC 0-t | AUC 0-inf | Tmax |
| No | μg/mL | μg · hr/mL | μg · hr/mL | hrs |
| 12 | 8.16 | 201.4 | — | 4.0 |
| 13 | 11.99 | 456.5 | — | 20.0 |
| 14 | 4.45 | 15.17 | — | 0.63 |
| 15 | 3.78 | 31.38 | — | 0.63 |
| 17 | 5.94 | 88.3 | — | 4.50 |
| 18 | 4.23 | 36.56 | — | 2.25 |

TABLE 6

| | Cmax | Tmax | AUCt | AUC_{0-inf} | Tlast |
|---|---|---|---|---|---|
| Compound | (μg/ml) | (hr) | μg · hr/mL | μg · hr/mL | (hr) |
| No | Oral PK @ 30 mg/ kg dose in mice | | | | |
| 24 | 9.1 | 2.0 | 70.0 | 123.3 | 24 |
| 21 | 5.9 | 7.0 | 95.5 | 185.0 | 24 |
| 56 | 5.9 | 7.0 | 95.5 | 185.0 | 24 |
| 25 | 6.2 | 4.5 | 108.5 | 122.3 | 48 |
| 22 | 10.2 | 1.8 | 63.9 | 79.0 | 24 |
| 26 | 6.9 | 3.5 | 99.4 | 151.3 | 24 |
| 23 | 11.0 | 4.5 | 323.9 | 628.0 | 48 |
| 27 | 7.0 | 3.5 | 101.3 | 105.8 | 48 |
| 29 | 11.7 | 16.0 | 428.0 | NA | 48 |
| 28 | 4.5 | 16.0 | 171.4 | 397.9 | 48 |
| 30 | 4.7 | 3.5 | 67.1 | 78.5 | 48 |
| 39 | 7.0 | 4.0 | 110.7 | 119.4 | 48 |
| 40 | 7.0 | 4.0 | 110.7 | 119.4 | 48 |
| 49 | 5.6 | 1.3 | 38.7 | 38.8 | 48 |
| 50 | 9.1 | 6.0 | 164.5 | 171.0 | 48 |
| 42 | 7.4 | 32.0 | 299.8 | NA | 48 |
| 41 | 9.2 | 6.5 | 214.3 | 240.2 | 48 |
| 31 | 9.2 | 6.5 | 214.3 | 240.2 | 48 |
| 33 | 9.2 | 6.5 | 214.3 | 240.2 | 48 |
| 34 | 9.2 | 6.5 | 214.3 | 240.2 | 48 |
| 32 | 9.2 | 6.5 | 214.3 | 240.2 | 48 |
| 54 | 11.6 | 8.0 | 295.5 | 448.6 | 48 |

### REFERENCES

1. Antiviral methods and protocols (Eds: D Kinchington and R F Schinazi) Humana Press Inc., 2000
2. HIV protocols (Eds: N L Michael and J H Kim) Humana Press Inc, 1999
3. DAIDS Virology manual from HIV laboratories, Publication NIH-97-3838, 1997
4. HIV-1 p24 antigen capture assay, enzyme immunoassay for detection of Human immunodeficiency Virus Type 1 (HIV-1) p24 in tissue culture media—Advanced bio science laboratories, Inc kit procedure.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above.

All publications and patent applications cited in this application are herein incorporated by reference to the same extent as if each individual publication or patent application was specifically and individually indicated to be incorporated herein by reference.

1. A compound of the formula (I) 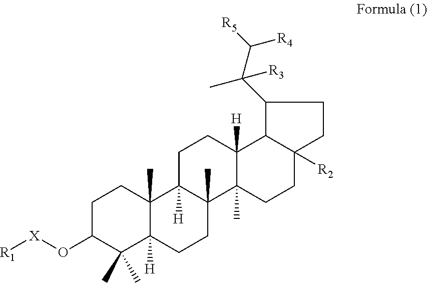
wherein,
X is —C(O)—;
R₁ is substituted or unsubstituted alkyl, 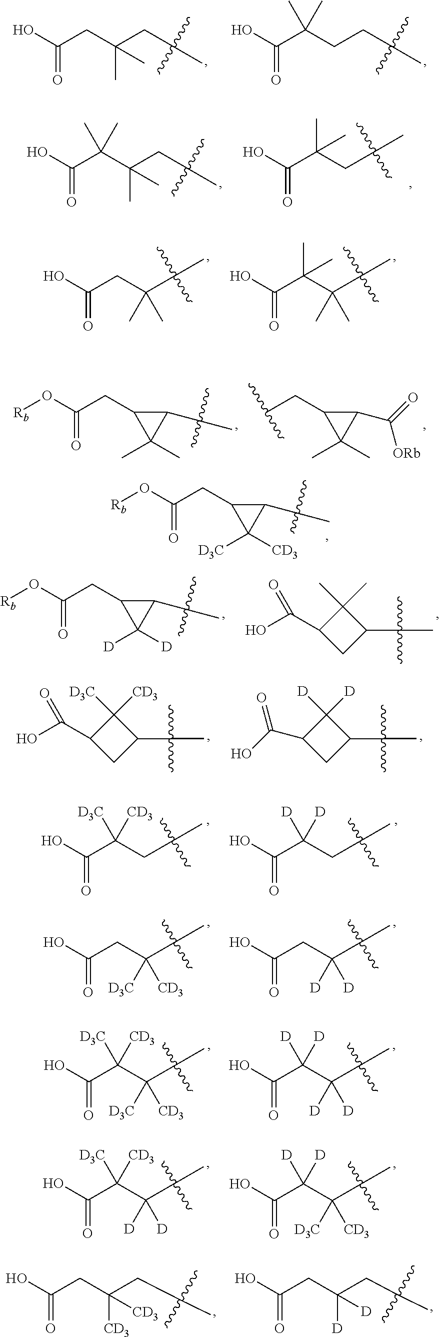 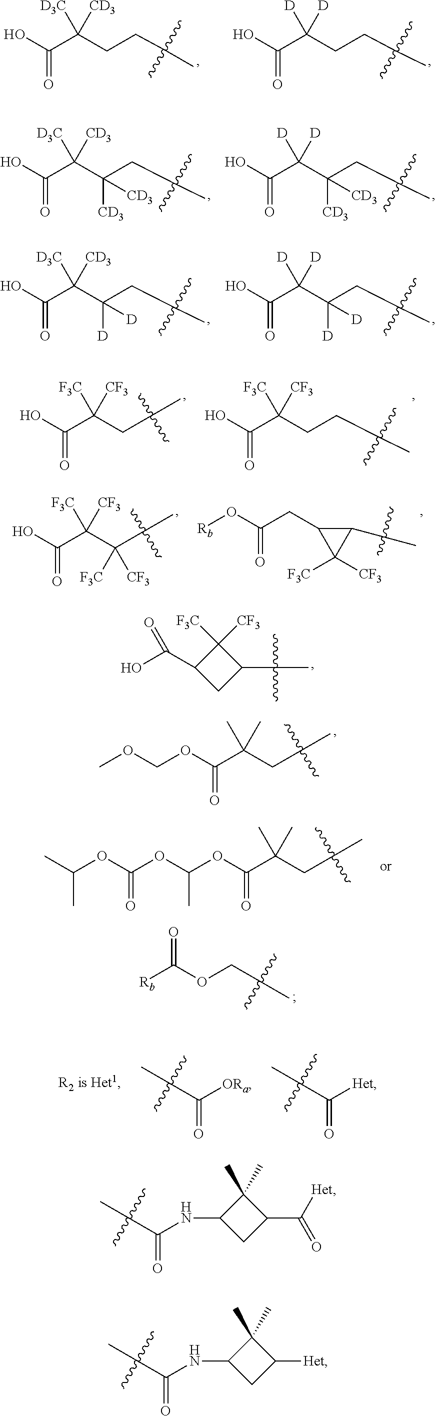
and preferably Het¹ and Het are independently selected from oxadiazole, triazole, isoxazole, pyrazole, pyridine, piperidine, Morpholine, pyrazine, or piperazine and preferably Het¹ and Het are substituted by R′.
R₃ and R₄ are independently selected from H, OH, or substituted or unsubstituted alkyl, or R₃ and R₄ are together with their adjacent carbons form a bond or R₃ and R₄ are together with their adjacent carbons form cyclopropl or epoxide;
R₅ is H, D, CD₃, CH₂CD₃, CH(CD₃)₂, CO₂Rd, or substituted or unsubstituted alkyl;
Rₐ, R_{b}, and R_{d} are independently selected from H, or substituted or unsubstituted alkyl;
each R′ is independently selected from H, CN, D, CD₃, CH₂CD₃, CH(CD₃)₂, CO₂H, CO₂-alkyl, C(O)-alkyl, OC(O)-alkyl, C(O)NH-alkyl, substituted or unsubstituted alkyl, substituted or unsubstituted alkoxy, substituted or unsubstituted phosphinate, substituted or unsubstituted oxime, substituted or unsubstituted amino, substituted or unsubstituted aryl, substituted or unsubstituted cycloalkyl, or substituted or unsubstituted heterocyclyl, an analog thereof, a pharmaceutically acceptable salt thereof, a pharmaceutically acceptable solvate thereof, a pharmaceutically acceptable hydrate thereof, an N-oxide thereof, a tautomer thereof, a regioisomer thereof, a stereoisomer thereof, a prodrug thereof or a polymorph thereof.

2. The compound according to claim 1, which is a compound of the formula (IA) 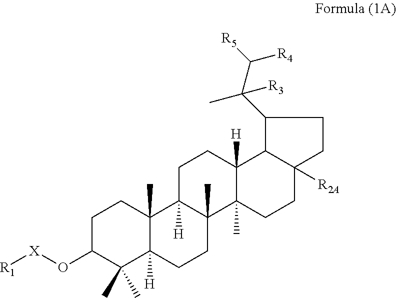
wherein,
X is —C(O)—;
R₁ is substituted or unsubstituted alkyl, 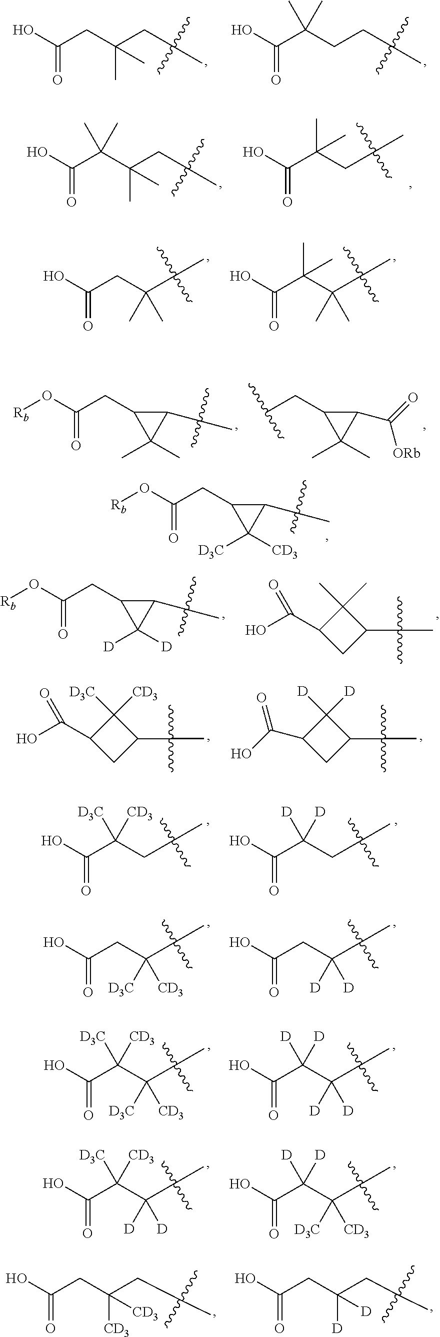 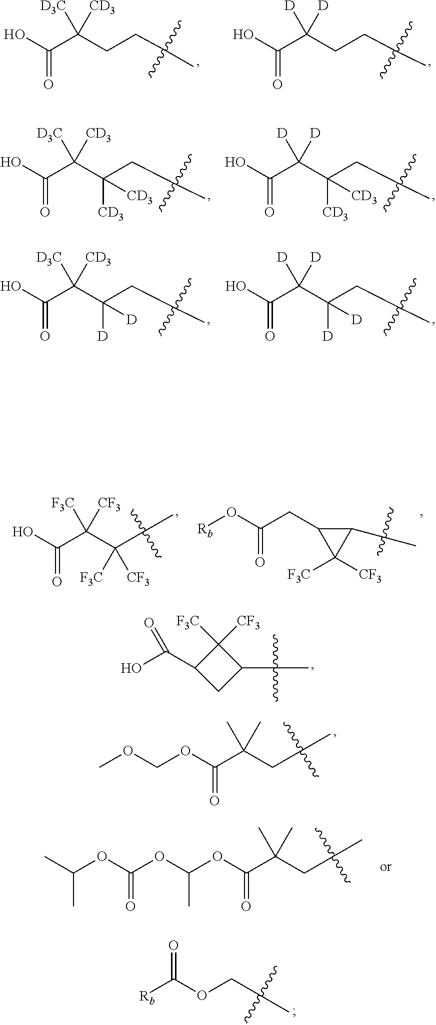
R_{2A} is 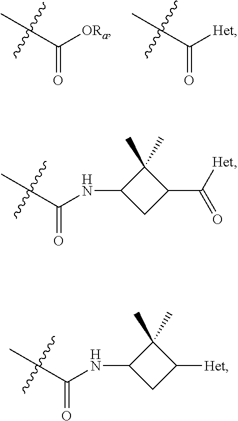
preferably Het is selected from oxadiazole, triazole, isoxazole, pyrazole, pyridine, piperidine, Morpholine, pyrazine, or piperazine and preferably Het is substituted by R′.
R₃ and R₄ are independently selected from H, OH, or substituted or unsubstituted alkyl, or R₃ and R₄ are together with their adjacent carbons form a bond or R₃ and R₄ are together with their adjacent carbons form cyclopropl or epoxide;
R₅ is H, D, CD₃, CH₂CD₃, CH(CD₃)₂, CO₂Rd, or substituted or unsubstituted alkyl;
Rₐ, R_{b}, and R_{d} are independently selected from H, or substituted or unsubstituted alkyl;
each R′ is independently selected from H, CN, D, CD₃, CH₂CD₃, CH(CD₃)₂, CO₂H, CO₂-alkyl, C(O)-alkyl, OC(O)-alkyl, C(O)NH-alkyl, substituted or unsubstituted alkyl, substituted or unsubstituted alkoxy, substituted or unsubstituted phosphinate, substituted or unsubstituted oxime, substituted or unsubstituted amino, substituted or unsubstituted aryl, substituted or unsubstituted cycloalkyl, or substituted or unsubstituted heterocyclyl, an analog thereof, a pharmaceutically acceptable salt thereof, a pharmaceutically acceptable solvate thereof, a pharmaceutically acceptable hydrate thereof, an N-oxide thereof, a tautomer thereof, a regioisomer thereof, a stereoisomer thereof, a prodrug thereof or a polymorph thereof.

3. A compound selected from the group consisting of:
(1R,3aS,5aR,5bR,7aR,9S,11aR,11bR,13aR,13bR)-9-((1S,3R)-3-(carboxymethyl)-2,2-dimethylcyclopropanecarbonyloxy)-5a,5b,8,8,11a-pentamethyl-1-(prop-1-en-2-yl)icosahydro-1H-cyclopenta[a]chrysene-3a-carboxylic acid,
2-((1R,3S)-2,2-dimethyl-3-(((1R,3aS,5aR,5bR,7aR,9S,11aR,11bR,13aR,13bR)-5a,5b,8,8,11a-pentamethyl-3a-(piperidine-1-carbonyl)-1-(prop-1-en-2-yl)icosahydro-1H-cyclopenta[a]chrysen-9-yloxy)carbonyl)cyclopropyl)acetic acid,
2,2-dimethyl-4-oxo-4-((1R,3aS,5aR,5bR,7aR,9S,11aR,11bR,13aR,13bR)-5a,5b,8,8,11a-pentamethyl-1-(1-methylcyclopropyl)-3a-(piperidine-1-carbonyl)icosahydro-1H cyclopenta[a]chrysen-9-yloxy)butanoic acid,
4-((1R,3aS,5aR,5bR,7aR,9S,11aR,11bR,13aR,13bR)-3a-(2,2-dimethyl-3-(piperidine-1-carbonyl)cyclobutylcarbamoyl)-5a,5b,8,8,11a-pentamethyl-1-(1-methylcycloprop yl)icosahydro-1H-cyclopenta[a]chrysen-9-yloxy)-2,2-dimethyl-4-oxobutanoicacid,
2-((1R,3S)-2,2-dimethyl-3-(((1R,3aS,5aR,5bR,7aR,9S,11aR,11bR,13aR,13bR)-5a,5b,8,8,11a-pentamethyl-3a-(5-phenyl-1,3,4-oxadiazol-2-yl)-1-(prop-1-en-2-yl)icosa hydro-1H-cyclopenta[a]chrysen-9-yloxy)carbonyl)cyclopropyl)acetic acid,
2-((1R,3S)-2,2-dimethyl-3-(((1R,3aS,5aR,5bR,7aR,9S,11aR,11bR,13aR,13bR)-5a,5b,8,8,11a-pentamethyl-1-(prop-1-en-2-yl)-3a-(5-(pyridin-3-yl)-1,3,4-oxadiazol-2-yl)icosahydro-1H-cyclopenta[a]chrysen-9-yloxy)carbonyl)cyclopropyl)aceticacid,
4-((1R,3aS,5aR,5bR,7aR,9S,11aR,11bR,13aR,13bR)-3a-(4-(1-methylethyl-2,2,2,1′,1′,1′-D6)piperazine-1-carbonyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid,
4-((1R,3aS,5aR,5bR,7aR,9S,11aR,11bR,13aR,13bR)-3a-((1R,3S)-3-(4-(1-methylethyl-2,2,2,1′,1′,1′-D6)piperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid,
4-(1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yl 1-methoxymethyl 2,2-dimethylsuccinate,
4-(1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yl 1-(1-(isopropoxycarbonyloxy)ethyl)2,2-dimethylsuccinate,
4-((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxo butanoicacid,
4-((1R,3aS,5aR,5bR,7aR,9S,11aR,11bR,13aR,13bR)-3a-(4-ethylpiperazine-1-carbon yl)-5a,5b,8,8,11a-pentamethyl-1-(1-methylcyclopropyl)icosahydro-1H-cyclopenta[a]chrysen-9-yloxy)-2,2-dimethyl-4-oxobutanoicacid,
(1R,3S)-3-(((1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylic acid,
4-((1R,3aS,5aR,5bR,7aR,9S,11aR,11bR,13aR,13bS)-3a-((1R,3S)-2,2-dimethyl-3-(piperidine-1-carbonyl)cyclobutylcarbamoyl)-5a,5b,8,8,11a-pentamethyl-1-(2-methyloxiran-2-yl)icosa hydro-1 H-cyclopenta[a]chrysen-9-yloxy)-2,2-dimethyl-4-oxobutanoicacid,
4-((1R,3aS,5aR,5bR,7aR,9S,11aR,11bR,13aR,13bS)-1-(1,2-dihydroxypropan-2-yl)-3a-((1R,3S)-2,2-dimethyl-3-(piperidine-1-carbonyl)cyclobutylcarbamoyl)-5a,5b,8,8,11a-pentamethylicosahydro-1H-cyclopenta[a]chrysen-9-yloxy)-2,2-dimethyl-4-oxobutanoicacid,
4-((1R,3aS,5aR,5bR,7aR,9S,11aR,11bR,13aR,13bR)-3a-((1R,3S)-3-(5-isobutyl-1,3,4-oxadiazol-2-yl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)-2,2-dimethyl-4-oxobutanoic acid,
(1R,3S)-3-(((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(1-methylcyclopropyl)icosahydro-1H-cyclopenta[a]chrysen-9-yloxy)carbonyl)-2,2-dimethyl cyclobutanecarboxylicacid,
2-((1R,3S)-3-(((1R,3aS,5aR,5bR,7aR,9S,11aR,11bR,13aR,13bR)-3a-((1R,3S)-2,2-dimethyl-3-(piperidine-1-carbonyl)cyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclopropyl)acetic acid,
2-((1R,3S)-3-(((1R,3aS,5aR,5bR,7aR,9S,11aR,1 bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yloxy)carbonyl)-2,2-dimethylcyclopropyl)aceticacid,
(1S,3R)-3-(((1R,3aS,5aR,5bR,7aR,9S,11aR,11bR,13aR,13bR)-3a-((1S,3R)-3-(4-ethylpiperazine-1-carbonyl)-2,2-dimethylcyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclobutane carboxylic acid,
(1R,3S)-3-(((1R,3aS,5aR,5bR,7aR,9S,11aR,11bR,13aR,13bR)-3a-((1R,3S)-2,2-dimethyl-3-(5-methyl-1,3,4-oxadiazol-2-yl)cyclobutylcarbamoyl)-5a,5b,8,8,11a-pentamethyl-1-(prop-1-en-2-yl)icosahydro-1H-cyclopenta[a]chrysen-9-yloxy)carbonyl)-2,2-dimethylcyclobutane carboxylicacid,
4-(((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-2,2-bis(methyl-d3)-4-oxobutanoic acid,
(1R,3S)-3-((((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(((1R,3S)-2,2-dimethyl-3-(piperidine-1-carbonyl)cyclobutyl)carbamoyl)-5a,5b,8,8,11a-pentamethyl-1-(prop-1-en-2-yl)icosahydro-1H-cyclopenta[a]chrysen-9-yl)oxy)-2,2-bis(methyl-d3)-4-oxobutanoic acid,
4-(((1R,3aS,5aR,5bR,7aR,9S,11aR,11bR,13aR,13bR)-3a-(4-ethylpiperazine-1-carbonyl)-5a,5b,8,8,11a-pentamethyl-1-(prop-1-en-2-yl)icosahydro-1H-cyclopenta[a]chrysen-9-yl)oxy)-2,2-bis(methyl-d3)-4-oxobutanoic acid,
5-(((1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl)oxy)-3,3-dimethyl-5-oxopentanoic acid,
2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yl)oxy)butanoic acid,
(1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(morpholine-4-carbonyl)icosahydro-1H-cyclopenta[a]chrysen-9-yl)oxy)carbonyl)cyclobutane-1-carboxylic acid,
(1R,3S)-3-((((1R,3aS,5aR,5bR,7aR,9S,11aR,11bR,13aR,13bR)-3a-(4-methoxypiperidine-1-carbonyl)-5a,5b,8,8,11a-pentamethyl-1-(1-methylcyclopropyl)icosahydro-1H-cyclopenta[a]chrysen-9-yloxy)carbonyl)-2,2-dimethylcyclobutanecarboxylic acid,
3-(1R,3aS,5aR,5bR,7aR,9S,11aR,11bR,13aR,13bR)-3a-(4-ethylpiperazine-1-carbonyl)-5a,5b,8,8,11a-pentamethyl-1-(1-methylcyclopropyl)icosahydro-1H-cyclopenta[a]chrysen-9-yl)oxy)carbonyl)-3-(methyl-d3)butanoic-4,4,4-d3 acid,
2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)oxy)butanoic acid,
(1R,3S)-2,2-dimethyl-3-(1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethyl)piperazine-1-carbonyl)icosahydro-1H-cyclopenta[a]chrysen-9-yl)oxy)carbonyl)cyclobutane-1-carboxylic acid,
2,2-dimethyl-4-oxo-4-(1R,3aS,5aR,5bR,7aR,9S,11aR,11bR,13aR,13bR)-5a,5b,8,8,11a-pentamethyl-1-(1-methylcyclopropyl)-3a-(4-(3-morpholinopropyl) piperazine-1-carbonyl)icosahydro-1H-cyclopenta[a]chrysen-9-yl)oxy)butanoic acid,
(1R,3S)-2,2-dimethyl-3-(1R,3aS,5aR,5bR,7aR,9S,11aR,11bR,13aR,13bR)-5a,5b,8,8,11a-pentamethyl-1-(1-methylcyclopropyl)-3a-(4-(3-morpholinopropyl) piperazine-1-carbonyl)icosahydro-1H-cyclopenta[a]chrysen-9-yl)oxy)carbonyl)cyclobutane-1-carboxylic acid,
4-(1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-(1R,3aS,5aR,5bR,7aR,9S,11aR,11bR,13aR,13bR)-3a-(4-(3-hydroxy-2-(hydroxymethyl)-2-methylpropanoyl)piperazine-1-carbonyl)-5a,5b,8,8,11a-pentamethyl-1-(1-methylcyclopropyl)icosahydro-1H-cyclopenta[a]chrysen-9-yl)oxy) carbonyl)-2,2-dimethylcyclobutane-1-carboxylic acid,
4-(1R,3aS,5aR,5bR,7aR,9S,11aR,11bR,13aR,13bR)-3a-(4-(tert-butoxy carbonyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-(1R,3aS,5aR,5bR,7aR,9S,11aR,11bR,13aR,13bR)-3a-(4-(tert-butoxycarbonyl)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(4-(2-(2-methoxyethoxyl)ethyl)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9 S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(4-(2-methoxyethoxyl)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(4-(2-(2-methoxyethoxyl)ethoxy)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(4-(4-ethylpiperazin-1-yl)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxyl)piperidine-1-carbonyl)icosahydro-1H-cyclopenta[a]chrysen-9-yl)oxy)butanoic acid,
(1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(4-(2-morpholinoethoxyl)piperidine-1-carbonyl)icosahydro-1H-cyclopenta[a]chrysen-9-yl)oxy)carbonyl)cyclobutane-1-carboxylic acid,
2,2-dimethyl-4-oxo-4-(((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)oxy)butanoic acid,
(1R,3S)-2,2-dimethyl-3-((((1R,3aS,5aR,5bR,7aR,9S,11aR,11bR,13aR,13bR)-5a,5b,8,8,11a-pentamethyl-1-(1-methylcyclopropyl)-3a-(4-((2-morpholinoethyl)carbamoyl)piperidine-1-carbonyl)icosahydro-1H-cyclopenta[a]chrysen-9-yl)oxy)carbonyl)cyclobutane-1-carboxylic acid,
4-(((1R,3aS,5aR,5bR,7aR,9S,11aR,11bR,13aR)-3a-(((1S,3R)-2,2-dimethyl-3-(morpholine-4-carbonyl)cyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
5-(((1R,3aS,5aR,5bR,7aR,9S,11aR,11bR,13aR)-3a-(((1S,3R)-2,2-dimethyl-3-(morpholine-4-carbonyl)cyclobutyl)carbamoyl)-5a,5b,8,8,11a-pentamethyl-1-(1-methylcyclopro-pyl)icosahydro-1H-cyclopenta[a]chrysen-9-yl)oxy)-3,3-dimethyl-5-oxopentanoic acid,
(1R,3S)-3-((((1R,3aS,5aR,5bR,7aR,9S,11aR,11bR,13aR)-3a-(((1S,3R)-2,2-dimethyl-3-(morpho-line-4-carbonyl)cyclobutyl)carbamoyl)-5a,5b,8,8,11a-pentamethyl-1-(prop-1-en-2-yl)icosa-hydro-1H-cyclopenta[a]chrysen-9-yl)oxy)carbonyl)-2,2-dimethylcyclobutane-1-carboxylic acid,
1-benzyl 3-((1R,3aS,5aR,5bR,7aR,9S,11aR,11bR,13aR)-3a-(((1S,3R)-3-(4-ethylpiperazine-1-car-bonyl)-2,2-dimethylcyclobutyl)carbamoyl)-5a,5b,8,8,11a-pentamethyl-1-(prop-1-en-2-yl)-ico-sahydro-1H-cyclopenta[a]chrysen-9-yl)(1R,3S)-2,2-dimethylcyclobutane-1,3-dicarboxylate,
4-(((1R,3aS,5aR,5bR,7aR,9S,11aR,11bR,13aR)-3a-(((1S,3R)-2,2-dimethyl-3-(piperidine-1-carbonyl)cyclobutyl)carbamoyl)-5a,5b,8,8,11a-pentamethyl-1-(1-methylcyclopro-pyl)icosa-hydro-1H-cyclopenta[a]chrysen-9-yl)oxy)-2,2-dimethyl-4-oxobutanoic acid,
4-((1R,3aS,5aR,5bR,7aR,9S,11aR,11bR,13aR,13bR)-3a-(4-cyano-4-phenylpiperidine-1-carbonyl)-5a,5b,8,8,11a-pentamethyl-1-(1-methylcyclopropyl)icosahydro-1H-cyclopenta[a]chrysen-9-yloxy)-2,2-dimethyl-4-oxobutanoic acid,
4-(((1R,3aS,5aR,5bR,7aR,9S,11aR,11bR,13aR,13bR)-3a-(4-((3-hydroxy-2-(hydroxymethyl)-2-methylpropanoyl)oxy)piperazine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(4-((3-hydroxy-2-(hydroxymethyl)-2-methylpropanoyl)oxy)piperazine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(4-((dimethylphosphoryl)oxy)piperidine-1-carbonyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(4-((dimethylphosphoryl)oxy)piperidine-1-carbonyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(((1R,3S)-3-(4-(ethoxyimino)piperidine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(((1R,3S)-3-(4-(ethoxyimino)piperidine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(((1R,3S)-3-(4-((dimethylphosphoryl)oxy)piperidine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(((1R,3S)-3-(4-((dimethylphosphoryl)oxy)piperidine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid,
4-(((1R,3aS,5aR,5bR,7aR,9S,11aR,11bR,13aR,13bR)-3a-(((1S,3R)-3-(4-ethylpiperazine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)-2,2-dimethyl-4-oxobutanoic acid,
(1R,3S)-3-((((1R,3aS,5aR,5bR,7aR,9S,11aR,11bR,13aR,13bR)-3a-(((1S,3R)-3-(4-ethylpiperazine-1-carbonyl)-2,2-dimethylcyclobutyl)carbamoyl)-5a,5b,8,8,11a-pentamethyl-1-(1-methylcyclopropyl)icosahydro-1H-cyclopenta[a]chrysen-9-yl)oxy)carbonyl)-2,2-dimethylcyclobutane-1-carboxylic acid, and pharmaceutically acceptable salts thereof.

4. A pharmaceutical composition comprising a compound according to any one of claims 1-3 and a pharmaceutically acceptable excipient.

5. The pharmaceutical composition according to claim 4, wherein the pharmaceutically acceptable excipient is a carrier or diluent.

6. A method for preventing, ameliorating or treating a viral mediated disease, disorder or syndrome in a subject in need thereof comprising administering to the subject a therapeutically effective amount of a compound according to any one of claims 1-3.

7. The method according to claim 6, wherein the viral mediated disease, disorder or syndrome is HIV infection, HBV, HCV, a retroviral infection genetically related to AIDS, respiratory disorders (including adult respiratory distress syndrome (ARDS)), inflammatory disease.

8. A method of treating HIV in a subject in need thereof comprising administering to the subject a therapeutically effective amount of a compound according to anyone of claims 1-3.

9. A method for preventing, ameliorating or treating a viral mediated disease, disorder or syndrome in a subject in need thereof comprising administering to the subject a therapeutically effective amount of a compound according to claim 4.

10. The method according to claim 9, wherein the viral mediated disease, disorder or syndrome is HIV infection, HBV, HCV, a retroviral infection genetically related to AIDS, respiratory disorders (including adult respiratory distress syndrome (ARDS)), inflammatory disease.
